SEQUENCE LISTING (33 sequences)

SEQ ID NO: 1 - DNA, 50000 bp, Mus musculus
cttttttgacagccaaatctttttttattgggggaacgggtctctagggggtaggcctaggccctcactgcacagcttgttcattggcactgcctccagaatcctgtggcttcatcacatctggaagctcgggagggctggagaagggctcaatgcggagagtttcgaaggtgtcatcttctcggaaggccaggcccactgtggctgtgctgtctggctagtgaagccacactcgcccagagttttgccatcatcaaggagctggtcatccttgtaaagcagccgctcctctggcggccgcttgaggatgtcctcgacgatgtgcttcaattcgaacacagtgctcaacttcttggcatccagaaagatggtggtcttgtggcaccggatcatgagaaacatgtccattctggcggctgcttctggcttgaggcgccagtgcagccccaattgtggctttctttgtttcttttttttttaaagctttatttatttattaattatatgtaagtacactgtagttgtcttcagacaccccagaaaaaggtaacatctcattacggatggttgtgagcaaccatgtggttgttgggatttgaactcaggacttccagaaaagcagtcagtgctcttaagtgctgagccatttctccagcccaattgtagctttctataatggtgtctgtctgtagcaaagaaaggcttctttttattgttattatatattgtatatataaaatttttctttttattgctattatgtattggatatataggattgtatatatatgcatatgtagttatatatttatatattacatacatacatatatatagttgttataattttattttatgtttatggatgttttgcctgcatgtatgtctgtaccgtgtgtgtgtgtgtgtgcgtgtgtgtgtgtgtgtgtgtgtgtgtgtgtgtgtatctggtgcctgaggaagtgagaagagggtgtcagatcccctggaactgtagttatatgttgcgcgcgctcaactggccaggaagaacgacgctgctacaggatccttctgcacacatttattcagtcctgtttcttctttctccatatatctcccttgtttatatctcccttgtttatatctcccttgtttatatctctcccttgtttatatttcccctgtttgtatctctccctcgtttatatctcccccttgtttatatttcccctgtttgtatctctccctcgtttatatctcccccgaaccctgggcctctcactctttttatactctctctcatccacgcactgcaggccacgccccctcgccagtcacgaggcttcagctaatcagggcagcaggggcaaatctccaccaaattggattcacctgtatcctggtacacctgcgcagcactcaagatgtttgtgtcttatatgaggaagtcaggtgcaagtcatatgacttagctgcagtccctggcgcctttaggactgccgccacacctgctcctaacaattcccccttttttcttttttggcagagagaatgcctgtagatagccccccgcagccatgccccttacccgtccttgggtgacaaacagcattggtttgatccctgtcttaggttggtgacatgcccagggagtcttatcactgactacctctctatcatgccaagccacacctggggagattgtgttttgcttgcggcaggtgcatcaaaaggccaggatgttaattaacctatggccagatcttaattgctgcaagcaagcctcatgggtgaaggtagaggtctgatccccagtgtgcaagcataggggccctacacaaaaccatcccttaggctcatcacccagagaggtcttggccagctcccgtgtcgtttttcctgggggaagggaactaggacactgaaccttcatgcaatcagacatgccttccacaggatgccaacagcaagctatcctctgtgcagtgcttagcctcgcaccccacggcataacgcagcataatttcttttagagcggcaaaccgaatctgaggagattgtcccgcctccactgcagcaaaagcctacgctaccatggcgaaagtgcgggtcgcacactctgcacctaacacatgtgccaaacacaaaacacacacacactataacaagcatacatcccagggctctcatccccactcatcttccctgaagcaagggatagatagccagaggggctatctctttaagaggaattcagggatcaaaaagcgtggaaaaatttgaatgtcatgtcgagctggtatcggcttctggaataccgaaaggatctcccatctctgctccatcctttgtgcttgtgggatcatccaccacatccacaggggcaactggatcaggagcctcattcttgcagtgtctcaccaatctctccggcacccaaagaggttccatctggtcctgtggaaacacacaaacagaccctctcgccctcgtcaacaccggatctgctcgatccccaggggagatggacacaatacctcgtggtaatgagaccataacctcaatcacctgtgtgttcatctggggagtcaatacgccaaatctccggacagaaggtccacctataggtggctgctgaggaggcatagggaggaggcacagaagctaattcgccttcctcctccacctcggctcttttattttgtcctttctgtccacctgataacactgtgtctcctttctttttcctttttctttttaagcccttctccttttccgacatactttcttgttgctctgtaaggattttctgacctgtcttgactgcctctacacacaaacagtaacagagtccatatatcagaacaaacaagaccaaaactatcagacctacccacaaagggtcaatgggagaaaaaagcataactacacagcgaggatctattgatcactacactgagcttatcatagttccttaacttgtcccaaagccaggaaccttaacttgtcccaaagccgggaacctttcgttaccttgtgcctgcttgctggcaactttatgctcacctctattttatcagaggtccttcccaaactcctgggttactttgtgcctacttcctggcaactttatgcttgcctctattttatccgaggtccttcccaaactcctggggttgcaagtttcactcaccgtgaacttaccctgcctgccggcaaccactgactgctgaaagttctgaactcggtgggggagtcggttccccgtacgggccaccaattgtcgcgcccactctcgaccagcaagaacgacgcgaccaccagtccttctaacagcagtttattcagtcttcatctctcttctttctcttcatcagtaccgttccccagctgaagagttctgaatccacgccggatccttctcaacagtctgtttcacgggaacctttattaaccgctccttccccgtgatgcagttctgaatcctccctgtagcagggggtcttcgctcatgcctgaagatgtttcttttcccgggtttcggcaccaactgttgcgcgcgctcaactggccaggaagaacgacgctgctacaggatccttctgcacacatttattcagtcctgtttcttctttctccatatatctcccttgtttatatctcccttgtttatatctcccttgtttatatctctcccttgtttatatttcccctgtttgtatctctccctcgtttatatctcccccttgtttatatttcccctgtttgtatctctccctcgtttatatctcccccgaaccctgggcctctcactctttttatactctctctcatccacgcactgcaggccacgccccctcgccagtcacgaggcttcagctaatcagggcagcaggggcaaatctccaccaaattggattcacctgtatcctggtacacctgcgcagcactcaagatgtttgtgtcttatatgaggaagtcaggtgcaagtcatatgacttagctgcagtccctggcgcctttaggactgccgccacacctgctcctaacagttatagatggttgtgagcctttgagtggggactgggaatcaaacttgtcctctagaagagtagccactgctcttagatacttagccatctttctagcctaaagaattttttttccctttggcttttcaatacacggtttcttggtgtagccctggctgtcgtgtcctgtacacacacatgcacatgcacacgcacacatgcacagtcatccatgatggagagggggactgagccccgggcctgagatgccaagcacacacactgtcattgaactgtacctttagtcactaaaaagccctggtctgacagccactgtgccgctgggcatggtggtgcaggcctttgactccagcacttggaactcagcgtctggctgacctctgtgagtttgctgcaagcttggtcaacatacagagttccaggccagccagggctacatagtgagtgtggctgtgtctcaaatgtaaacaaaaaggccttacacaaccaagtcaaactcaaccaccctttcttactgttttggtgtaagtgacaggacctcactttgtgagaagctgtcagctgttgccctaataattaaggttgaagtctatcattgtctgggtagccttctgggctccatgctaatggtgaactttctctgtcagactctcttcttaacctgggggatccctgtgatggtttgtatgttcttggctttatctcctgggattaaaggcatgcactaccttgcctgggcctaagcttttcatagctgctgtgcctcaagatctccatgtcaagatctaggtcagaaacttgtgtcttccagcctcaagatctggatcacatgtgagccctccaattctggattgtagttcattccagatatagtcaagttgacaaccaggaatagtcattacaatccaacccttgtcttgtttgtttttttgtttgtttgtttttgttgttcttgtttttttcggtttttcgagacagggtttctctgtgtagccctggctgtcctggaactcactctgtagaccaggctggcctcgaactcagaaatccacctgcctctgcctcccatgtgctgggattaaaggcatgcaccaccactgcccagctaatccaacccttgtcaatttgacacaaatatctcatgtccacatgaaacaataacaagatcataaatatgcctaacatgatataactattccttgtacaatcacaaaaacatttgtaaaattacagtggggcaatgtccctcgggaacattcttttagtatctcaacttaaatacagattgatgttaaaaaaaaaatgggagaaagcacaaatagctatacaaatgtgttcttaacaatataaaccagaagcattgatattactttataatcctcatttctgcaactggtcatgtggtcttagatggtatttataactacctccctctactacccattctgtattttctccatcctctgcaagcacctcagctggtcttcttggctcttttcctggaggagtgacccataccttcacccctgatgggtctgtgtcctttgtcatcctgcttggattaggctgttttgttttctattgactttaatcacaggacttggtagtactaggagacaccctaagggatctcctgcactccagacataatcccttttaccttcattgtggtagttgatccaatttccccatggtaatctggatctatcacccctcctaacactgttattcttttcttagcctgttggtttaagggcattagaagcccaaaatgacaagggggaagtttgagcttccagttaaatgaaatgttagttgtggctcctggcaggaacacactccactcctggtaccaatttgtattagtcaggatttgtcagagaccataccgtgagaaatcgagcttctcacaatctcccaccctgacaggccaaatggcctcgtgataggagccggttgtcactccctcctccctgttcccttcctggcacctgaggctgtgaaagctgaattatagtccccgcttccctatctcttcctgactccatgacatccaaggacatgagttacacctgagcccggcctgacacctcaaggctgttaaggaggatctatgttctggagataagatgcagagtgcccaccgcctggagcctgactttggcccttatgtcagcagatgtccacttgtttgttctttgttaaattcccccttgacccctccctattccccaagatgtatgctttaaaaccaggcatctcagtataatagatggagaccttgataggcacccttcttggtctccgcttctccttcccttcttcccattttcttccaggtttgcggtccctctcacgaataactgaatcctgcgggacgggataagtggcacccaacatgagggtgaggattgtatttccttccagtggaggttccagagaggtttgtcacgaccccaagaatttagaagtagtgaaggaccccttctgccgctcacggaagagtgagaagtccttggtgagttgagtcatctccacttcaggttatgggaaataagctttctaaagaggcagccttcatcaaaggcttaaagatagctctcagggaaagaggagtacgagttaaaaagaaagatttgataaacttttattttcatagaccaggtatgtccatggtttattatagatgaagcagagatacgttgtaaaaaatggtgaaaggtaggtagagacttaaatgataaactagctaatgagggtcccgatgcggtccctgcaactgtcttttcttattgaggagtaaatatcagaagctgccactgtgcctcctctcccttctctggcagaattcccagaagaaggagataaagaattagagtctgaacatgagaaagagaaaataagtttcagaaaagcagttatcccctgtttgggaccttttagcaaaaaagagggaaaatgaaaataagctatatcagagctctcctagggacagaaggaaaatgggagacgtcctgcttcctctctggctcctatggagatattcctagttctggttaggatgtctgggtccctttgagacatcaagttctattccctgtctgtctattgtctggttttggtgcaccaaaatgtgtccatatatctgtctatttatgtttgcttttgttttgttgtttgaatgatagttgtactgtgtttcatgttgaaaaaaatggttaaaattttatctgctggctgtccattctttgatttagtttaacttgtttaaaaagcagtctcaatttgcacagcagaaacagataactgtggctgggagtcaagtatagctggaagagccctgctgagagccgggacgggataaggattctctagagtcacagaatttatggtatgtctttctatattaagggaatttgttgtgatgacttacagtctgtggtatagctaccccaacaatggtcagctgtgaatgggaagtccaagaatttagtagttgcccactccctcaaggttagtgaggctagttgttgtagctgatcttctgtagaagtagattccaacagatgtgttggcaagtaaatgcaagcaggtgaaggagagcaaatcttccttcttccaatgtctttacgtaggtctccagcagaaggtgtggcccagattaaaggtgtgtaccaccatgcctggatgggatttgttttatcctaggatgatcttgaactcagagatctccttgctttagtctcctgggattaagggcgtgtactaccttgcctgggcctaagctttgctttgctttgcttttttttcttttttcttttttcttttggtttttcgagacagggtttctctgtgtagccctggctgtcctggaactcactctgtagaccaggctggcctcgaactcagaaattcgcttgcctctgcctcccaagtgctgggattaagggcctaagctttttcatagccactgtgcctcaagatttctatgtcaagatccaggtcagaaacttgcatcttctaccctcaagatctgaatcacagatgagacctccaattctggattgtagttcattccagatatagtcaaattgacatccaagattagccattacagtcccagacctcacactctatacctgcaaatggaatgccattccttggttaagatcataggttcagcttcctttctcctggtagaacccattaagctagaacctgagattcctagagtccttccctatggtaacgaggcatctcagtaccttaagccttcagctaatgacttttgcttaccctggatattcctaccccttgacctaaaactatataaaccttgaatcaccccaagttaagttgatctgtctccctgaagctggtcctggtgtgcccttattactcactgggcttctggacacctctcccaccctccacaccctttctaaccatcatctctgctcctgggaggggacaggtgcagggagggtcacatttagtcttcttgcctcaaccttttgaatgctgtccaccgcctgcctcaccacatctgtcatttcttcttttgcaatatatgtagttgaactcaaaatttccctgttagtacgactttcctgcacacacatttggattgctaggttttttttactttagtagttttatcattacttcgaagccctatgaagatatttatgttcttccttggcccctgggtcatctctcagccaggtccctaggtacaacctctatattataggacccaggctccctgtctggtttcactttcttaacctttgaagttatcattctaggtaatatttaaaaaaaatagtagtgcactaagaggctgggtgtggtggctcatacctgcaatcccaaccttcaggctaagacaggaggattactgtaaattcagagtcatctgtgggatacacagggaattccaggtcaatctggtctccagagcaggcaggcctacacagagaaactttgcatatatatatattttaaaagaaagaatggaaggaaggtcagaccacattttattagtgagcttgggaatctgcatgctttgtgactgctggatgagatgtgatagtgtatcctaaatggatggattttatcatcttttcttccagaacacaaacagctgcttttcatcttcctgcttgcctagcttctttgggctagatggttctttgtagctctctacctttaaactctaccccaggtaaagttccgagtagtggcctatctttatgttgatcaatgtttgatcatccctgaacagagagagggaatcccctctgtggtttgtttgtttgtttgtttgtttgtttgtttgtttttgttttacaagacagagtctctctgtgtagcactggctgtccaggaactcactttgtagaccaggctggccttgaactcaaagagatccgcctgcctctgcctccccaagcactgggattaaaggcgtgcgccactaccaccaccacccagctccctctgtgttgtaaaacagctgctcctgttgctttaaggctgaggcaaggcgctacactggggaagagaaagaggcaaaggtgaataaagcaagataaaactgccatagaatttttcaggccaactttttttttgtttgcctggttgctatggacttttatctatttttttttttgagtgcctacaaagtttgttctgacatctcagcttattttatcagtgtttctatggagagatactatcgtagagcttccttgtttactgaattcctgatgtcactcctgaatggcgtattttaaaaatcattatttactgatccttcgggaccacaagataagtgagaactccagatattcagtctctctgtctaaagcacaagaggtgggcaagactaggttagcacctcgactgtgcacgttgcatttaatgcagatgtctggaagcagatcacagagccgctgcctgggacatgcacggtggtcagcagagataatgttccctgcctttcacatagacctccaaactctgaatgctgctggagaacagagaggtcagttaagtgaactctcttcacaccccagggcctgcttgaacagctttcgttaaagaactaccaagcaaacaccctggtcccagggcactgccttgccccaaccccaaactgccccctgacatagtatgaaatgctgctctgggctgcagactgactacactgtagccagagataattgtatgaataataaaaacaaattaaaaagagatattatggctggctgtacagtttagtagtgtagcacagacttaccacgtgcaagtccagggttcaaccgtcagtaccaaaccctccctccacaaacccggaggtatcttcttatcataccgcagttgcttggttagtaaagcctggaaaatataatacataactataaaagtgtgatgagacactgttatgattaggtgagtatatgatgagacactgtgcttgctggcatcactgatgtctggcctgtgagagttgagactgacccttagctagataccagtaacagattctgataattgtctgataatcctcatttagcccaaggtatgaaattgctaactgtgcacttcagagtcataaatacatttaaaatcataggtttcaggtctgggaacacatttgaaggagacacattccaaaaataaaaaagaagagggggaggagagatgagggggagaaggagggagaagaggaagaggaaggagaaggaggacatgtcttagacataaaggggtcaatgggaacttatttataatcatgaaatcttggcaaccagcccgattttcaatgatgggagagtagacccctctgctgctccatggatgataattccaaataaacatggtgatcagagattgagggcaatggagttttaaaatcaagggaaaaacagcaagcgtgcatgcttgtgtcacttgttcgtgactgaactagtgtgccctggcaaggcctacagggacccccacacaggcagtgatgtgagaccaggtgaccctccagtgtgactgtgtgtttctcatgcttttgggggcttcagaaaagccctcagaccaaggatctggacctcatctctctggagtctggtctggggacagctggacagccctcgtgagaactgatgtggagaaggcagggctcaatgcccctcactggggctcttggggttttcatgtggcagcagtatctgtagaccaggctggctttgaactgcctgcctcttcctcctgagtgctaagattaaaggtgtgcaccaccaatgttcctattccaggaatgtcctcaatcaatgacttgtaagtgtggatggtgccacacccttatccactggtggggatcccctagggcgggtccccttatgtcctggaggtctcagggacaggtgatattcttgaatccatcttgatcatcacatctcatggttctcagtctgctcagccctctgtcgtcacctgaagtcatcttccaaataaccctgattttcatttgtcctaggatacttgtctcagggtctgcatctggagaaatcacatgagaacatttgggacaagacaagaagaggactgggtggcatccagggagcaacaagggaagcaggtgatgttgtgtggcccagggcccttctcctcagcctctcttgttccctgcctaagcttgggcggattcccctctgagcccacccgagcccctgggacactggtggaactcagtaggagcccctccctgcagctgtctcaacaggtagctgcatgagtggccttgaagcaattatcagcaattcagccctggcaatagaggccaaggtcctggcctgtcttggtgatagcaagagcccaaggaaagactggaagtttcctactggaaagaagcagaggatgaaccatgtacctgggcccaggttgggtgggacttgccactcagagcccctaaccagggttgttcagaggactaggccagggccaggaccaagaaagggatagaacgggcatgaggaggaagggtgaagggatccaaggaatctctggtcctgttccctgttaggacatttgtcatggaatcactctcgcttagtgtctctgttatctgggtgctaatagcaactattcagttgctaggatgttaggtgagtctgaacctacccttgatgttgatctgaagaggcgatgcgttagactgcaggttggaggccaagtccaggacagtgttgatattctggatctccaagaagcctccaaggccaaagccaggccagtgtctggtctcgcagaggaacagctctgcatctcttgcccggttggctctaactaccacattagacttcagttgcgtcaaaaaacgaggggaccccagcgccttcactaggaagttgacctcagaaggaggagatggaatggcaccatctgatgtaagggaagagaaaataaattattaaccagtacggcccagtcctattggccccatgacagacgagggttatcactaagaggaggaagctgccttaatgtgcaaactcaggggccagtcctcagcttccccggctgtctccaaggcctggtcctgcttttccttgatcacttcctggctctgggatggcagctgcctggcagggatggctgctctgggccctgctcctgaattcggtgagtctgttgcttgtggctactccttggtgcctcccattagggcaaagtgatacctgatacctgatactgggtacctgataacggggaagggtcccacggctgtgggagggttcctatgcccaaagataagtgctggtggaggggtctccaggtcaaggggttgaagggatagaggtcagagaggcaaagggatggggcctttgtctgaggttaaatggggaccaagtcaggtgctagaggtggatcccagtgaacagcgcctgaaatattctgggcttgggaggaggttttgctaccatccttgtttgctctcaggcgatagcattggccaatgcaggatgtaggagtggggggctcttatacagactcttgtacaaggaaccctgacctcggggtagagctcagcctggagactcaaactgacagcaataaaggtcgctatctcctactctcccctgcagcacgaccctttaaagccacactctattggatcacttccttttctgaatagccccctcactgtccattgggggagtgcccctccattggcaccctaagcatagcacgagcccccacaagcctcccgcagcactcccagccccttactgctggccttcttacccatagactccctagcctctcactctccagacagtccctggctgtgccaaccagccttagggcttatggatgctatcggttctttctgcaggcccagggtgagctctacacacccactcacaaagctggcttctgcaccttttatgaagagtgtgggaagaacccagagctttctggaggcctcacatcactatccaatatctcctgcttgtctaataccccagcccgccatgtcacaggtgaccacctggctcttctccagcgcgtctgtccccgcctatacaatggccccaatgacacctatgcctgttgctctaccaagcagctggtgtcattagacagtagcctgtctatcaccaaggccctccttacacgctgcccggcatgctctgaaaattttgtgagcatacactgtcataatacctgcagccctgaccagagcctcttcatcaatgttactcgcgtggttcagcgggaccctggacagcttcctgctgtggtggcctatgaggccttttatcaacgcagttttgcagagaaggcctatgagtcctgtagccgggtgcgcatccctgcagctgcctcgctggctgtgggcagcatgtgtggagtgtatggctctgccctctgcaatgctcagcgctggctcaacttccaaggagacacagggaatggcctggctccgctggacatcaccttccacctcttggagcctggccaggccctggcagatgggatgaagccactggatgggaagatcacaccctgcaatgagtcccagggtgaagactcggcagcctgttcctgccaggactgtgcagcatcctgccctgtcatccctccgcccccggccctgcgcccttctttctacatgggtcgaatgccaggctggctggctctcatcatcatcttcactgctgtctttgtattgctctctgttgtccttgtgtatctccgagtggcttccaacaggaacaagaacaagacagcaggctcccaggaagcccccaacctccctcgtaagcgcagattctcacctcacactgtccttggccggttcttcgagagctggggaacaagggtggcctcatggccactcactgtcttggcactgtccttcatagttgtgatagccttgtcagtaggcctgacctttatagaactcaccacagaccctgtggaactgtggtcggcccctaaaagccaagcccggaaagaaaaggctttccatgacgagcattttggccccttcttccgaaccaaccagatttttgtgacagctaagaacaggtccagctacaagtacgactccctgctgctagggcccaagaacttcagtgggatcctatccctggacttgctgcaggagctgttggagctacaggagagacttcgacacctgcaagtgtggtcccatgaggcacagcgcaacatctccctccaggacatctgctatgctcccctcaaaccgcataacaccagcctcactgactgctgtgtcaacagcctccttcaatacttccagaacaaccacacactcctgctgctcacagccaaccagactctgaatggccagacctccctggtggactggaaggaccatttcctctactgtgccaagtgagtagatctgaggggaacaggtgagagctgctatgcccccaggaaccaggccagaacctagctccacccttgggagccagggacagctcgtatgtgcacatatcagggccatggcctgtccaagtctatttaagtcccttcttggagctcactcccatcttattcctgcaggaattttgtcctaccagtctttccagctccaatccatatgatctttccatccatgatgctcctggtatcaacttaataatttttagaattactttaacttcacatgaatgaatattttgcttgtgcatatgtatacgcactgcttatatatgtgcctggtgctgaagaagccggaagaagttgctagattttcaggaactggagttgaggtcagtcatagcggccaggtgggtcctgggaaccgggctctggccctttgcagaaataccatgaaacgtctgcgtcctctctcctgccctcatgtagtcttagtttaaatctcaaagcgatgtctcaggtagtgtttgtgtttgtgatgcttccttttctggactctgtgtcttggtctgtagggactttggtagcctcacaactggctagaaatatgttcatctgggcttaggtggaactgtggttagtctccagtcccaggcatcagcacagttttttctacaaccttatgctgttgaggttctgcttctggctttgtccattttggctggcacaaacaggatgccagtgagctcatcagacagagggaaggttggtgagagggccagaggtagaggaggctcctggagaacatcatggagagtgaagtgcctcaaatggccttgtccactctagagcaggcgagggttacagcaggtaaccacagctgagtgttcttatgaaaacagttttgaccctgcaagccccagacttcatagtctttagagccatcagatgagagcagaaagcttttgctggctctcattgctactggctgtctatccccgtttgagtctccagtgcaagctacttcctagagtatccatgctgtcccctagatcggacagcagagaagggctgtggagaggcatcggggatcagccacgcaaaagacaatttaaaaaatattatttatttttatttatacaggtacactgtacctgtcttcagacacaccaggagagtacatcagatcccattacagatggttgtgagccaccgtgtggttgctgggaattgaactcaagacctctagaagagcagtcagtgctcttaacctctgagccatctctccagccttacagtgtagcttttgtttttgttgcttttatgttgtttttagacagagtcacactatttgagacggtcctcaaattcacgcccttgcctcagcctcctgagggctgtggctgcaagcctaagccatcatacttggcttgtactacctttattttgattttgaatgctcccgactcctggtgagtcagttatgttaattctatagactggaaacctgaggctcagagtggtatggtaagacgggcaaggccacacaggaatccagctctctttgacggctctgttgatcaatatactcacttgttcagacctcagaatgtgtataaagagcttggtgttggtagtctattcagtctccacagaggtgtgctctgtagaaagggtttagttgaagggcacaggcccctgtccaagggcattctctgggtctgtgagctccagggctcaactcaacatttaggggtgattctagctctgggaggggaaagtgaagaacagcattgagatctgtgagggagatgggcatggctcagttctgggctcatcacttaatggtgatgctcatttgacaggtctggaaggtttggctatgtgagggggcataggaagcatcacctgcccaagggaaccacattcagtggactaggggaccatatgagactaccttgtgaggagatagtcattttgaactctctgggcctggtattgtggagacactgctcctccaatagcggggagaggagctggggcagggaggggccaaagagtccaggcagggccaggaaaggttctttccctttgtggtttccccctagtgcccctctcacgtacaaagatggcacagccctggccctgagctgcatagctgactacggggcacctgtcttccccttccttgctgttgggggctaccaaggtaagtgaggtagctgggggggctactgaagggataattttggcacagagataataggtaggaggagggagaagccatggtgagtgtatccaggatctgggggcctggcataagggggctgcaggcaatgcttcctacctcactgctctcatctctcaatgctacccaggagctctggttttgtgcctttggctgggaaagggaaatgaagcatgggataaggctgttattggagtgaggaagcaatagaaggacaggaatgggagaaggttacaccctgaggggaggaggggaaaagggttcaacaggagggaggctcagggtttctcttcccagggacggactactcggaggcagaagccctgatcataaccttctctatcaataactaccccgctgatgatccccgcatggcccacgccaagctctgggaggaggctttcttgaaggaaatgcaatccttccagagaagcacagctgacaagttccagattgcgttctcagctgaggtaggggccctgcagagtccctggttctatgcttgcaatccctaatggtgtgggtctattccagtcaaatctacaaactggctctacttgttcctgactggccccgggcagtgaacacctgtgcctagctgtggcgcttgtgttagaggctcctgcagttcattcctagagtgtgtggccactcagtatgtggtccgtgagctggctgtgtgcttgcagcgttctctggaggacgagatcaatcgcactaccatccaggacctgcctgtctttgccatcagctaccttatcgtcttcctgtacatctccctggccctgggcagctactccagatggagccgagttgcggtgagagcaagagggacacagtgagagtgactcagagcctaggacacctccagaaggcttttcaaagcttcccgagtgtgggcacattaaaatagcaagttggacacatccagatggaatcccttgaagggtagcgtttcttgggtgtgttctatgttgaaaggctttcttcctgctctctaatatattccaactgtctacatgcaaagctaccatttaaaaggccatgcaatgcagttctgggaaggtgcagccaggtacccctgcattctttggttccatgggcttgcccctgagagcatggtttagcatagagacttagatgtgggttcttcattgaggtgggtggtgtgtgagcaccaatgatgcctgcccactcctcagccaccctggagagtacaaagggtctgggcaggtgtcctggtagccagccctcctcactgaattgcaggtggattccaaggctactctgggcctaggtggggtggctgttgtgctgggagcagtcgtggctgccatgggcttctactcctacctgggtgtcccctcctctctggtcatcattcaagtggtacctttcctggtgctggctgtgggagctgacaacatcttcatctttgttcttgagtaccaggtaagaagggaggggttcttcatactcaacatcctcattagacaaagttctgcacagactcactggaattctggtcaatttatacgtgtaggaaatagcctgggttggcacaaattcattcacactcattgagccatcttgaacttgcttccagttaaacccatacagcatccagtaagctttgtaatggattagaggtacctctttcctgcctttacattaccaggggcggcattccatggtataggcacaagccagagtccagatagtctctctttgctgtcaaacacttggcgtgacatgaacacttggtcgtttccacatctagaacgcaccagtggttctttacatcccaacatagaagcagagagcgtggctgtgagctgttagtaggctcttctgtccacggaaggtctggaagttcctcagatttggccaggaatccaaaccctaaccaccccaatgctgacctctaaagtttggtgaccttgggctggagaaatggctcagcagttaagagcactgactgctcttccaaaggttctgagttcaattcccagtaactacatggtggctcacaaccatctgtaaagggatgtgatgccctcttctggtgtatatggaaacagctacagtttactcatatacataaagtttggtgaccttggcacacccgtgtactctgtctctttgcccatgcagaggctgcctaggatgcccggggagcagcgagaggctcacattggccgcaccctgggtagtgtggcccccagcatgctgctgtgcagcctctctgaggccatctgcttctttctaggtgagcaagggctgtccttctccacccgggatgggatttgctaggttattctaagagggagcccaggctttcagaaggcagtgggtgttccctgctttaagctgtctgtgctggcatgtggcccatgatgccagaatgcccgacagaccctgtgccctcgacaggggccctgacctccatgccagctgtgaggacctttgccttgacctctggcttagcaatcatctttgacttcctgctccagatgacagcctttgtggccctgctctccctggatagcaagaggcaggaggtaagttcaactgggccaggacaagggacttaccctgccagtgtccctatattctctggaagatgtggcacagaggtagccagaagagtttgatgggaggcagggacagtattctgagagagaatgtttggggctctgtgctcaccaatttcctgtaaaaagagaatttctttttagttatttgtggtaacatcatcaacgcccctaaaagtatgtaaagtttacaaaataaattgtaaataaaaagttaacataaattttttgatgacggaaaattcagtatttgattaagacaggaagtaagctgggtgtggtggcccatgcctttaatcccagcacttgggaagcagaggcaggcggatctctgagttcgaggccagcctggcctataaagtgagttacaggacagccaaggctacacagaggaaccctgtcttgaaacaaacaaacaaacaaacaaacaaacaaacaaaaaccaaaaagacaggaagtaaaagcaacaaaaaactgcgtgggggctgtagagacggctcagtggttaagagcactggctgttcttccagagatcccgagttcaattcccagcaactacatggtggctcaccatccatactgggatctgatgccctcttctggcagcaggtgtccatacagttagaccactcatacaaaatcttctgggcttccttgaatgaggtggatcctgtagtctgccttggacccagtcttgagggcctgtcattctctaggcctctcgccccgacgtcgtgtgctgcttttcaagccgaaatctgcccccaccgaaacaaaaagaaggcctcttactttgcttcttccgcaagatatacactcccttcctgctgcacagattcatccgccctgttgtggtacgtgggctgaagggctgttccacttttgtaccactttgggagggaaaccgggcagagcatggtggcatgggaggctgcccaggcccggagcagacacttggagctagagcttgagcctgtccaactctaggacgtttcccaggatgcccaacaaagccattcaaatttgagggaagatgaaggctgtttggggagaggttctcacgtgccagtttttccctcagctgctgctctttctggtcctgtttggagcaaacctctacttaatgtgcaacatcagcgtggggctggaccaggatctggctctgcccaaggtgagcctggccttttctcagccctttgtcctgggaggggcagcagtgcccaataggtggagcggtggtggtggtggtggtggtggtggtggtggagcttgagagggggacatagcacaaggcttagccccatgcagagttgctctaagtggaccgtgagagagaaagcacatccatgttgtaagtgtgagcgctgagtgctggctcagggtcacagtagatgtcctgtgctggaggcctatccacatggccattcacacagggtggggcgccacttccttctatgtcagttcctcaccaatagctggtttcggatttattactttatctgtacgagtgttttgtctgcgtgtatgtttttgtgccatgtgggtgcctggtgcctgcctgcagaagtcaaaaggagggtgtcagatcccccgggactggaattacagatggctgtgagccaccctgtgggtgctgggaactgaacccgggcattctgccgagccaactctccaacctcagcacttgttatttttctgtgttttttttttttttttttttttttttttgtgtaggggaatcaaatctgggatctcccatttgtcttgtttcgatctcttgagagtcctagcaacaccgctgtctggctttatagtttcgatttgcattttctttctttttctttttaaagatttatttatttattatatgtaagtacactgtagctgatttcagacaccccagaagagggcatcagatctcattacaggtggttgtgagccaccatgtggatgctggaatttgaactcagaacctttggaagagcagccagtgctcttaactgctgagccatctctccaggcccctcaatttacattttcaacaattagaaatgttacataccttttcatgtacatgttgatcactatatatcttatttaagaagaaatgtgctgactttgctcggtttttgaattggctttttgttgttgctgagccttggagagttccctgtggattctggaggttggtgtcttctcagagacctgattatcaaatggttttctttttctgtgggctgctctgttattctagtggtggtgtgccttggtatgccaaatatttaagcatatccatggattctttttctcttttattgtctgaatttgatggcatattaaagacataattgataaacagaaagttattaagtttgtctggtttctattaaggttttttatgactttagaacttctgtttaagtctttgattcatttgagatttgctcatttgttttttgaaatagggtttgtctgtgtagccctagctgctctggagctcactctgtagatcaggctggccttgagttcagagatccacctgcctctgcctccaagtgctgggattataggtgtgtgccactaccccactttgaattgacctttatatatgatgttatgaaagtggacaaattttaattccatccagctttcccaggactgtactaagaagtacagctctcctccatccgatggtttggcagccctgccagaggtcattcaagcatgtctgtgcatgactctttattctgctccattgaaatttcatgccggcttccgtgtcagcagggccctgctttgattcatacggagttgcaaaccagaaaatgtgagacttgcaaatttgttctttgtcgatttctcttgggctatttgagttcttgtgagattacacttgaattttagcttgacatttttagattccttcaaaaaccatccttggcatttatgcagggattgcactgaatctgcagatggctttgccttgatagtactaataccgtcacaatatttgtcatccagcccatggacacacgatgtatttttttttcatttttttctttaatttcttctaagaaccacatctccaaatttttaattttttttttttgagacagggtctcactacgtagccctgcctcactgtgtactcacatgtagatcagactggccttgaacccacagacatcgcctggctctgcctctcaagtcctgggaccaaaggtgtgtgccaccacactaggtctgagccactggctttccacatgctaagcatgcactcttaccactcagatgcaccctgagccctcctctctgaaggatagttttgctgtaaataaggttttttcttccctttagtactttgaatacatgaaccgcagtctccaacggcagatgggaaaggtggaagcagctgcgttagtcctttgtgacaagccattttttgtgtgttgtccccagagctctctgaggttggcttttgacagctgtactacagcctgccttggtcaaggtttgagcttgtccttttagacgtcccaggaattcctttaaggttgatattcgtgcctctctttcatcgatttgggggagttttggctactgcttcttcaaagatcacatccagattctttcatttttcttccttttctgattttgtttttgtttttgttttttgtttttcgagacagggtttctctgtatagccctggctggaaactcactttgtagaccaggctggccttgaactcagaaatctgcctgcctctgcctcccgagtgctggaattaaaggcgtgtgccaccaggcctggcttttttctgaaattcttacagtgcatctgtgggcctattggcatcgcctggggccctctctgtgtgtatcttggtgtgcccttttatgtttgtatctgggcatgttcctcgaagtacatagtgctgtgtgtacctcagcctgtatcttgtatatatgtacacatgtagtctgtacatttctgagtaggtttctgagcatgtgtctctgagtgttctgagcatgcatctctgagtgttctgagcatgtgtctctgagtgttctgagcacgtgtatctgagtgcgtccctgagtctgcctccgagcatctcatctacctcgtgtacctctgagtgtgtcttctgcttccagatacatctgcatggacttctgagactgtgctctgacctggggcggacccactgtggatatttcctacactcacagatcctcttctttcccaggattcctacctgatagactacttcctctttctgaaccggtacttggaagtggggcctccagtgtactttgacaccacctcaggctacaacttttccaccgaggcaggcatgaacgccatttgctctagtgcaggctgtgagagcttctccctaacccagaaaatccagtatgccagtgaattccctaatcagtaagtggttggtctccccgacaccctggcttgttccttctctgctttctctctccattcctcttctctcttcctgcatgctctgtttctgcagctaacaaagccaggggaggctccagtgcaagggtaaggaaggagtccccagcagactcattggctccacctcctcctctccactgtctggcctcaggtcttatgtggctattgctgcatcctcctgggtagatgacttcatcgactggctgaccccatcctcctcctgctgccgcatttatacccgtggcccccataaagatgagttctgtccctcaacggatagtaagtttggggctacaggaggctcactgcccattacagcttagggaaactgaggcaggagaaaagaaaggctctcagtctcccatcaaacccatagggtccaggtggtttaggggttaggcactcacactatcagtgtcccctggagtattacacctttgtttgcagaacatgttggttgtgggcagtgggctatggagttggaagtggagctatggccctgcatatggagctgctgtgtttaacaagtgtgggagatcccatttcttgaccccacaactgggggtggcaggtgtaaacctcttagaactggggactttagatttgggcacagaatgggagtcaggacaggagctgccttgcctggtgtgtcactgcccagagtcctccctctctgcagcttccttcaactgtctcaaaaactgcatgaaccgcactctgggtcccgtgagacccacaacagaacagtttcataagtacctgccctggttcctgaatgatacgcccaacatcagatgtcctaaagggtaggttccgagggtggctcttgctggagactggggagactagtgggttctagaaatggtagacacagaggaggcaagagtgcctagccaagccctttctggggcacagtgagtggactgacaggacaaggtctcgttccctctaagcctctactctgtcctccactttgcaggggcctagcagcgtatagaacctctgtgaatttgagctcagatggccagattataggtaagtgtgatatggtttggggaggagatctcaagtcagtcagctgttttagagtcctctaagagcacccatgcatgtggctgacgtgtgtgtgtgtgtgtgtgtgtgtgtgtgtgtgtgtgtgtgtgtgtaagttagcagggtgtgagggtaggtgtataactgtcctgggcctgtaagcaccggttctccttctcaactccacgagctaatacttcacccattctttcaccccaagtccattgccactgtgaaatgtgtggcagcctttgtaacagctgacgtcattattgagagccacccacttcaacacatattcactctcgtgtgtatcatatgtccatgtcagcagcaacttctggctaaatgaagtaggatgcctttgtgtagtggaatctcaagaggcatcaacagtaactagggagtgactctgacaggtggggaggagcttaatccagcaggcagcagaaccaaccaatcatctgcatggagggagccagctgttgttagatctgtgcacaccactatgagtgcaggagctggcaagacgggtctgggtgctttggatgaattcagttttttacctaaataactccaagttggaatcactagctgtaaactgatggcagcacatctagtccaccctctaagctaactcttctatgaagttcctatcttccaagaagtcacaccaccttaccctaggcaacagaggcatgtggtcaaaagggatcggggtatggcagacagaggaatggattgtttcttggagggcaggtctgcatctgtcatggccctaagtatcccacagcagcgcttttctttcttttttaatttttaatttttttggtttttcgagacagggtttctctgtgtagccctggctgtcctggaactcactctgtagaccaggctggcctcgaactcagaaatccatcagcctctgcctcccaagtgctgggattaaaggcatgcgccaccactgcccggcagcagcacttttcaaaatgagagttcccctctctcctctaacagtatcagcattatcaggagatggctagccatgcccaccccttgcctagccatgcccaccccttgcctgagccatgccagaccatatctcggcacgaggatgaccatccttctgggtgtgagcaactagtaccttaagattacgcattggcgactatttctcatctgtctttgttttccctgtgtgtgcctagccctcctttctttagcggatgcaacactgctgaacacaattcacagttgtttattgtacttacaggcaggatcccaaagtcacagctttaataattcagtcatttttgtttgcctgtcttcctttctagtctgttcccacaggaataggcaaactgaaaattaatttttttgagacagagtctcatgtagcccacactagtctagaactttctatgtagtgctgaactcctgaatctccagcctccccaagtactgggaccacaaatatgaaacaacacactccaacaagaaagtaggctcaatttttgattaaaatccagtgcatattctgaaagcacacatagaaggatttggtttcaccagggagatattttagtactttagtttagttttttttttttcttttttctttttttgagtttctctgtgtagccctggctgtcctggaactcactctgcctcgaattcagaaatccgcctgcctctgcctcccaagtgctggcattaaaggcgtgcgtcaccactgcccggagagattttagtttttgctttgcttttgaaacagtatcttactctctagcccaagctggccttgaatttgaagtaatttccctacctgtctgctggctgctggaatttcaggtggtacataaggcatgatcattcaagcttcatctatcaaagtcaggccttggtctcagtggcagaagtagagtttgaggatgttgatgcaaaatgtaaatgcaatccttgaggctggagagatggctcagaggttccagaggacccagctctgactcctgcactggtgcagcagatcatgactgcctttaagtccagttacaggcgatcccatgccctcttctggcctctatgggcaccaggaatgcatgtggtacacagacatacatgcaggcaaaacactcatacatgttaaaaataaataaggaaatgcaggtctcttgtagagaatcgatcaggaatttcaagtgctgctggtggcaccctgaacccaatgagatcacatctgtgttaatttctccgggatacgccaatgggcactgatctttgactttcagcctcccagttcatggcctaccacaagcccttacggaactcacaggactttacagaagctctccgggcatcccggttgctagcagccaacatcacagctgaactacggaaggtgcctgggacagatcccaactttgaggtcttcccttacacgtgagaactagagggctgagtcaggggtgtgggaggaaacagccacacaagagatgttggggggactgtaggctttgagtgatcctgtgggatgaggaccaactttgtctcagctgtctcctggggtagcttgcggcctgtccatttcttacacaggtcagcacccaactcaaatctgggtcccttgttttccttgggagatgggatgctcatatcacctgaagggaggattgatgctttgaccacaccctgatctttggcaggatctccaatgtgttctaccagcaatacctgacggttctccctgagggaatcttcactcttgctctctgcttcgtgcccacctttgtggtctgctacctcctactgggcctggacatacgctcaggcatcctcaacctgctctccatcattatgatcctcgtggacaccatcggcctcatggctgtgtggggtatcagctacaatgctgtgtccctcatcaaccttgtcacggtaacccacagagcgggccttggaagttgacgatctacactcataagctaccctcattctatgtagaatctagacatccggctggcttgctttcttgtacccacaccccaccttctcccttgtttcctgaagttcatttctcttgcctgtaggcagtgggcatgtctgtggagttcgtgtcccacattacccggtcctttgctgtaagcaccaagcctacccggctggagagagccaaagatgctactatcttcatgggcagtgcggtgagtggggagggatggcctcaccctgcgatccacctgagcctttatgtcctcctgtgctgactcctggctgtgactcctgccaggtgtttgctggagtggccatgaccaacttcccgggcatcctcatcctgggctttgctcaggcccagcttatccagattttcttcttccgcctcaacctcctgatcaccttgctgggtctgctacacggcctggtcttcctgcccgttgtcctcagctatctgggtgagtacctgtgcacacccggccaagatgtcacaactgtgagcattgatcaaaatggtgcctgctcccctggaaaacttagagatttcaggctgagggttttaccatacatcctactttgggagcttttgttttacatttaatatgcagcaagcatctttctctgtgagttgattgtgtcttaaacactgtgtgggctgctgaacttttctgatagacgtttatgcacatacaaacacacacacaaatggacacacatgcacataaacacacatactcacatagacacacactcagacacacatgtacacattcacacagacacacatagacacacatatatacagagacacacagacacaagcatacactcatacacacagacacacatatagacactcacagacacactcacacaagcacagatacaaatacacacacacactcacaatttgaatgcacagacacacagatacagatacatacactcatactcacatataaacactcacacacagacacacaaaaacatgtacaggggctggagagatggctcagtggttaaaagcagtagctgcttgctcttccagaggtcctgagttcaattcccagcaatcaaatggtggctcacaactatctataatggtaaccaatgccctcttcaggtgtgtctaaagacagctacagtgtacttatataaatgaaataaataaatctttaaacacacacatacaaagacacatactcatatacacaaacacacacacacacatacagacacacgttcacagaagcacagacacacactcacagatacacacagacatgtacataaaagacacacacatacacagacacacacacacacacacacacacacacacacacacttctctgtagatggaacccagaatattgcacatgctaggcaagtactgtaccactgagtcacaccttagcacagaatacatattttacaatgagaattgatgagtgaggtccatgagattgtctgagtagattctgagcctcttgctcatatagtaagagaaggtgtattttggccaatcacagtatatatgtttggaatctcagggtcctcatggcatcaactgtagtcactgaggctctgttttggaccaattacagctttcctcttggcatcaatttcatcctggattgtgctttaggccaatcacagtcttgcctcttagcctcacaacagtctccagctacatcaccaggggtggtggtggtggtattcaacctacagctgatgaaggcctaagggccgtctggctgatagttcttcagggcagacagaacagcaaggccgagtcccacaggtggtgtttaggaaagcagattgccccatcctgcagcatcttagcttgcttacagggacatgggcatcaggagcgcttacaatataatctaaagagattattaggacgaataatgatcttaatatattgttaatggtgctgcacatgcttaactcaccaagtaccccaggaagaagtgtttcccttatctgcatcacttcctccactctctatttaagcagcctacaacttctggtcattagactatttctgatgctatactactgttgctagcactacagtaactgaccttgttgctgaatctttgaccttgtcatccagtattttcttagagtaaacctggagatgttgattattgatgtcggttgttaatactccccacaaagttcaatggtgatgataatggtggtggtggtggtggcattagagtcacctacaggaactcactgactatctttgtggagaagaatgtgtatgttggggacagtgagggaacagccctgggagatgttgccagcccagagcctcagagacacaggctggaagtttctagacctatatggggtggagagtactgaggactgcaagcctcccatccccagtgatgaagctgtagtcaagaataccctgaagctagggctatgcaagcagagtcccgaagggcatgtggtgagtatagagccctactccctggttgccttgtgccttgggtttgtagttacaatataaggtatgctttttagaggagagttgaccatggtgtcagtaccagattgtcccaggaacaaagggagagagagggtgtcagggatcctgttgagagagcatgccagctgaggcaggctggtgagggtggtggaaataccagctgaaacagctgttggtaatgtgaggcaagcgcaagcaggtagagccgggctctatctagatttgcataccgctgtgatacctgcgtcatctgtatgcctctcagaccatagatgtatgttctttctcttccacagggccagatgttaaccaagctctggtactggaggagaaactagccactgaggcagccatggtctcagagccttcttgcccacagtaccccttcccggctgatgcaaacaccagtgactatgttaactacggctttaatccagaatttatccctgaaattaatgctgctagcagctctctgcccaaaagtgaccaaaagttctaatggagtaggagcttgtccaggctccatggttcttgctgataaggggccacgagggtcttccctctggttgtttccaaggcctggggaaagttgttccagaaaaaaattgctggcattcttgtcctgaggcagccagcactggccactttgttgtcataggtccccgaggccatgatcagattacctcctctgtaaagagaatatcttgagtattgtatgggatgtatcacatgtcaattaaaaaggccatggcctatggcttaggcaggaaatagggtgtggaacatccaggagaagaaaggattctgggataaaggacacttgggaacgtgtggcagtggtacctgagcacaggtaattagccatgtggcgaaatgtagattaatataaatgcatatctaagttatgattctagtctagctatatggccaaggtatttataaatatatttcgagtctgagtcttatttctgggagcatggggctgggtgggaagaacagggcccaacaatcctccttcttgcccagggtcttgtagttgccgggaacatgtttgtatctctcacccagcatttcctccccttatcaaaactatttccagggctggagcacttgttcttagagagaacatgggttcagttctcagtggttcacaatcatctacaattccaatgtcaggaaatttgacaccttctgatgttcacagacaccaggcatgtggtgcacatatgtacaggcaagacactgatacacacaaaacaaacaaatacatctaaaaatgatttaaagaaaacatctttagggccagtgagatggctcagtggttaaaaggtgattggcatcaatcttgagtttgaccccctggaactcatatgatgggaggagggaaccaactcttggaagggctcctcttacctctacatccatgcattggcacccctagcccccagaaggtaacaacatattaataaagtctccgttctaggatggggctgtagctcagtgctggagcagcagcatggcagcctcatgtacatgcagtgtctgtcacctgccatcctcagtactgaaaaggacagagagcaagagccccgaccttgtccctagatgttaccacttccagtgacaataactgcctttgtttaccactgtccctgagtacatttaaaaaaaaaccctccattccatatcagcatgactgttaaatgactgttaatatttacctatagccctaggacagagtgtgacccaccctgggctgtaatgttttagaagagcagggaaggcaaaggggacctaatgtcttcctggcttgaggaggtcacagtacgctgggagtggttgacctcatctggaaaatggcattcagtttggcctccagtttcctcagctacagagcatgttgcaggcgctgtgtgtctgtctgaaggcagacagctctgggctgggcaggttttctggcatgggtcttatggctggagcacaacctgaatctggtgccttggttgcaacagagacagagaagaagataccttgtttgtgaagcacagactttgttgaatagtgtcgtagagagtgttttactgctgtgagcagacaccatagccaagacaactcctagtgtcttagagagggttttactgctgtgagcagacaccatagccaaggcaactcctagtgtcttagagagggttttactgctgtgagcagacaccatagccaaggcaactcctagtgtcttagagagggttttactgctgtgagcagacaccatagccaaggcaactcctagtgtcatagagagggttttactgctgtgagtagacaccatgaccaaggcaactcctagtgttgtagagagagttttactgctgtgagcagacaccatgaccaaggcaactcctagtgtcttagagagggttttactgctgtgagcagacaccatgaccaaggcaactcctagtgtcttagagagggttttactgctgtgagcagacaccatgaccaaggcaactcctagtgtcttagagagggttttactgctgtgagcagacaccatgaccaaggcaactcctttaaggacaacatctaattgggtttggcttacaggttcagaagttcagtccattatcatcaaggtgggaacatggcaaaatccaggcaggcatggtgcaggaggagctgagggttctacatcttcatctgaaggttgctagaagactggcttccaggcagctagaatgagggtcttaggctcacatctacagtgacacacctactccagcaaggccacgccctctacccccccaccctctcccgggcaggatacattcttggagctggaagtttactgaatgggtcccttgatcttgaatgcactccctgggccaagcatatgcaaagaaaaatgatgcttttatcacgtgtcctgctctggctgcctctgggttaaggataacttttgtacaggatacaatcacaatgacatgcacatcagggacatttatggaaatattgtttttgttctattcctttccattttcaagaggatagtcttgtgacatgttcaactgcctatgagagcctgtgatgggcaggggtactgtcctccactgtgaggcacagggttagaacatggggcctgtgaggcgggcacatttggagaatctactggagaatgccagagtgcattcaagatcaagggacccattcagtaaacgtccagctccaagaatgtattctgcccggtgtgtgtgtgtgtgtgtgtgtgtgtgtgtgtgagagagagagagagagagagagagagagagagagagagagagagagagtgatgggggacattccagatcccctttgagcctcttcactctctctgtccagctcctgtttggctgaagagcagctgtggttcctcctgtggaaggaggaaaggagctagcatctctggaaactgctgcctactttctagtctgccagccccctgtgtattattactagctggtctttacaagggtccctggaaggtcaggaagtatgtgaaatctggttaaagaagctctgcctcagaacagagggtgaatctcagctattccatgttaaatgacaccttgtcattagctgtcctgtgcatgttactggggaggaaagtgtttctcagtcctagccagctgtaaacctggcaagatatgtacacaggcgtaagggagacatgaatgttatggggccaaccaaccattttctaatttttgtaaacaagtcttttaatttgtttatttagtttgtaaatgtgtatgcacactcaacttgcatttatgtttttgcactgtgtgtatccctggtgcccgtgaatgccagacaaaaagtgtcaggtctcttggaactggagtacaggctgttgtgaaccaccatgtgaatttctcttcaaggaacagcaatgttcttttttttttttttttttaagatttatttattacatgtaagtacactgtagctgtcttcagacacaccagaagagggcgtcagatctcgttacagatggttgtgagccaccatgtggttgctgggatttgaactctggaccttcggaagagcagtcgggtgctcttacccactgagccatctcaccagcccaacagcaatgttcttgactgctgaaccttttctccagcctcccacaaaccactttctggttggacttgaagccagctccacaaggtagaacccatgcctggtaccattaacggggctaaaaactgtggctaactacattataggccatggggagaacctagtcttattatgttaaatggacatagtaaaagacttcccttcaagatctcatcttcatactcagagatcagttcattcctcaacttttatcagagaaccgtctttttgagtaggtggtgattgatacagcaactggtcaccatgcagagactaagactgtggacagctcagctctaaataggatgtctatgtcatgtgtcctccccacaaggctcagagagggaagagagggcagaaagtctgtaagagacagagggagtgaaccaatgcagtgagactgtgtttgccagacacgataggaccattgcatatgtgaactcacaggggctgggaaggcatgtacaagacctgcttgcctaagatcaagccaaccacaaccatagcaagataagtgagggcccaagaagtcccaccccatctgaggcactactgacagctgagggctacataatctcacccttctccagggatgcaggctgtggtggtttgactaggatcagctcccacagactcatgtgtttgaatgctttgctcataaggagtggcactattaggaggtgtggccttgtcggagtaggtgtggttttgaggtctttgcttaagccatacctagtgtggctcacagtcacttcagctgcctcttgatcaagatggagaactctcagctccttctccagaaccatgcctgcatgcatgctgccatgcttcccaccatgacaataatagactaaacctctgaaatgacaagtttggctccaattaaatgttttccttataagagttgctgtggttatgatgtttccttaaagcaatagaaacccacattaagagacggtccctgagaggctacccatgttccagtaaacaggtccacactgaatggatgaatggacagcaatgaatggactcagtgggcatcaaattgaaaagaaagggggtggggtagaaaacatttgtacatgaagttgggagggaaaaattgtgtggagctagggaggatctggaagggagaggatagggggtagattgaatccaaacacactatataatatttacgaatcataaaactaaacaacctcaagaagaacctaggggagcctgtataatctgggagggacagtttccccaagcatagatccagaagccgtaaactaaaagcaagggggccctggggaagtggggaagggaaaagacccacaccccgccagagttccacctactctctggtcagtcaggtgtgggaggggtgggcattcctctatcccactctttagggagtggccaggggcagccctacctggggaccctggagctactttgctaaagccaccagggttataggagagagggatgagggaagagattcccaacacctgtgagagtacatgcagccttgatggagcagagactctctatggtttaagagctttattatagaaaggcagggagagaggggggggctagaaagagtaagagggagagaggagagaagtcaaagagagaggagagaggagaagacaaagagagggtgagagagagggtgagagtgaggggtaagaagaagacaagtaagaggagtaagagagcgaggtggggctgaacagccctttttatggtcttcactgttgctaggtaactggggaggagtttagtctgaaggtcagaagcttgggccattgcctaagtgactactgaccatgcttctcttgttggggctgtgggggacagtagcttaggcaggagccagagttccaggagcataagggaacgcctaccgtgtcatgaaggtgaattatgactttggggttcagaactcagcttaactggagaccagcctatctttgtatagcccaatgccccacgcatattcaaataggaaccctctgtaatacaaaccaaaataaacaaaccaaaatccaaaagtaagctggaaaatgcatagtggtctgaaaaaacgtttgccctacgttcaacatacgaggactaaaatcaagaatacacaaagagacaaattagtgcagtgagtatgtggataactgtcccatgggaaacaacatctctggctaaaggaggctggggaggttgctcagttactaaagtgcttcctgttcaaacatgaggagctgagttcagatcctcagcatccatggaaaaagcctgtgtgacagcatatgcttatgatcccagtgctccagaggcagaggaagaggatcccagggcttgctctcattcagtggagccaaatccaagtgcagttgaaagacctgccccctactccccaacaaaccaaagccaaaacaaaataaaaaccaagctgggcagtggtggcacatgcttttaatcccaacacttgggaggctgaagcaggcggatctctgagtttgaggccatcctggtctacagagtgagttccaggacagccagggctacacagagaaacgctgtctccaaaccaaaccaaccaactaaccaaccaaccaaccaaccaaccaaccaaccaaccaaccaaccagtggagaataattgagagagacacctgatgtgacttctggcctccatctgtatgtgggaatgcacagtatcacaaatgtatttatttaacacacacacacatacacacacacacacagcagttaataaaaaagataagctccccttttactgttgcttgatagctcattccttcttgttgctaaatagtatttcctttcatgaaccattctcttggcaaactcttggctgcttctaatttttgcagttatgaggaaggcaatgaaggtttctttgtaggtttttgtatgatcacagttttcaaatgctgggcaaatatatggtagcatgtttgctatgctgtaaagttacctttagcttctcagtttttttaggtccttccttctttcttttcctttctctttccttccttccttccttccttccttccttccttccttcctttcttccttcctggtggtaagtgggactcactttgtagctcaggcttgtcttgatactcttcctgtctcagccttccaagtgctggaactttaaccataagccaccccaccagactactaactattatttattggtttgtttaattattgctttttttcttttcttttagacaaataactcactgtatttagcctcagtgggcctgccacttgctgtatagacctggctggccttgaactcacagaaatcagcctgtctctgcctcctaaatactagagttaaacctgtgtgccaccattcccagcttccactaattttatttactttttttttttttttttttccgagacagggtttctctgtgtagccctggctgtcctggaactcactttgtagaccaggctggcctctgcctcccaagtgctgggattaaaggcttgtgccacccctgcccggcactttatttactttttgagtgtgtaattaatgcaaatgcattcagtagtaccctttccttctattttgtaccctatttctccttccttgatgtccctattccagaggcagactctgttctttctctcttttttgtttttgttggtttgttttggagagagggtgtcatgtgattcagtctggccttaaagtctctgtgtagctactgttggcattcacgttctaatccttctgcctctgcatacaaatgctagcatgccaggtgtgtaccactatggctgatttctgctctcttccctgtgataccgtgtagatagtaaagaattattcaaagtggctgggaagattcctcggtggttaaagcacttgccatgcaagtgtgaggactagaacttggatccccaagaaccaatcaatgatcaatgggcgtggttgcctatacttccagcctcagcagagaaagccggctggcatgaccagctaaagcagcgaactttgtatttgactgagagacccttcctcaatgaatggtagaagagtgggcaaaggtgattcctgacgttagcaagtgctcttcaccagaaagccttttctttagcattcacattatttctttttaaaagtctgttgacagcaagcagcactgattcagtgaattacataaaaaaagtaaatgaggtcgaaggggctcatgttgggggtttggaatgtaggaattgtggttcatatggtcaagatacatcgtatatatgcattaaattgtgaaaaaatattcattttatatttttggttgtgagcctagcctttaatggctgagccatctctccagcccaaagatattcttttttttttttgtttttttttgtttttttgagacagaagatattcttttttaaaatatgttgcctgttgaggcctgctccttttaatatagcagtagccattttgtattctgtctccattttgctcctaaggtgaaatgaagttcaggttctcagactctgcttcccagaagtgagcatccagagctgacactaagtatgttactaataagccaaaaagttacggccgaatcacttgtccctgttatctcaatgttctgaaattccctgctcagtacctgtccaccacccttcttacctcagtcaggaccactcagcttacaggttggctaataatactttatctagttagacaaaactgctgtaccacttcactgcttgcctttgaacctttttttaagatatatttattatttatatgtaagtacactgtagctgtcttcagacgcaccagaaaagggtgtcaaatcttattacggatggttgtaagccaccatgtggttgctgggatttgaactcaggaccttccgaagagcagtcagtgctcttaaccgctgagccatctctccagccccatctttgaacttttgaacctggtttttcctataaaaagcctgccctgaggaccggctggtgccacagttaggtttttccttcttgtggacctagatgtccagtattatgctgtgtgttcaataaactattcctgtttaactgaaattggtgtacgtatggtttgtggcaagtctcagaccccgacactgacatgtgatgtgtatggttatttgcaaattaataaatttaagcattaactttcagtatagtaaataatgatgaataaaacataaaaactgtttggattgtcaacaaaattttctcatcgtctgtgtatgggtgttttgcctctaggcatgtatgtacttcatatgtgcatgcagtgtcctctaacgtcagaagagggtagcagattccctgggtttatagatgattgtgagccaccatgtgggtgctgggaatcaaatctgggtcctctggaatatcagctagtgttctttgtttttgtttttctgagacaggatttctccatgtagccctggctgtcctgaactctcttggtagaccaggctggccttgaattcatagagatctgcctacctcactggcattaaaggtgtgtgccaccaccaactgtctgagccatcgcggttgctccatgggttcttgaaacaaaatttaagatcataattttttgtttggttggttttttttgacacagggtttctctgtgtagctctggccgtcctggaacttactctgtagattaggctggcctcgaactcagaaatccgcctgcctctgccttccaagagcacgatcataaattctaagttgaaaaaatttacatcaatttatctgtatggctttacttaaaatttgctaaggcccaacactattaagttatttgttaagcctggaatgtctcttactggaaaagcattttcctaacatgttcaagaccctgagtttgtctcctagaactacaagaaaacaaaagtaaaatcagtattttgctctgtgtggtggcatatatctttacttctgcacttggcaggcagaaacaggcatatttctgtgaatttgaggacagtttggtctatacagcgagttccagggcagccaaggctgcagagtaagacaatgtcttttaaaaaagttaatttgtagatgttagttagtctagtagagatgaaattcattaaaatcttttttgttgttgttgttgttttttttccggtcaggatctccttgcagagcccaagttggtcttgaactggctatgtggatgaataaacatttctggttctgtcgccaccttcccagtgctaggtttacaggtatgggttactacacagtttatacaacattcaggacacattagtcacacatgtgggttactacacagtttatacaacactcaggacacattagtcacacactttaccaatttagctacattgaatgaaaaaaaaacaaaaaggaggacatgatggcttctaggtatggtggaggaggtttattgtagacaggagggagcagacagccagaagcagaggcatctgggagaattcagggtggaagtggctgtagaatgagctgggccatgtgagaagggttaggggagagggtagaagagacctggagtcaagaggccaggagaccaagaggccaaagggtaaaaaggacctcataaccaaaatggctgggttacataggaatcagagaagcttggggaggaaaaggccagctcagactctggactggagaagtttagggtagaggtcaggattagtatgccagccagaaggatcctgtaccagaaggtactgagggagactggtggccagagtctgctttgatattttattaggcatctcagccatttgtcttcggtttgtgacctagcatatgttcctaatctgtctgtctttcgtctccccccaaccctcttctgagactgggtttgactctatagcccaggttggccatgatttggatgcggggattacaggtataaaccacagattgattgtgtgtcaaccttacataaacgttttcttaaaatgtctatatgcacgtattagtaacggcacgtgtatacacacatgctatacatacggatgcacacacacatatttacagtatcatacttctttttttccttcactgagcttttcacacttttcttgtcctttcaagtcaccttgaaatctggtgcaaccttctgagtttcactcatagctctgttaataggaattccatacaattctacaatattccttttccgttccctccgctaacaaacaaaatgtggtattataagaaggcgcaccagacacctacgcaggaattcaatccagaaagaagaaaggctcccagaccacgtgacacctccagggactacgtcaagtggccgtcaccacaatgcttccgccctcttcaaacatggttggcaagcgctctccgcatcgtgaccatggttattcttgcatgtaggaaccgtactgagcgcataccaatctcctttaggcaagtgtcgcggcggaggagatccagcagagccgcaagaacgacgatcggttaccgccggtagtaacaagcgcggaccggaagttccgcgtcttcgctgtccggggggagccgttaggcgcgcacgccggaagtggccaatcagccggtgtgaggcggtgcccactgtgttcgcgtccctcgggcagcagagccatggagcccggggctgctgagctttatgaccaggccctgttgggcatcctgcagcacgtgggcaatgtccaggactttctgcgcgtgctcttcggctttctctaccgcaagaccgacttctaccgcctgctgcgccacccttcggaccgcatgggcttcccgcccggggccgcacaggccctggtgctgcaggtgaggtggagagaggcggcgggccgttggggtccagcaggtccttaccccagttccacctcccagcgccagaggtgccccggctcgcgtcctacgtctgggaactgcgccactccgtagcccacccttcagggtttgtgattctctctggggtgccaccaggtgatttgagtaaatggccaggcgttatctgacccacggatccgttggcaggaatgcgcttctttgggtacaggctgggtttgtgcgggagatgcttaggtgttggacctgtcagcctgggtttgagggcctcccagcgccctgtgggcatccccagaacggtgcaaggggcttgttaggcagggttcaaaccgtgcaggctgtaggaggaggactttcacagcgggagaaactagtagatttcagaattcccgggctcggaggtggcaggaatgactggtatgtattgattggtgggtgtggcctcttgccagtgacttactaagttggtggacaattgtaatgtttacgaaatttacatttggattaaaatttatatgtcctaggttatatttattgtttttgaagcgttactaatgactgactcaagttgtttggacacctttttaaaaactgttttctttcgagacaagatcttgctgtgtagccctggttgacttggaacttgccaggtagatcaggatggcttcgaacttatggtcctccttcccgacgctcccgcgctgggattgttgacattgttgagttagtaataagcaaacatttaatgaatatatttaacactctcagctgcggtatagactctgatggtatggatatttaaataatggtctgtctacttttgacactgtacagagagtaaagcacagacactaatagagagctctgctagttctctgtgtggtagagctctttggaaggaagtgatagaagtggtagttaggaaattgagacagttttgtaggagacgttgaagtaggttttgaggggcgtgtagagtctgattcacaaacctataaagtgcttcatgtcacctttgctgtttgttgacccctgcatcagccccagacagggctttcctgtgtagctctgaagtcctggaactcactctggcatcaacctcagatatctgcctaggattaaagactcgaattgccaccacctggcttaccttccatcttaatctgttctgtgggcttgtcaggggttactttcttcagttcctctcagtggaagaagccaaagcttagaatgttctagtaactcttaacataatgaccatgaggtgatcagtgggaaggtttgctactgagtttcagtttcagaatggaaaccgatgagtccctggggtagctcatccttatcagtggcccaagtgctgctggttgagtaattggagagggtgagaggtggcaccttgtgtttctttataatgaagtcttgccacctgatcgccatgctctctgaatctgacgtagtagttcaacaaggttagatgacaacaagaacttcactctgtttcctgcaactcaggacttcagttttctaattccaaatctttttgccctttttgtcatttagccactccatagtatggtgagaacttttgttttggttatgaaaggaggaaagagtatcccagtggctggctggcatttgaatttcttctgtataacatcttattttagctttaacacaatttgagaggttggttcttgtttgtctgatgaactgataaaggcaagatagatgcacttatcgtacaactttataaaacagctacctctgaaaggttaagatagctctataggttacagtgtggtgcccatgctatgggaggtagggagccgaggaagggacagggtcaaccaaatatttcaaggttccaaatctgggtaactggaagagtgaggtcattaactgtattgtcacttggatttggggggtgggaaagttttagataagttgaatttgagatcgcagacaggttctggcttcagagctgttggaatggtaggactggatcttgcaggagatggaaggactgtgaagtctgaagaagagcccagtgtagaggacagctggggaacggtcaccgtaaggggcattgggttctaccgcagccaaggttacagtcccaagtacaggtttaagagttgtttgttcttaccctttctccaaccccaggtggttctcaaccatcagccttgaccgagagcccttttatcttctcagtttctttcttttgtggggtgggaggttgaagacaaggtttctctgtgtagctttggctatcctgaaacttgctttgtagtccaagctggccttgaactcacagagatccgcttgcctctgccttctgagcgccggcattaaataaaggcatgtgccaccactgcctggctcagtttctattttcaaacaagtatttattgagtctccactataatatacactgatttgggccatgagaaacagaggcttataagttgttgtggggttttggattttttttttattctgagaaaaggtctcaccatgtagccttgactggcttggaacctactatgtagatcaggctggtctcgtattcaaagacatctgcctgcctctgcttcttgagtgctgggactaaaggcgtgcgccaccacatccagccaaccaagagactttttactatgtatacacttgaatactattttcatcaggtagatcatagtaagtcccccgaggatctgcttttttttttttttttttaatttactgaaagcctttgcaagaggcttgtaagctacatttagtattggtaagggttttggtatcttttctactcatttactgctttttctgttcttgattcagtgatcctaagactccttcctgtgacttcctctctcaaagtgtggttgctgcccagggtctgtgaacaggccggagtggtttgggtggtttctgtggcgcttgggagctccctaccgatgtcttggggagtagctgctgctgtgtgtgcttctttatgtcaactgaatccaaaccgagaccaaggcgtctagaaagacataatctggggctggtgagatggctcagtgggtaagagcacccgactgctcttccgaaggtccgaagttcaaatccctgcaaccacatggtggctcacaaccatccacaacaagatctgactccctcttctggagggtctgaagacagctacagtgtacttacatgtaataaataaataaataaacctttaaaaaaaaaaaagacataatctcagcccaggacttgccttcatcagatgattgatgtgggagggcccactgtgggcagtgccatccctgggcaggtggtatggggtgtataagaaagcaggctgcgtgagttagtaggtgtcattcctccgtagtctctgctttagttcttgcttcaagttcctgccttggcttcacctgatgatggactggatcctttaagccatgtaaaccctttcctccccaggttgtttgtgatcatggtgtgttttacacagccacagaaagcacacaagtgcagtgctgctgacagaggctggctgtctgtttcccttctcagggtttagagcatgagtgaagtgagtaaagattttgttctgttcattagtattagaaaatgtttttgttttgttttgtttttttttgttttttgtttttcgagacagggtttctctgtacagccctggctgtcctggaactcactctgtagaccaggctggcctcgaactcagaaatccacctgcccctgcccctgccccccccccccccccccagtgctgggattaaaggcgtgcactgccacgcccggctagtattagaaaatgttaagaacaaaagtagtctagtcagtcaatgactaaacagacaatgactcctggagtcagtgtgaaaagcatggctaagtggaagtcttccttataggagtcaggagccacctgtttctcacttctcttgtaacttattggaaaatcttagaggagtcagttcccctcttgtcttctgagatggcatattgaaggcaatggacttctattccaacaaggacactgcctctagggtgagtctgtgaacataggcagctggagctctggagtgttgtgagaactgggtagtgtagatgggcaggtggaaggggagtcgcaggaaggctgagtgaggactgtcaggccttggtttggagcaccctgaggttacaggagagtctttactgcctgacttcttgtagttaagttgaagatttaaggtgttagagatagagtgcttttgagtacaaaggactcagacactggtagcatggatgctagagagagtcaaaccttcaagctaccagcagattagaagtggtttttgactgtgttttgttttgttgttgttgtcgtcgtccttgtcgtcgtcatcatcttcttcttctaaagatttatttatttattttatgtgtatgagtacactgtagctgtacagatggttgtgagccatcatgtggttgctgaaaattgaactcaggacctctgcttgctccagccccacttgatcctgccccgcttgctcctgtcctaagatttattatatgtaagcacactgtagctgtcttcagccacaccagaagagggtgtcagatctcattacggatggttgtgaaccaccatgtggttgctgggatttgaactctgctcttccgaaggtcttgagtgctctgagccatctctccagccctgtcttttttttttttttttgaaacagggtctgactgtacaccctggctgggctggaactcactatatatcaatcacaatttatatcaggctagcctctgcctcctgggtgctgaattaaagatgtgtgctaccatacctggcctttgtctccagttttacatcttttaggattctgtctgtctgtctgtccatttattttggctagattgacttttattgtttgttgtgtcttgtgtaacttctctgaatctgcattttcttcatttgaggatgttggtggtgctcttcacagggtttttgtgagttttgaaactgtagaagcacacagtatagctagcactgtgttttgtcttccgagttgtgctcagacatgttagtgagtactcagtgccccagccatgtcccagcttacttcctcaagccttattacctctgcctagtgcaggggtcttgctctttcttggtgatactgcttcttggagctctgctgggcacactccttcattaaggaccacttgagaacccgatccttcttcctctgtagatttctttctctagaaatgttctcaccacagtctgccagggcatcagggttccatgatgccccacctgtatagacttctatcagagtgagtctgtacttgtgcagtgttgaagataaaacccactctcctgtcatgaccagccaagctgacctacaccccaagccctatagctgtatggaccatccatgatagtctcagcacctttctggatgttaagggtgtgtcttcataccaaaggaggactgtcaggccccagtcttggaaaagcctgtgttagaagtcccaggtaataggagtgagtttgtattcttttgctttttagagtttttattccttgcacactgtaggcccagggtgggtgtttatggagtcaagtactcactcctacatcgtatcctcagctgtcagttggggaagtggtggcaagaatctgataagcctgagtgcatcttgtagattttcatctttcactttttaaacctgaattgctggcaccttccggaatccacagcctgagtgtgttcttcacattgccagcaaggttggcaaaagtaatgacaactctggtgtcggcctttaatctcagcactgggaagacagaggcaggcagatcgctgaggccagcctggtctacagagcaaattccaggacatccagggctacacagagaaactctgtcttgctacaacctcccccttctcctaccttggtcccaaaaagtaatgacaactaaagctgtctagttttgcatcctctaggtttgtacatacagttagatttgacttattttgagtttattcattttggaaacttcttgaggaagagcaattcctaccagcttttggtgaagttgtagagtgtttccattttgctttggtttactgttatttaattttatacttagaagattttgttatatttctgtggtggtgtggtctgatagggtgcagatgaatttatttatttatttatttatttatttatttatttatttatttatttttgagacagggtttctctgtgtagccctggctgtcctggaactcactctgtagaccaggctggcctcgaactcagaaatccatctgcctctgcctcctgagtgctgggattaaaggtgtgagccaacactgcccagctgcagatgttgtattgatgtttgtttcatttttaggtctttaaaacatttgatcacatggcccgccaggatgatgagaaaaggaagaaagaactagaagagaaaataagaaaaaaggaggaagaggccaaggccttgccagctgctgaaactgagaaggtagcggtgccggtcccagtgcaggaggtagagatcgatgctgctgcagacttgagtgggcctcaggaagtagagaaggaggagcccccaggctcccaggaccccgagcacacagtgacccatggcctggagaaggcggaagctccaggaacagttagcagtgctgctgaaggccctaaggaccctcctgtgctccccaggtaggagcatctcctgcagtgtcgtcctctctgctgtgcttaagtttgcctatgagtggtttttgttttgtgtggtttgtaaaaaaatatcagctctgttttggtgggcagtggttaactatagaaattcatttcttaatagttctggaggctggaaaccccagattaaagtatgatctgggttgtttgtttgaaggcttctatcagtggtttgcagacagccatcttcctgtgtctcgtcacattcctttgttcttgcctgtgtcttattctcctattctcaagcagcactcaaacaccttagtgagccagattgccttccatcctggtgcttcagggacctcctttcggaactcagtgttgaattc

SEQ ID NO: 2 - DNA, 4002 bp, Mus musculus
atggcagctgcctggcagggatggctgctctgggccctgctcctgaattcggcccagggtgagctctacacacccactcacaaagctggcttctgcaccttttatgaagagtgtgggaagaacccagagctttctggaggcctcacatcactatccaatatctcctgcttgtctaataccccagcccgccatgtcacaggtgaccacctggctcttctccagcgcgtctgtccccgcctatacaatggccccaatgacacctatgcctgttgctctaccaagcagctggtgtcattagacagtagcctgtctatcaccaaggccctccttacacgctgcccggcatgctctgaaaattttgtgagcatacactgtcataatacctgcagccctgaccagagcctcttcatcaatgttactcgcgtggttcagcgggaccctggacagcttcctgctgtggtggcctatgaggccttttatcaacgcagttttgcagagaaggcctatgagtcctgtagccgggtgcgcatccctgcagctgcctcgctggctgtgggcagcatgtgtggagtgtatggctctgccctctgcaatgctcagcgctggctcaacttccaaggagacacagggaatggcctggctccgctggacatcaccttccacctcttggagcctggccaggccctggcagatgggatgaagccactggatgggaagatcacaccctgcaatgagtcccagggtgaagactcggcagcctgttcctgccaggactgtgcagcatcctgccctgtcatccctccgcccccggccctgcgcccttctttctacatgggtcgaatgccaggctggctggctctcatcatcatcttcactgctgtctttgtattgctctctgttgtccttgtgtatctccgagtggcttccaacaggaacaagaacaagacagcaggctcccaggaagcccccaacctccctcgtaagcgcagattctcacctcacactgtccttggccggttcttcgagagctggggaacaagggtggcctcatggccactcactgtcttggcactgtccttcatagttgtgatagccttgtcagtaggcctgacctttatagaactcaccacagaccctgtggaactgtggtcggcccctaaaagccaagcccggaaagaaaaggctttccatgacgagcattttggccccttcttccgaaccaaccagatttttgtgacagctaagaacaggtccagctacaagtacgactccctgctgctagggcccaagaacttcagtgggatcctatccctggacttgctgcaggagctgttggagctacaggagagacttcgacacctgcaagtgtggtcccatgaggcacagcgcaacatctccctccaggacatctgctatgctcccctcaacccgcataacaccagcctcactgactgctgtgtcaacagcctccttcaatacttccagaacaaccacacactcctgctgctcacagccaatcagactctgaatggccagacctccctggtggactggaaggaccatttcctctactgtgccaatgcccctctcacgtacaaagatggcacagccctggccctgagctgcatagctgactacggggcacctgtcttccccttccttgctgttgggggctaccaagggacggactactcggaggcagaagccctgatcataaccttctctatcaataactaccccgctgatgatccccgcatggcccacgccaagctctgggaggaggctttcttgaaggaaatgcaatccttccagagaagcacagctgacaagttccagattgcgttctcagctgagcgttctctggaggacgagatcaatcgcactaccatccaggacctgcctgtctttgccatcagctaccttatcgtcttcctgtacatctccctggccctgggcagctactccagatggagccgagttgcggtggattccaaggctactctgggcctaggtggggtggctgttgtgctgggagcagtcgtcgctgccatgggcttctactcctacctgggtgtcccctcctctctggtcatcattcaagtggtacctttcctggtgctggctgtgggagctgacaacatcttcatctttgttcttgagtaccagaggctgcctaggatgcccggggagcagcgagaggctcacattggccgcaccctgggtagtgtggcccccagcatgctgctgtgcagcctctctgaggccatctgcttctttctaggggccctgacctccatgccagctgtgaggacctttgccttgacctctggcttagcaatcatctttgacttcctgctccagatgacagcctttgtggccctgctctccctggatagcaagaggcaggaggcctctcgccccgacgtcgtgtgctgcttttcaagccgaaatctgcccccaccgaaacaaaaagaaggcctcttactttgcttcttccgcaagatatacactcccttcctgctgcacagattcatccgccctgttgtgctgctgctctttctggtcctgtttggagcaaacctctacttaatgtgcaacatcagcgtggggctggaccaggatctggctctgcccaaggattcctacctgatagactacttcctctttctgaaccggtacttggaagtggggcctccagtgtactttgacaccacctcaggctacaacttttccaccgaggcaggcatgaacgccatttgctctagtgcaggctgtgagagcttctccctaacccagaaaatccagtatgccagtgaattccctaatcagtcttatgtggctattgctgcatcctcctgggtagatgacttcatcgactggctgaccccatcctcctcctgctgccgcatttatacccgtggcccccataaagatgagttctgtccctcaacggatacttccttcaactgtctcaaaaactgcatgaaccgcactctgggtcccgtgagacccacaacagaacagtttcataagtacctgccctggttcctgaatgatacgcccaacatcagatgtcctaaagggggcctagcagcgtatagaacctctgtgaatttgagctcagatggccagattatagcctcccagttcatggcctaccacaagcccttacggaactcacaggactttacagaagctctccgggcatcccggttgctagcagccaacatcacagctgaactacggaaggtgcctgggacagatcccaactttgaggtcttcccttacacgatctccaatgtgttctaccagcaatacctgacggttctccctgagggaatcttcactcttgctctctgcttcgtgcccacctttgtggtctgctacctcctactgggcctggacatacgctcaggcatcctcaacctgctctccatcattatgatcctcgtggacaccatcggcctcatggctgtgtggggtatcagctacaatgctgtgtccctcatcaaccttgtcacggcagtgggcatgtctgtggagttcgtgtcccacattacccggtcctttgctgtaagcaccaagcctacccggctggagagagccaaagatgctactatcttcatgggcagtgcggtgtttgctggagtggccatgaccaacttcccgggcatcctcatcctgggctttgctcaggcccagcttatccagattttcttcttccgcctcaacctcctgatcaccttgctgggtctgctacacggcctggtcttcctgcccgttgtcctcagctatctggggccagatgttaaccaagctctggtactggaggagaaactagccactgaggcagccatggtctcagagccttcttgcccacagtaccccttcccggctgatgcaaacaccagtgactatgttaactacggctttaatccagaatttatccctgaaattaatgctgctagcagctctctgcccaaaagtgaccaaaagttctaa

SEQ ID NO: 3 - protein, 1333 aa, Mus musculus
MAAAWQGWLLWALLLNSAQGELYTPTHKAGFCTFYEECGKNPELSGGLTSLSNISCLSNTPARHVTGDHLALLQRVCPRLYNGPNDTYACCSTKQLVSLDSSLSITKALLTRCPACSENFVSIHCHNTCSPDQSLFINVTRVVQRDPGQLPAVVAYEAFYQRSFAEKAYESCSRVRIPAAASLAVGSMCGVYGSALCNAQRWLNFQGDTGNGLAPLDITFHLLEPGQALADGMKPLDGKITPCNESQGEDSAACSCQDCAASCPVIPPPPALRPSFYMGRMPGWLALIIIFTAVFVLLSVVLVYLRVASNRNKNKTAGSQEAPNLPRKRRFSPHTVLGRFFESWGTRVASWPLTVLALSFIVVIALSVGLTFIELTTDPVELWSAPKSQARKEKAFHDEHFGPFFRTNQIFVTAKNRSSYKYDSLLLGPKNFSGILSLDLLQELLELQERLRHLQVWSHEAQRNISLQDICYAPLNPHNTSLTDCCVNSLLQYFQNNHTLLLLTANQTLNGQTSLVDWKDHFLYCANAPLTYKDGTALALSCIADYGAPVFPFLAVGGYQGTDYSEAEALIITFSINNYPADDPRMAHAKLWEEAFLKEMQSFQRSTADKFQIAFSAERSLEDEINRTTIQDLPVFAISYLIVFLYISLALGSYSRWSRVAVDSKATLGLGGVAVVLGAVVAAMGFYSYLGVPSSLVIIQVVPFLVLAVGADNIFIFVLEYQRLPRMPGEQREAHIGRTLGSVAPSMLLCSLSEAICFFLGALTSMPAVRTFALTSGLAIIFDFLLQMTAFVALLSLDSKRQEASRPDVVCCFSSRNLPPPKQKEGLLLCFFRKIYTPFLLHRFIRPVVLLLFLVLFGANLYLMCNISVGLDQDLALPKDSYLIDYFLFLNRYLEVGPPVYFDTTSGYNFSTEAGMNAICSSAGCESFSLTQKIQYASEFPNQSYVAIAASSWVDDFIDWLTPSSSCCRIYTRGPHKDEFCPSTDTSFNCLKNCMNRTLGPVRPTTEQFHKYLPWFLNDTPNIRCPKGGLAAYRTSVNLSSDGQIIASQFMAYHKPLRNSQDFTEALRASRLLAANITAELRKVPGTDPNFEVFPYTISNVFYQQYLTVLPEGIFTLALCFVPTFVVCYLLLGLDIRSGILNLLSIIMILVDTIGLMAVWGISYNAVSLINLVTAVGMSVEFVSHITRSFAVSTKPTRLERAKDATIFMGSAVFAGVAMTNFPGILILGFAQAQLIQIFFFRLNLLITLLGLLHGLVFLPVVLSYLGPDVNQALVLEEKLATEAAMVSEPSCPQYPFPADANTSDYVNYGFNPEFIPEINAASSSLPKSDQKF

SEQ ID NO: 4 - DNA, 30 bp, artificial
  primer
gcgggatccgaaccggtccagctacaggta

SEQ ID NO: 5 - DNA, 32 bp, artificial
  primer
gcggaattcctcgaggatgggcaggtcttcag

SEQ ID NO: 6 - DNA, 26 bp, artificial
  primer
gcttcttccgcaagatatacactccc

SEQ ID NO: 7 - DNA, 27 bp, artificial
  primer
gaggatgcagcaatagccacataagac

SEQ ID NO: 8 - DNA, 24 bp, artificial
  primer
tatcttccctggttcctgaacgac

SEQ ID NO: 9 - DNA, 22 bp, artificial
  primer
ccgcagagcttctgtgtaatcc

SEQ ID NO: 10 - DNA, 25 bp, artificial
  primer
cctccctattccccaagatgtatgc

SEQ ID NO: 11 - DNA, 22 bp, artificial
  primer
ggagaggctattcggctatgac

SEQ ID NO: 12 - DNA, 25 bp, artificial
  primer
ctgggctccctcttagaataaccta

SEQ ID NO: 13 - DNA, 22 bp, artificial
  primer
ggagaggctattcggctatgac

SEQ ID NO: 14 - DNA, 21 bp, artificial
  primer
ctctgagcccagaaagcgaag

SEQ ID NO: 15 - DNA, 23 bp, artificial
  primer
gaccagagcctcttcatcaatgt

SEQ ID NO: 16 - DNA, 22 bp, artificial
  primer
gagaatctgcgcttacgaggga

SEQ ID NO: 17 - DNA, 32 bp, artificial
  primer
gcgaattctatgtctgggggcaaatacgtaga

SEQ ID NO: 18 - DNA, 37 bp, artificial
  primer
gcggatccttatatttctttctgcaagttgatgcgga

SEQ ID NO: 19 - DNA, 57 bp, artificial
  synthetic sequence
ttggggtcattgtcgggcattggggtcattgtcgggcattggggtcattgtcgggca

SEQ ID NO: 20 - DNA, 88029 bp, Homo sapiens
gatcatgaggttaggagttcgagaccagcctggctgatatggtgaaacgccgtctctactaaaaatacaaaaattagctgggcgttgtggcaggtgcctgtaatcctagctacttgggaggctgaggcaggagaattgtttgaacccaggaggcggaggttacagtgagccgagatcacgccattgtactccagcctgggcgacaagagtgaaactcccatctcaaaaaaaaaaaaaaaaaaaaaaaaaaagacatgtattctctctctcagtcacggacggcagaagtccgaagtgaggagtgggcagggctgcacttcctaggctctcggggagactttttttcctgtcccttccagtttctggtggctccaggcatgccttggcttatggcagcattattattccagtgtctgcctctgtgatcatagtgcctccttttcttttttttttttttacattttttttttgtatttagagaaaaaaacacttaacataaaatttaccatcttaaccttttttttgagactctgttgcccaggctggaatgcagtgttacaatcacagctcactgcagcctcaacctcctgggctcgtgacatcctcccatctgactctcccaagtaactggggaccactggcatgtgccaccacacttggctaatttttacattttttgtagagacagggtttctctatgttgcctaggctggtctcaaactcctcagctcaagcaatcctcctgccttggcctcccaaagtgctgggattataggcgtgagccaccacgcctggccatgttaaccatttttaggtgtgcagttcagtatgttaaatatattcacattgttatgaaacagatgtccagaactttttcattttgctaatctgaaactctgtacccattagacaacagctccccccgcaggtaaccattctactttttgcttctatgattttgactactttagacactttatctaaatggaatcatatggcaattgtctttctgtgattgacttatactacttagcataatgttaagtttcatccatgttgtagcatgaatcagaatttcatccctttttatggcttgataatactgcattgtatgtatataccacattttgcggtaggtacaatgtatatttacattgcttccacctcttggctactgtgaataatgctgctatgaaaatgggcgtgtaggtatcttttccagatcctgactttacttcctttggataaatacttacaggtgggactgctggggtatatgattgttctacttttaattatttaacactcttctacaatttattttctgtttttgttgttctaatagtagttattattaggtgaggtatttcttatctcttataaggacacctgtcattggatttagggtccacctggttaatccaggattatcaagtctcaaaatcctgaattacatctgcaaagactctttttccacataaggtcacattcacaggttccagtgattcaaacatggacatgtcttctggtcccccattatgtccactatactctcttttttttttttttttttaagatggagtctcgctgtgtcgcccaggctggagcgcagtggcgcgatcttggcttcctgcaagcccacctcccaggttcacgccattctcctgcctcagcctcccgagtagctgggactacagacacccgccaccacgcccagctaatttttttgtatttttttagtagagacggggtttcaccatgttagctaggatggtcttgatctcctgacctcgtgatccccctgcctcagcctcccaaagtgtcgggattacaggcgtgagccactgcgcctggcctaagtccactatactttcttcttccctgccttatttttattcttgatacttatctccatctgacatgctctatatttctttatttatcttgtttggcagacgacaatcaagataaagccatggagacaaggatttttgttgctgttgttcttgttttttgagacagagcctcactctcacccaggcctagagtgcagtggcacaatctcggttcactgcaacctctgcctcccaggctgaagtgatcctcccacctcagcctccagagaagctgggactacaggtgcttgccacatgcctggctaattttttgtatttttggtagagacggggttttgccatgttgtccaggctagtcttgaacttctgagttcagatgatccacccaaagtgctgggattacatgcgtgagccactgcgcctggcctagacaaggatttttgttttggtcacctgtgttttcccattagaacagtggctggcacaaatggctgcacagcacatactggttgaacaaatgaaggacggggtggctggtctagacaaagagcctagacaaacatcggcagaaattgcttcatggcttctgagcagaaaaatctctcatctggggaattagactccctaagttaaattttctttcttttttggagatggggtctcactctgttgcccaggctggagtgcagcagcaccatcacagctcactgcagcctcaacctcctgggggtcaagcaatcctcccacctcagcctcccgagtagttgggactacaggcccatgccaccatgcccagctaattttttttttggtagagacagggtgtcaccatgtagcccagactggtcttgaacttctggactcaagcgatcttcctgcctcggcctcccaaatgctgggattacaggcatgagccacagtgcctcacctcctaagttaaattttctgcagtggagaatacaatctctttaatattatctctcagttaagacaaatttcaggatcctccttaaaaaaaaaaaaaaaaagaaagaaaataagtttgccaatacaaataccatttctcactaaagtgaattagggttccttggagaaatggttggttttgtttctgggcagtaaatgtataaaacggaaagcaaggaagtccagggtgtccaatcttttggctttcctgggccacactggaagaagaagaattgtcttgggccacacataaaatacactaacaatagctgatgagctaaaaaaaaaaaaatctcataatgtttttagaaagtttatgaatttgtgttgggctgcatgggccacaggttggacaagcttgacctaaagactactaggattgtggattactaggattgtgccagaaggacacagcagcaactaaatatttgatgagacaatctgaacatttaaaaaaggacaatgactgtaatggattaaagcacatcaaatatctaaacatccatcaattcatgatagcactgccccttctccccaaagaacccaaagtggtcacagttagaggttgctggggcatccatccatccatctttattattattactattatttgagacaaggtctcactcagtcacccatgctagagtgctgtcgtcccatcacggctcactgtatcctcaacctcctgggctccagccatttccctgcctcagcctcctaagtagcgggaattacaggcatgcatcaccatgcctgtctaatttttacatattttgtaaagatcttgccatttcctgggctcaagcagtcctcctgtcttggccccccaaagtgctgggattataggcagagccactgtgcgtgggaaagcatcaagcatacatcctggctatcccgaatggattgtatttcaaagtaaccagagagatgagggaatgctcacctttgtagaagaatctcatcttataaatgcaggagaaatgagagcatttgaaattaccactttgcacacctaaggaacatcataaaactacactagggtttctcaacctgggtactactgacattttgagctggataattcttcgctgtgggggggaggtgtgctctgtgaattatacaatgtttagcagtattccttgcattcattttctagataacagcagtaccacccgccaccccccacccagttgcaacaatccaaaatatctccagacattgccaaatttccccttggtaggacagggcagaatcaaccctcgttgagaaccaatggtctaatgatcatcaacgtttgctagactattagaagaaaggctgatggtaaacttcatggataatcaggatgacaacccccaaattgagagatgaattacaatattactaagagacaaacccgccttgtgcctcagtagaagtacatagtgccagccacgaagcgttattgcaaacaaaacaaaacaaaaaaacccaaacctcaacattacacctaaacctaatgaagcttctagccaggggcaaatccaagctttgtggggccttaaactatacaaatttcacagtcctctttaagaaaaagacacaaaattataaatgcgaaattaggtacgggggtctatgcaagggagggcctgaagattaagcttcattagtttcactgtaaacctcccctgactctagaattaactgtgattacaggacataccagggacaaaaaacgttaaatgacacctgaagatacaatcagcaaaacccagaaagtggaaaattctgttggtcaaatgacccagtttcttcaataagtaaatgccatgaataacaaacaacaaaaagagaggggaaatttatatatataatatatatataatatgtataatatatatatatgttgttatatggtttgtttttttttttttggacacgagtctctctctcacccaggctggagtgcagtggcatgatctcggctcactgcaacctctgcctcctgggttcaagcgattcttctgcctcagtctcccaagtagctgggactacaggtgagcaccaccacacccagctaatttttgtatttttggtagaggtagggtttcaccatattggccaggctggtctcgaactcctgacctcgtgatctgcccaccttggcctcccaaagtgctgggattacaggtgtgagccactgcgcccggccctgtttttgtttgttttgagatagaatctcactctgttgcctaggctggagtacagtggcatcatctcagcccactgcaacctccacctccctgattctagcaattctcctgcctcagcctcccaagtagctgagattacaggtgtgcaccaccacacctggctaatttttgtatttttagtagaaacagggtttcaccatgttggccaggctggtctcaaactcctgacctcaagtgatccttccacctcagcctcccaaagtgctgggattacaggcatgagccactgtgcccagccaaaattgttatatattaagagacatatatttgtatgaaatgcagtaagtaaaccttgtttggaccctaaatatctaatgtacaaaattttttaaggcaatggggaaaattaaacacatactaggtattaagtgatgttaaataatttttaaaattttggtgggtgtgataatagtataaagtccttatctgttagagacacacactgacgtatttataggtgaaatgacatgatgtccaggatttgctttaatatacagcacttcaaaaaaaaatgcagaaagggatacatgaaatgagaaaggcagcaaactgttgttgaagttggatgatgagtacagccctccgttattatccaagggggataattcctggacccctacggataccaaaatccaggtatgctcaagttctttatgaaagttcattgtaattattctattatataaaagtttcgaaattctgttgataaaatgtatttttagtagagacgggggtttcaccttgttggcccaactggtctcgaatttctaacctcagatgatccaccagccttggccgcccaaagtgttagcattacaggcgtgagccaccgcgcccgcctggccttgataaaaacagttttaaccttccgttgcttcgattccatgcccactaagtaacattccagtttgtttttcactttcaaaaggatgtgctgtaactaggggatgtaaacaagctccatgaccctacttattccaagttttcgttccactctcccaccttttttttaagacaaggtatcaccctcggtcgcccaggctggagcgcgatcactgcttaatgcatcctcgacctcctgggttcaagcgattctcctgcctcagcctcccaagtagctgggactacatgcgcacaccaccacaccggttaattttttgtagagacgggggtttcaccatgttgcccaggctggtcccgaactcctgggctcaagggatccgcccgcctcagcctcccagagtgctgggattacaggtgccagccaccgcgcccggccccagcttcttaaaagaatgatccgaaactatggcagcactgggcttttggtccccacccaagaaatgcccgctcgcagaggctcgccgcggcaggctctcccgacgtgacagagtgtgggtctggattcagcctcggttcttacgagtcagataggtggacacgcaaagcaaaacatcacagggctttttgtatttagcacagaaaacacttgtgagcccgagctgagaacccaaaaggcacgcttcaggccatcgtagccaccaagcctggtcagattccgtccaccgtctccttggtgctccgagacccaaatcgctgactggggccgagggcgggcgtgactgcgcaggcgtgcctcccctgcgagatgccggaggtaagctgcggggtaaggggcgagaaattaagggcgaacgtcattgcgcatgcgccctctactctcgttgcgggggtaggcgggcgccgggctgtgtgagggggcggggcgcggcagtgttcggtacggatggagttgcaggagacggcgagtacatatcactgcgcaggcgtcctcttcccctaactctcagggtcgctagggtggcgcgcaggcgcagagcgatgcgcaaatgtgcgcaggcgcttaggggctgaggcgcgatggcaggtgtcggggctgggcctctgcgggcgatggggcggcaggccctgctgcttctcgcgctgtgcgccacaggcgcccaggggctctacttccacatcggcgagaccgagaagcgctgtttcatcgaggaaatccccgacgagaccatggtcatcggtcaggcgggctgagggtggggaggccctttgtacccagctcagccctcggcggcgctccctcctcccgagcccagccgggtcgctggctcccccagtacctagcctgagggtgccccgaggacgccaggccccctgcctagagctccgggccgcacgtcggagggggccgggcggagaggcggcccactagggccggtcgtgactatgtgtctgccccgcaggcaactatcgtacccagatgtgggataagcagaaggaggtcttcctgccctcgacccctggcctgggcatgcacgtggaagtgaaggaccccgacggcaaggtaaggctggcgttggcccacgcagccgttcttcagtggagctcccgtggggtgtaaagcactgcctggaggaggcctcaagggacaggaacttgcacttggagagcctgcggtataaaggtggggccttcactcacatatgttgcaggtggtgctgtcccggcagtacggctcggagggccgcttcacgttcacctcccacacgcccggtgaccatcaaatctgtctgcactccaattctaccaggatggctctcttcgctggtggcaaactggtaagaggattttctctttggcttcagcttagaatctctcacttgtttccaaattttgatttatcaagattgtgaaactttgtagcacagtcagaattggggagacagatgttgccttctgctccacagccagggacaatagtgggttccataccctggaacagacaactggaggccccaccactcatacattccatgtttccttgtagcgggtgcatctcgacatccaggttggggagcatgccaacaactaccctgagattgctgcaaaagataagctgacggagctacagctccgcgcccgccagttgcttgatcaggtggaacagattcagaaggagcaggattaccaaagggcaagtgcatatctccttgtaatttgagagggcagttgacctttatacccactatacctactcaagtttctgcttgggagatcagctctgcagagaatggaatgagaagtattggtttagataggttgtttgtttgttgtttttgagacggagtttcactcttgttgcccatgctggagtgcaatgccatgatcttggctcactgcaacctccgcctccccaggttcaagcgattctcctgcctcagcctcctgagtagctgggattacaggcatgcgccaccatgcctggctaattttgtacttttagtagagacgggggtttctccacgttggtcaggctggtctcgaactcccgacatcaagtgatccgcccgcctcagcctcccaaagtgctgggattacaggtgtgagctaccgcgccctgcctgttttgcttttttatcaaaacattttattgtggtaaaatataacaccaaatgtgtcattttaactgtctatatagttcagtggtattaagtgccttcataatgttgtgctaccaacaccatcatccagctccagaactttttcatcttctcaaactaaaaatctgtacttattttgttttgtttttgagatggagtctcgctctgttgcccaggctggagcgcagtggcgccatctcggctcactgcaccctccgcctcccaggttcaagcgattctcctgcctcagcctcccaagtagctgggattacaggcaagtgccaccatgcgtggctcatttttgtgtttttagtagagactgggtttcaccatgttggccaggctggtcttgaactcctggcctcaggcaatccactgccgcagcctcccaaagtgttgggattacaggcgtgagccactgcacccagcaaatctgtacttattataaacaataacttcccgtttccttttgtcctgacacccaccattctactttctgtctctatgatcctgactaccctatctcatataagtggaatcattcagtatttgtccttttgtgactggcttatttcactgagtataatgttctcacagttcatccatgttatagcatgtgtcagaatttcttaaggctaatattccattgtatgcatgtgccacatttcgctttcagtagtcatttttaagctctataaaataaaatgaagaaaggacagttcacaatctagtaatagccattgcctacctgtttttcttggactcttgttggaaatggtaggatcatgatttcagtcctaacagagatgcttgtggagggacagcctgtccctttcttggggcagcctcagtggggagaccatagcactcctaatggagtcacagatagtattccaaaaggagtttggtcctggagttgagtaattacacgcagggagggacctcacaacagccagactgtttctcctgctcacttaaccctgtgttgccccacacagtatcgtgaagagcgcttccgactgacgagcgagagcaccaaccagagggtcctatggtggtccattgctcagactgtcatcctcatcctcactggcatctggcagatgcgtcacctcaagagcttctttgaggccaagaagctggtgtagtgccctctttgtatgacccttcctttttacctcatttatttggtactttccccacacagtcctttatccacctggatttttagggaaaaaaatgaaaaagaataagtcacattggttccatggccacaaaccattcagatcagccacttgctgaccctggttcttaaggacacatgacattagtccaatctttcaaaatcttgtcttagggcttgtgaggaatcagaactaacccaggactcagtcctgcttcttttgcctcgagtgattttcctctgtttttcactaaataagcaaatgaaaactctctccattaccttctgctttctctttgtccacttacgcagtaggtgactggcatgtgccacagagcaggccctgcctcactgtctgctggtcagttctgggttcacttaatggctttgtgaatgtaaataaggggcaggtcttggccctagaggattgagatgtttttctaaatcttagaactatttttggataaattatatattttccttcctagtagaagtgttactgcctgtaactagctcaaaataccaatgcagtttctgcattctgggttttgtttttccttttttttttttttttttttttttgagttttgctcttgtcgcccaggctggagtgcaatggcgtgatctcagctcactggcaacatctgcctcccgggttcaaatgattctcctgcctcagtctcctgagtagctgggattacaggtgcccgccaccacgctcagctaatttttgtatttttagtagagatggggttttaccatgttggccaggctggtcttagactcctgacctcagttgatccacctgcctcagcctctgcattcagtttattcacatatttttggtaactcccatggcagctcctaggatttcagcggtctgtgggccagaaagcaggcaccagggctgacctcaaggccgtatcagagggccaagcagagttcttttggatacctgcttttcatcccacagggccttagagtcagaggtaaggtagcaacagagctagaatggggcaatgcactcttaccctccttctcaacttttatttaagctgtgctaaatgttttcttcaagggaaccagatttagttctttacagaattttccagtgaaataaaacatgttgtaatagctgtgtttgagatgaaataagaggttgtgggtagaggggaggcacctaaaggaaaagaggaaaggtgcctgggctacctatgcagataacctggagtggacttcactgtggactcgtggtactaaggcttggcctggacaggcagtctagggggtatgggaatacacggtgtggttgttcaactatttgcaaaggtcaaccaaatagaccacatgttcgcaaagtatcatctgaggaaattaagtaccttcttagccctctcagtcataaatttgaacaaattttaatacacttccctcatgcccttctatataaaacttaataccattagttccccattcttgacattttatttcagtttttattatatatttatttgaaatatttattaaattatctgacctacagaactaaattcttctccttttgttatttcttatgtcctataccatatatgtacctatttatatatatatttatgtatttttaaaatttttatttattttattttttgagacagtcttgctctgtcgcccaggctggagtgcagtggcatgatcttggctcactgcaacctctgcctcccgggttcaagcagttctgcctcaacttctgagtagctgggattacagacacccaccactacacccggctaatttttgtatttttattttatcttattcatttatttatttttgagatggagtctcactctgtcgcccaggctggagtgcagtggtgcaatcttggctcactgcaacctccacctcctgagttcaagagattctcctgcctcagactcccgagtagctgggattataggcgcccgccaccatgcccagctaatttttgtatttttagtagagacagggtttcaccatgttgaccaggctggtcttgaattcctgaccgcaggtgacccgtctcgcctcccaaagtgctgggattacaggtgtgagctggccgggcacaggtgatggggtcttgctctgtccccaaggctggagtgcagtggtgccatcacagctcaaagcaaccttgagctcccaggttcaagtgatcctcctaccttaccctcccaagtagctggtactacaggtatactccactgtgcctggctatttttactcttaaaaatacatgtgggctgggcacggtggctcacgcctgtaatcctagcactttgggaagccaaggtgggtggatccctatagcccaggagttcgagaccagcctgggcaacatggcgaaatcttgtctctgcaaaaaatacaaaaaatttagctggtggcacatgcctatagtcccagctacttgagaggctgaggtggaaagatcacttgagcccgggaggtcaaggctgcggtgagccatgatcgtgccactgcactccagtctgggcaacagtgatcccatctgaaaaaaaaaaacaaaaaaaaaaatgcaatttagggccaggtggggtggctcacgcctataatcccagcactttgggaggccaaggcagggggatcgcctgaggtcagcagtttgagaccaggctggccaacatggtgaaaacccctctctactaaaagtataaaaattagccaggcatggtagtgtgtgcctgtaatcccagctattcaggaagctaaggcaggagaatcgcttgaacccgggaggaggttgcagtgagcagaaatcgagccactgcactccagcctggggggcagagggagactctgtctcagaaaaaaaaaaaaaaatgcaatttagttctctaggcttttccatttaatagttttatatcctcctgtttctaaatctggatgacagtgtaacactccagtaaggtgaattgtgaattgctgaaattcttcagatgtttaaaagagttttcagtattcctcatgttagaattaatgcagagaaaaattttatcctttgaactagttacatgttgtggacttctggcctgaggctcttggggattatgtgacatattgggaagggacacatttctgctctgtggctgttactagaaatctagccagcaaatcagactacgtttgtgagaagacaggaaggcacagattagggttgagccagccttcaacaggtttggctggcagtagacacagtggagcacatcttaactattttggtaggtcctgggtttctcttggtagtttttgatagaaaggggaatggtgtgaggaaaaagtgggcatacatttcacctttccactgataaggcaggtggaattgggatagtcagtggatgggccaatagctggtggctgtgagaagaataaggatttccatactggtgtgtcatatttacagataggttgtgacctaaaaagttttttaaaaaacagcagttagggcctgggcgcggtggctcacgcctgtaatcccagcactttgggaggccgaggcgggcggatcacaaggtcaggagatcgagaccatcctggctaacaacggtgaaaccctgtctctactaaaaatacaaaaaattagccgggcgtggtggcaggtgcctgtagtcccagctactcgggaggctgaggcaagagaatggcgtgaactcgggaggtggagcttgcagtgagctgagatcatgccactgcactccagcctgggcgacagagtgagactccttctaaaaaacaaaaacaaaaccaaaacagtagttagggtacacacacacaaattctagtgattttccccccaatactacccttgacttttgaaattcttgctttctcagagtttacaacatccttaccaaacagccttctccctccttaccacaaaaaaagaaaaaaaagttctggggttgaggggacactccattcttaacatcctctattatcccagcccaattccccagctctcactgggactagttgtacctatcttcatcatttggtcccagcatgactacctgttggtgcatgagctgatctctcctaacctaacagccagatgctagtctctggtactcagatgctgggctgcatcagataggatgcacaggatcatcctggaagcttgttgacatagattcctgtgcaacactcagatatagtcttaatgtagatttgtgttgggtggtatggtaggtagaataatggcctaccactctgaaacatatgaatatgttacctaacatgacagaagagaattaagttgctaatcagatgactgtaaaataaattatcctggatcatctggatgggcctaatgtaatcacaaaggttgtttccttgccttttccagcttggctctggctcccttcctccagcaagggtgggttgagctctcacatggcaccactttgacctcttctgcttccctcttctacactgaaagacttatgggccaggagcagtggctcgcacctgcaatcccagcactttgggaggccgaggaaggcagatcgcttggccccaggagttcaaaaccgtcctgggcaacgtggcgaaaccccatctagaaagaaaagaagagaaggggaggggggaggggaggaggagttacatatatacacatacacacacacacacacgtacgtacatacatacatacacgctaactggacgtggtggtgcgtgcctgtagtcttagctttccaggagactgaggtgggaggaccacttgagcctgagatcgcgccagcctgggtgacagtcagaccatgtctcaaaaaaaaaaaaagatttgtgattaggattcttagtcctcacctgtattattttcctattgctactgtaacaaattaccacaaatttactggcttaaaacgacgcaagtctgtaggtcagaagtctgacacgggtcttaactggtgacccgagtcagatttgggacacaaagaacagaaaccaagctgtgcaggtttctgacaggcagtccggttagggagccctacagcaacccgccggtcctctctctcaggcagttgctgccatggctcattattccaaccggttctcctcagcccagtctatctcagtggctccattcatagggtgatgtgcccggcgggacactaaccctaaccaagcagagagacggtcatgcccgtcacgacctcggccctcgccccggccgaggcttctcctgcaggtcgcgagaatcaggtgcgtcagcggcgtccgggaacgccggaagagccagtggagcggctctgtagtccaaagtaccccgtcgaccccagcacggccgctccaccgcctcctactagacccagtcctagggactgcgcagtcgcagagctccgtccgagtaccggaagcctaggccgccagcacttccgggaagtgacttcgtctccgaagccgattggttgttgctttgctcccgctcgcgtcggtggcgtttttcctgcagcgcgtgcgtgctgcgctactgagcagcgccatggaggactctgaagcactgggcttcgaacacatgggcctcgatccccggctccttcaggtacacgcgagggctggggagccggcttacgggctctgcggggcgcgccatcgctcttcacgccgcttaaaccgcactcctggtctcctaggctgtcaccgatctgggctggtcgcgacctacgctgatccaggagaaggccatcccactggccctagaagggaaggacctcctggctcgggcccgcacgggctccgggaagacggccgcttatgctattccgatgctgcagctgttgctccataggaaggcggtgggtaacgagagagctgaggggaggaaggaggcaagctccaaaagcctgggaagggcggttcccgtttgtctgaggttttctcttggccctgtacccgtgcaggccggcctgagaacctggtgctgttgtggcaaacactctgggctggagttcaggttacctggatccttgtccggccctgctaccaccaacctttgcgtaatcttcgacaaagcactttcttttctttcttacataaaaagggagcacatctatcttttctacttacagaattattgtgagaatttagcttcataactagtatatttaaagtagcttcataaacatcagagtacgttattctttttgagggtcagtgcctggggaaagaactctccactctgcattctgaggcgggcagagtgatagatgatcaaagtactgctaagtagtgttgcagcagatgggtcaggtaggctggaaggggtagagacacgtggacacagtgatgtgcactgctggctaaagtctttaattcatattcttacagacaggtccggtggtagaacaggcagtgagaggccttgttcttgttcctaccaaggagctggcacggcaagcacagtccatgattcagcagctggctacctactgtgctcgggatgtccgagtggccaatgtctcagctgctgaagactcagtctctcagaggtgggtaaaagcagcaaagctgtacctgaatgaagctacacagtgttgtggggttgggtttgtgtgtggcaaaaaagagagcaaatccagggtgagatcccagctgctacattctgcctgatactgatgtcttgtccacctccagagctgtgctgatggagaagccagatgtggtagtagggaccccatctcgcatattaagccacttgcagcaagacagcctgaaacttcgtgactccctggagcttttggtggtggacgaagctgaccttcttttttcctttggctttgaagaagagctcaagagtctcctctggtaaggcagaggtgggtgtgattcctagtggaaacatctgtgagtaggagttgggacgagagcggggtggctggaagccagttactacaattagcggcccttggagctggaatctgattggattctttcatttcagtcacttgccccggatttaccaggcttttctcatgtcagctacttttaacgaggacgtacaagcactcaaggagctgatattacataacccggtaagaggcaccatggaagtgtctggagctgcagacatgggggcactcaaagatcttgatgctccttcttaggggattctttggtgttttgggtgggacagttgtcacttagtgtctcatccctggtcctgaggcactaaaagccagtggtctaaaatcactatatatttccaagtgtccacaagggatgtctcccatttcaggccatgctttgcctaaaatcctgagcaaggacctcccctaaggggcagctttgagcagcagagccaaaattctaaggccaaggttctcatcttaagtaaactttacctttcagaaggcctgttgctgtaggccttcccttctcaatgtagtcctttattgatgtgtttctctttgttctgtgcttggaagtattttatatatggtttatatggtatactctatataccacaacaataagggcattttggggttttaggttacaaaactggaggagagttagggtgccaggaatccttaaatgcatctctgccctgcactaaaatgttgatgctttggttggtgagtaagtggccatacatctctgtgttcttttcctttctgaccacaggcctgttttctcccccaggttacccttaagttacaggagtcccagctgcctgggccagaccagttacagcagtttcaggtggtctgtgagactgaggaagacaaattcctcctgctgtatgccctgctcaagctgtcattgattcggggcaagtctctgctctttgtcaacactctagaacggagttaccggctacgcctgttcttggaacagttcagcatccccacctgtgtgctcaatggagagcttccactgcgctccaggtctgccacagccaacatcttggttgaaataagttgaagatagagatggaaaggggacccagttaatgttctgtttcttaagcacttagtaggggccaggttctagatgtgactgatactgacttctcccaactccaaaatacctatcatggccgggcaccatggcttatgcctgctgtaatctcagcactttgggaggccgaggtgggcggatcgcctgaggtcgggagttcaagaccagcctggccagcatggtgaaaccccgtctctactaaaaatacaaaaattagctggacatggtggcaggcacctgtaatcccagctactcaggaagctgagataggagaattgcttgagcccgggaggtggaggttgcagtgagccaagatcgtgccattgcactccagcctgggcaacaggagtgaaactctgtctcaaaaaaacaaaaccctataattatttccagctgaggaaactgaggcacaatgattaagtagggaaagagattaagaagaggaaaaaggaaagggtgatggttactgtgatactagggatggcagaggggccttgagcttgctctgctgagctgattctctgtccgctcttggctgcaggtgccacatcatctcacagttcaaccaaggcttctacgactgtgtcatagcaactgatgctgaagtcctgggggccccagtcaagggcaagcgtcggggccgagggcccaaaggggacaagtgagtccatgcctctttttccatccctccccagaaatgcctgtgtttttagctttttggaagactaaaaccagagtgcacagagcagggagccaaaccttccaggcctggctggtagtgtagcccagagagccccacaggttcttgctcagctgcctggatatagagaagggagtggatggtgcacactgcacatgcaccacgaagggcaaaactgccggggttgttggcatgcagagccctgcaggggagatggcccatcctgcattggtggtatggctgtgacttgcagggagcatatttctgaagggaaaaggaaccccccaactctccagtctctgtccagctgaaggcttgactagctcagagttggttttcagatcaccatgtagggcaatgagttctgctgttgtcccagaacagaggtcaggccgagatttgggtacatgtcaaagctccaggctgccccaggaaaccctgactcctggaacggttccattgttggagagtcctctgtatgtcagggtcttatgatctacaggcatttagaggaagttttgctgattcagcgtgtgaatacgtgcccagaggagaggaagggtccggctgacattgagttatctctgcagggcctctgatccggaagcaggtgtggcccggggcatagacttccaccatgtgtctgctgtgctcaactttgatcttcccccaacccctgaggcctacatccatcgagctggcaggtagtagtgtgacggcccaggcatctgcatggtaggcacactgagggacttggggtgtgctggacagagcctgcgggttggagatgcaagctgcactgtcttcccttgcaggacagcacgcgctaacaacccaggcatagtcttaacctttgtgcttcccacggagcagttccacttaggcaagattgaggagcttctcagtggaggtaagagcctggctcttgtggtcctgggccagggtcaggcttcttccacaatgctttaaaactccatgataatgatgacagaggtcacaacatagtgtgacaggccacttccaccatccatccttgttctgccctgagtggcaggcactgtcccccttgagagataaacaaattgaggtaatttgtccaaagttgtgtttactgtctgcctcatgagcgttgagtgacctgacaggctgctgtgacagctcaggacagcacctgaccccagggtgctgggtggtcctggactgctctctgtggccgtcgtcatgggggtaccttgactcccaaggaataccatggggtactccttgggagaggagaagagagtgggtgacgggttcttgggcttggggccacacaggccacccccatccacacacggggacagatgggtcatcactgtaagaggcccaggtgcagctaacctgcatgttcggcatcccaggaaggcggtgggtcccctgctgctttcccccaagggggaggtgcaggaggcctccaatgaagaccctatcctaaggcctcagcctgtgggaccctcgctgctttcttctccacagagaacaggggccccattctgctcccctaccagttccggatggaggagatcgagggcttccgctatcgctgcagggtgagctgctgtggtggggaggggaatgagaggggaggggctgtggcccagggattgcaccgtcttgctgagcatccaggtgtgaagggaggatttggggcagcctcactgtcttgaccttcagtgtccacccccaggatgccatgcgctcagtgactaagcaggccattcgggaggcaagattgaaggagatcaaggaagagcttctgcattctgagaagcttaaggtgagtggatgggaggtgagaaggggatagatcttagacggctgccctttttggagactggctgagctccgagtggtgagaagcagagaactgggcagttttctggcctttggcacggaaggggaggaaatggacccagaatcatggaaggaagccagtctgttctgcttggtggtaaattggcacaaccttatggtggacactgtccagcagaattacgagctcatgtgtcctttcatccgaaattccacttctggaacttaatcctggtcacgcttgtgaatgtgcacagtcaagcatgtgcctgcattcatccatccatggcattatcatggaaccaaaagatggaaacagcctggggccaccatagggggcttgctaggtaaactcaggtgcattcagagccgaaggttacatgggaaggaatgaggttggttgcgtgtccatatggaacagtctgtaagatgatgcccagcaaaaaggggtacagggtactgccatgtgtgtcatggagaagggaaaatggaaacatccactcccgggaggttctgagaaatgcacagaagcagctgcctcatgccttttgaaacacatgagtgtgttatcctttgaaaagctaggtctgtgaagtcacagaagaaagatgctcactctgtggctctccctcttcccccggcagacatactttgaagacaaccctagggacctccagctgctgcggcatgacctacctttgcaccccgcagtggtgaagccccacctgggccatgttcctgactacctgggtgagtgtggcctgacagggcaggaggcagcaggctggggaagtggcattaatttctccactgctgggtcagcccctgtgcttggtgctggggatgctcaggcagaatagaacctggagaccctggcagcacgcgggcatgtaaacaggcacacccctgtgtttctaaacttgtttgcttggtcccacgggttagctgttgctgtctccattttagagatgaggaaattgaggtagtgcagggtgggtggcagacccagcatttcaggccaggtcgtctccagagctgggccaaatggccatccatgggtcgaagggagtgaacaggtttgggagagagtcacgggcaggaggcagagagagccacctgtgctgcaaaagactcaagattagcagctgctgaagaggcatctgtggagtctctgggtaagaacagtcagcagggagacagactctgtaaggcctaaaccgacaaggtagcaagagaagagccagtggtggtgcgagggatgcaggggttggcaggcatgaggtcaggaccctgggattggtttctgtagtgcagcccaggctagagctttatgtggccattaatactgggcacctctcctcatctttggcaggctctgggtaatgacttctttcagtgtctcataggaggtgtttttggtaatgagtatgtgtgacttttatgcctaaaatggattgaaggaggagagtggtggagaggaggctgtgggcagcaagtgcaggacccttcccaatgccacagggtctgctcagcctggacctgcagccacccagcgggtgtggtgttgctgctatggaggtgacaaagggtggagatggaatgttccagggcaggaaaagcctgggcactgggaaaggaaggatccagaagagatgggaacatgaaaatgccagagagagcggtgggggccgggttcccatgggacagtgagctggaggagaccccagtccaggtcctggcctgagatgtgaggaggggagttgggagggtgggtagggagggagaaggtaaggctagaactttggcctcaggaacccagtctgctcgtatagcggagtcatttgccaaggtgtggccaggaggtttagaagggccaggagaaggtggaaaggtgtcaggatgtgggatgtttgacatttgaaggggagggcccaggtgtggttggcctgggggagtccatggggtgggcgaggtgaagatagagccaagatcaggtgcagctgggatgcggggccccctgtatcggtagtaatgggccacaggtgaagaaactacctgttgacttttatttcagctgcattttctttctttaaggatgtctgtctttttctttcttgttacatgtttgttgtaacaaatctaaacaatataggagagtgatttaaatagtggaagtctaaggtgctcacattctcctggccctgtgcagatgtggtagtgaatagatgtatgtcataggctgccagttgggtcagaattggagaatttgctgcagaatcagcgggagggcagggatgggagcagtagcggtgagcccactgctcaggcaagcatctcttccagttcctcctgctctccgtggcctggtgcgccctcacaagaagcggaagaagctgtcttcctcttgtaggaaggccaaggtacggctcctggggactgcggacagccccaggactcctcccaacctgctcttttgtcatcaccagaatgtggaggcgccttgccctagggaggggaagagagggtgccctagggaggggaagagggggcaccctagaaccgggccccaaaaatctggtgtgggataggggtacttttgcagccgcctgcaggccctgcttttctttccccagctgcctttccccatttccttatctgcagcaccttctggtcgtgttggccagttgccggcacggctccctttgtgtctttctcagttgggtgggtgggtgggtggattgtctgtcggcctgattcccccaactaacctgtgactttgcctccttagagagcaaagtcccagaacccactgcgcagcttcaagcacaaaggaaagaaattcagacccacagccaagccctcctgaggttgttgggcctctctggagctgagcacattgtggagcacaggcttacacccttcgtggacaggcgaggctctggtgcttactgcacagcctgaacagacagttctggggccggcagtgctgggccctttagctccttggcacttccaagctggcatcttgccccttgacaacagaataaaaattttagctgccccagtttgtgcctccagcatatgaaaaggactatttgaatccccaaaacatcaggagtcgggaaacttcggaagacagctgtgcctggctctgtggctgcatgcagtgcttcacttggccagcagaggtcagctgtgccgagctgccccagccatgagaagagaagcctgcccttgctggcaggtggctatggccggcccagagccttcctgcccagctcctgcagccctgctgcctgggatcaggctgggagatgggccttcctgaccgccagccttcctctccccgagcacacgcacatgtagattcggggggaagctgcctgctcttccttagaggagccggggcagctatctgctggtccctttctgaacaactgttgatgtgtgagctgtgtctgtgtgttatgtgcataagcggtggtgtgacatacacacatgtgtactgtcccttatgccctggcctgagctctccagctgccttctcagcctgaaggctgggcttctctgctggcttggggtcctagattgcatgtcacctgcttaccaggcgtcacaaggccatgctgggggcatgaggaggttggggcagcaggagagtggggagaaactaggagagtgcctgagtattttagaaagaaccaagttttttctcggcaaaagcttatacagagacgaaggagtctgtgtctttggtcatggtaggactgaagctagcaggacccgagatttggggcctccatgatccctgctcctcttctgttaacacccaaggatttccacgaagccagtgtgtatgatgggggcaggacagtggtactttctgggcaggtgtgaactagagctgctaaggagctgcagacgatattcttgcagtttggtggttagcagtattcagaaggacaaagagttaatggaactggagataaagagcaaccatttgagcatctgctgggagacatctgtcaactgcacagaccctatcagtgggcatcgctgccacctcttggaagacaagacagggcagagagtgcctgcagtgctgaggcctggtccttgccctaggttggccttcccacctggcttcatggagtgctgaggctggtcctggggacagtgagtgctctggatgttttagccaagctgtgttctaaagtgatgcacagtctgtctccactatgtttatctctctgaccctgtcacttccaagcacaccctaccaagagcttgtatcccagagccaccctgatggagaggagattggtttccccagtgatttccttctttggggggtggggtagaggaacatggagccagccttatgctgtattcgtgcctggggatagcagggtctgggcccggagcagaggagcttgggtaaagatatggaggctgtttgttcaaagtgtacattccttcctcctaacggcatccctgggggaagctatttctatgttttaggtggggaagatgaggcttagaagttgcctggtgaatgaggttctttgcaagatttgggttctggcctgtccaccctggtggagtagctggtaccacgggggcttttgctgtggggttaggcaccatgtgggcgctctggggccagggcattggaaagaatgggaggattgcttgagcccagaagttcgaggctgtatgatcacgcaactgcactccatcctgggcaacatactgagacactctctctctttttttttgagacagagtctcactttgttgcccaggctggagtgtggtggtgcaatctcagctcactgcaacctctgcctcctgggttcaagcaattcttcccgcctcaacctcctgagtagctgggattacaggtggccgccaccacgcctggctaagttttttatatttttagtagagacagagtttcaccatgttggtcaggctggtcttgaacttctgacctcaggtgatccacccacctcggcctcccaaagtgctgggattacaggcgtgagccaccatgcctggccgtgagactctatctttaaaaaataaagaacaggaaggtccatcttcgtgtcctgagactacagagagaaagtaagtataaatggctcgttcaacaccccacctgggaggcaggtaccatgtgcccatttacgtgtgaacaaacaggcactcagggttggcctcttggacttagtctggccaaagcctgtgccctttgcacaaatgtgcaaatcaggactggggcaggccttggatgagggtatgtgtgctatgggcaaatgaacctagggctgtccagggccaaacagcacagagggcatgtgggcctggaagggaggaaggaggtgtggcacatgctgcgtggaagcctaaggcttcactaaacagcagagaagcttggatggttttcaggctggtgacgccctgggctgaagcaggaaggtcaggagaatgcagtggcctctccactctgggctggcacagttttgcccacatgtatacctgaatgggtgcctggctgtgtggactgtgctatggtctggaatcagatggacaaggcacagtctatgaggcaaggagcagagatggtcagccaatgcagactgctcaatagtcatgttgggagttcagggtactggagggctataaggggccctcacccagttggagagaatgctgccttccttgagaaagtgaggtttatgctgagatgggaagggtgggagggaacagcaatcctagcaggggagacagcatgtgcaaattccctggggtgggagggatccctgcacatttgagggtgaaaagaccagagggttgttaccaaaatatgcaatgggggctgaaaatttgattttttaaaaaaatgtaatagtcacatattaaaaattcaaaggatacagaagatggagggtttttgaaaacaagggcattctttttttttttctttcttttttttttttttttttttgagatggagtcttgttctgtcacccaggctagagtgcagtggcgccatctcggcccagtacaacctccgcctcctgggttcaagcgattctcccgcctcagcctcctgagtagctgggattacaggcacccaccatcatgcctggctaatttttgtatctttgtggagatgggatttcaccatgttggccaggatgaactcctgacctcgggtaatccacccgcctcggcctcccaaagttctgggattacaggcgtgagccaccacacccggccaacagcattctgatgatatagctggtgatagcagatgggaggacagtggtgtgcccagaaacctctggacctggagcacaaaaggctcagggtgcattcccagcccagcggattctctgcctgcatctgctaaggatgaatgactcagctttgggggaattagttgtgagactttggacttcaggggcggggccccaagaggcaatggtgataaagttgaatttgagcagggaaggtgccccgtcagctgtcatccttttccccaggaacatcattatgtaagactcctgccttgtggaacaggctgtgagttgctgctcttccattcctcacagccatgtttacaagggtaaggaagggaaggagtggttcatcattgcagagaggaaggtgccttggccaagcagacctgctctgtgccaggcatgacactgggcaaatgcacagtatttagtttatttatcctgaaatgctttcagaactcaatgcatccagtgtcttgttatccctccagcctatccgcaaacctgctgagatgcagtaggtttggcgtagaatgcactgagggtatctgtggcaacagtgggctaaagaacaaggcacatcaagggggtcttccgacgaacaccccaagggttgcctccaccacgcagcatcctgctgtggctcgcctcaatgtcccaggtgctctgtgggcacagtggccaggtcagaccatgatggccactttctgactgtatggtctcatacagggaaggcatgtcacttttcttggccttctctaggttctcacctgtaagtgggggtaaaatgtcccctccaagggttcctgtaggggccgaagtggctcaggcacctggcacgcctgttgcagcccagctctgtgctagcacctcccaatgcctgttgaatccaccattgccttctgggacgtgtctccatacttccacgagaatacgtctagggcacagcctggggttctgcatttgagttctcacctccgtcccaggtgagcccaaaggtgctggtctctagtccacatttgagaggcaaggctttaatatccaccacacacatttattttgcagattggcaaaagcttagattaactagccagcccaatgtcacttggctaagtggtagaggggaggtgctgtgtctgtcagactctagtctggaattggaggtgggatacctaggttcaagtcctggatatgaaacttccctgtcacatcccttctctgagcccaacacttaatccgaaagtcatggtgacgtgggaggccaggtaaagtaggagatgtctagctagattggaatttcaaataatgaggaattttacagcataaccgtgtcctgtccaatatttgggacatatgctaaaacagatattcactgtttctctgaaattccagtatagctgggcatcctgctttttatttgctaaatgtgcaaccctaggttgtgagacctctctgacccacgcgtggcctcctccaggaagtcttcctgagcgccagcctgggctgggcatcctcctctgtgtgtctgatgctgctctgacctcatgaagctctaattgctggggcctgtccccaccttgatgtgggagctcttcggaggcaaggaccatgccaagttgcatatctgtgtgttcctgagtccagtggttcattaaaagcttttccctgagagtatccttaatgccccggaggtaattctcttttcacaacctttctactgcctgaggctcttgaggactaattccagttaaaagcagagggaaggatgtggtaggaatagcaccgcatggagctggactctgtgccccccgtgcagcaggcaggaccctccctctgtgtcacctccatgactcagggctcagacaggaagccctcatcctcgtcctccacgggtctcatgtttgtcaaggccaggggtatcaggcgtagtaggcaggggcccatgcctgctcccatctgggagaccctgcttcaggcccctctcatggccccttttgttccccacagctcactgtacaacttgttggttcaaggttagaaaatgcagttttgtgttgagggggaccatcacagaagacaaagggtccaggatgaaggctacccatcagcttgcagggctgggacgaatgtgagaagcaactgatgcttgtacagtagagagtaagcatgtagggccagtccccagaccttgcctcccctcagccttgacatgtgatctccatttctggtggctacccctgctagtggtctggcttcaccatcttagccctgcccaggctaagaccctcttccatcagaacctgcagctgggatgctgggagcaacagtcagggcagagctgcccatcctcccattcagggagccctcaggaagtacttgggaccccccgaccctttatagattcagcctgcctcatcccctccatggaccaacacgcccttctcctcagcagtgggctgggggaccaggctcctgaactgcttgtggctgttccagcagtggggagatggagggtcacacagtcctgagtctatggctttgacagcaacgggtcctgactgcagctgtattcgtgaagcgaagtacctaatacaatcaccgaaatgtacaaattggacccctataggttcaaggattcttggtgtaggaggtatggcccccgccccgggaaccaggacctcagcttttagaagcaaaatgcatgaatgcagtgatggttaggccaagtgccaagggagacagccaacccctgctatcatggccagaggagggagagaccactcaggcctgggtggtggtagaaatcctcactgccaaggagattgtatgcgcaggcgttgggaaggcccggagaagcctgagagacaggcttggcttggttatagcagagctgggtggagggagcagattagttggcttagcacaggcttcctgcagggtggtggttcttggccagatttgccccagtggggcttttgggaaagtatagaaatattgttgtttttttttttttgagacagagtcttgctccgttgcccaggctggagtgcagtggtgtgttcttggctcactacaacctccatccacctcccgggttcaagtgattctcctgcctcagcctgccaagtagttgggactacaggcatgtgccaccacacccagctaatctttgtatttttaggagatggggtttcaccatgttggccaggctggtcatgaactcctaacctcaagtgatctgcccgcctcagcctcccaaagtgctgggattacaggtgtgagccactgtgccagccaagaaacattttaggttatcatatctgggcaatggatgctactggctttaggtgggaagaggccggggatactgttaaaatcatgcaatgtacaaggcagcccccacaagagagttctgtggttgaaaatgtccgtagtgttgaggttgaggactctgctgtggggcaacagtaggagaaggggtgctaatagtcaggtggtggacagcagggaattacaggtacatcagttaggagtgtatacagctgcaagtaagagaccaccagatggcaggaacagtgggaacagaatggtttatctttttcatgtgtcaagataagtggtgctaaagtcaggcatggtggccctcacttataatcccagcaattcaggaggctgaagagtgaggatcacttgaggccaggagttcaagaccagcctgggcaacacagtgaaatgccatctctaaaaataaaaattaaaaaaattagccaggggctgggtgcagtggctcacgcctgtaatcccagcactttgggaggctaaggcgggtggatcacctgaggtcaggagttcgagaccagcctggccaacatggtgaaaccctgtctctactaaaaatataaaattagctgggcatggtggcacacctgtaattctacttactcgggaggctgaggcaagggaatcacttagaactggggaggcgtaagttgcagtgagctgagtcacgccattgcactccagcctgggcgacagatcaagaccctgtcaaggaaaggaaaggagaggggaggggaggggaggggggaggggggaggggggaggagaaggagggagggggaagattagctaggcatggtgatgagcacctgtagtccctcctagctactccgaaggctacggtgggagaactgcttgagcctgggaggtcaaggctgcagttagtgatgatcgtgtcactgcactccagcctgggtgaaaaagtgagaccctgtctcaaaaagaaaaaagacagataggaagaaagaaagaagaaaagaaagaaaaagagagagagagagagagggagggagggagggaaaggaaaggaaggaaggaaatgcatctgatttttgtgtattgattttgtatcctataattttgccaagttcatttattagttctagtaattttttttattaaaaaaaattttcaagatagggtctcaccctgttgtctaggctggagtgcagtggcacggttatagctcactgcagtctccattgccaggactcaaacagtcctcctgcctcagcctcctgaatagctgggactacaggcatgccagcatgcctggctaattattttattttttgtagagatggggtctcacgttgttgcccaggctggtcttaaactcctgggctcaagtgattgtcctgcctcagtctcccaaagtgctgggattataggcatgctccaccacactcagacaagttataatacattttcagtggcgtatttactgttttagaatataaaatctatctgcaaatagagataagtttaatttttttctaatttggacactcttttccttcctccctccccttcccctttcccttccccttcccttttccctccctccctctctccctctctttctctctctctctttctctcttttctcttttcttttcatttcatttttgccaaattgctctggttagaacttttaacactatgttgaatagaagtggtgacagtatctcattcctagacacttttcgaaagaagacatacatgcaaccaacaaacatttgaaaaaaaactcaatatcactgatcattaaagaaacagaaatcaaaaccacaatgagataccatctcacatcagccagaatggctattattaaaaagtaaaaaaaagaaaaaataacatgctggcaaggtcatagagaaaagggaacacttgtacagtgttggtgggagtgtaaattaggtcaaccattgtggaaagcagcgtggcaattcctcagagacctaaaggcagaactaccattcgacccagcaatcccattactgggtatatacccaaaggaatgtaagttgttctgccataaggacacatgcacacgtctgttcattgcagcactattcacaatagtaaagacatggaatcaacctaaatgcccatcaatgacagattggataaagaaaatgtacatatatgtcatggaatactattcagacataaaaaaagacatgtgattatgtcctttgcaggaacatggatggagctggaggccattatccttagcaaactaacgcaggaacagaaaacaaaataccacatattctcacttatattctctcactaaatgatgagaactcataggcactaagaggggaacgacagatgctggaacccagtggagggtgggaggagggagaggagcaggaaaaataactattgggtactagacttagtacctgggcgagaaaataatctgtacaacaaacccccgtgacacaagtttacctatataacaaacctgcatatgtacaacttaatgtaaaataaaagttaaaaaacaaggccaggcatggtagctcatgcctgtaatcccagccttttgggaggccgaggtgggcggatcacttgaggtcaggagtttgagaccagcctgcccaacttggtgaaaccctgtctctaccaaaaaaggaaaaaaaaaaaaaaagccaggtgtggtggtccatgcctgtaatcccagctactcaggaggctgaggcaggagaattgcttgaacttgggaggcagagtttgcagtaggctgagatcgctccactgcactccagtttgggtgacaaagcgagactctgtctcaaaaaaacaaaaaacaaagttaaaaaacaaaacatcggacaccacacaccacatggcaggatccaggatccaatcagatcaagctctggcatcaccccacggcaggatccagtcagatattaccttccagcatcacctcattgtgagatccaattagatcatgcctcattattaccctgtgcttataaaacccaacccaacccctagctcaggaaaagagattgagcattccctccttccttgccagttgactttaaataaagcttttcttatctcaaaatataaaaaagaaagtatctcccctgggcatggtgggctcgtgccggtaatcccagcactctgagaggcagaagtgggcagatcaactgaggtcaggagttcaagaccagcctggccaacatagcaaaaccctgtctctactaaaaatacaaaaattagccaggtgtggtgcctggctaatttccacgcccggctaatttttgcatttttagtagagacggggttttgccatgttggttaggctggccttgaacttctgaccttgtgatccacccacctcagcttcccaaagtgctagcattacaggcatgagccaccacccccagccctcttctgcctgatcttagaggaaaaaccttcagtctttcatcattaaaaaaaaaaattatttttcgagacagagtcttcctctgttttccaggctggagtgcagtgatgtaatcgtggttcacagcagcctcaaactcctgggctcaagtgatcctcctgtctgagcctcctgagtaactaggactacaggcatgcaccactacaccaagattttttttggtagggtcttgctttgaccttcctttgaccttgctttgacccttgatttgaccttgctttgacagtgtcttgtaatgttgcccaggcttctcttgaactcctgggctccagtgattctaccacattggcctcccaaagcagtgggattatgagcatgaatcattgagcctgccagccttctgtcactgaggatgatataaactgtggggttttttggttgtttttgtttttgagacgaaatctcactctgtcgcccaggctggagtgcaatggcacaatctcagctgactgcaacctctgcctcctgagttcaagtgattctcctgtctcagcctcccgagtagatgggattacaggcgtgtgcaaccacgcctggctaattttttgtatttttagtagagatggggtatcaccatgttggccaggctggtctcttaactcctgacctcaagtgatctacccgcctcagcctcccaaagtgctgagattacaggcatgagccaccacacctggacattttttttcatacatggcctttatcatgttgagagagttacctgtattccttgttttctgagtggttttattatgaaaggatgtcggatattgtcagatgtcttttctgcatcggttgagagaatcatgtgattttttcccttcatcctgttaatctggtatagttcattaattgatttccatatgttgaaccatccttatattccaggaataaagtctacctggtcatgatgtatactctttttttgttttgtttttttttggagagggagtcttgctctgtggcccaggctggagtccagttgcatgatctcagctcattgcaacctctgcctcccaggtccaagtgattcttctgcctcagcctcctaagtagctgggactacaggcatgtaccaccacagccggctagtttttgtatttttagtagagacgaggtttcaccatgttggccaggctggtctcgaactcctgacctcaagtgatctgcctgcctcggcctcccaaagtactgggattacaggcttgagccactgcgcctggccaatgtgtataatctttttaatacgatgttcagcttggttcgctagtacttttactcagtattcatgtatattttattcaatatttatgagatctgtagttttcttgtagtgcctttggttttgatatcagtataccataggatcaggatactatgaacatgccctcatagaataagttaggaagtgttctttcctcttcaatttagggaagaatttgaggaggattgatattatttctttttctttttctttttctttcttttttttttttgagatggagttttgctcttgttgcccaggctggagtgcaatagcgtgatcttggctcacagcaacctctgccaactgggttcaagcgattctcctgccccagcttcctgagtagctgggattacagacatgtgccaccatgcccagctaattttgtatttttagtagagacggggtttctccatgttggtcaggctggtctcaaattccgacctcaggtgatccgcctgcctcagcctcctaaagtgctgggattacaggcgttgagccaccatgcccagctgatattaattctgctttaaatgtttgctagaattcgccagtgaagccatctgatcctgggcttttcttttgggggagtttaaaaattactgattcaatttccttactagttatatgtctatttagattttctgcttcttcatgaatcagttttggtatgcaatgtctagcaatttgtccatttcttctagattatcgtttgttatacagtcatttatagtattatattgtattttttatttctgtaaaattgtaaagttcccactttcatttgtgattttggtaatatgagtcttctctttttcttagtcaccttacctaaaggtttgtcaattttgttgatttttttcttttaaaatttatttttctgtattattttatttgtattatagattgctccttcagaatatttttttcaagaaatcaactttttgtttcatagctgttctctatagttttctattccctatttcacttatctcagttgtagtctttattatttttaaaaattctagctttgagtttagtttttctttttctagctccttaaggtgtgcagttagaaaatttcaaatctttcttcttctcttctcctccccctcctccttcttcctcctcgcccttcctcctcctccccctcctcctgttggggtgatcagacccaacaccaggtcgtgggggtgacaaagtccggtggagtcaaaggattgagacaaagacagtttgagagataaaggtgggacaccaaggggccatcgtgatcatggaggctgcgaaagccctgcgctctgggagtccacagtatttattggtaatccaacaaagaaacaggtggtgaggcatgttctcactcataggtgggaattgaacaatgagaacacttggacacaggaaggggaacatcacacaccggggcccgttgtggcgtgaggggagggatagcattaggagatatacttaatgtaaatgacgacttaatgggtgcagcacaccaacatggcacatgtatacatatgtaacaaacctgcacgttgtgcacatgtaccctagaacttaaagtattaaaaaaaaaaaaaagaaacaggtggtgagaatgtggaggtcaaaagggcaggcgcatgatctacagctgtgacagtttagcatttatatggaacatgttctgctacttgagataatgggaataggagcctaggagggctagaagcaaggagccagcaagtctagacacattccagaggacattatgcaagtcctgcctcagtttccctcccaacactcagctttttcccaacatcctcctcctccttcttctttttttctttctcttcctcttcctttctttcctccttctctcttttttgtagagatggggttttgctatgttaatccaggttggtcttgaactcctggcctaatatgattctcctgccatggactcccaaagtgttgagattacaggcatgagccaccacacctggccctttctttaaaaaaattttttttttttaatttttaaaaatttttttgagacaaggtcttgctctgctgcccaagctggagtacagtgctgtaatctcagctcaccgtagcctcgacctcttgggctcaagtgattctcatccctcagccttccaagttactgggactacaggcacgtgccaccatgcctggcgaatttttcctatctttcttgtagagacagggtttagccatgttgcccgagctggtctccctcaatcctgcccccttggcctcccaaagcactgggactacaggcatgatccaccgcgccagggtgctttcttcttttttgattgtgtttatggctaaaaattttcctcttagcacagctttgctgcatcccataagttttggtatgttgtgttttcattttcatttgtctcaaggtatttttatatttcctttgtgatttttgctttgatccattggttgttaagcatgtgttgtttaatttccaaatatcatgaattttcagggtttttttcctgtaatttatttactttttttttttttgagccaggatggagtgcagtggtgtgatcatggctcactgaagcactgatctcctgggttcaagtagttctctcgcctcagccttctgagtagctggtaccataggtgtgtgccaccatgcctggctaatttttgttttgaaacagggtctcactctgtggcccaggctggagtgcagtggtgcgatcatggctcactgcagccttgatctcctaggttcaggtgatcctcccacctcagcctcctgggaagctgggactacaggtgcacaccactacaccagctaattttttgtatttttagtagggatgggatgtcaccatgttgcccaggaagttctgaactcttgggctcaagcagttcatttccctcagcctcccaaagtattgggattacaggtgtgagccaccacacccaacttatttttatttttagagatggggtttctctatgtttcccaggctgatcttgaacccctgggcccaagagatcctcccaacttctcctcccaaagtgctgtgattacaggtgtgagtcaccgtgctcagccccttctattatcgagttctagtttcattccattgtgactggaaaatatactttctatgattttaattatttaaaatataacaaggcttgttttgtcgcctaacgtactgtctgtcctgaggaatattccatatgcacttgaaagaaatgtgtatcctgctgttatggagtggaatgttgtatatatacaagtgtccaagtgttttataaatgttcaagacttctatttccttactggtcttgtggctagttgttccatcaattattgaaaatggagtattgaagtctccaactacttattgttgcattgtctatttctcctttcaatgatgtaatgtttgctttacatattttaaggtcacattgtttggtgcatatatattattacttattcttgatgaattgacccttttagtaatgtataatgtcatttttgtcttttgtaacaattctttatttaaattctattttgtggtcaggtgcaatggctcatgcctgtaatcccagcactttgggaagctgaggtgggcagatcacttgaggtcaggagttcaagaccagtctgtccaacatggcaaaaccccgtctctactaaaaatacaaaaaattagctgggtgtggtgggacacgcctgtaatcccagctgcttgggaggctgaggcacaagaatagcttgaacccgggagacagaggttgcagtgagccaagattgtggcactgcactccagcctggacaacagtgagaccctgtctccaaaaataaataaaataaaaattctattttgtcagatattagtgtagcaactccagctctcttttggtgactatttgcgtggaatatctttttctattcttttattttcaaactatttgtgtcctcagatctaaagtgagtgtcttagacatcatatagttggatcctatctctaaaacaatgtattctgcattctccaactttgactacagagttgaatccatttaaatttgaagtaattactgataaggatttatgccattttaccttttcttttctgtatgtctcatagatttttgtctttcattttcttcattattgacttctgtatttatttatttatttgctttttgttttattttattaattttttgtagagacagaatctcactatgttgcccaggctggtcttgaactcctggcctcaaatgatcctcctgcctcagcctcccaaagtgctgggattatagacatgagtcaccttgcttggctgggttttaaaaattgtttttgtagtgacacattttgattctcttttcatttccttttgcatatattctatgtattattcttcgttgttaccctggggattacaaataaaatcctagagttataaaaatctaatttgaattgataccaacttaacagcatacaaaactctactcctatacagctttgtccctgctttaggttattggtgtcaaaaattccatctttacacattgtttgctcaaaaatatagaattatgtttttttggctgggtgtggtggttcacacatgcaatctcagtgctttggaaggttgaggtgggaggattgctcgaggccaggagtttgagaccagcctgggcaacataacaagatcccagctttacaaaaaagggaaaaagaaagagtgacttggcaggcatggtggcttagacctgtaatgccagtactttgaaagtctgaggtgggagaattgcttgcctccaggagtttgggaccagcctgggcaacacagtgagaccccacctctacaaaaaatacaagattttgccaagcgtggtggcatgtgcctgtaatgggattccagctatttgggaggctgaaatgggaggatcagttgagcccagaggtcgaggctgcagtgagctgtgattgcactaatgcactccagtctgtctcaaaacaaacaaaaaacaccccaaaaaaaccccaaagttaaaataattctggcttttatatttacctatgtaaatacctttattgaggatttttatttcttcaaacatctttgagttactgtctagcatcctttaatttcaacctgaaagagtccccttagcatttcttataaggcgggtctagtggtaatgaactctctcagctattatgtatctgaaaatgtcttaatttctcacttatttttgaaggatagttttgctgaaataggatttttggttgataattttgtttcagcgctttaaatatatcatgctcactgccttttgacctccaatgtttcttatgagtaatcagctataatcagctgataatcttattgaggacaccttgtatgtgatgagtcacttttctcttgttttcaatattctctcttagtatttgcctttcaactgtttgattataatatggctcaatataagtctctttgtatttatattccttggcgtttattggacttttcagatatttaatattcatgtctttcatcaaatttggaaagttttaggccattatttcttcaaataatctgtctcattctccctttcttctccttattgaactcccataacacccacgttattttgtttcatggtgtaccataagtagcagtctctgttcacttttcctcactctttttcatgtctgttcctcagacctgatgatttcaattgtcctaccttcaggttcacagattctttcttctgctttctcaaatctgctcttgagcccctctagtgaagttttttatttcagttattatccttttcaggtccagaatttctgtgtggttcttttttataatttctctttattgatatcctcattttgttcatgcatagttttcctaatttactttagtcctcatccatttttgctcttagctctttaagatagctattttaaagttttttgtctaataagtgtaatgttgggctgccttggacacagtttttgtcaacttttttttttttttcctttgaataggccatcttttcccatttgtctgacttgtgattttgctgttgctgttgaaaactggacatttgactattataatgtgataagtctggaaatcagattctctctcttcctcagcatttttttttaatttctgaagactgtagtaatgtttgtttttatactttcccaagctatttttgcaaagactattcattgtttttttgtggtcaccaaagtgtctgtttcttcagcttgtgtttagccagtgttttgacagagatttccttgaatgccaggagctaaaaaacaacaccaacacacacacacacacacacacacacacacacacgtacacacacaagcatacctctcctatcttttgcaaattggggttgggactcttttaacacttagctaggcttgttctgagcctaggatcagcctgcgacaaaagtttcagggcttttctgaacatgtgttttgccttgtacatgcatgcggcattctcaatttcctgtatacatagccgttttatttttgtttgagttggatctcactctgtcgtctaggctggtgtgcagtgacatgatcatggctcactgcagccttgaactcctgggctcaggtgatcttcttgtttctgtttctcgagtggctgggactacaggaatgcaccaccatgcccagctaagtttcccttcccttcccttttctcctctccccttcctttcccttcctcctttcttttctcttttcttttctctctctttctttccttttctttcttctttctttctttcctttctttttctttctttctttctttctttctttctctctctctcctccctcccttccttccttctttccttccttcctttccttttttcttttgcttttttctttcctcttctttcttcttttctttctttctttctttctttctttctttctttctttctttctttctttctttctttctttctttctttctctttctctctctctgtctcctccctgccaccctcccttccttccttcctttccttttttcttttgcttttttctttcctcttctttctttctttttctttttctctttctttttctctctctctttccttctctccctccctccttcccttccccctccctccccctctcctcccctcccctccccatcctgtccttgtgtgaacatagctcacagcagccttaaccttgagggctcaagtgatcttcctgtgtctcttccaagtagctgggacagcaggtgcctaacctccgtctaattatttatttttttctgctcatcctctgtgggttggacccactgccgaaccagtcccaatgagatgaactgggtacctcagttggaaatgcagaaatcacccaccttctgcactggtctcactggaagctgcagatgggaactgttcctattcggccatcttggccccttccaattatttatttttttgtagagacagggtctcatcatgtttcccaggctggtttcaaactcctgggatcagggcaggatcttcccacctcaacctcccaaattgctgagattacaggtgtgagccaccatgcccagcctgcttttctattttgttgtagagacaggctctcactaccttggccaggcttgtctcaaactcctggcctcaagcagtcctcttgccttggtcttccaaactgctgtgattacaggcatgagccactgcacctggcggcttcttcttcttctttttttttttcttttgagtcaatgtccagcctggagtgcaatggtgcggtatggcttactgcagcctcaaacccctaaactcagatgatcctcccacctcagcctcccaaatagctgggactacaggtacatgccaccatgccagctaactttttttacattttattttttgtagagatgggggtcttgcaattattgcccaggctggtctcaaactcctggcctcaagtgatcctcccaccttggcctccaaaagcattgggattacaggcatgagccactgtgcttggctcaaagctgctttaaaaatttatgtacatatatatattttaagacagagacttgctctactgcactggctgtagtgcagtggcacaatcatggctcactgcagtctcaaacttctgggctaaagcaatcctcccgcttcagcctcccaagtagctgggactacagttgcatgccaccacccccagctaatttttaaattttttgtagagacagggtcttgctatgttgtccagactggtctcaaactcctgggctcaagcaatctgcctgcttcagcatccgcaagtgttggggttacagatgtaagccactgcgcccacgagttgctgctgaatatccaaattgtctaagcttctcctctgggtttaaaatggtctatggcatgtctctacctataacctcttgccccaggcatcttttctgagcaatgtcctgattttaggtaagagatacagcatcttgcatcagttcttccaggatcccccagacaagaacagatgcacgtaatagtttgcaaataaggcctgctctctttggaggagggagctgagaactgtactactgttgtctcaattccaaaactgttgactgagtgcagtggctcacgcctgtaatcccaacactttgggaggccaaggcaggaggatcacttgaggccaggagtttgagaccagcccagacaacatagtgagaccctatctctacaaacaatttaaaacactagctgggtgtggtggcacatacatgtaattctagcttctcaggagacggaggttggaggattgcttgagcccaggagtttgaggctgcagtaagccatgattgtaccaatacattccagcctgggctacagaatgagaccctgcttcaaaaagaaaaaaaaaaaaaaaagaccaagactgctgccatgctggggaaggggtggggcaagactaagtaaaaacaccacaaaactttgctactgttttgaagatggccttttttaaattgagtgtttgcctggttgctgtaggcctttgttttctagagtgacaacaaagttggttctgacagtttggcttgtttattcagtgtttcagtttggaaatgagagcttggagcttcctaggccaccattttgctgatgtcatttccaatggcattttttgcatctcgactttttcctcgcgttcaatgcttcaggaccacaagatggttgctacagctctagaccttccatctgtctagtgtggcgaaaagtggggaaggctagaatatcatgccagctgcatacctcccctttcatgagggaagaaaaagccttcccacggggatcacagggcccctgctagctgcaaaggggtctgggagaacagggagagcctctctcacctgagcagtggacacaatccttcaccaaagagtgcaggttctgatggcaagaaagacaaaggggccaccggcaggctcgttaccccaaagagcgagaagtaggggatgtgattacttacatctgtaccagttagagtgttgtacacatatccagccaaggtacctgtggcccaggtcaggtgactggcttagcaatttcacctaccttcctctcagcccagatccccaaattctttgaatgctgttgggatgcagaacagcaagtcagcgagtgattttttttaatttaatttttatgagtacacagtagattatatatttatggggtacatcagatattttgatacagatatacaatgtgtcataatcacatcaggttgtaaatggagtgaccgtcacctcaagcatttgtcacttctctgttacaaacattttaattacacccttttagttattttaaaatgtactgctgattgtaattaccctgttatgctatcaaataccagctcttattcattctatctaattatatttttgtacccaccaaccatctccgcttccccctacctccccactactcttcccagcctctggtaaccatcgttctacctactgtctattgccatgcgtttgttttcatttttagctcctataaatgagtgaaaacacatgaagtttgtctttctgtgcctggcttatttcagtgagtgatcctcatgtctccagggcttgtctgtacatgactcacctggggcagcctctgccaggtgtcaccccggagccagcaacaaagggctgctctgctgatggctgcctcacccccggctgctccctcagtgaactggcacagctctgggcccctctcgggaccttctcagagtagccacatttcagacctgtcttatgattctaacatcaaacttataatatcaatcttactaataccaatagaaagtggaaaatgaggtattatctggcagtcattaaattagtaagttctaatgacaaacataatacacgatgaaggtgagactgtgggaagatggtgcctttgcgagttgcccatgtcagtggtaagagtcacggccctcgggaaatcaccgagtcttcattacccaagactggcatcaacccttcaccaaattccaataactgagaatctgataattacccaataaatcctagattagcctgaggaaagaaatgagctgtccacgtaagagtcgtaaacattgggccggggctggtggctcatgcctgtaatcccagcactttgggaggccgaggcaggcggatcacgagatcaggagatcgagaccatcctggctaacatggtcaaaccctgtctctactaaaaatacaaaaatgaaccaggcatggtggcacatgcctgtagtcccagctactcgggaggctgaggcaggagaatcatttgaacccaagaggcagaagttgcagtgagccgagattgcgccactgcactccagcctggcaacagagtgagactctgtctcaaaaaaaaaaaaaaaaaaaaaagaatggtaaacattgtactctgactcacaaatctcatctaggggaacttgttttaaggaaataaattcaaagaaggaggaaacattgttaggtgcaaagaagtcaaccagaaacttatttatcaaaaatgaactattgggaaccggctcgacagtcagcaccagaagaggagaagatccacgcgttctgtggaccataacctagtcacggacgtgctgatcagagattgaaggcaacagggaggatttatgtgaaaagtcaagagaaaaagcaggatgcatgtacatatcatatggttacagctcggcacgtgtgtccagaggcaccggcagctgggttgggagatcgggtgtgaaattttcactgtcattccgagccggattgtgccgctgttatgctgcgtgtgtttcacaaatgaccccaggagaccacatagctggactctatctctctgtggtgctagactgggcacagctgggctccaggggcttagcctagacagcccccatgggaagaaacatatgaaaggcagggtgggcctttcatatctttgttctgacacagctctgtgcatgccgacagtgtcttcttgtcgcaagtgcccacggccctgcctaaggccctttgacactgaaggtgcccgccacgtgctggggcgaaatcttccaggaatgtcctctaccagtgacagatgaatgtggtggaaagctgtctgtgtccttattccttggaggggaccttcttgggcacgtccccaccagttcccggaggtccctgggggcaggagcaagctcttggatgcattctggtcagctttcttccatcccctggctcattccccattcaccgactgctgtcatctggggtcatctccccaataaactctttgcactgggatccttgtttcaggatctgtttctggaggaactagatgacaacaccgggaacagaggacctagagaggcagcttcatgggtggtggggtgtccgcctctgccggccagggacttgggagcagtgctgggaaggtgctggatggagctgtcactcacaggggcaggtccttggctgctgactgtcttcctctccactatggctgtcttgagaacttaggggtcagcctgaccctgccttggcccccttcctctcagcctctgtcttctcctgcatgaggctgggtggctcccctgtgaatcaggcaggggtccacagaacactagagacaggtcccttcctgcagctgtctccagtaggtggccacgcaggagatgttcccaacaagctgcccttatctgcagctcagctttggtaatgggggcccattaccaaatgggggtaaaggtcatggcccatcctggtgatagtgagaacccaaggtaggccttgaagattcctatcaggagggagcagaaagtgtgtaccacacccctgggcccaggtggagcagggctgctgctcaaggctcccagccatgctctgtcccttgctaggggtgaccggtgggacaggcctgggcaagggacaagagggagaaggtcggggggaagaggggatgaagagcaaagtgagcaaaggagagtcttccactatctggggtctctgtcaactgtcaggccctagagtgagctgttctttccctttgcttcctggaggaggggacttttgtcactgcgtcactccaccctgcctgcccctccgttatcaggctgttaatattaattaacaacagttgctagggatgacagtgcagagggttcctctgagcccattgctggccctggtcccaagagggggtagggcagagctggggtctgaggctgagccagggagggtgcggaggttcctcggccatgctgagctcctgaggccgggtcccagccagtgcctggtcccatctgtgcctccaggccctggcaccaactccagcagtgttaggggctaatagcgtggtctctcccctagctgactcagccctctggcttcggtcgctttgggaagtgagtggagaccctagcacctgcgtgatgaggctcatctaaagcgggggcctgtggactggggccaaacagtgggagtggtggatcattaaccagcagggctcagcctcattggtccctaacccagtcaggccagggttgtcatcgaaggggaggaggctgccttaatgtgtgttcagcccttggctgttcctgaggcctggcctggctccccgctgaccccttcccagacctgggatggcggaggccggcctgaggggctggctgctgtgggccctgctcctgcgcttggtgagtcccagggcttggctccacctcccctgcggcctccagttagggaccctggggccagccgtgtaccaggcgagcgttactgggtgacagcaagggagcctcagggcctgcgggctgggcaagtctctggacacatgagggatgccaggccccacagaggaggggtgcaggtggagggtttccaggttacaggcttgaatgcacacaggggtgaaagaggctgctggactggggtgctccaagtccctcctgtcactggccctactgtggggtccaggcctgcagttgagggaggtctgaggcaaggaggtgctgggatggggttacctggtgagcatcacctagggaggactgagcactctggaggctgggagaagatccagcgctggcacctcttaagttcctcgcttactttgtgtctgggaggtgggtgacagcttttggcctcaagcaggtggtggtagtggtggtgggagtcggggggcctcctgaacagactctccatgagagaccctggcctctggatgtggtgtacagtgtggggactcaggctgactttgacgtgggcagagcccgggaccttggagtcagctttgcctccttacccatctctggcctctccagcatgactttcctaagctgcaggtctatcaggccacccccaggaagaaaggccagtgttgtcactccaacactggctggctggcacatgcctccaggaggcttcctactccccacactccccgcttccctgcccctgctccatgtccttcttaccctcacaccctccctggctgcctgctgcctggatggcacccagctgtgtcagggcccacgcgtgatgttgctgtgctctgcaggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctcgactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaagtgagtccatggtggggcccaagcgaggagtgggctggggctggggctgggctgccatggcctcctgggaacctggccgggcatacagctggtcctgaaggaccagaggtagctattcctacggctctggcctggggccgcccagatgattatctctgcccctcgtccggccgccatttcctttggtcagagttcctgctcatggctgcaggtttgtgcgtggccatcgctggcccttcaaccccgagtccactctgtctttctgcagatttcttgacatgtgggagctccctgccacactcttgctttaagtctgacagaggagcccgattggcagagtacatatttatatttgctatgttttgcttcttgtttctgtgccaggggccgtagggccatcagtaacccatgaggtaccatggtatgcattggaaaaggtgccctcaggccagaggtcgtggctggtctcaggcacctgggccgggtgtcctggggtaggccacagccacacacacttctattgattggggttcggtctttggttctgtccactctggtgtgctgccaacaagatgccaacaacgctgctgggccaagggggccaagagccaagggcagcagcagggccttggcagtggaggctccttgaggttggagtagagcagaggtcctcaagatgaacgtttagtactccatactccagagcaaatgagagttaaaaggggcaaatagcatcttagtgttattatgaaaacagttctgaccttacagaccctggaaagggtctccaggacgcctaagggccccaggccacactttgagaaccactggattggaagagagtgccgacactttctgtcccctgctacctggctctgcatccctcagctgggccccaagtttgggctgcttcccagagtgtctgtgccaggaacccaagggctctctcttggaaatagcaggaacgagaggagccattgtttgctctggggaggcatcatggtctgacctcagactcatgtctgacggtagctttatagtccattatagggtattatctttattttgacttcggatgctcacaacaactctcgggtggtccaattatctccattttacagacaggaaaactgaggttcagaggggtgtggtaagctgctcaaggtcacacagcaaccagcactcgcttgctgagatctgagagaggggggtagagagctttgctcaggtgtcccactgcatcttcgcaatgacgggctttgcagaaagggctaagctgaaggacctacagacttgcctgagggcaccagtctagtaaactgtgaaaacattggctgctgggctccagggttccaaatctaacctcaatacctaaagggtttcgggggccctaggcaggagaaggaggctgagagggcaacgtttgagacagcccatgccagaccccatggctcaaatcccagctcttccaccctcacgggacttcaggtgtgacgctcaatccagagtcagataatgtcagagccaggaaggtcaggccagtgtgtggagacatgagaggctcagagggacaggtcccggagcagcccctgcctgccacagagaaggcactcagggcagctccaactcactccgtgggtgggggcctgcaggagatcttgctggatgggagccatttaggacccactcggctgggtcctaaatagctaaatggcctaaatgcagatagctgggctatctgcagccagtgtcccccaccccaccagctcaccctccatagtgctgtgggtctggggtgggaggggaagggaggggccatagggactgggcagggccaggaaaggccctttccctttgcggtcatctccctctagtgccccgctcaccttcaaggatggcacagccctggccctgagctgcatggctgactacggggcccctgtcttccccttccttgccattggggggtacaaaggtaagctaagtgggccctgagaggaagccaaggaagatgcagtattggggcaggaaccatagacgggagggtgggagtggtgctggggattctcgcggcctgggggtagcctggcttctggaagctgtaggccaaccctgtcctgtttcctctctctgccatctcctttatcttctagtagtgttactcaggcactgtggtttttctgcctgggcccaaaggtctcgcctttggctgagagaagtggggtgtaggaggtaaggccatgtatcagatgaggaaggagtgggggagaaggagcaaggggtgatgggaggggtgcagctagatagggggagggaatataggggtgcagctggagggggagggaggcacgggtgcagcaggaagggtctgagtatttcttatcccaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcatggctgaggtaggggctgcagggtccctggctctgggggtgcaacccaggtggtcttgggtcagttcctgtgtccccatcctggccctggcccttcctaagtgaccctgggcagtggctgcctgctcagaacggggtgattgtgatggctgttcttatagcctcacctgcgattatagggggccatcaggccctatgacacaacacacaattagtgcccagtgaccgagctattgagagctggcctggctgaagcaggcacggtcagtgggggctggtcgggtgtgtgtccacagcgctctctggaagacgagatcaatcgcaccacagctgaagacctgcccatctttgccaccagctacattgtcatattcctgtacatctctctggccctgggcagctattccagctggagccgagtgatggtgagaagcgggagggacacagctaagtgggctagcccaggaccccaggcatcttcagtaggccttctacaactttcctaaccacagcacctcagaacagcaaagtggacacacccaagtggctgccccaaagggtaatacctcttgcaagtgttctgtgctgaaaggtcaagagcaattttcttttcttttcctttctttttcttctcttttctttgcttttcttttctcttctcttttccctcctaccctctctttctctttcttttctttctctctctgtttctctttttctctctttctttcttttgagacagggtcttgctctgttgcccaggctggagtgcagtggcatgatcttagctcactgcaacctcaaaactcctgggcacaagtgatcctcctgcttcagcctcccaagtagttgggactataggcacttgccattgtgcccagctattttttttttttttttgagacagagctttgctcttgttgcccaggctgcagtgtaacggcgcgatctcggctcactgcaacctccgcctcctgggttcaacaattgtcctgcctcagcctcccgagtagctggcattacaggcatgtgtcaccacgcctggctaattttgtgtttttagtagagatggggtttctccatgttggtcagactggtcttgaactcctggcctcaggtgatccgcccacccaaagtgctgggattacatgcgtgagctaccacgtccggccattttttttgttttgtagtttttgtagagatggggtctcgctttttgcctaggctggtctcaaactcctgggctcaagtgattcttcctcatcagcctcccaaaatgttgagattacaggtgtgagccagcacacctggcctaagagcagttttctgtctgttacatgccataccctcacttgcccaaatgcaaagctaagacttaaaatctcttgcaatgcatgctcaaggaagatggagtaggctcacccatgcctttgggtttcctggacctccccttgggaggatggctctgcagaggggctttaatgtgagatgtgagctcctcaccactgggggcagtatcgggcacctgcaggcactgagggtgcctgccggctactttgtctggcctagctgaggctggtgggcatactgggtaggtgctaagtggctagggggctgagcctgtttgcattgcaggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccaggtaagaagggaggagctctccacacccccaactgcccactcttctcccaacctcacctcctggcctgatgggactctggcgtgaatttgctgggtctccctgcagactctttctgttcatcgacacgcatgtttacaatatctgtagaaactagagtgtgttgacataaatgacttcatcctgcctctaccatctggaattagctttctgttaaccccttgcaatgtctagtaaaacctctccatgttagtacattacagcctcctcctgtctttatgctgctaggtagcattccatggtaaggataaatcagagtcgatttcacctctccctgttggtgaacaattagggttccaacagtgcttggaacagggatgctatagacatctcaaatgcaccaaccatttctcccagccagaccctggaagaagaatattggccatggagagtatgagagtctctgatgattcaggaaggtcagagcagctcctcaggcctggctgcagctctgggcacttgccaactccctgctggcctttgaggggcggtgcccttggagggccctggctcttatccctgctgttcccacacagaggctgccccggaggcctggggagccacgagaggtccacattgggcgagccctaggcagggtggctcccagcatgctgttgtgcagcctctctgaggccatctgcttcttcctaggtgagcctgggtgagacctccccactcggcattaggcttgctgggttagtgccggggcctaggagttcccagagggcagtgggtatagtgcagattcccttccccctgcaccctgtcaatgtcggctaccactctgcccttgaagccagggtgccctgacagccctctgctccctcacaggggccctgacccccatgccagctgtgcggacctttgccctgacctctggccttgcagtgatccttgacttcctcctgcagatgtcagcctttgtggccctgctctccctggacagcaagaggcaggaggtaggggcagctgggccagtactgagggacctgcccctgggttcccaccatggcagggagatggggtggctttaccaccacagagatggcccagagaatggggtgggggacaggggcattgtgccaggagagtaatatttaggccatgtattctccaatttcctacagaaaaataaatttgttttgacaattttttaaatataatcaaacctcctaaagtgcatgatgttgagaaataaaatacagttgacccttgaacaatgtggagattagggcaccgactgtctaagcagttgaaaatctgcatgtaactttttttttttttgagacggagtttcactctgtcacccaggctggagtgcaatggcgtgatatcagctcaccacaacctctgcctcccgggttcaagcgattctcctgcctcagcctcccaattactgggattacaggccccctcctcctgcacgcctggctaatttttgtgtttttaatagagatggggtttcaccatgttggtcaggttggtctcgaactcctgacctcaggtgatctgcccaccttggcctcccaaagtgctggcgtgagccaccatgcctggtctgcatgtaacatttgacccttctaaacttaattcctactagcctactattgactggaagccttaatgataacataaatagtcgataacacatcttttgaatgttatatgtattataaactgtattcttacaataaaggaagcaagaaaaaagaaaatgttagtaagaaaatcataaggaagagaaaatctatttactattcacgaagtgaaagtggatcatcatgagggtcttcatcctcgtcgtcttcaggttgagtaggctgaggaagaggaggaagaggagggcttgatcttgctgtttcaggggcggcagaggtggaagagaatccagggataagtgagcccaggcagttcaaactcgtgttgttcaagggtcagctgtataaatgagaggtcgacaggagttgatctgttggttcccatgatggtgtaaaatttaaagatattttatcaagattaaaataaaagcaaagaaaacagcacactggtatgtctccatgagggcactggcacgggccacccacagaaggtgacactccctgggggcaagaaggtggtccctggggccttgtctgctctgggactaccttgagggggtgcctcccactccaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtggtgagtgggcctcgaaccacacgagagcaggggcactaggtggggacctcgcctcagggagagcagggttggaggtggggaggttgcctaggcccaaatgctgatacttggggctggcacgcaagtctgctcaactccagaatgttgcccatgacaccctgactgacttaaatttgtggggagatgggggacggctgttgggcagggtggtctcatgcagcaggtcccttctcagctgctgctgtttctcgccctgttcggagtgagcctctactccatgtgccacatcagcgtgggactggaccaggagctggccctgcccaaggtgagcccaggcccttctcaacccttaggcccctgggatttggggaggggcagtagcaaccagcagggatgggttggggggtcctccggccaggggcttggccagaggtgcagaattgttcattactctggaggcacctccagcagtcctggggagtgaagccacattcgtgtatgaacagcacaacagccaggtgccagccccaggccacagtaagagagatggcccaggcatcggagggctgtccatgtgagatggcaggccacaaagaatgactgccactttgctgagtgcctgcccagtgtccagccctgcgaattctctgggcctgaagcccggggagggcaggggttcaggggaaggaaagccccgtggttggaggggacctccaaggtcacataggatttgcagaggaaagtgatgacagactcgccagtgggaggctagggtgagcccaggtgtgtttcctgggcgtggcagcgactgtgggggtgggatgagctggaggccaagggcatggtcggggagagtgctgattgcccagcctggaccagtaagtgtgcgggccaacaggcacaatgcatcagccaaggctggggacccggctcctctggatatgcatcagcggtggccatgggctggtggccaagaggaagcagccacagacaacaaagtctgagacacatggtcagactgcatgagcaagctctagggagagggaaggcatcgaggggactcgatgtctaggtcccatctggggaactgtgatggaggtttgggcaagggtctgggtactggcaggagccccagtggaagcagccaggcctgagcccacaacagggctgagtggggtgcggctggggtaggtgtgttaggcagtactggcctggggtcctggaagccaggtgagggaggacaagagcagatggctcaggactgtactttgggtgactttatggagggagagcaggtgaggagtcacagaatgaacctgccacctgcagaagccctgggggctatgtcacagggctgaggtgaagagggtctctagtgccccaagagcaagaaggaaggatgtgatgggctgccagaccctgctgaggttttatgttgatgtcttttgtttatttttctgttggggacatttgtttcttactgcttttaaaaattttatcattttttttccgttttttattgtggtaaaatacacataatagaaaattaccattataaccatttttaagtgtacagttcagtgatattaagtacactcatactattcaactatcaccaccatccatctccaaaactctttcctttttgcaaaattgaaactttacccaacaaacagtgactccccattctcccctcccctcagcccctgacacaaccaccttttatttatttatttattttgaaacagagtttcactcttgttgcccaggctggagtgcaatggtgtgatctcggctcaccgcaatctccgcctcccgggttcaagtgattctcctgcctcagcctcccaagtaactgggattacaggtggccgccaccacgcccagctaatttttgtatttttagtagagacagggtttcaccatgttggcctggctggtcttgaacttctgacctcaggtgatccaccagccctggcctcccaaagtgctgggattacaggtgtgagccaccgcacctggcctctactttttctttttttttttgagatggagtcttgctctgtcacccaggctggagtgcaatggtgcagactcggcttactgcagcctccacctcccaggttcaagcgattctcctgactcaacctcctgagtagctgggactacagccgtgtgccaccactcccagctaatttttgtacttttagtagagacagggtttcgccatgttggccaggctggtctcgaactctggaccttgtgatctgcctgctttgccctcccaaagtgctgggattacaggcatgaaccactgtgcccggcccatttactttctgttctatgagtttgaccactctaggcacctcaggtaagtgaactcatacaatatttatttttttggctgggagtggtggctcactcctgtaatcccagcactttgggaggctgaggcaggcagatcacctgaggtcaggagtttgagaccagcttgaccaacatggagaaaccccatctctactaaaaatacaaagttaactgggcatggtggcacatgcctgtaatcccagctactcaggaggctgaggcaggagaatcacttgaacctgggaggcagaggttgtggtaaactgagatcacgccattgcactccagcctgggtaacagagtgaggattcgtctcaaaaaaaaaaaaaaagtatattttgtctgatcttagtatagctacccctattctcttttggttactatttacatggaatatcttttttctgttcttccactttcaatctatttgtgtttttggacctaaggtgagtctcttggagacagcatatagttagatcacgttttgctgttttttagcagatgggggctgcctagggcacagtatgctgactctcacaatctcgatcgtgtgtgtgtgtgtgtgtgtgtgtgtgtgtgtgtgtgtgtgtgtgtgtgtgtgtttaattcattctaccactcttttttttcttttttttttttgagatggagcctcagtctgtcacccaggctggagtgcagtggagcgatctcagctcactgcaacttacacctcccgggttcaagcaattctcctgcctcagcctcctgagtagctgggattataggtgcatgccaccatgcctggctaatttttttgtattttttgtagagacagggtttcaccatgttggccaggctggcctcaaactcctgactttgagtaatccacccacctcggcttcccaaagtgctgggattacaggcgtgagccaccatgcctggtcctactactctcttttgattggagagtttaatccatttacatttacagtaattattgataaggagggatttacttctgtcattttgctatttgttttctatatgccttgtagattttttgtttctcatttcctgcattactgacttattttgtgcttagttgattgctactagtgaaattttacattttccttctcattttcttttgtgcatagtctacagctaattttatttgtgattaccatggggattatcttaaatgtgctgaagttataacactctaaatttatgccaactttgtttccatagcatacaaaaactctgccctataacaactccatcttacctccctttcagttattgatgtcacaaaattatatcttgagctagccatggtggcttatgcctgtaatcccaatgctttgagaggtggaggcaagaggattgcttgaggccaggaatttgaggccagcctagccaacacagtgagatcccatctctagaaaaaatttaaaatttagctgggcaagatggcacgtgcctgtagtcccagctatgtgggaggcttgcttgagtccaggaattcaagtatgcagtcagctatgatcatgccactgtactccagcctgagcaacagagagacaccttgtctcaaaaaattttatttttcagctgggtgtagtggctcatggctgcaatcccagcactttgtgaggtggttggatcacttgaggccaggaggtcaagattagcctggccaacatgacaaaaccccatctctactaaaaatacaaaaattagccaggcatggtggcacacaactgtaaacctagctacttgggaggctgagacatgagaattgcttgaatctaggaggtagaggttgcagtgagctgggatcgtaccactgtactccagcttgggcgacagagcgagactatgtctcaaaaacttttgtatttttatgcattatgtatccaaaatcataggctaatgattttttttgcatgagtctcttaaatcatgtacaaaaaggtggagttataaatcataacatttataactgcccatttatttacctttgccagggatttatttatttatttaaagaggcagagtcttgctctgttgcccaggctgagatgcagtggtgtgatcatagctcactataacctcaaactcctggcctcaaaagatcctctcacctcagccacctgaagtactgggattacaggtgtaagccactatgcctagccaagggatttttatttcttcatacatctttgagttactgctgatgtctttttttttttttttttttttttttgagaagttgttttgctcttgttgcccacccaggctggagtgcagtggcatgatctcagctcaccgcaacctctgcctcccgagttcaagcgattctcctgcctcagccccccgagtactgggattacaggcatgtgccaccacgccaggctaattttgtatttttagtagagatggggtttctccatgttggtcaggctggtcttgaactcccaacctcaggtgatctgcccgccttggcctcccaaagtgctgggattacaggcatgagccaccatgcctggcctgtctaatgtctttttatttcaacctacaggattccttttagcatttctttcagggaaggtctagtgataacgaattccttcagcttttgtttatctgagaatgtcttaatttcaccctcattttaattttttaaaattttttatttattttgagatggagtttcactcttgtcgcccaggctggagtacaatggtgtgatctcagctcactgcaacctctgcctcctgggttcaagtgattctcctgcctcagcctcctgagtagctgagattacaggtgcatgccaccatgccaggctaatttttgtatttttaatagagacggggttttaccatgttggccaggctggtcatgaactcctgacctcaggtgatccacccaccttggcctcccaaagtgctaggattacaggtgtgagccactgtgcccggcccatttttattttttaattaaaacaatttttttgagatgggggtctcactgtgttactcaggctggtctcgaacttttgggctcaggtgatcctcgtgtctcagcctcccaaagtattgggattataggacgaatcacctcatctggcatctccctcatttttatttttaatttttagttttttttttttttttttgagatggagtctcactgtcacccagattggagtgcggtggtgtgatctcggctcactgcaacctccacctcccaggttcaagagattctactacctcagcctccaaagtagctgggattacaggtgcatgcctccacgcctggctaatttttgtatttttagcagagatggggtctcaccatgttagtcaagctggtctcaaactcctggcctcaaataatctgtctgcctcggcctcccaaagtgctgggattacaggcatgagccaccatgcctggccttctccctcatttttaagtgacagttttgctggaattaggattcttcattgacaattgttttttcttcagcacttgttttttgttgttgttgtttgtttttgagacagagtctcactctgtcatccaggctggagtgcagtggcatgatctcagctcactgcaacctctgcttctcaggttcaagtgattctcctgcttcatcctcctgagtagctgggattacaggtttgtgccaccatgcctggctaatttttgtattttcagtagagatggggttttgccatgttggccaggctggtctcaaactcctgacctcaggtgatccacctgcctcagcctcctgaagtgctgggattacaggcatgagccatcatgcccagcattcttcagcactttcaatctacaaacccactgccatctgggcttcaaggtttctgatgagaaatatgctgataatctttttgaggatcttttgtatatgccaagtcacttcttttttttcaatattttctattttttaaaaaacttattttattttactttttatttttattttttagaggcagggtcttgctatgttgcctagaatggacttgaaaccctgggctcaagcaatcctcccacctcagcctcttgagtagctgggactacaggtatatgccaccatgcctggcttgtctttggtttttgacagctaaattataatatccagctgggtgcagtggcttatgcctgtaatcccagcactttgggaggccaaggtgggtggatcacaaggtcaagagatcaagaccatcctggccaacatggtgaaaccccatctctactaaaaatacaaaaattagctgggcatggttgtgcgcacttgtagtccaagatacttgggaggctgaggcaggagaatcacttgaacccaggaggcagaggttgcagtgagccgagattgtgccactgcactccagcctggcaacagagcaagactccatctcaaaaaaaaaaaattataatatccgttggtgtgggtttctttagtttatcctattggagtttattgagtttcttgaatgtttatattcatgtctttcatcaaatttggggagttctggccataattttttcaaataatctcacttcccctttctcttttcttctggaattcttacaattcatattttggtctatttgatgatgatgatgtctgacaggtcccttaggctctgctctgttcactttcgttattttttttcctttctcttcttcagactcagtaatttcaatggtcttatcttcagtttgctaattctttcttctgactgcttttgaatccctctagtgaatttttcatttaagttactgtactttttagctccagagtttttttgctcttttttatgtttcctcctcattgatatttccattttgttcataaatttttccttgactttgttttcttttagctctttgagcaactttaaggcaattgttttattcatttattttattatttatttatttattttttgagacagagtcttgctctatcacccaggctggagtgcaatggtgtgatctcgggtcactgcaacctctgcctcctggggttaagcctcagcctcccaagtagctgggattacaggtgcctgccaccatgcttggctaatttttgtatttttagtagagacagggtttcaccatattagccaggctggtctcgaactcctggcctcatgtgatctgcctgccttggccttctgatgttgtgggattacaggcatcagccactgtgcctggctgagacaattgttttgaagtctttgtctagtaagtctgctgtctggtcttacccaggaacagtttctgttggttaatattttccctttgaatgggccatgtttttctttttcttggtgtgtttttggttgaaaaatggacatttgattcttataatgtggtagctctggagatcagattctccttctttcccagggcttgctttattttatttattgctgttggtgtttctgtgctggggatcagccaaaggcacagagttaatgtcttctcaggtatttttgagactgcatttttctctgagcatttatgcagtgtggtgactgtctaaatatccctatatttatggttgcttttgaatgtccttgtccttatatgtatggttcccaaaaggagaaaaagggaaaaatgaaggtgtcggggataggtgcttactctttaaatctcctggaagtcactttagtaagatgtggaggtggttgcaacaacggtggtgggagttgcattagtggctgcctgcctgtgtatctgtaccaccaatatcagaagtaatgatcaattatcagaactcagatccttgatatttgaacttatttatttatttattagagacagggtctggctctcttgctgaggctgaagtgcagtggtgcaatcataggtcactgcagcagcaaacttccaggctcaaatgattctcctatttcagcctcctgagtagctaggactacaggcatgtgccaccacacccagctaacttttgtatttttttttgtagagacagggtgtcgctatgtgcccagatcggtctcccactcttgggctcaagtgaccctcctgcttgccctcccaaagttctgaaattacaagtgtaagccatcatgcccagctgatatttggtggatggtgtccttgcctacctggctcctgcaagctgtgtacaagctgcttctggaaagcatacacagctgcatgccttgaggctgggagtggcaaatgggtagctgctactgtactaaagctgagattgcctgaaattaaccacaatttactgtccaagccttatcctggaagcttccagccctcaatagactccagagttccaaaatcgttacactagggccggtgtggtggctcatgcctgtaatcccagccatttgggaggccgagacgggtggatcacttgaggtcaggagtttgagacaagcctggccaacatggtgaaaccccatctcttttaaaaatacaaaaatcagctgggagcggtggcacatgcctgtaatcctagctactcaggaggctgaggcacaagaatcgcttgaacccaggaggcggaggttgcagtgagcagagatcgcgccactgcactccagcccagaagactccatccatctcaaaacaaaacaaaacaaaacaaaaacaaaatagttataccagacaaattgttgtctagctggggagagggattcctgacacttcctactgtgccattttccctaatgtcactctgagcctttatgttatagaagggagcagaccatgaggatgcctggtgcatggctttgagggtgtgcacactgacatttatatgtgcacacaaatatgggccgttgtcacaggccagcttgttagacggtggctgtgccatattgggggtgataggaaggggtacaattatgtgtctgtgcatgtttgtgtgtgtcagtgtgtgttcatgtgaggtgataggtgttgctctgtgtttgtacctgcataagtgtacttctgtttgcacctgtgattatacctattctgtgaaccttggagtatgttcatctgggggtacacctaaaactgtgttccggtgtaactgtacagtgcacatacatcttgagggtacccctgagtgtgtgtgtctgtgcatgtccttctctatatgtaccttgtgtgtgacctctgagcatgtacatctctgtgtatattttgtgtacttgtgtgcatgtacctctgtgtacctctaagcatgtatctacgtgtatatctctgagtgtcccactgagcacatccctttgagtgtgtaactgcatgtgtgtctctgaacatgttcctctgtgtgttcctctgatcatggacctctgaacatgtgccttttagcatgtacctctgtgtgtaccttcgagagtgtgagctggattgagccctttaggggtgtgcatagcgaaccaaagctcactgaccctcctccactcctaggactcgtacctgcttgactatttcctctttctgaaccgctacttcgaggtgggggccccggtgtactttgttaccaccttgggctacaacttctccagcgaggctgggatgaatgccatctgctccagtgcaggctgcaacaacttctccttcacccagaagatccagtatgccacagagttccctgagcagtgagttcctggcccgccccaaaccccagcctactccctgtttgagtccctccagtcctctccagtcccctcttcctgatgttctatccctgtcctgctgccctgctgccttgctgccgtatgcctggggagggctgcgtgggggttgggccacgagaaggacccaccaccctgcccagctggccttttcacccttcctcccacctgccccttaggtcttacctggccatccctgcctcctcctgggtggatgacttcattgactggctgaccccgtcctcctgctgccgcctttatatatctggccccaataaggacaagttctgcccctcgaccgtcagtgagtgtggggccatggggactcactgtccaccacagctcgggcaaactgaggcaacagaaaggagaggactggagaggctccctcaacctctcccacgcatcctgcagggtctgtcgggggcatgggtgcagatgtggcctgagggacaggcactctgtgagaagcacctgtgtgggtgaccgtgctggcccgtgggcatcacacatgtatactgctgtgtactgtgcccccattttcagagcacatggtgctcccgggtggcagggcagtggggagtcaggaggggagagctgctgaggttagcacatggccctgccgcccaaagcagtggcatttgtaggtggagaggcctttgtggggcctgtttttctgccccaaacttcctttccccttctgcctgtaggtgcccacagtttctatagccaagaggagaacttctcccacaaatgacaaatgcaaatccccctagaagcgactggttgaggctggagtgcccaggacctttgatgggattgttggggaaggaggggcacaaagcaggagctgctggccctggggtgtcactgcccagacccctgctttctctgcagactctctgaactgcctaaagaactgcatgagcatcacgatgggctctgtgaggccctcggtggagcagttccataagtatcttccctggttcctgaacgaccggcccaacatcaaatgtcccaaagggtaagcttgggagggccttctgctggggaggacagacatgtgggacacaggatggggttgaatatagagaggcaggaggaggctatcaggggcctctctggggtggctgtgggctgggcagatgaaagaagcttcgtccctggctaagcctttgccctgaccttcttgcagcggcctggcagcatacagcacctctgtgaacttgacttcagatggccaggttttaggtaagcatggccttgcctggaggggaggacataaatcggttgctctggagggcccccgaaaaccccagggaacagcctgtcacatgttgtctccctcctttgtcaggaggttctcactgcgctggccctgtcagcaggggtcttgtttcccagctccacatctcagacttcaccccttctctcactcccaagtccatggtcagtgctaagtttgtggaattgattcagcagttgataccatacttgggagttctccacaccctggctaagcacctttcttaccagcacaaattacacccaaagggcagctggttaaatgaattaggatgcttggcacagcacaatcctagcagtcatttaaagtaacaagaggctgggcgcctgtaatcttagcactctaggaggccaaggcgagaggatctcttgaatccaggagtttgagaccagcccaggcaacagtagggagaccctcttttttttttttcgagacggagtctcgctttgttgcccaggctggagtgcagtggtgcaatctcggctcactgcaacctccactttccgggttcaagcgattctcctgcctcagcctcctgagtagctgggactataggagcataccatcatgtctgggtaatttttgtattttcagcagagatggaattgcaccacgttggccaagctggtctcaaactcctgacctcaggtgatatgcctgccttggcctcccaaagtgctagtattacaggcatgagccactgtgcccggcctcctctacaaagtaaaatttaaaaaattgcccgggtgtggtggcgtgtgcctgtagttccagctattcagaaggctgggcgggaagaatgcctgagtctgggaggttgaggctgtagtgaactgtgatcgcaacactgcactccagcctgggcaacaaagtgagaccctctctcaaaaaaaaaaagaaagaaaaaagtaacaagagagatgcagttggactgacaggaaaaggacccacaacatgctgtcagcttatacagcagatggcagaacaagacagccatctgtgtaaaggagctggccatagctccgtgcagacatgctcggtgtaggggccctaagggagctcgtgctggagatggacatgggggtcgtcggtgggtgggggagtttttgaaggatgatctcactttgtactgaaataattcatagtttgaactgctggctgaaagctgcctcaagttcgctcaccccacccttccagctatgaagttcccatgtttccagaagggcaatgcaccctgcccagccctggtagctgagcacaacaggctctgtgaggccagtgtggtggggctggtgtggacagatgggagtggatgtgtcagtcagggaatgaggagcagggcctggaaggagcacacagtagagccaagcccccataaccgggggcaagtctgcaccatctctgacctttgtcttcttgtgtgtgcactaggttagtctagagcagcacttcccaaaatgaggtcccccagccagcagcatcagcataacctggaaattgttcaaaatgaagttccagctaggtgctgcagctcacgcctataatcccagtacgttgggaggccaaggtgggaggatcacttgagcccaggagtctagtctgtctgagaccagcctgggcaaaaaagccagatattgaaagaaaagaagagagaagaaaaggaaagaaaagaaaagaaaagaaagaaagggagaaagagagagagagaaagagagagaaagagaaagaaagaaagaaggaaggaaggaaggaagaaaaagaaagaaagaaagaaaaagaagaaacgcaagttctcagccctcacccaagactttgcagaccccgaattgctgggctgggctgggcatttgtgtgtgaactaccctccaggtggtcagaggcctggtgggaagttctccaggcacctcccctgctctgagattgtatgtatccaagaacatttctcttcttttttctccacacctatgtagcactattgtttctttttcagatacacatgctcactgtacacaataaagaaataactttttttttttttttgagacacagttgccattctgtcacccaggctggagtacagtggcacaatctcggctcactgcaacctctacctcctggattcaagtaattctcctgcctcagcctccctagtagctgggattacaggcacatgccactatgctcagctaatttttgtattattaatagaggcagagtgtcgccaagaaacaacctttttgggccaggtgcggtggctcacacctgtaatcccagcagtttgggagaccgaggccggcgaatcacttgaggtcaggagtttgagaccagcctggccaacatggtgaaaccctgtctctactaaaaatacaaaaattagccaggcatggtggcatgcacctgtaatcccagctacttgggaggctgaggcaggacaatcacttgaacccgggaagcagaagttgcagtgagccaagatcgcaccactgcactccagctgcggtgacagtgagactctgtctcgaaaacaaaaacaagaacaaaaaaccctttattgtataaaggtcttaataaccttaatttcttcttttttttttttgagatgggatcttgctctgttgcccagctggagtgcagtagcatgatctcagctcactgcagcctctgcctcctgagttcaagaattctcctgcctcagccccccaagtagctgggattacaggggtgtgccaccacgcctggctaatttttgcatttttagtagagacagggtttcaccatgttgggcaggctggtcttgaactcctgacctcaggtgatcgacctgccttagccttccaaagtgctgggattacaggcatgagccaccacacccggccaataaccttaatttcttaaaagtcattaagaaataacctttatctggcaggagccctaagccacagctctaataatccaaccgttctcatttttctgtcttcctttctagtcctttcctataggaatatgcaaattaaaaaccaattaagttaattttaaaaatccaatgcatatcttgaaaccatacagagaagaatctcggttcactagggagatctctgtaggcttcactcatcaaaggtcaggcctgggtctcccacagcagtggggccagctatggagtttgcagggctggtgcaaaacaaaaatatgggcctcttgcacaaaatttactaagaatttcaaatggtggtggcagagccctgaaccccgcttgatcacatgcctgtgccactgcgtctgcggtgttctgaagttgtcctggaaagggctctgacctttgcccttccatcttctgtgtgccatggctgtccagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgtgaggacctgagtggctgggctggagggaggtggggtatggttgctggagactggaggttagggtggagggcttgcaaggagttgcatgagatgaggaccagttttaggtcaggaggctctggctgcagccttgggcctatttcttaggctggtttgtaccccaatataagcctgcctgaccctcagcattctccttctgaagtggggtgtcccacccaccatgagggccccagaggcctgagcctgtgaccatgctctgtgctctggcaggatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtggggcatcagttacaatgctgtgtccctcatcaacctggtctcggtaacccagcagacacaggcaccagggggcctctggaggggtggttggggatccagcctcatagaatactcctagttcttttttgtttctttttttagaggcagggtcttgctctgttgctcaggcttgagggcagtgacatgatcacagctcactgtagcctcgaacccttgggctcaagcgatcctcctacctcagcctccaaagtagccaggactacaggcacgtgccactgcgtccagctaatattttaatttttgttgtagagacagggtctcactttgttgcccaggctggtctcaaactcctgggctcaagtgatcctctcacctcggcctcccaaagtgttgggattataggcatgagccactgcacccggccaaatactcccagttctgtctagaatctagatgcctgccccacgctggtcctggtggaggcctcatctccctagttccttccccacctctgcctttcttggcttatgccccctctctgcccataggcggtgggcatgtctgtggagtttgtgtcccacattacccgctcctttgccatcagcaccaagcccacctggctggagagggccaaagaggccaccatctctatgggaagtgcggtgagtggagaggagtgggccaccctgtgccccactcgacaccctgtgccctgcctgatgccctgtgccctgcctgatgccctgtgccctgcctgacacctggctctgaaccccccaggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctacgtgggtgagtgcccaggcctgttcctaccagactgtcatgattatgctgacgacaacagtaacagtgcatgctcaccacaaaagctcaggaagtgcaaacgagccatgggcagatgtcagaagccaggactatgaccatgtggcaattctgtcttggaagctactattattcatttaatgtgctgtgaacatctttttttgtcagctatgtatgtctcaaacaacgtttctgtggccctgtacactgtggatcttcactgcactgctgttggacttttaagcatgcccttcagcaagaaatatattttacacagagaggtgacatgcacgggcacacatagacatgcctgcctaaaacaaatgcttcactaaataatattaatacttcctttatacatgtgaagcattctgatattgctggttccattctattattattattaatattttttggagacagggtcttgctctgacacccaggctggagtgcagtagcatgatcacagctcactgccaccttgacttcccaggctcaagtgatcctcccacctcagcctcccgagtagctgggaccacaggtgcacaccaccatgcccagctaattttttattttttgtagagatggggtctccctatgttgcccaggctggtctcaaactcctgagctcaagtgatccaccatggccttccacagtgctaggattacaggtgtgagccactgcgcttggcttttattttactttaaatttgttatttattttattttactttacattattttatttttattttttgagatggagtctcgctctgttgcccaggctggagtgcagtggtatgatctcagctccctgcaacctctgcctcccaagttcaagccattctcctgctttagcctcccaagtagctgggattacaggtgcgcaccaccacgcctggccaatttatttatttattttttatttttagtagagacggggtttcaccatgttgggcaggctggtctcgaactcctgacctcaggtgatccaaccgccaaggcctcccaaagtgctgggattacaggcgtgagccactgtgcccagccctatcattaatttgtttttaattattttaattatttttatttttattatttttagacagagtctctctctgttgcccaggctggagtgcagtggcgcaatctcagctcactgcaacctctgcctcctgggttcaagcgattctcctgtctcagcctctcgagtagctgggatatcggtgtatgccaccatacctggctaatttttgtatttttattggagacaggtttcaccatgttggtcaggctggtctcgaactcctgtggcctcaggtgatccatctgccttggcctcccaaagtgcagggattacaggcgtgggccaccgcacccggtctcattaatattttgaaatgctggccaggagtggtggctcatgtttgtaatcctagcactttgggaggctgaggcacatggaagctcaaattgagcctcccaggatgaaggtgtttctggctctcagggtgggcaagctgggaggagttcaattttacctcccaccagatggtaataatattattagaggacatttatagaggggtgtgtttgtgcatcaacatatgtgtctgtaattctcttactacccccgaggcaggtattattatccttcccattttacagatgagggaactgagacacctgccccaggttacagacttggtcaaaggtagtaggggttggagcccacacagctctgtggttcctaaccatgtctcttgtggggactccctgaccctcttggaaggagtagagtgtgtgcgctgggggtggtggatgagacataagagaggggcaaggaggagcagtcgtggggtgtgcttggacaaaggatatccagggccttggagctgcaggtggtggctattccttggaggttcccaaaatgcttgggggatggagggaccaggacatccctgaagcttgggctgtgaacatagtgaccctggaaggcacatggcacagatcccccctgggacccttcctgccctgggtttgttgtacagaaccaggaatagcttctcacctgtgtcccctgcccacctctctgactgtggttctctgtctctccgcagggcctgacgttaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctgatacagccagaggccctgtctaggctctatggccctgaaccaaagggttatggggatcttccttgtgactgccccttgacacacgccctcctcaaatcctaggggaggccattcccatgagactgcctgtcactggaggatggcctgctcttgaggtatccaggcagcaccactgatggctcctctgctcccatagtgggtccccagtttccaagtcacctaggccttgggcagtgcctcctcctgggcctgggtctggaagttggcaggaacagacacactccatgtttgtcccacactcactcactttcctaggagcccacttctcatccaacttttcccttctcagttcctctctcgaaagtcttaattctgtgtcagtaagtctttaacacgtagcagtgtccctgagaacacagacaatgaccactaccctgggtgtgatatcacaggaggccagagagaggcaaaggctcaggccaagagccaacgctgtgggaggccggtcggcagccactccctccagggcgcacctgcaggtctgccatccacggccttttctggcaagagaagggcccaggaaggatgctctcataaggcccaggaaggatgctctcataagcaccttggtcatggattagcccctcctggaaaatggtgttgggtttggtctccagctccaatacttattaaggctgttgctgccagtcaaggccacccaggagtctgaaggctgggagctcttggggctgggctggtcctcccatcttcacctcgggcctggatcccaggcctcaaaccagcccaacccgagcttttggacagctctccagaagcatgaactgcagtggagatgaagatcctggctctgtgctgtgcacataggtgtttaataaacatttgttggcagaaatggtgttttatgtcacatgtcctaccctggcttcctcctctcggtttaagataatttttgtgaatgacacaaataatacatgtgtgggagagtgatttgtggagatactagtctgtgttttgttctatttctcctccctcttttcaagaaagtagccaggccattgtgtgctcatgccttacaagggcctttgaggagtgggagtaatttctcttcaaactgggagggcacagagcctgagagtcagtcaggagtaggatgtgcagcccctccttttctggaagagactgtgaagtaggcaacacctggaggagctacaggagaaccacggtgcattcaaggagggaagaacccaccgtacaaacaaccagctcccaggagggccccaggccagggcagtgggtggaaatgtcaaggaacattccagatcccctcgagtctttctgccccatgctgggtccagcccttgtttggctgaggggctgctgttgctttgaggctcagagggactgtcagcatgtaaagggaagacaagcaaaaaggggtggaaaggagctggcgtttctggagcctactatctacttttgggtcctcataagagccccatgtgccagcatcattagcccacctttgggagggttgctggctgaccatgatggacaggaggtttggtgaagggacagctacgagggaatagaggctgaggagaaatcgcacaattcaccctgttaaaaactccacaggtgcagaataaacagatagatttgaggaacaaaatagcttttgacagcagacatttcaaatcagaggaaagggtagatccttcagtaaacggtgtgagagtagtgagcaaattatttggatcaaaataaagttatatctatacttcacacaatacacaaaataaaagtacagacagattaaagcactaaacacaaaaatgaaactatacaactatcggaaggaaacacagaagagtatgttataatcttggagggggaaaagtttcctaagcacaaagtccagaagccataaaggtaaacactaaggtatgaccatataataatggaaaacatctgaaaacacacaaaaaattaaagaaagttgaaagacacatatgagctcagaaaaatagttgcaacatatttaacagcaaataaaatcaagaaaacacaaagagtgccaatagtgctcctgcaaacatggtgaacactcctaaaacccactggactttctgtaagaagtgtgggaagcaccagccccacagagtgacacaggacacatttccctgtatgcctagggaaagccatgttatgacaggaagcagaggggctatggtggggagactaagccaattttccagaaaaaggctaaaactacaaagaagattgtgctaagttttgagtgcatgaagcccaactgcagatctaagagaatgctggctattaagagatacaagcattttgaactgggaggaggtaagaagagcaagggccaagtgatccagttctaagtgtcatcttttgttttattatgaagacaataaaatattgagtttatgtttaaaaaaaaaaagaatatacaaagagagtccaggtacggtggctcatgcctgtaatcccagcactttgggaggctgaggcaggagaattgcttgaggccaggagttcaagaccagcctaggcaacatagcgagatactgtctctacaaaaagtttaaaagttagccaggctagctatttggaaggctgaggtgggaggattgtttcagctcgagtttgaggctgcagtgagctatgatggcaccactgtactccagcctgagtgaaagagtgagcttctgtctcaacaaaaaaaaaaaaaaaaaagaatatacaaagagaggaaggagtgcaggggggaggtctgggttatgtggctaaccttcccattagaaacaagacattctagctaaaataaatcttagccgtgtgtgtgtgtgtatgtgtctgtgtgtgtgtatgatgcatacaagtttagggtgttttaaccttcttgataaattgagacttttatagtttgaaatgactataaaaatatccctttttatctctagtatttatttttgtctgtttaagagatggggttctcactttgttgcccaggctggtcttgaatacttggcctcaagggatcctcctacctcagcctcccaagtacctggaattacaggtatgagccaccatgccagtcctatctgtagtatttgttcaactgtataatgttattatacacacacacacacacacacacacacacacacagacacacacacacatataaaataacatacggttgaacaaattttatacttaatagtcaaacattgaaaccctttcccctgagattgggaatgagacaaagttgcccacttttacccaacattgcactggaggtcttagccattgtaataaggcaagaaaaagaaactaagtttataaggattagaaataaataaaattgacatcattcacagataacataaatatgtataaaaaagattcagtctgggtgcagtggctcatgcctgtaaccccagcaatttctgaggccaaggcaggaggatcacttgaggccaggagttcaagacatagcaagaccccacctctacaaaaaaaaattttttttaaagatccaaaagaatctatatataaactattggaattactctaacaaaaggtggtcaagaaaactatgaaaaataataactttgtattttaatttgtataatattgagagaaattaactgtcaaaagaaatggaggaatataccatgaattgagggctctatactacagagatgtcaattctcttcaaattaattactagtttcactgtaatttcaataataaccccagaaaattttttgtggaaactgataagctgattcaaaaattcatatagaaccacaaaagatgaaaattcacgaaagcaatcttgaagaaaaacaaagtcagagaacttacactactagaaatcaagataatataaatatatagaaataaagatagtgagattttggcacaaggaagaacaaatagaaaaatggaaagaatagaaagtccagaaacagatgatacccacaaggacacatgatttatgatggaggaggcatgcagagcattgggtaaaggaggtttttcaatgtaggatgctgacctagttgggtatccacacagaaagaaatgaatcatgaccctctcccccaagatacacaaaaatcagttcctgatagattgtcaatctaaatgtgaaagataaaatgatagagttctaaaaggtaacataaaagagtatccccaagactgaaataggaaaaacttttcttaggaaacaaaagccttacttatagagaaaaagattgataaattgaactgtattggaataaaaaaaaacttctgttcttcaaaagacatccttaggaaagataaaattcaaaccatagagaggaaaagatatttgcacatatctgaaatacacacatatctgagaaagggcctgtgcttagaatgcataaaaaatctcctacaactcagcaagaaaaagacagacaaccaaaagaaaagctaggctggctactcaaataagcaaatggccaatacaagttcctcaattttgtcagtcaccagagcaaggctgagtaaaagcacagtgagagttcttcctcttctcttccctcacaatttggcctacaggccatggggtaaggtggggccaggcagcacatgtggggtgtcagaatccaggtggtgtggggagcgtttccacattggatctgagggaggagaggagggcattccacacagaataggaactacataggcccagtatggggctaagatgtcagaactgagctctgatgtgcctttctccatgagcagagggactggatgctggagatggagggtggaggaaaggttcagagccatctagagatggcaattcagaggaaatgggagggcagatagtctcactcttcacagtgaggcagagtttccaagctggttttgtcactcctttgctgggcctctttgggtaacatatttgacttatctgggctttagtttcttttttgcttttttttttttttgagacagagtctcactctgttggccaggctggagtgcactggtgtgatcttagctcactgcaacctctgcctcccgggtttgagtgattctcctgcctcagcctcccgagtagctgcaactacaggcgcctgccaccatgcctggctaatttttgtatacagatagggttttgccatgttgaccaggctggtcttgaactcctgacccgaggtgatatgcctgcctcagcctcccaaattgctaggattacaggtgtgagccaccacacctggcatgggtttggtttctttacctgtaaaaactgggatagtttagctgggcacagtgatgctaattgttgtcccagctacttgagaggctgagatgggaggatcacttgagcctaagaatcgcaggtcagcctgggcaacatagcaataccccatctgtgaaaaaaaaaattagtggctgagcacagtggctcactccagcaatcccagaactttgggaggccaaggtgggaagattacttgagcccaggagtttgaaactggtctgggaaacacacagagaccacaatctctgcattaaaaaaaaaattagctgggttggtggcactcacctgtggtcccagctacttgggagggtgaggtgggaggataatttgatcccaggaagtggaggctgcagagagctgtgatcatgccactgcactccagcctgggtcacagagtgagaccctgtctcaaaaaaaaaaaaaaaaattaggaaaatttgccctgactccccacgttttttttaaaggatgaaatgagatattatatgtgaaagcatctagtacttgtgacatagtaggtgcttaaaaagtgtttccacttcacttctgcctaaaacccagttcagttcctgagttccagatatctaactgtgatgagaagagacgcagccagaggtacctcaaagatagcaacacccccctccgccccgatacctgatgtactgaagtcagaaatttaaaaaaaaaccttgttcttccttcagttttaagttcagtatactgatgaactatcggtcacatttgacgatttactttaaaaataaacaggcttccaaattaacctacttatatggtttgtctgtgtcgccacccaaatctcatcttgaattcccacccgttgtgggagggacctggtgggaggtaattaaatcatgggggcaagtctttcctgtgctgttctcgtgatagtgaataagtctcaagagatctgatggttttaaaaagaggagttcccttgcacaagctctctctctttgcctgctgccatccatgtaggatgtgacttgctcttccttgccttccaccatgattgtgaggcttccccagccacatggaactgtaactccaattaaacctctttctcttgtaaattgcccagtctaggctatgtctttatcagcagtgtgaaaacagactaatatacttaccttggaaaggccttgtgatccatggtgacatcttgtccctaaggaaagcatcttaccatgagttcctcaaattgttgatgtactgattaatgtgtaaccctctgacactgggaagaacactgatttatttctgaatcataaagttttattgattgtcttgcatgtagacattttagcttgtatgttgcaatctgtatccaacaattgtaacctctgtattgtaccctcaaatgaaagaggaaaaaactcttgtatgaggagtcccctcccttctcctaaactttcctataaaagccttctaccttgtaacagactggaacattcctaacattgttggtgtgtttcctaagcggattctcacatttggcttcaaataaaccttgatcaaattagtgctgcctcaacagccttaatttcaatcaatagtacaagcctctgtttttctatttaatcactactttaaaggtaacctttggaaaatatttaggctctttacaaatttaattaattgaacatattttaactgcatttataaaggtaatagtctccattttcttcctaaatactctgcataagaaacaaaatcttcccatatacttaactcttttaaacctaataaattaaatttatggaatatcattaatataaagtttttatagatgttgtaacactgcacatagatttagcaacatttcaatttacaatcttaagcttatatgaaataccattttaaattggaattatacaattcttacactaatagaccaaatactttaaatgttacaagcatataaaatacgaaatatacaaaaatttccccccatcacacaaatattcttactaaggttttgcttctttgaaacctttctatacacattgtattagtctgttttcatgctgctaataaagacataccccagactgggtaatttataaaggaaagagttttaattgacttatagttcagcatggctggggaggcctcaggaaacttacaatcatggcagagggggaagcaaacatgcccttcttcatatggcaccagtggagagaagaatcagtgcccagtgaaaggggaagccccttataaaaccagcagatctcgtgagaactaaatcactaccacaagaacaggatgggggaaaccgctctcatgattcaacgatctccacctggtccctcccacaacacatggggattatgcaaactgcaagtcaagatgagatttgggtggggacacagtcaaaacctatcaacctaacatccttttcctctccccttccttccttcctcccttccttccttccttccttccttccttccttctttccttccctccctccctccctccccctctctctctttttttctttttcttttctttctttctctctctctctctccctccctccctcccaggctggaatgcagtggtgcgatctcggctcactgcaacctctgcctcccagggttaagctatcctcccacctcagcctcctgagtaactggtgggactacaggcgtgtgacaccacacccagctgatgtttttgtatttttagtggagatggggtttcagtatgttgtccaggctgtccatacccatttttaagtgagttataaatggggttcaaaggtcatactccccttgaggaagacaatcatcatctcagataaccaaggttgcctatgcagtaaggaagaagtaagtcatcattccgggtaactaaatttacctaagaccaaagacatcagctgagagtgagacctggagtctcaggcatcgggagtagttatctcactgctaactaagtttacatggtgagtcaaaagacccagaatacccaacacaatattgaaggaaaacaaagtcagaggactaacactatctgacttctagacttactataaagttatagtaatgaagacagtgaaagaactggtaaagaacagataaataaatcactgtaacagaatatagagtctagaaatagacccaaataaatatagtgaagcaaaggtagactttttttttttttttttttttttgagacagagtctctctctgtcacccaggctggagtgcactggtatgatcttggttcaatgggacctatacctttaccatgagaatcactgggttcaagtgattctcatgcctcagtgtcctgcatagctgggactaaaggcctgcaaacatgcctggctaatttttgcatttttagtagagatggggtttcaccatgttggctaggctggtctcaaagtcctgacctcaggtgatccacccgccttggcttcccaaagtgctgggattacaggtgtgagccaccatacccagccaaagggcagtctttccaacaaatgatacagatacaactggacatctatgtgcaaaaacataaatttagacacagactttgcacccttcacaaaaactaactgaaaatggatcatagacctcaatgtaaaattcaaaactataaaactcctaaaagacaacatagggtaaaacctagatgaccttgggtgtagcgaccttttgatacaacaccaaagacataatccatgaaataaataactgataaactgtaattaataaattttttttagcagtaatagaatgatgagtgttatttcattaaaatttaaaacttctgctctgcaaaagacaatgtcaagaagaagaagacaatggccaagtgcggtggcttatgcctttaatcctagcactttggaaggccaaggcgggtggatcacttgaggccaggagtttgagaccagcctggctaacatggtgaaaacctgtctctactaaaaatagaaaaattagctgggcgcagtggtgcacacctgtaatcccagctacttgacaggctgatgcacaagaatcgcttgaacccaggaggcagaggttgcagtgagctgaaattgtgccactgtactccagcttgggcaacagagcgagactctgtctcaaaaaatatataaataaataaaatttaaaaaggatgagaagacaagccactgcctgggagaagatattagcgaaagacacatctgtgctggcttcagcagcacacatactaaaattacaatggtacagagaagattaccatggcctgtgcacaaggatgacatgcacatttgtgaagtgcttcagaatataaaaaagaaaaagatctatccgataaagaacttttatttaaaatctaaatggactctccaatacaataataagaaaacaaataactcaattaaaaacttagccttaccaaagaagatgtacagatggcaaacaagcatatgaaaagatgctacatgtcatatatcatcagggaaatgataattaaaacaacaatgtgatactgctacacatctattagaatgtccaaaatctggaacactgacaacatcaaatgctggtagggatgtggagaagcagcaactctccttcattactgataggaatgcaaaatggtacagccactttggaagacagttcttcagtttcttctaaaactaaatatatcttaccatatgatccagcaatcacatttcttggtatgtacccaatggagttgaaaacttatgtctacacaaaaaccaacacatgggtgttcatggcagccttattcataattgtcaaaacttggaagtaaccaagatgtccttcagtaggtgaatgggttaatccccacaatggaatattattcagcattaaaaacaaatgagctatcaagctaagctatgaaaagacatggaggggccgggcacggtggctcaagtctgaaatcccagcactttgggaggccgaggtgggcagatcacaaggtcaggagtttgagaccagcccggccaatatggtgaaaccctgtctctactaaaaatacaaaaattcgccgggtgtggtggcaggcgcctatagtcccagctactcagatggctgaggtaggagaggagattcacttgaatctgggaggcagaggttgcagtgagccgagatcacaccattgcactccagcctgggcaacaagagcgaaactccatctcaaaaaaaaaaaaaaaaaaaaagaaaaagaaaaagaaagaaaagaaaagaagtggaggaacttcaaatgtatactactaagtggaaaaagcaaatctaaaaagtctacatctgtctgattccaactatatgacattctgtaaaaggcaaagctataaacacaataaaatgatcggtagtttctagggtttggggttaggggagttgaatgggcagagcacaaaagatttttaggccagggaaaccactctatatgatattataatcatggatgcatgtcattatacatttgtccaaatccatagaatgtacaacaccagagtgagccctaatgtaaactaaggattctgggtgatattgtaacaaatgcaccattaattgtaacaaatgtatcattttgtaccttctgatggggaatgttgagaatgagagaggctatgcatgtgtggaggcaggagtgggtatatgggatatctctgtatcttcctctcaattttgctgtgaacctataactacctaaaaaagtcttttagaaagcccagtagttttttgcttctctttatgggttggtttccttctctcaagtgaaaaatgggcttcctccatgtagcagatgatatggcttctctcatcccagagaagagagttctttcttgtcaattacagccagaaaaatctccaagaaggatttagatggtcctagtttgctccctcccatccctcttcctttggatctcagatcagaagtgacttctactgggatgctgccctgttaccccagtcttggtcgggtccctgttatgtgctcccactataccatatccttctccttcctagtcttcatcacagtttgaagatgaaaattcattggtggggttacatggctcccccatgtctgattcctcctctaaactgtaagctatagggggcaatgactttatttttttgcttaccattgtgtttctagcacctagcatctggcacataggcacacaataaatatccattaaataaatgactgaaataaacagagggctcttttgctctgattactctgaagagcaattattacatagcagtgacagcttagtgtattctcagaaaatattcttttgttttaaaaccacttatttttctggccaggcatggtggctcacgcctgttatctcagcactttgggaggccgaggtaggcggatcacaaggtcaggagatcgagaccatcctggataacatggtgaaaccctgtctctactaaaaatacaaaaaaatgagccgggcttggtggcgggcgcctgtagtcccagctactagggaggctgaggcaggagaatggcgtgaacccaggaggtggaggttgccatgagccaagatcgcaccgctgaacttcagcttgggcgacagagcgagattccatctcaaaaaaaaaaaatttttttttctgataataaacacaacagactgggcacagtggcgcatacctgtaatcctggtacattgggaggccaaggtgggaggatcacttgagtccaggagttcaagaccagcctgggcaacattgtgagacatcatctctatttaaaaacaaacaaacaaacaaacaaacaaacaaacaaacactccttaaatccccacacacttatgacagaataattgtaagacaaagaaaagtacagttaagaaaacaaaaaacaaaaattacttatatctgtaaccc

SEQ ID NO: 21 - DNA, 5092 bp, Homo sapiens
cttggctgttcctgaggcctggcctggctccccgctgaccccttcccagacctgggatggcggaggccggcctgaggggctggctgctgtgggccctgctcctgcgcttggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctcgactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaatgccccgctcaccttcaaggatggcacagccctggccctgagctgcatggctgactacggggcccctgtcttccccttccttgccattggggggtacaaaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcatggctgagcgctctctggaagacgagatcaatcgcaccacagctgaagacctgcccatctttgccaccagctacattgtcatattcctgtacatctctctggccctgggcagctattccagctggagccgagtgatggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccagaggctgccccggaggcctggggagccacgagaggtccacattgggcgagccctaggcagggtggctcccagcatgctgttgtgcagcctctctgaggccatctgcttcttcctaggggccctgacccccatgccagctgtgcggacctttgccctgacctctggccttgcagtgatccttgacttcctcctgcagatgtcagcctttgtggccctgctctccctggacagcaagaggcaggaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtgctgctgctgtttctcgccctgttcggagtgagcctctactccatgtgccacatcagcgtgggactggaccaggagctggccctgcccaaggactcgtacctgcttgactatttcctctttctgaaccgctacttcgaggtgggggccccggtgtactttgttaccaccttgggctacaacttctccagcgaggctgggatgaatgccatctgctccagtgcaggctgcaacaacttctccttcacccagaagatccagtatgccacagagttccctgagcagtcttacctggccatccctgcctcctcctgggtggatgacttcattgactggctgaccccgtcctcctgctgccgcctttatatatctggccccaataaggacaagttctgcccctcgaccgtcaactctctgaactgcctaaagaactgcatgagcatcacgatgggctctgtgaggccctcggtggagcagttccataagtatcttccctggttcctgaacgaccggcccaacatcaaatgtcccaaaggcggcctggcagcatacagcacctctgtgaacttgacttcagatggccaggttttagacacagttgccattctgtcacccaggctggagtacagtggcacaatctcggctcactgcaacctctacctcctggattcagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtggggcatcagttacaatgctgtgtccctcatcaacctggtctcggcggtgggcatgtctgtggagtttgtgtcccacattacccgctcctttgccatcagcaccaagcccacctggctggagagggccaaagaggccaccatctctatgggaagtgcggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctacgtggggcctgacgttaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctgatacagccagaggccctgtctaggctctatggccctgaaccaaagggttatggggatcttccttgtgactgccccttgacacacgccctcctcaaatcctaggggaggccattcccatgagactgcctgtcactggaggatggcctgctcttgaggtatccaggcagcaccactgatggctcctctgctcccatagtgggtccccagtttccaagtcacctaggccttgggcagtgcctcctcctgggcctgggtctggaagttggcaggaacagacacactccatgtttgtcccacactcactcactttcctaggagcccacttctcatccaacttttcccttctcagttcctctctcgaaagtcttaattctgtgtcagtaagtctttaacacgtagcagtgtccctgagaacacagacaatgaccactaccctgggtgtgatatcacaggaggccagagagaggcaaaggctcaggccaagagccaacgctgtgggaggccggtcggcagccactccctccagggcgcacctgcaggtctgccatccacggccttttctggcaagagaagggcccaggaaggatgctctcataaggcccaggaaggatgctctcataagcaccttggtcatggattagcccctcctggaaaatggtgttgggtttggtctccagctccaatacttattaaggctgttgctgccagtcaaggccacccaggagtctgaaggctgggagctcttggggctgggctggtcctcccatcttcacctcgggcctggatcccaggcctcaaaccagcccaacccgagcttttggacagctctccagaagcatgaactgcagtggagatgaagatcctggctctgtgctgtgcacataggtgtttaataaacatttgttggcagaaaaaaaaaaaaaaaaaaaaaaaaaaaaaaaaaaaaaaaaaaaaaaa

SEQ ID NO: 22 - protein, 1359 aa, Homo sapiens
MAEAGLRGWLLWALLLRLAQSEPYTTIHQPGYCAFYDECGKNPELSGSLMTLSNVSCLSNTPARKITGDHLILLQKICPRLYTGPNTQACCSAKQLVSLEASLSITKALLTRCPACSDNFVNLHCHNTCSPNQSLFINVTRVAQLGAGQLPAVVAYEAFYQHSFAEQSYDSCSRVRVPAAATLAVGTMCGVYGSALCNAQRWLNFQGDTGNGLAPLDITFHLLEPGQAVGSGIQPLNEGVARCNESQGDDVATCSCQDCAASCPAIARPQALDSTFYLGQMPGSLVLIIILCSVFAVVTILLVGFRVAPARDKSKMVDPKKGTSLSDKLSFSTHTLLGQFFQGWGTWVASWPLTILVLSVIPVVALAAGLVFTELTTDPVELWSAPNSQARSEKAFHDQHFGPFFRTNQVILTAPNRSSYRYDSLLLGPKNFSGILDLDLLLELLELQERLRHLQVWSPEAQRNISLQDICYAPLNPDNTSLYDCCINSLLQYFQNNRTLLLLTANQTLMGQTSQVDWKDHFLYCANAPLTFKDGTALALSCMADYGAPVFPFLAIGGYKGKDYSEAEALIMTFSLNNYPAGDPRLAQAKLWEEAFLEEMRAFQRRMAGMFQVTFMAERSLEDEINRTTAEDLPIFATSYIVIFLYISLALGSYSSWSRVMVDSKATLGLGGVAVVLGAVMAAMGFFSYLGIRSSLVILQVVPFLVLSVGADNIFIFVLEYQRLPRRPGEPREVHIGRALGRVAPSMLLCSLSEAICFFLGALTPMPAVRTFALTSGLAVILDFLLQMSAFVALLSLDSKRQEASRLDVCCCVKPQELPPPGQGEGLLLGFFQKAYAPFLLHWITRGVVLLLFLALFGVSLYSMCHISVGLDQELALPKDSYLLDYFLFLNRYFEVGAPVYFVTTLGYNFSSEAGMNAICSSAGCNNFSFTQKIQYATEFPEQSYLAIPASSWVDDFIDWLTPSSCCRLYISGPNKDKFCPSTVNSLNCLKNCMSITMGSVRPSVEQFHKYLPWFLNDRPNIKCPKGGLAAYSTSVNLTSDGQVLDTVAILSPRLEYSGTISAHCNLYLLDSASRFMAYHKPLKNSQDYTEALRAARELAANITADLRKVPGTDPAFEVFPYTITNVFYEQYLTILPEGLFMLSLCLVPTFAVSCLLLGLDLRSGLLNLLSIVMILVDTVGFMALWGISYNAVSLINLVSAVGMSVEFVSHITRSFAISTKPTWLERAKEATISMGSAVFAGVAMTNLPGILVLGLAKAQLIQIFFFRLNLLITLLGLLHGLVFLPVILSYVGPDVNPALALEQKRAEEAVAAVMVASCPNHPSRVSTADNIYVNHSFEGSIKGAGAISNFLPNNGRQF

SEQ ID NO: 23 - DNA, 21 bp, artificial
  synthetic sequence
tggtctttacagaactcacta

SEQ ID NO: 24 - DNA, 21 bp, artificial
  synthetic sequence
tccggacaataccagtctcta

SEQ ID NO: 25 - DNA, 76 bp, artificial
  synthetic sequence
ggatcccgtagtgagttctgtaaagaccattgatatccgtggtctttacagaactcactattttttccaaaagctt

SEQ ID NO: 26 - DNA, 76 bp, artificial
  synthetic sequence
ggatcccgtagagactggtattgtccggattgatatccgtccggacaataccagtctctattttttccaaaagctt

SEQ ID NO: 27 - DNA, 960 bp, Homo sapiens
atctgcagctcagctttggtaatgggggcccattaccaaatgggggtaaaggtcatggcccatcctggtgatagtgagaacccaaggtaggccttgaagattcctatcaggagggagcagaaagtgtgtaccacacccctgggcccaggtggagcagggctgctgctcaaggctcccagccatgctctgtcccttgctaggggtgaccggtgggacaggcctgggcaagggacaagagggagaaggtcggggggaagaggggatgaagagcaaagtgagcaaaggagagtcttccactatctggggtctctgtcaactgtcaggccctagagtgagctgttctttccctttgcttcctggaggaggggacttttgtcactgcgtcactccaccctgcctgcccctccgttatcaggctgttaatattaattaacaacagttgctagggatgacagtgcagagggttcctctgagcccattgctggccctggtcccaagagggggtagggcagagctggggtctgaggctgagccagggagggtgcggaggttcctcggccatgctgagctcctgaggccgggtcccagccagtgcctggtcccatctgtgcctccaggccctggcaccaactccagcagtgttaggggctaatagcgtggtctctcccctagctgactcagccctctggcttcggtcgctttgggaagtgagtggagaccctagcacctgcgtgatgaggctcatctaaagcgggggcctgtggactggggccaaacagtgggagtggtggatcattaaccagcagggctcagcctcattggtccctaacccagtcaggccagggttgtcatcgaaggggaggaggctgccttaatgtgtgttcagcccttggctgttcctgaggcctggcctggctccccgctgaccccttcccagacctgggatggcggaggccggcc

SEQ ID NO: 28 - DNA, 970 bp, Mus musculus
cctgcctaagcttgggcggattcccctctgagcccacccgagcccctgggacactggtggaactcagtaggagcccctccctgcagctgtctcaacaggtagctgcatgagtggccttgaagcaattatcagcaattcagccctggcaatagaggccaaggtcctggcctgtcttggtgatagcaagagcccaaggaaagactggaagtttcctactggaaagaagcagaggatgaaccatgtacctgggcccaggttgggtgggacttgccactcagagcccctaaccagggttgttcagaggactaggccagggccaggaccaagaaagggatagaacgggcatgaggaggaagggtgaagggatccaaggaatctctggtcctgttccctgttaggacatttgtcatggaatcactctcgcttagtgtctctgttatctgggtgctaatagcaactattcagttgctaggatgttaggtgagtctgaacctacccttgatgttgatctgaagaggcgatgcgttagactgcaggttggaggccaagtccaggacagtgttgatattctggatctccaagaagcctccaaggccaaagccaggccagtgtctggtctcgcagaggaacagctctgcatctcttgcccggttggctctaactaccacattagacttcagttgcgtcaaaaaacgaggggaccccagcgccttcactaggaagttgacctcagaaggaggagatggaatggcaccatctgatgtaagggaagagaaaataaattattaaccagtacggcccagtcctattggccccatgacagacgagggttatcactaagaggaggaagctgccttaatgtgcaaactcaggggccagtcctcagcttccccggctgtctccaaggcctggtcctgcttttccttgatcacttcctggctctgggatggcagctgcctggcagggatgg

SEQ ID NO: 29 - protein, 8 aa, artificial
  synthetic peptide tag
DYKDDDDK

SEQ ID NO: 30 - DNA, 23 bp, artificial
  primer
ctatacgaagttatgtcaagcgg

SEQ ID NO: 31 - DNA, 25 bp, artificial
  primer
cttgcacctgacttcctcatataag

SEQ ID NO: 32 - DNA, 23 bp, artificial
  primer
aaagaaggaaagcggccgccagg

SEQ ID NO: 33 - DNA, 25 bp, artificial
  primer
aggaaccgtactgagcgcataccaa

(19) United States
(12) Patent Application Publication
(10) Pub. No.: US 2009/0035784 A1
(43) Pub. Date: Feb. 5, 2009

(54) NPC1L1 AND NPC1L1 INHIBITORS AND METHODS OF USE THEREOF

(75) Inventors: Yiannis Ioannou, New York, NY (US); Joanna P. Davies, Long Island City, NY (US)

Correspondence Address: DARBY & DARBY P.C., P.O. BOX 770, Church Street Station, New York, NY, 10008-0770, US

(73) Assignee: Mount Sinai School of Medicine of New York University, New York, NY (US)

(21) Appl. No.: 11/572,227

(22) PCT Filed: Aug. 1, 2005
(86) PCT No.: PCT/US05/27579

§ 371 Date: Feb. 28, 2008

Related U.S. Application Data
(60) Provisional application No. 60/592,592, filed on Jul. 30, 2004.

(51) Int. Cl. G01N 33/53; C07H 21/04; C07K 14/435

(52) U.S. Cl. 435/7.1; 536/23.5; 530/350

(57) ABSTRACT

The present invention provides a novel gene, designated herein as “NPC1L1”, that is associated with lipid or glucose metabolism. The invention further provides the use of the NPC1L1 gene and its corresponding protein to diagnose a lipid condition in a cell or tissue and to screen for novel therapeutic compounds useful for treating lipid disorders and other NPC1L1-associated or mediated diseases or disorders. The invention further provides specific inhibitors of NPC1L1.

### RELATED APPLICATIONS

The present application claims priority to provisional application Ser. No. 60/592,592, filed on Jul. 30, 2004, the contents of which are expressly incorporated by reference herein.

### FIELD OF INVENTION

The present invention relates to the identification of a Niemann-Pick C1 Like 1 (NPC1L1) gene. The present invention further includes NPC1L1 nucleic acids and polypeptides, as well as transgenic animals with disrupted NPC1L1 function. In addition, the present invention relates to methods of use for NPC1L1 molecules, including drug screening, diagnostics, and treatment of disorders relating to aberrant lipid and glucose metabolism.

### BACKGROUND OF THE INVENTION

### Lipid Metabolism and Hyperlipidemia

Diets high in lipids, such as fat and cholesterol, are important factors in the development of many human diseases, including obesity, diabetes mellitus, atherosclerosis, and coronary artery disease. In addition, aberrant regulation of lipids can contribute to many other conditions, such as arthritis, cancer, hypertension, and vascular disorders. Modulating the biochemical and molecular mechanisms of lipid metabolism is therefore a crucial goal of contemporary research and medicine.

The control of lipid metabolism is highly complex, reflecting a delicate balance between the processes of ingestion, synthesis, and mobilization. The mechanisms underlying cholesterol control, for example, include absorption of dietary cholesterol in the intestine; de novo production of cholesterol in the liver; secretion of cholesterol into the blood and lymph via lipoprotein carriers, and transport of cholesterol-lipoproteins from the serum to target tissues for use and elimination. Each of these steps represents a potential point for regulation as well as potential target for medical intervention.

In addition, chemical modifications of lipids play a key role in regulating metabolism. One key step is the addition of ester groups to cholesterol in the endoplasm reticulum, a modification that renders cholesterol more hydrophobic and competent for assembly into lipoprotein complexes. Lipoprotein complexes are essential for the transport of lipids to tissues; free lipids are virtually undetectable in the blood. There are least five distinct families of lipoproteins, each distinguished by their density as well as functional role in lipid metabolism.

Cholesterol esters are not just critical in intestinal absorption of cholesterol and its subsequent deposition into lipoprotein carriers. They are also the major component of atherosclerotic plaques, which underlie vascular disorders such as coronary artery disease—the leading cause of death in industrialized nations. Accordingly, the aberrant regulation of cholesterol metabolism can lead to elevated levels of serum cholesterol and promote cardiovascular disease.

While the pathways underlying de novo synthesis and breakdown of cholesterol are well understood, the specific mechanisms that mediate cholesterol transport across the intestinal epithelium remains unclear. Finding new ways to block the absorption of cholesterol may lower serum cholesterol and have significant clinical implications for conditions such as diet-induced obesity, diabetes, and cardiovascular disease. There is a need in the art for further investigations of lipid metabolism, especially with respect to cholesterol absorption.

### Niemann Pick C1

The human Niemann-Pick C1 gene (NPC1) encodes a transmembrane transporter that is defective in the rare cholesterol storage disease, Niemann-Pick C1. NPC1 localizes to late endosomes and plays a pivotal role in intracellular transport of cholesterol and other lipids. Cells lacking NPC1 have a number of distinct trafficking defects: (i) unesterified cholesterol derived from low-density lipoproteins (LDLs) accumulates in lysosomes; (ii) cholesterol accumulates in the trans-golgi network; and (iii) cholesterol transport to and from the plasma membrane is delayed.

The present invention provides a novel Niemann-Pick C1 Like 1 (NPC1L1) gene that is also involved in lipid metabolism.

### SUMMARY OF THE INVENTION

The present invention provides an isolated nucleic acid that comprises a nucleotide sequence encoding a non-human NPC1L1 polypeptide, and fragments thereof. In one embodiment, the isolated genomic nucleic acid comprises a nucleotide sequence set forth SEQ ID NO:1.

In another embodiment, the nucleic acid comprises a nucleotide sequence set forth SEQ ID NO:2.

The present invention provides an isolated NPC1L1 nucleic acid which encodes a polypeptide having an amino acid sequence set forth in SEQ ID NO:3.

The present invention also provides NPC1L1 polypeptides encoded by the NPC1L1 nucleic acid sequences described above. In one embodiment, the NPC1L1 polypeptide is a non-human NPC1L1 polypeptide. In a specific embodiment, embodiment, the NPC1L1 polypeptide has the amino acid sequence set forth in SEQ ID NO: 3.

In addition, the present invention encompasses isolated nucleic acids with mutations in NPC1L1 coding sequences, and which encode NPC1L1 polypeptides having altered amino acid sequences.

The invention also provides recombinant vectors and host cells comprising the NPC1L1 nucleic acid molecules, as well as methods for producing an NPC1L1 polypeptide using such host cells. In one embodiment, the host cells are bacterial or eukaryotic cells engineered for studies of NPC1L1 function.

The invention further provides non-human transgenic animals comprising such a recombinant vector. In one embodiment, the animal is a mouse.

The invention also provides an oligonucleotide, such as a primer or probe, wherein the oligonucleotide has a sequence identical to a contiguous nucleotide sequence in the NPC1L1 nucleotide sequence, e.g., SEQ ID NO:2. The oligonucletide has a length at least 10 bases, preferably at least 20 bases, and more preferably at least 30 bases.

The invention further provides antibodies that bind specifically to an NPC1L1 protein having an amino acid sequence shown in SEQ ID NO:3, or fragments thereof.

The present invention includes methods of screening to identify an antagonist or agonist of a NPC1L1 nucleic acid or polypeptide. Such agonists/antagonists are thus designated candidate compounds for the treatment (e.g., therapeutic and prophylactic) of NPC1L1-mediated disorders, such as hyperlipidemia, and other diseases and disorders associated with or mediated by NPC1L1, including, but not limited to, body weight disorders such as obesity, diabetes, e.g., type II diabetes, cardiovascular disease, including, for example, ischemia, congestive heart failure, and atherosclerosis, and stroke. NPC1L1-mediated disorders include those disorders which are mediated by the expression or activity of NPC1L1, including plasma membrane uptake and transport of various lipids, including cholesterol and sphingolipids.

In one embodiment, the NPC1L1 antagonist is selected from the group consisting of a small molecule, an anti-NPC1L1 antibody, an NPC1L1 antisense nucleic acid, an NPC1L1 ribozyme, an NPC1L1 triple-helix, or an NPC1L1 inhibitory RNA. In another embodiment, the NPC1L1 antagonist inhibits transcription of NPC1L1 by targeting an NPC1L1 promoter transcription factor. In this embodiment the specific agonist or antagonist is identified by its ability to downregulate the expression of a reporter gene (such as luciferase or green fluorescence protein) driven by the promoter for NPC1L1. In another embodiment, the inhibitor is selected from the group consisting on: 4-phenyl-4-piperidinecarbonitrile hydrochloride, 1-butyl-N-(2,6-dimethylphenyl)-2 piperidinecarboxamide, 1-(1-naphthylmethyl)piperazine, 3 {1-[(2-methylphenyl)amino]ethylidene}-2,4(3H, 5H)-thiophenedione, 3 {1-[(2-hydroxyphenyl)amino]ethylidene}-2,4(3H, 5H)-thiophenedione, 2-acetyl-3-[(2-methylphenyl)amino]-2-cyclopenten-1-one, 3-[(4-methoxyphenyl)amino]-2-methyl-2-cyclopenten-1-one, 3-[(2-methoxyphenyl)amino]-2-methyl-2-cyclopenten-1-one, and N-(4-acetylphenyl)-2-thiophenecarboxamide.

The invention further provides a mammal, preferably a mouse, comprising a homozygous or heterozygous disruption of endogenous NPC1L1, wherein the mouse produces less functional NPC1L1 polypeptide or does not produce any functional NPC1L1 polypeptide.

The invention further describes transgenic mammal, preferably a mouse, in which the mouse NPC1L1 genomic gene or cDNA is into the mouse genome in multiple copies, which is a model for hyperlipidemia. In one embodiment, the hyperlipidemia is hypercholesterolemia.

The present invention also provides a method of inhibiting the cellular uptake of a lipid by inhibiting the expression or activity of an NPC1L1 nucleic acid or polypeptide.

Further provided is a method of treating hyperlipidemia or other diseases and disorders associated with or mediated by NPC1L1, including, but not limited to, obesity, diabetes, e.g., type II diabetes, cardiovascular disease, or stroke in a subject in need thereof by administering to the subject a therapeutically effective amount of an agent which inhibits the expression or activity of an NPC1L1 nucleic acid or polypeptide.

In one embodiment, the NPC1L1 nucleic acid or polypeptide which is inhibited is that set forth in SEQ ID NOs: 2 and 3, respectively.

In another embodiment, the hyperlipidemia is hypercholesterolemia.

The present invention further provides a method of decreasing the plasma glucose by administering a therapeutically effective amount of an agent which inhibits the expression or activity of an NPC1L1 nucleic acid or polypeptide.

In one embodiment, the NPC1L1 nucleic acid or polypeptide which is inhibited is that set forth in SEQ ID NOs: 2 and 3, respectively.

In another embodiment, the hyperlipidemia is dietary hypercholesterolemia.

The present invention also provides a method for identifying a test compound that binds to and modulates the activity of an NPC1L1 polypeptide, which compound is therefore a candidate compound for the treatment of hyperlipidemia, obesity, diabetes, e.g., type II diabetes, cardiovascular disease, or stroke.

### BRIEF DESCRIPTION OF DRAWINGS

FIGS. 1A-1E. FIG. 1 demonstrates the subcellular localization of murine NPC1L1 by immunofluorescence. FIG. 1a shows localization in human NT2 cells. FIG. 1b shows localization of tagged NPC1L1 in transfected COS-7 cells. FIG. 1c shows localization in Caco-2 cells transiently transfected with an NPC1L1 fusion protein. FIG. 1d depicts the lack of localization of NPC1L1 on the plasma membrane. FIG. 1e demonstrates the effect of NPC1L1 on fatty acid transport in bacterial cells.

FIGS. 2A-2F. FIG. 2 shows the tissue distribution of human and mouse NPC1L1 in various tissues in human (FIGS. 2a and 2b) and mouse (FIG. 2c) tissues using quantitative real time PCR (FIGS. 2d and 2e). FIG. 2f demonstrates reduced activation of reporter genes in cells from NPC1L1-deficient mice (L1) compared with control mice (WT), under the expression of three response elements: ABCA1-RFP (FIG. 2f(1-4)); DR4-RFP (FIG. 2f(5-8)); and SRE-GFP (FIG. 2f(9-12)).

FIGS. 3A-3E. FIG. 3 demonstrates impaired uptake of multiple lipids (i.e., oleic acid, cholesterol) in mouse cells from NPC1L1 deficient mice using radioactively labeled lipids (FIG. 3a-b), fluorescently-tagged lipids complexed with cyclodextrin (FIG. 3c) or BSA (FIG. 3d). FIG. 3e demonstrates expression of a caveolin-mYFP fusion in mouse wild-type or NPC1L1 null cells.

FIG. 4. FIG. 4 demonstrates resistance to hypercholesterolemia in NPC1L1 null mice subjected to a high cholesterol diet. FIG. 4 shows plasma assays for glucose, triglycerides, total cholesterol and HDL-cholesterol after 14 weeks.

FIG. 5. FIG. 5 demonstrates the AcrAB-TolC complex in E. coli and the homologous MexCD-OprJ complex from Pseudomonas aeruginosa.

FIG. 6. Immunofluorescence of lysosomal cholesterol of normal human fibroblasts treated (6B) or untreated (6A) with NPC1 inhibitor 4-butyryl-4-phenylpiperidine.

FIG. 7. Immunofluorescence of lysosomal cholesterol of normal human fibroblasts treated with weaker NPC1 inhibitor 4-cyano-4-phenylpiperidine (7A), or 4-methylpiperidine (7B).

FIG. 8 is a graph illustrating that inhibitors 4-Phenyl-4-piperidinecarbonitrile Hydrochloride (#1), (1-Butyl-N(2,6-diemethylphenyl)2 piperidine carboxamide) #7,2-acetyl-3-[(2-methylphenyl)amino]-2-cyclopenten-1-one, 3 {1-[(2-hydroxyphenyl)amino]ethylidene}-2,4(3H, 5H)-thiophenedione and gave a positive signal compared to control (none). Note that Ezetamibe did not inhibit NPC1L1 in this assay.

FIGS. 9A-9B. FIG. 9A is a graph depicting body weights of mice fed a high fat diet for 0-245 days (Mouse set 1). FIG. 9B is a graph depicting body weights of mice fed a high fat diet for 0-95 days (mouse set 2).

FIG. 10 is a graph depicting results of a glucose tolerance test on mice fed with regular chow (mouse set 1).

FIGS. 11A-11B. FIG. 11A is a graph depicting results of a glucose tolerance test on mice fed a high fat diet for 102 days (mouse set 1). FIG. 11B is a graph depicting results of a glucose tolerance test on mice fed a high fat diet for 262 days (mouse set 1).

FIGS. 12A-12B. FIG. 12A is a graph depicting results of an insulin tolerance test in mice fed a high fat diet for 105 days (mouse set 2). FIG. 12B is a graph depicting results of an insulin tolerance test in mice fed a high fat diet for 252 days (mouse set 1).

FIGS. 13A-13B. FIG. 13A is a graph depicting insulin measurements in mice fed a high fat diet for 72 days (mouse set 2). FIG. 13B is a graph depicting insulin measurements in mice fed a high fat diet for 220 days (mouse set 1).

FIGS. 14A-14B are graphs depicting plasma lipoprotein profiles in mice at 120 days (FIG. 14A) and 268 days (FIG. 14B) of high fat diet.

FIG. 15 is a graph depicting results of real-time PCR of NPC1L1 in mouse tissue and 3T3L1 cell line.

FIG. 16 is a graph depicting results of real-time PCR of NPC1L1 in mouse white and brown adipose tissue.

FIG. 17 is a graph depicting results of real-time PCR of NPC1L1 in human liver and adipose tissue.

FIG. 18 is a table illustrating weight gain and food intake over 210 days for NPC1L1 knockout mice fed a high fat diet as compared to wild type mice fed a high fat diet.

### DETAILED DESCRIPTION OF THE INVENTION

The Niemann Pick C1-like gene and gene product (NPC1L1; also known as NPC3; Genbank Accession No. AF192522; Davies et al., (2000) Genomics 65(2): 137-145 and Ioannou et al., (2000) Mol. Genet. Metab. 71(1-2): 175-181 was first isolated in humans, based on its 42% amino acid identity and 51% amino acid similarity to human NPC1 (Genbank Accession No. AF002020).

The present invention is based on methods of using NPC1L1 molecules including screening assays for identifying modulators of NPC1L1, inhibitors of NPC1L1 including small molecule compounds, antibodies, and siRNA molecules, NPC1L1 knock-out animals and transgenic animals, as well as therapeutic methods for the treatment of NPC1L1 mediated disease and disorders including, but not limited to, lipid disorders such as hyperlipidemia, and obesity, diabetes, and cardiovascular disease using modulators, e.g., inhibitors of NPC1L1. Methods for treating disorders associated with decreased NPC1L1, e.g., anorexia, cachexia, and wasting, using agonists of NPC1L1 are also included in the invention. The present invention also includes diagnostic methods using NPC1 L1.

### DEFINITIONS

The term “subject” as used herein refers to a mammal (e.g., a rodent such as a mouse or a rat, a pig, a primate, or companion animal (e.g., dog or cat, etc.). In particular, the term refers to humans.

The terms “array” and “microarray” are used interchangeably and refer generally to any ordered arrangement (e.g., on a surface or substrate) of different molecules, referred to herein as “probes.” Each different probe of an array is capable of specifically recognizing and/or binding to a particular molecule, which is referred to herein as its “target,” in the context of arrays. Examples of typical target molecules that can be detected using microarrays include mRNA transcripts, cDNA molecules, cRNA molecules, and proteins. As disclosed in the Examples section below, at least one target detectable by the Affymetrix GeneChip® microarray used as described herein is a NPC1L1-encoding nucleic acid (such as an mRNA transcript, or a corresponding cDNA or cRNA molecule).

An “antisense” nucleic acid molecule or oligonucleotide is a single stranded nucleic acid molecule, which may be DNA, RNA, a DNA-RNA chimera, or a derivative thereof, which, upon hybridizing under physiological conditions with complementary bases in an RNA or DNA molecule of interest, inhibits the expression of the corresponding gene by inhibiting, e.g., mRNA transcription, mRNA splicing, mRNA transport, or mRNA translation or by decreasing mRNA stability. As presently used, “antisense” broadly includes RNA-RNA interactions, RNA-DNA interactions, and RNase-H mediated arrest. Antisense nucleic acid molecules can be encoded by a recombinant gene for expression in a cell (see, e.g., U.S. Pat. Nos. 5,814,500 and 5,811,234), or alternatively they can be prepared synthetically (see, e.g., U.S. Pat. No. 5,780,607). According to the present invention, the role of NPC1L1 in regulation of conditions associated with hyperlipidemia may be identified, modulated and studied using antisense nucleic acids derived on the basis of NPC1L1-encoding nucleic acid molecules of the invention.

The term “ribozyme” is used to refer to a catalytic RNA molecule capable of cleaving RNA substrates. Ribozyme specificity is dependent on complementary RNA-RNA interactions (for a review, see Cech and Bass, Annu. Rev. Biochem. 1986; 55: 599-629). Two types of ribozymes, hammerhead and hairpin, have been described. Each has a structurally distinct catalytic center. The present invention contemplates the use of ribozymes designed on the basis of the NPC1L1-encoding nucleic acid molecules of the invention to induce catalytic cleavage of the corresponding mRNA, thereby inhibiting expression of the NPC1L1 gene. Ribozyme technology is described further in Intracellular Ribozyme Applications: Principals and Protocols, Rossi and Couture ed., Horizon Scientific Press, 1999.

The term “RNA interference” or “RNAi” refers to the ability of double stranded RNA (dsRNA) to suppress the expression of a specific gene of interest in a homology-dependent manner. It is currently believed that RNA interference acts post-transcriptionally by targeting mRNA molecules for degradation. RNA interference commonly involves the use of dsRNAs that are greater than 500 bp; however, it can also be mediated through small interfering RNAs (siRNAs) or small hairpin RNAs (shRNAs), which can be 10 or more nucleotides in length and are typically 18 or more nucleotides in length. For reviews, see Bosner and Labouesse, Nature Cell Biol. 2000; 2: E31-E36 and Sharp and Zamore, Science 2000; 287: 2431-2433.

The term “nucleic acid hybridization” refers to anti-parallel hydrogen bonding between two single-stranded nucleic acids, in which A pairs with T (or U if an RNA nucleic acid) and C pairs with G. Nucleic acid molecules are “hybridizable” to each other when at least one strand of one nucleic acid molecule can form hydrogen bonds with the complementary bases of another nucleic acid molecule under defined stringency conditions. Stringency of hybridization is determined, e.g., by (i) the temperature at which hybridization and/or washing is performed, and (ii) the ionic strength and (iii) concentration of denaturants such as formamide of the hybridization and washing solutions, as well as other parameters. Hybridization requires that the two strands contain substantially complementary sequences. Depending on the stringency of hybridization, however, some degree of mismatches may be tolerated. Under “low stringency” conditions, a greater percentage of mismatches are tolerable (i.e., will not prevent formation of an anti-parallel hybrid). See Molecular Biology of the Cell, Alberts et al., 3^{rd} ed., New York and London: Garland Publ., 1994, Ch. 7.

Typically, hybridization of two strands at high stringency requires that the sequences exhibit a high degree of complementarity over an extended portion of their length. Examples of high stringency conditions include: hybridization to filter-bound DNA in 0.5 M NaHPO₄, 7% SDS, 1 mM EDTA at 65° C., followed by washing in 0.1×SSC/0.1% SDS (where 1×SSC is 0.15 M NaCl, 0.15 M Na citrate) at 68° C. or for oligonucleotide molecules washing in 6×SSC/0.5% sodium pyrophosphate at about 37° C. (for 14 nucleotide-long oligos), at about 48° C. (for about 17 nucleotide-long oligos), at about 55° C. (for 20 nucleotide-long oligos), and at about 60° C. (for 23 nucleotide-long oligos)).

Conditions of intermediate or moderate stringency (such as, for example, an aqueous solution of 2×SSC at 65° C.; alternatively, for example, hybridization to filter-bound DNA in 0.5 M NaHPO₄, 7% SDS, 1 mM EDTA at 65° C., and washing in 0.2×SSC/0.1% SDS at 42° C.) and low stringency (such as, for example, an aqueous solution of 2×SSC at 55° C.), require correspondingly less overall complementarity for hybridization to occur between two sequences. Specific temperature and salt conditions for any given stringency hybridization reaction depend on the concentration of the target DNA and length and base composition of the probe, and are normally determined empirically in preliminary experiments, which are routine (see Southern, J. Mol. Biol. 1975; 98: 503; Sambrook et al., Molecular Cloning: A Laboratory Manual, 2^{nd} ed., vol. 2, ch. 9.50, CSH Laboratory Press, 1989; Ausubel et al. (eds.), 1989, Current Protocols in Molecular Biology, Vol. I, Green Publishing Associates, Inc., and John Wiley & Sons, Inc., New York, at p. 2.10.3).

As used herein, the term “standard hybridization conditions” refers to hybridization conditions that allow hybridization of two nucleotide molecules having at least 75% sequence identity. According to a specific embodiment, hybridization conditions of higher stringency may be used to allow hybridization of only sequences having at least 80% sequence identity, at least 90% sequence identity, at least 95% sequence identity, or at least 99% sequence identity.

Nucleic acid molecules that “hybridize” to any of the NPC1L1-encoding nucleic acids of the present invention may be of any length. In one embodiment, such nucleic acid molecules are at least 10, at least 15, at least 20, at least 30, at least 40, at least 50, and at least 70 nucleotides in length. In another embodiment, nucleic acid molecules that hybridize are of about the same length as the particular NPC1L1-encoding nucleic acid.

The term “homologous” as used in the art commonly refers to the relationship between nucleic acid molecules or proteins that possess a “common evolutionary origin,” including nucleic acid molecules or proteins within superfamilies (e.g., the immunoglobulin superfamily) and nucleic acid molecules or proteins from different species (Reeck et al., Cell 1987; 50: 667). Such nucleic acid molecules or proteins have sequence homology, as reflected by their sequence similarity, whether in terms of substantial percent similarity or the presence of specific residues or motifs at conserved positions.

The terms “percent (%) sequence similarity”, “percent (%) sequence identity”, and the like, generally refer to the degree of identity or correspondence between different nucleotide sequences of nucleic acid molecules or amino acid sequences of proteins that may or may not share a common evolutionary origin (see Reeck et al., supra). Sequence identity can be determined using any of a number of publicly available sequence comparison algorithms, such as BLAST, FASTA, DNA Strider, GCG (Genetics Computer Group, Program Manual for the GCG Package, Version 7, Madison, Wis.), etc.

In addition to the NPC1L1 nucleic acid sequences and NPC1L1 polypeptides (as shown in, e.g., SEQ ID NOS: 2 and 3, respectively), the present invention further provides polynucleotide molecules comprising nucleotide sequences having certain percentage sequence identities to any of the aforementioned sequences. Such sequences preferably hybridize under conditions of moderate or high stringency as described above, and may include species orthologs.

As used herein, the term “orthologs” refers to genes in different species that apparently evolved from a common ancestral gene by speciation. Normally, orthologs retain the same function through the course of evolution. Identification of orthologs can provide reliable prediction of gene function in newly sequenced genomes. Sequence comparison algorithms that can be used to identify orthologs include without limitation BLAST, FASTA, DNA Strider, and the GCG pileup program. Orthologs often have high sequence similarity.

The present invention encompasses all non-human orthologs of NPC1L1. In addition to the mouse ortholog, particularly useful NPC1L1 orthologs of the present invention are rat, monkey, porcine, canine (dog), and guinea pig orthologs.

As used herein, the term “isolated” means that the material being referred to has been removed from the environment in which it is naturally found, and is characterized to a sufficient degree to establish that it is present in a particular sample. Such characterization can be achieved by any standard technique, such as, e.g., sequencing, hybridization, immunoassay, functional assay, expression, size determination, or the like. Thus, a biological material can be “isolated” if it is free of cellular components, i.e., components of the cells in which the material is found or produced in nature. For nucleic acid molecules, an isolated nucleic acid molecule or isolated polynucleotide molecule, or an isolated oligonucleotide, can be a PCR product, an mRNA transcript, a cDNA molecule, or a restriction fragment. A nucleic acid molecule excised from the chromosome that it is naturally a part of is considered to be isolated. Such a nucleic acid molecule may or may not remain joined to regulatory, or non-regulatory, or non-coding regions, or to other regions located upstream or downstream of the gene when found in the chromosome. Nucleic acid molecules that have been spliced into vectors such as plasmids, cosmids, artificial chromosomes, phages and the like are considered isolated. In a particular embodiment, a NPC1L1-encoding nucleic acid spliced into a recombinant vector, and/or transformed into a host cell, is considered to be “isolated”.

Isolated nucleic acid molecules and isolated polynucleotide molecules of the present invention do not encompass uncharacterized clones in man-made genomic or cDNA libraries.

A protein that is associated with other proteins and/or nucleic acids with which it is associated in an intact cell, or with cellular membranes if it is a membrane-associated protein, is considered isolated if it has otherwise been removed from the environment in which it is naturally found and is characterized to a sufficient degree to establish that it is present in a particular sample. A protein expressed from a recombinant vector in a host cell, particularly in a cell in which the protein is not naturally expressed, is also regarded as isolated.

An isolated organelle, cell, or tissue is one that has been removed from the anatomical site (cell, tissue or organism) in which it is found in the source organism.

An isolated material may or may not be “purified”. The term “purified” as used herein refers to a material (e.g., a nucleic acid molecule or a protein) that has been isolated under conditions that detectably reduce or eliminate the presence of other contaminating materials. Contaminants may or may not include native materials from which the purified material has been obtained. A purified material preferably contains less than about 90%, less than about 75%, less than about 50%, less than about 25%, less than about 10%, less than about 5%, or less than about 2% by weight of other components with which it was originally associated.

Methods for purification are well-known in the art. For example, nucleic acids or polynucleotide molecules can be purified by precipitation, chromatography (including preparative solid phase chromatography, oligonucleotide hybridization, and triple helix chromatography), ultracentrifugation, and other means. Polypeptides can be purified by various methods including, without limitation, preparative disc-gel electrophoresis, isoelectric focusing, HPLC, reverse-phase HPLC, gel filtration, affinity chromatography, ion exchange and partition chromatography, precipitation and salting-out chromatography, extraction, and counter-current distribution. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting (FACS)). Other purification methods are possible. The term “substantially pure” indicates the highest degree of purity that can be achieved using conventional purification techniques currently known in the art. In the context of analytical testing of the material, “substantially free” means that contaminants, if present, are below the limits of detection using current techniques, or are detected at levels that are low enough to be acceptable for use in the relevant art, for example, no more than about 2-5% (w/w). Accordingly, with respect to the purified material, the term “substantially pure” or “substantially free” means that the purified material being referred to is present in a composition where it represents 95% (w/w) or more of the weight of that composition. Purity can be evaluated by chromatography, gel electrophoresis, immunoassay, composition analysis, biological assay, or any other appropriate method known in the art.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to +5%, and more preferably still up to 1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

The term “degenerate variants” of a polynucleotide sequence are those in which a change of one or more nucleotides in a given codon position results in no alteration in the amino acid encoded at that position.

The term “modulator” refers to a compound that differentially affects the expression or activity of a gene or gene product (e.g., nucleic acid molecule or protein), for example, in response to a stimulus that normally activates or represses the expression or activity of that gene or gene product when compared to the expression or activity of the gene or gene product not contacted with the stimulus. In one embodiment, the gene or gene product the expression or activity of which is being modulated includes a gene, cDNA molecule or mRNA transcript that encodes a mammalian NPC1L1 protein such as, e.g., a rat, mouse, companion animal, or human NPC1L1 protein.

An “antagonist” is one type of modulator, and includes an agent that reduces expression or activity, or inhibits expression or activity, of an NPC1L1 nucleic acid or polypeptide. Examples of antagonists of the NPC1L1-encoding nucleic acids of the present invention include without limitation small molecules, anti-NPC1L1 antibodies, antisense nucleic acids, ribozymes, and RNAi oligonucleotides, and molecule that target NPC1L1 promoter transcription factors. Specific NPC1L1 antagonists are set forth herein.

An “agonist” is another modulator that is defined as an agent that interacts with (e.g., binds to) a nucleic acid molecule or protein, and promotes, enhances, stimulates or potentiates the biological expression or activity of the nucleic acid molecule or protein. The term “partial agonist” is used to refer to an agonist which interacts with a nucleic acid molecule or protein, but promotes only partial function of the nucleic acid molecule or protein. A partial agonist may also inhibit certain functions of the nucleic acid molecule or protein with which it interacts. An “antagonist” interacts with (e.g., binds to) and inhibits or reduces the biological expression or function of the nucleic acid molecule or protein.

A “test compound” is a molecule that can be tested for its ability to act as a modulator of a gene or gene product. Test compounds can be selected, without limitation, from small inorganic and organic molecules (i.e., those molecules of less than about 2 kD, and more preferably less than about 1 kD in molecular weight), polypeptides (including native ligands, antibodies, antibody fragments, and other immunospecific molecules), oligonucleotides, polynucleotide molecules, and derivatives thereof. In various embodiments of the present invention, a test compound is tested for its ability to modulate the expression of a mammalian NPC1L1-encoding nucleic acid or NPC1L1 protein or to bind to a mammalian NPC1L1 protein. A compound that modulates a nucleic acid or protein of interest is designated herein as a “candidate compound” or “lead compound” suitable for further testing and development. Candidate compounds include, but are not necessarily limited to, the functional categories of agonist and antagonist.

The term “detectable change” as used herein in relation to an expression level of a gene or gene product (e.g., NPC1L1) means any statistically significant change and preferably at least a 1.5-fold change as measured by any available technique such as hybridization or quantitative PCR.

As used herein, the term “specific binding” refers to the ability of one molecule, typically an antibody, polynucleotide, polypeptide, or a small molecule ligand to contact and associate with another specific molecule, e.g., an NPC1L1 molecule, even in the presence of many other diverse molecules. “Immunospecific binding” refers to the ability of an antibody to specifically bind to (or to be “specifically immunoreactive with”) its corresponding antigen.

The term “obesity” or “overweight” is defined as a body mass index (BMI) of 30 kg/m² or more (National Institute of Health, Clinical Guidelines on the Identification, Evaluation, and Treatment of Overweight and Obesity in Adults (1998)). However, the present invention is also intended to include a disease, disorder, or condition that is characterized by a body mass index (BMI) of 25 kg/m² or more, 26 kg/m² or more, 27 kg/m² or more, 28 kg/m² or more, 29 kg/m² or more, 29.5 kg/m² or more, or 29.9 kg/m² or more, all of which are typically referred to as overweight (National Institute of Health, Clinical Guidelines on the Identification, Evaluation, and Treatment of Overweight and Obesity in Adults (1998)). Body weight disorders also include conditions or disorders which are secondary to disorders such as obesity or overweight, i.e., are influenced or caused by a disorder such as obesity or overweight. For example, insulin resistance, diabetes, hypertension, and atherosclerosis can all be influenced or caused by obesity or overweight. Accordingly, such secondary conditions or disorders are additional examples of body weight disorders.

The term “cardiovascular disease” (CVD) is any disease or disorder that affects the cardiovascular system. A cardiovascular disease or disorder includes, but is not limited to atherosclerosis, coronary heart disease or coronary artery disease (CAD), myocardial infarction (MI), ischemia, and peripheral vascular diseases.

“Amplification” of DNA as used herein denotes the use of exponential amplification techniques known in the art such as the polymerase chain reaction (PCR), and non-exponential amplification techniques such as linked linear amplification, that can be used to increase the concentration of a particular DNA sequence present in a mixture of DNA sequences. For a description of PCR, see Saiki et al., Science 1988, 239:487 and U.S. Pat. No. 4,683,202. For a description of linked linear amplification, see U.S. Pat. Nos. 6,335,184 and 6,027,923; Reyes et al., Clinical Chemistry 2001; 47: 131-40; and Wu et al., Genomics 1989; 4: 560-569.

As used herein, the phrase “sequence-specific oligonucleotides” refers to oligonucleotides that can be used to detect the presence of a specific nucleic acid molecule, or that can be used to amplify a particular segment of a specific nucleic acid molecule for which a template is present. Such oligonucleotides are also referred to as “primers” or “probes.” In a specific embodiment, “probe” is also used to refer to an oligonucleotide, for example about 25 nucleotides in length, attached to a solid support for use on “arrays” and “microarrays” described below.

The term “host cell” refers to any cell of any organism that is selected, modified, transformed, grown, used or manipulated in any way so as, e.g., to clone a recombinant vector that has been transformed into that cell, or to express a recombinant protein such as, e.g., a NPC1L1 protein of the present invention. Host cells are useful in screening and other assays, as described below.

As used herein, the terms “transfected cell” and “transformed cell” both refer to a host cell that has been genetically modified to express or over-express a nucleic acid encoding a specific gene product of interest such as, e.g., a NPC1L1 protein or a fragment thereof. Any eukaryotic or prokaryotic cell can be used, although eukaryotic cells are preferred, vertebrate cells are more preferred, and mammalian cells are the most preferred. Transfected or transformed cells are suitable to conduct an assay to screen for compounds that modulate the function of the gene product. A typical “assay method” of the present invention makes use of one or more such cells, e.g., in a microwell plate or some other culture system, to screen for such compounds. The effects of a test compound can be determined on a single cell, or on a membrane fraction prepared from one or more cells, or on a collection of intact cells sufficient to allow measurement of activity.

The term “recombinantly engineered cell” refers to any prokaryotic or eukaryotic cell that has been genetically manipulated to express or over-express a nucleic acid of interest, e.g., a NPC1L1-encoding nucleic acid of the present invention, by any appropriate method, including transfection, transformation or transduction. The term “recombinantly engineered cell” also refers to a cell that has been engineered to activate an endogenous nucleic acid, e.g., the endogenous NPC1L1-encoding gene in a rat, mouse or human cell, which cell would not normally express that gene product or would express the gene product at only a sub-optimal level.

The terms “vector”, “cloning vector” and “expression vector” refer to recombinant constructs including, e.g., plasmids, cosmids, phages, viruses, and the like, with which a nucleic acid molecule (e.g., a NPC1L1-encoding nucleic acid or NPC1L1 siRNA-expressing nucleic acid) can be introduced into a host cell so as to, e.g., clone the vector or express the introduced nucleic acid molecule. Vectors may further comprise selectable markers.

The terms “mutant”, “mutated”, “mutation”, and the like, refer to any detectable change in genetic material, (e.g., NPC1L1 DNA), or any process, mechanism, or result of such a change. Mutations include gene mutations in which the structure (e.g., DNA sequence) of the gene is altered; any DNA or other nucleic acid molecule derived from such a mutation process; and any expression product (e.g., the encoded protein) exhibiting a non-silent modification as a result of the mutation.

As used herein, the term “genetically modified animal” encompasses all animals into which an exogenous genetic material has been introduced and/or whose endogenous genetic material has been manipulated. Examples of genetically modified animals include without limitation transgenic animals, e.g., “knock-in” animals with the endogenous gene substituted with a heterologous gene or an ortholog from another species or a mutated gene, “knockout” animals with the endogenous gene partially or completely inactivated, or transgenic animals expressing a mutated gene or overexpressing a wild-type or mutated gene (e.g., upon targeted or random integration into the genome) and animals containing cells harboring a non-integrated nucleic acid construct (e.g., viral-based vector, antisense oligonucleotide, shRNA, siRNA, ribozyme, etc.), including animals wherein the expression of an endogenous gene has been modulated (e.g., increased or decreased) due to the presence of such construct.

As used herein, a “transgenic animal” is a nonhuman animal, preferably a mammal, more preferably a rodent such as a rat or mouse, in which one or more of the cells of the animal include a transgene. Other examples of transgenic animals include nonhuman primates, sheep, dogs, pigs, cows, goats, chickens, amphibians, etc. A transgene is exogenous DNA that is integrated into the genome of a cell from which a transgenic animal develops and which remains in the genome of the mature animal, thereby directing the expression of an encoded gene product in one or more cell types or tissues of the transgenic animal.

A “knock-in animal” is an animal (e.g., a mammal such as a mouse or a rat) in which an endogenous gene has been substituted in part or in total with a heterologous gene (i.e., a gene that is not endogenous to the locus in question; see Roamer et al., New Biol. 1991, 3:331). This can be achieved by homologous recombination (see “knockout animal” below), transposition (Westphal and Leder, Curr. Biol. 1997; 7: 530), use of mutated recombination sites (Araki et al., Nucleic Acids Res. 1997; 25: 868), PCR (Zhang and Henderson, Biotechniques 1998; 25: 784), or any other technique known in the art. The heterologous gene may be, e.g., a reporter gene linked to the appropriate (e.g., endogenous) promoter, which may be used to evaluate the expression or function of the endogenous gene (see, e.g., Elegant et al., Proc. Natl. Acad. Sci. USA 1998; 95: 11897).

A “knockout animal” is an animal (e.g., a mammal such as a mouse or a rat) that has had a specific gene in its genome partially or completely inactivated by gene targeting (see, e.g., U.S. Pat. Nos. 5,777,195 and 5,616,491). A knockout animal can be a heterozygous knockout (i.e., with one defective allele and one wild type allele) or a homozygous knockout (i.e., with both alleles rendered defective). Preparation of a knockout animal typically requires first introducing a nucleic acid construct (a “knockout construct”), that will be used to decrease or eliminate expression of a particular gene, into an undifferentiated cell type termed an embryonic stem (ES) cell. The knockout construct is typically comprised of: (i) DNA from a portion (e.g., an exon sequence, intron sequence, promoter sequence, or some combination thereof) of a gene to be knocked out; and (ii) a selectable marker sequence used to identify the presence of the knockout construct in the ES cell. The knockout construct is typically introduced (e.g., electroporated) into ES cells so that it can homologously recombine with the genomic DNA of the cell in a double crossover event. This recombined ES cell can be identified (e.g., by Southern hybridization or PCR reactions that show the genomic alteration) and is then injected into a mammalian embryo at the blastocyst stage. In a preferred embodiment where the knockout animal is a mammal, a mammalian embryo with integrated ES cells is then implanted into a foster mother for the duration of gestation (see, e.g., Zhou et al., Genes and Dev. 1995; 9: 2623-34).

The phrases “disruption of the gene”, “gene disruption”, and the like, refer to: (i) insertion of a different or defective nucleic acid sequence into an endogenous (naturally occurring) DNA sequence, e.g., into an exon or promoter region of a gene; or (ii) deletion of a portion of an endogenous DNA sequence of a gene; or (iii) a combination of insertion and deletion, so as to decrease or prevent the expression of that gene or its gene product in the cell as compared to the expression of the endogenous gene sequence.

In accordance with the present invention, there may be employed conventional molecular biology, microbiology, and recombinant DNA techniques within the skill of the art. See, e.g., Sambrook, Fritsch and Maniatis, Molecular Cloning: A Laboratory Manual, 2nd ed., Cold Spring Harbor Laboratory Press, Cold Spring Harbor, N.Y., 1989 (herein “Sambrook et al., 1989”); DNA Cloning: A Practical Approach, Volumes I and II (Glover ed. 1985); Oligonucleotide Synthesis (Gait ed. 1984); Nucleic Acid Hybridization (Hames and Higgins eds. 1985); Transcription And Translation (Hames and Higgins eds. 1984); Animal Cell Culture (Freshney ed. 1986); Immobilized Cells And Enzymes (IRL Press, 1986); B. Perbal, A Practical Guide To Molecular Cloning (1984); Ausubel et al. eds., Current Protocols in Molecular Biology, John Wiley and Sons, Inc. 1994; among others.

### NPC1L1 Polynucleotides

The present invention provides an isolated nucleic acid molecule comprising a nucleotide sequence encoding NPC1L1. More particularly, the present invention provides an isolated NPC1L1 nucleic acid sequence having a nucleotide sequence encoding mouse NPC1L1.

In one embodiment, the NPC1L1 nucleic acid has nucleotide sequence of SEQ ID NO:1, or a degenerate variant thereof. In another embodiment, NPC1L1 nucleic acid has nucleotide sequence of SEQ ID NO:2, or a degenerate variant thereof.

The present invention also provides an isolated single-stranded polynucleotide molecule comprising a nucleotide sequence that is the complement of a nucleotide sequence of one strand of any of the aforementioned nucleotide sequences (e.g., SEQ ID NO: 2).

The present invention further provides an isolated polynucleotide molecule comprising a nucleotide sequence that hybridizes to the complement of a polynucleotide that encodes the amino acid sequence of the mouse NPC1L1 protein of the present invention, under moderately stringent conditions, such as, for example, an aqueous solution of 2×SSC at 65° C.; alternatively, for example, hybridization to filter-bound DNA in 0.5 M NaHPO₄, 7% SDS, 1 mM EDTA at 65° C., and washing in 0.2×SSC/0.1% SDS at 42° C. (see the Definitions section above).

In a preferred embodiment, the homologous polynucleotide molecule hybridizes to the complement of a polynucleotide molecule comprising a nucleotide sequence that encodes the amino acid sequence of the mouse NPC1L1 protein of the present invention under highly stringent conditions, such as, for example, in an aqueous solution of 0.5×SSC at 65° C.; alternatively, for example, hybridization to filter-bound DNA in 0.5 M NaHPO₄, 7% SDS 1 mM EDTA at 65° C., and washing in 0.1.x SSC/0.1% SDS at 68° C. (see the Definitions Section 5.1, above).

In a more preferred embodiment, the homologous polynucleotide molecule hybridizes under highly stringent conditions to the complement of a polynucleotide molecule consisting of a nucleotide sequence selected from the group consisting of SEQ ID NO:1 and SEQ ID NO:2.

The present invention further provides an isolated polynucleotide molecule comprising a nucleotide sequence that is homologous to the nucleotide sequence of a NPC1L1-encoding polynucleotide molecule of the present invention. In a preferred embodiment, such a polynucleotide molecule hybridizes under standard conditions to the complement of a polynucleotide molecule comprising a nucleotide sequence that encodes the amino acid sequence of the mouse NPC1L1 protein of the present invention and has at least 75% sequence identity, preferably at least 80% sequence identity, more preferably at least 90% sequence identity, more preferably at least 95% sequence identity, and most preferably at least 99% sequence identity to the nucleotide sequence of such NPC1L1-encoding polynucleotide molecule (e.g., as determined by a sequence comparison algorithm selected from BLAST, FASTA, DNA Strider, and GCG, and preferably as determined by the BLAST program from the National Center for Biotechnology Information (NCBI-Version 2.2), available on the WorldWideWeb at <www.ncbi.nlm.nih.gov/BLAST/htm>). In one embodiment, the homologous polynucleotide is homologous to a polynucleotide encoding mouse NPC1L1 protein of the present invention, e.g, SEQ ID NO: 2.

The present invention further provides an oligonucleotide molecule that hybridizes to a polynucleotide molecule of the present invention, or that hybridizes to a polynucleotide molecule having a nucleotide sequence that is the complement of a nucleotide sequence of a polynucleotide molecule of the present invention. Such an oligonucleotide molecule: (i) is about 10 nucleotides to about 200 nucleotides in length, preferably from about 15 to about 100 nucleotides in length, and more preferably about 20 to about 50 nucleotides in length, and (ii) hybridizes to one or more of the polynucleotide molecules of the present invention under highly stringent conditions (e.g., washing in 6×SSC/0.5% sodium pyrophosphate at about 37° C. for about 14-base oligos, at about 48° C. for about 17-base oligos, at about 55° C. for about 20-base oligos, and at about 60° C. for about 23-base oligos). In one embodiment, an oligonucleotide molecule of the present invention is 100% complementary over its entire length to a portion of at least one of the aforementioned polynucleotide molecules of the present invention, and particularly any of SEQ ID NOs: 1 or 2. In another embodiment, an oligonucleotide molecule of the present invention is greater than 90% complementary over its entire length to a portion of at least one of the aforementioned polynucleotide molecules of the present invention, and particularly any of SEQ ID NOs: 1 or 2.

Specific non-limiting examples of oligonucleotide molecules according to the present invention include oligonucleotide molecules selected from the group consisting of SEQ ID NOs: 4 and 5.

Oligonucleotide molecules can be labeled, e.g., with radioactive labels (e.g., γ³² P), biotin, fluorescent labels, etc. In one embodiment, a labeled oligonucleotide molecule can be used as a probe to detect the presence of a nucleic acid. In another embodiment, two oligonucleotide molecules (one or both of which may be labeled) can be used as PCR primers, either for cloning a full-length nucleic acid or a fragment of a nucleic acid encoding a gene product of interest, or to detect the presence of nucleic acids encoding a gene product. Methods for conducting amplifications, such as the polymerase chain reaction (PCR), are described, among other places, in Saiki et al., Science 1988, 239:487 and U.S. Pat. No. 4,683,202. Other amplification techniques known in the art, e.g., the ligase chain reaction, can alternatively be used (see, e.g., U.S. Pat. Nos. 6,335,184 and 6,027,923; Reyes et al., Clinical Chemistry 2001; 47: 131-40; and Wu et al., Genomics 1989; 4: 560-569).

The present invention further provides a polynucleotide molecule consisting of a nucleotide sequence that is a substantial portion of the nucleotide sequence of any of the aforementioned NPC1L1-related polynucleotide molecules of the present invention, or the complement of such nucleotide sequence. As used herein, a “substantial portion” of a NPC1L1-encoding nucleotide sequence means a nucleotide sequence that is less than the nucleotide sequence required to encode a complete NPC1L1 protein of the present invention, but comprising at least about 5%, at least about 10%, at least about 20%, at least about 30%, at least about 40%, at least about 50%, at least about 60%, at least about 70%, at least about 80%, at least about 90%, at least about 95%, or at least about 99% of the contiguous nucleotide sequence of a NPC1L1-encoding polynucleotide molecule of the present invention. Such polynucleotide molecules can be used for a variety of purposes including, e.g., to express a portion of a NPC1L1 protein of the present invention in an appropriate expression system, or for use in conducting an assay to determine the expression level of a NPC1L1 gene in a biological sample, or to amplify a NPC1L1-encoding polynucleotide molecule.

In addition to the nucleotide sequences of any of the aforementioned NPC1L1-related polynucleotide molecules, polynucleotide molecules of the present invention can further comprise, or alternatively may consist of, nucleotide sequences selected from the sequence depicted in SEQ ID NO: 1 (genomic) that naturally flank a NPC1L1-encoding nucleotide sequence in the chromosome, including regulatory sequences.

### NPC1L1 Polypeptides

The present invention also provides an NPC1L1 polypeptide encoded by an NPC1L1 polynucleotide. In one embodiment, the NPC1L1 polypeptide is encoded by an NPC1L1 polynucleotide comprising the sequence as set forth in SEQ ID NO: 2.

The present invention also provides an NPC1L1 polypeptide encoded by an NPC1L1 polynucleotide that hybridizes to the complement of the polynucleotide sequence set forth in SEQ ID NOS. 1 or 2.

In one embodiment, NPC1L1 polypeptide comprises the amino acid sequence set forth SEQ ID NO:3.

The present invention further provides a non-human polypeptide that is homologous to the NPC1L1 protein of the present invention, as the term “homologous” is defined above for polypeptides. In one embodiment, the homologous NPC1L1 polypeptides of the present invention have the amino acid sequence identical to the amino acid sequence of SEQ ID NO:3, but have one or more amino acid residues conservatively substituted with a different amino acid residue. Conservative amino acid substitutions are well-known in the art. Rules for making such substitutions include those described by Dayhof, 1978, Nat. Biomed. Res. Found., Washington, D.C., Vol. 5, Sup. 3, among others. More specifically, conservative amino acid substitutions are those that take place within a family of amino acids that are related in acidity, polarity, or bulkiness of their side chains. Genetically encoded amino acids are generally divided into four groups: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan and tyrosine are also jointly classified as aromatic amino acids. One or more replacements within any particular group, e.g., of a leucine with an isoleucine or valine, or of an aspartate with a glutamate, or of a threonine with a serine, or of any other amino acid residue with a structurally related amino acid residue, e.g., an amino acid residue with similar acidity, polarity, bulkiness of side chain, or with similarity in some combination thereof, will generally have an insignificant effect on the function or immunogenicity of the polypeptide.

The NPC1L1 polypeptides of the present invention (including those encoded by the homologous polynucleotide molecules above, i.e., homologous NPC1L1 polypeptides) have the following functions including, but not limited to: (i) endocytosis and intracellular trafficking of multiple classes of lipids, including fatty acids such as oleic acid, sterols such as cholesterol, and, sphingolipids such as lactosylceramide; (ii) regulation of caveolae formation and/or internalization; (iii) the sensing of sterols through a sterol sensing domain; (iv) conferring localization to the ER and Golgi; and (v) regulating serum levels of total cholesterol, LDL-cholesterol, HDL-cholesterol, triglycerides, insulin, and glucose. (see also Davies et al., 2005, J Biological Chemistry, Vol. 280, No. 13, pp. 12710-12720, the contents of which are expressly incorporated herein by reference).

Also encompassed by the present invention are orthologs of the specifically disclosed NPC1L1 polypeptides, and NPC1L1-encoding nucleic acids. Additional NPC1L1 orthologs can be identified based on the sequences of mouse and human orthologs disclosed herein, using standard sequence comparison algorithms such as BLAST, FASTA, DNA Strider, GCG, etc. In addition to mouse and human orthologs, particularly useful NPC1L1 orthologs of the present invention are monkey, dog, guinea pig, and porcine orthologs. As with the homologs discussed above, these orthologs can have the same functions as the NPC1L1 protein.

The present invention further provides a polypeptide consisting of a substantial portion of a mouse NPC1L1 protein of the present invention. “Substantial portion” has the same meaning as defined above under NPC1L1 polynucleotides.

The present invention further provides fusion proteins comprising any of the aforementioned polypeptides (proteins or peptide fragments) fused to a carrier or fusion partner, as known in the art. For example, NPC1L1 can be fused with green fluorescent protein (GFP), V5, and Ig.

Recombinant Expression Systems Cloning and Expression Vectors

The present invention further provides compositions and constructs for cloning and expressing any of the NPC1L1 polynucleotide molecules of the present invention, including cloning vectors, expression vectors, transformed host cells comprising any of said vectors, and novel strains or cell lines derived therefrom. In one embodiment, the present invention provides a recombinant vector comprising a polynucleotide molecule having a nucleotide sequence encoding a non-human NPC1L1 polypeptide. In a specific embodiment, the mouse NPC1L1 polypeptide comprises the amino acid sequence of SEQ ID NO: 3.

Recombinant vectors of the present invention, particularly expression vectors, are preferably constructed so that the coding sequence for the NPC1L1 polynucleotide molecule of the present invention is in operative association with one or more regulatory elements necessary for transcription and translation of the coding sequence to produce a polypeptide. As used herein, the term “regulatory element” includes, but is not limited to, nucleotide sequences that encode inducible and non-inducible promoters, enhancers, operators and other elements known in the art that serve to drive and/or regulate expression of polynucleotide coding sequences. Also, as used herein, the coding sequence is in operative association with one or more regulatory elements where the regulatory elements effectively regulate and allow for the transcription of the coding sequence or the translation of its mRNA, or both.

Methods are known in the art for constructing recombinant vectors containing particular coding sequences in operative association with appropriate regulatory elements, and these can be used to practice the present invention. These methods include in vitro recombinant techniques, synthetic techniques, and in vivo genetic recombination. See, e.g., the techniques described in Ausubel et al., 1989, above; Sambrook et al., 1989, above; Saiki et al., 1988, above; Reyes et al., 2001, above; Wu et al., 1989, above; U.S. Pat. Nos. 4,683,202; 6,335,184 and 6,027,923.

A variety of expression vectors are known in the art that can be utilized to express a polynucleotide molecule of the present invention, including recombinant bacteriophage DNA, plasmid DNA, and cosmid DNA expression vectors containing the particular coding sequences. Typical prokaryotic expression vector plasmids that can be engineered to contain a polynucleotide molecule of the present invention include pUC8, pUC9, pBR322 and pBR329 (Biorad Laboratories, Richmond, Calif.), pPL and pKK223 (Pharmacia, Piscataway, N.J.), pQE50 (Qiagen, Chatsworth, Calif.), and pGEM-T EASY (Promega, Madison, Wis.), pcDNA6.2/V5-DEST and pcDNA3.2/V5DEST (Invitrogen, Carlsbad, Calif.) among many others. Typical eukaryotic expression vectors that can be engineered to contain a polynucleotide molecule of the present invention include an ecdysone-inducible mammalian expression system (Invitrogen, Carlsbad, Calif.), cytomegalovirus promoter-enhancer-based systems (Promega, Madison, Wis.; Stratagene, La Jolla, Calif.; Invitrogen), and baculovirus-based expression systems (Promega), among many others.

The regulatory elements of these and other vectors can vary in their strength and specificities. Depending on the host/vector system utilized, any of a number of suitable transcription and translation elements can be used. For instance, when cloning in mammalian cell systems, promoters isolated from the genome of mammalian cells, e.g., mouse metallothionein promoter, or from viruses that grow in these cells, e.g., vaccinia virus 7.5 K promoter or Maloney murine sarcoma virus long terminal repeat, can be used. Promoters obtained by recombinant DNA or synthetic techniques can also be used to provide for transcription of the inserted sequence. In addition, expression from certain promoters can be elevated in the presence of particular inducers, e.g., zinc and cadmium ions for metallothionein promoters. Non-limiting examples of transcriptional regulatory regions or promoters include for bacteria, the β-gal promoter, the T7 promoter, the TAC promoter, λ left and right promoters, trp and lac promoters, trp-lac fusion promoters, etc.; for yeast, glycolytic enzyme promoters, such as ADH-I and -II promoters, GPK promoter, PGI promoter, TRP promoter, etc.; and for mammalian cells, SV40 early and late promoters, and adenovirus major late promoters, among others.

Specific initiation signals are also required for sufficient translation of inserted coding sequences. These signals typically include an ATG initiation codon and adjacent sequences. In cases where the polynucleotide molecule of the present invention, including its own initiation codon and adjacent sequences, is inserted into the appropriate expression vector, no additional translation control signals may be needed. However, in cases where only a portion of a coding sequence is inserted, exogenous translational control signals, including the ATG initiation codon, may be required. These exogenous translational control signals and initiation codons can be obtained from a variety of sources, both natural and synthetic. Furthermore, the initiation codon must be in-phase with the reading frame of the coding regions to ensure in-frame translation of the entire insert.

Expression vectors can also be constructed that will express a fusion protein comprising an NPC1L1 polypeptide of the present invention. Such fusion proteins can be used, e.g., to raise anti-sera against a NPC1L1 polypeptide, to study the biochemical properties of the NPC1L1 polypeptide, to engineer a variant of a NPC1L1 polypeptide exhibiting different immunological or functional properties, or to aid in the identification or purification, or to improve the stability, of a recombinant NPC1L1 polypeptide. Possible fusion protein expression vectors include but are not limited to vectors incorporating sequences that encode β-galactosidase and trpE fusions, maltose-binding protein fusions, glutathione-S-transferase fusions, polyhistidine fusions (carrier regions), V5, HA, myc, and HIS. Methods known in the art can be used to construct expression vectors encoding these and other fusion proteins.

The fusion protein can be useful to aid in purification of the expressed protein. In non-limiting embodiments, e.g., a NPC1L1-polyhistidine fusion protein can be purified using divalent nickel resin; a NPC1L1-maltose-binding fusion protein can be purified using amylose resin; and a NPC1L1-glutathione-S-transferase fusion protein can be purified using glutathione-agarose beads. Alternatively, antibodies against a carrier protein or peptide can be used for affinity chromatography purification of the fusion protein. For example, a nucleotide sequence coding for the target epitope of a monoclonal antibody can be engineered into the expression vector in operative association with the regulatory elements and situated so that the expressed epitope is fused to a NPC1L1 protein of the present invention. In a non-limiting embodiment, a nucleotide sequence coding for the FLAG™ epitope tag (International Biotechnologies Inc.), which is a hydrophilic marker peptide, can be inserted by standard techniques into the expression vector at a point corresponding, e.g., to the amino or carboxyl terminus of the NPC1L1 protein. The expressed NPC1L1 protein-FLAG™ epitope fusion product can then be detected and affinity-purified using commercially available anti-FLAG™ antibodies. The expression vector can also be engineered to contain polylinker sequences that encode specific protease cleavage sites so that the expressed NPC1L1 protein can be released from a carrier region or fusion partner by treatment with a specific protease. For example, the fusion protein vector can include a nucleotide sequence encoding a thrombin or factor Xa cleavage site, among others.

A signal sequence upstream from, and in reading frame with, the NPC1L1 coding sequence can be engineered into the expression vector by known methods to direct the trafficking and secretion of the expressed protein. Non-limiting examples of signal sequences include those from α-factor, immunoglobulins, outer membrane proteins, penicillinase, and T-cell receptors, among others.

To aid in the selection of host cells transformed or transfected with a recombinant vector of the present invention, the vector can be engineered to further comprise a coding sequence for a reporter gene product or other selectable marker. Such a coding sequence is preferably in operative association with the regulatory elements, as described above. Reporter genes that are useful in practicing the invention are known in the art, and include those encoding chloramphenicol acetyltransferase (CAT), green fluorescent protein and derivatives thereof, firefly luciferase, and human growth hormone, among others. Nucleotide sequences encoding selectable markers are known in the art, and include those that encode gene products conferring resistance to antibiotics or anti-metabolites, or that supply an auxotrophic requirement. Examples of such sequences include those that encode thymidine kinase activity, or resistance to methotrexate, ampicillin, kanamycin, chloramphenicol, zeocin, pyrimethamine, aminoglycosides, hygromycin, blasticidine, or neomycin, among others.

### Transformation of Host Cells

The present invention further provides a transformed host cell comprising a polynucleotide molecule or recombinant vector of the present invention, and a cell line derived therefrom. Such host cells are useful for cloning and/or expressing a polynucleotide molecule of the present invention. Such transformed host cells include but are not limited to microorganisms, such as bacteria transformed with recombinant bacteriophage DNA, plasmid DNA or cosmid DNA vectors, or yeast transformed with a recombinant vector, or animal cells, such as insect cells infected with a recombinant virus vector, e.g., baculovirus, or mammalian cells infected with a recombinant virus vector, e.g., adenovirus, vaccinia virus, lentivirus, adeno-associated virus (AAV), or herpesvirus, among others. For example, a strain of E. coli can be used such as, e.g., the DH5α strain available from the ATCC, Manassas, Va., USA (Accession No. 31343), or from Stratagene (La Jolla, Calif.). Eukaryotic host cells include yeast cells, although mammalian cells, e.g., from a mouse, rat, hamster, cow, monkey, or human cell line, among others, can also be utilized effectively. Examples of eukaryotic host cells that may be suitable for expressing a recombinant protein of the invention include Chinese hamster ovary (CHO) cells (e.g., ATCC Accession No. CCL-61), NIH Swiss mouse embryo cells NIH/3T3 (e.g., ATCC Accession No. CRL-1658), human epithelial kidney cells HEK 293 (e.g., ATCC Accession No. CRL-1573), African green monkey COS-7 cells (ATCC Accession No. CRL-1651), human embryonal carcinoma NT2 cells (ATCC Accession No. CRL-1973), and human colon carcinoma Caco-2 cells ATCC Accession No. HTB-37.

The present invention provides for mammalian cells infected with a virus containing a recombinant viral vector of the present invention. For example, an overview and instructions concerning the infection of mammalian cells with adenovirus using the AdEasy™ Adenoviral Vector System is given in the Instructions Manual for this system from Stratagene (La Jolla, Calif.). As another example, an overview and instructions concerning the infection of mammalian cells with AAV using the AAV Helper-Free System is given in the Instructions Manual for this system from Strategene (La Jolla, Calif.).

The recombinant vector of the invention is preferably transformed or transfected into one or more host cells of a substantially homogeneous culture of cells. The vector is generally introduced into host cells in accordance with known techniques, such as, e.g., by protoplast transformation, calcium phosphate precipitation, calcium chloride treatment, microinjection, electroporation, transfection by contact with a recombined virus, liposome-mediated transfection, DEAE-dextran transfection, transduction, conjugation, or microprojectile bombardment, among others. Selection of transformants can be conducted by standard procedures, such as by selecting for cells expressing a selectable marker, e.g., antibiotic resistance, associated with the recombinant expression vector.

Once an expression vector is introduced into the host cell, the presence of the polynucleotide molecule of the present invention, either integrated into the host cell genome or maintained episomally, can be confirmed by standard techniques, e.g., by DNA-DNA, DNA-RNA, or RNA-antisense RNA hybridization analysis, restriction enzyme analysis, PCR analysis including reverse transcriptase PCR(RT-PCR), detecting the presence of a “marker” gene function, or by immunological or functional assay to detect the expected protein product.

Expression and Purification of Recombinant NPC1L1 Polypeptides

Once an NPC1L1 polynucleotide molecule of the present invention has been stably introduced into an appropriate host cell, the transformed host cell is clonally propagated, and the resulting cells can be grown under conditions conducive to the efficient production (i.e., expression or overexpression) of the NPC1L1 polypeptide.

The polypeptide can be substantially purified or isolated from cell lysates, membrane fractions, or culture medium, as necessary, using standard methods, including but not limited to one or more of the following methods: ammonium sulfate precipitation, size fractionation, ion exchange chromatography, HPLC, density centrifugation, affinity chromatography, ethanol precipitation, and chromatofocusing. During purification, the polypeptide can be detected based, e.g., on size, or reactivity with a polypeptide-specific antibody, or by detecting the presence of a fusion tag.

For use in practicing the present invention, the polypeptide can be in an unpurified state as secreted into the culture fluid or as present in a cell lysate or membrane fraction. Alternatively, the polypeptide may be purified therefrom. Once a polypeptide of the present invention of sufficient purity has been obtained, it can be characterized by standard methods, including by SDS-PAGE, size exclusion chromatography, amino acid sequence analysis, immunological activity, biological activity, etc. The polypeptide can be further characterized using hydrophilicity analysis (see, e.g., Hopp and Woods, Proc. Natl. Acad. Sci. USA 1981; 78: 3824), or analogous software algorithms, to identify hydrophobic and hydrophilic regions. Structural analysis can be carried out to identify regions of the polypeptide that assume specific secondary structures. Biophysical methods such as X-ray crystallography (Engstrom, Biochem. Exp. Biol. 1974; 11: 7-13), computer modeling (Fletterick and Zoller eds., In: Current Communications in Molecular Biology, Cold Spring Harbor Laboratory, Cold Spring Harbor, N.Y., 1986), and nuclear magnetic resonance (NMR) can be used to map and study potential sites of interaction between the polypeptide and other putative interacting proteins/receptors/molecules. Information obtained from these studies can be used to design deletion mutants, and to design or select therapeutic compounds that can specifically modulate the biological function of the NPC1L1 protein in vivo.

### NPC1L1 Antibodies

The present invention also provides antibodies, including fragments thereof, which specifically bind to an NPC1L1 polypeptide, or fragment thereof. Antibodies to NPC1L1 have a number of applications, such as detecting the presence of NPC1L1 in a biological sample, determining the intracellular localization of NPC1L1, and modulating the activity of NPC1L1, e.g., in a subject, for treatment (e.g., therapeutic and prophylactic) of diseases and disorders associated with or mediated by NPC1L1, such as hyperlipidemia, obesity, type II diabetes, cardiovascular disease, and stroke. The present invention contemplates a number of sources for immunogenic NPC1L1 polypeptides for use in producing anti-NPC1L1 antibodies. These sources include NPC1L1 polypeptides produced by recombinant technology and chemical synthesis; and products derived from their fragmentation or derivation.

Various antibodies against NPC1L1 are described in published U.S. patent application 2004/0161838, to Altmann et al., hereby incorporated by reference in its entirety. Such antibodies are designated A0715, A0716, A0717, A0718, A0867, A0868, A1801 or A1802. Additional commercially available antibodies include NPC1L1 rabbit polyclonal antibodies (Novus Biologicals, Littleton, Colo., Cat # BC-400 NPC3).

As used herein, the term “antibody molecule” includes, but is not limited to, antibodies and binding fragments thereof, that specifically binds to an antigen, e.g., an NPC1L1 protein. Suitable antibodies may be polyclonal (e.g., sera or affinity purified preparations), monoclonal, or recombinant. Examples of useful fragments include separate heavy chains, light chains, Fab, F(ab′)₂, Fabc, and Fv fragments. Fragments can be produced by enzymatic or chemical separation of intact immunoglobulins or by recombinant DNA techniques. Fragments may be expressed in the form of phage-coat fusion proteins (see, e.g., International PCT Publication Nos. WO 91/17271, WO 92/01047, and WO 92/06204). Typically, the antibodies, fragments, or similar binding agents bind a specific antigen with an affinity of at least 10⁷, 10⁸, 10⁹, or 10¹⁰ M⁻¹.

The present invention provides an isolated antibody directed against a polypeptide of the present invention. In a specific embodiment, antibodies can be raised against a NPC1L1 protein of the invention using known methods in view of this disclosure. Various host animals selected, e.g., from pigs, cows, horses, rabbits, goats, sheep, rats, or mice, can be immunized with a partially or substantially purified NPC1L1 protein, or with a peptide homolog, fusion protein, peptide fragment, analog or derivative thereof, as described above. An adjuvant can be used to enhance antibody production.

Polyclonal antibodies can be obtained and isolated from the serum of an immunized animal and tested for specificity against the antigen using standard techniques. Alternatively, monoclonal antibodies can be prepared and isolated using any technique that provides for the production of antibody molecules by continuous cell lines in culture. These include but are not limited to; (i) the hybridoma technique originally described by Kohler and Milstein, Nature 1975; 256: 495-497; (ii) the trioma technique (Herring et al. (1988) Biomed. Biochim. Acta. 46:211-216 and Hagiwara et al. (1993) Hum. Antibod. Hybridomas 4:15); (iii) the human B-cell hybridoma technique (Kosbor et al., Immunology Today 1983; 4: 72; Cote et al., Proc. Natl. Acad. Sci. USA 1983; 80: 2026-2030); and the EBV-hybridoma technique (Cole et al., Monoclonal Antibodies and Cancer Therapy, Alan R. Liss, Inc., 1985, pp. 77-96). Alternatively, techniques described for the production of single chain antibodies (see, e.g., U.S. Pat. No. 4,946,778) can be adapted to produce NPC1L1-specific single chain antibodies.

Antibody fragments that contain specific binding sites for the NPC1L1 polypeptide of the present invention are also encompassed within the present invention, and can be generated by known techniques. Such fragments include but are not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab expression libraries can be constructed (Huse et al., Science 1989; 246: 1275-1281) to allow rapid identification of Fab fragments having the desired specificity to the particular NPC1L1 protein.

Techniques for the production and isolation of monoclonal antibodies and antibody fragments are known in the art, and are generally described, among other places, in Harlow and Lane, Antibodies: A Laboratory Manual, Cold Spring Harbor Laboratory, 1988, and in Goding, Monoclonal Antibodies: Principles and Practice, Academic Press, London, 1986. The art also provides recombinant expression systems in bacteria and yeast, enabling the production of functional antibodies that are analogous to those normally found in vertebrate systems. (Skerra et al. (1988) Science 240:1038-1041, Better et al. (1988) Science 240:1041-1043, and Bird et al. (1988) Science 242:423-426, Horwitz et al. (1989) Proc. Natl. Acad. Sci. USA. 85:8678-82.) Antibodies or antibody fragments can be used in methods known in the art relating to the localization and activity of NPC1L1, e.g., in Western blotting, in situ imaging, measuring levels thereof in appropriate physiological samples, etc. Immunoassay techniques using antibodies include radioimmunoassay, ELISA (enzyme-linked immunosorbant assay), “sandwich” immunoassays, immunoradiometric assays, gel diffusion precipitation reactions, immunodiffusion assays, in situ immunoassays (using, e.g., colloidal gold, enzyme or radioisotope labels), precipitation reactions, agglutination assays (e.g., gel agglutination assays, hemagglutination assays), complement fixation assays, immunofluorescence assays, protein A assays, and immunoelectrophoresis assays, etc. Antibodies can also be used in microarrays (see, e.g., International PCT Publication No. WO 00/04389). Furthermore, antibodies can be used as therapeutics to inhibit the activity of a NPC1L1 protein.

Recent advances in antibody engineering have allowed the genes encoding antibodies to be manipulated, so that antigen-binding molecules can be expressed within mammalian cells. Application of gene technologies to antibody engineering has enabled the synthesis of single-chain fragment variable (scFv) antibodies that combine within a single polypeptide chain the light and heavy chain variable domains of an antibody molecule covalently joined by a pre-designed peptide linker. Intracellular antibody (or “intrabody”) strategy serves to target molecules involved in essential cellular pathways for modification or ablation of protein function. Antibody genes for intracellular expression can be derived, e.g., either from murine or human monoclonal antibodies or from phage display libraries. For intracellular expression, small recombinant antibody fragments containing the antigen recognizing and binding regions can be used. Intrabodies can be directed to different intracellular compartments by targeting sequences attached to the antibody fragments.

Various methods have been developed to produce intrabodies. Techniques described for the production of single chain antibodies (see, e.g., U.S. Pat. Nos. 5,476,786; 5,132,405; and 4,946,778) can be adapted to produce polypeptide-specific single chain antibodies. Another method called intracellular antibody capture (IAC), is based on a genetic screening approach (Tanaka et al., Nucleic Acids Res. 2003; 31: e23). Using this technique, consensus immunoglobulin variable frameworks are identified that can form the basis of intrabody libraries for direct screening. The procedure comprises in vitro production of a single antibody gene fragment from oligonucleotides and diversification of CDRs of the immunoglobulin variable domain by mutagenic PCR to generate intrabody libraries. This method obviates the need for in vitro production of antigen for pre-selection of antibody fragments, and also yields intrabodies with enhanced intracellular stability.

Intrabodies can be used to modulate cellular physiology and metabolism through a variety of mechanisms, including blocking, stabilizing, or mimicking protein-protein interactions, by altering enzyme function, or by diverting proteins from their usual intracellular compartments. Intrabodies can be directed to the relevant cellular compartments by modifying the genes that encode them to specify N- or C-terminal polypeptide extensions for providing intracellular-trafficking signals.

### NPC1L1 Applications

NPC1L1 polynucleotides and polypeptides of the present invention are useful for a variety of purposes, including for use in cell-based or non-cell-based assays to identify molecules that interact with NPC1L1 relevant to its in vivo function, to screen for compounds that bind to NPC1L1 and modulate its expression and/or activity and are therefore useful as therapeutic compounds to treat or prevent NPC1L1-mediated diseases or disorders as described herein, or as antigens to raise polyclonal or monoclonal antibodies, as described below. Such antibodies can be used as therapeutic agents to modulate the activity of NPC1L1 activity, or as diagnostic reagents, e.g., using standard techniques such as Western blot assays or immunostaining, to screen for NPC1L1 protein expression levels in cell, tissue or fluid samples collected from a subject.

A polypeptide of the present invention can be modified at the protein level to improve or otherwise alter its biological or immunological characteristics. One or more chemical modifications of the polypeptide can be carried out using known techniques to prepare analogs therefrom, including but not limited to any of the following: substitution of one or more L-amino acids of the polypeptide with corresponding D-amino acids, amino acid analogs, or amino acid mimics, so as to produce, e.g., carbazates or tertiary centers; or specific chemical modification, such as, e.g., proteolytic cleavage with trypsin, chymotrypsin, papain or V8 protease, or treatment with NaBH₄ or cyanogen bromide, or acetylation, formylation, oxidation or reduction, etc. Alternatively or additionally, a polypeptide of the present invention can be modified by genetic recombination techniques.

A polypeptide of the present invention can be derivatized, by conjugation thereto of one or more chemical groups, including but not limited to acetyl groups, sulfur bridging groups, glycosyl groups, lipids, and phosphates, and/or by conjugation to a second polypeptide of the present invention, or to another protein, such as, e.g., serum albumin, keyhole limpet hemocyanin, or commercially activated BSA, or to a polyamino acid (e.g., polylysine), or to a polysaccharide, (e.g., sepharose, agarose, or modified or unmodified celluloses), among others. Such conjugation is preferably by covalent linkage at amino acid side chains and/or at the N-terminus or C-terminus of the polypeptide. Methods for carrying out such conjugation reactions are known in the field of protein chemistry.

Derivatives useful in practicing the claimed invention also include those in which a water-soluble polymer such as, e.g., polyethylene glycol, is conjugated to a polypeptide of the present invention, or to an analog or derivative thereof, thereby providing additional desirable properties while retaining, at least in part, the immunogenicity of the polypeptide. These additional desirable properties include, e.g., increased solubility in aqueous solutions, increased stability in storage, increased resistance to proteolytic degradation, and increased in vivo half-life. Water-soluble polymers suitable for conjugation to a polypeptide of the present invention include but are not limited to polyethylene glycol homopolymers, polypropylene glycol homopolymers, copolymers of ethylene glycol with propylene glycol, wherein said homopolymers and copolymers are unsubstituted or substituted at one end with an alkyl group, polyoxyethylated polyols, polyvinyl alcohol, polysaccharides, polyvinyl ethyl ethers, and α,β-poly[2-hydroxyethyl]-DL-aspartamide. Polyethylene glycol is particularly preferred. Methods for making water-soluble polymer conjugates of polypeptides are known in the art and are described, among other places, in U.S. Pat. Nos. 3,788,948; 3,960,830; 4,002,531; 4,055,635; 4,179,337; 4,261,973; 4,412,989; 4,414,147; 4,415,665; 4,609,546; 4,732,863; and 4,745,180; European Patent (EP) 152,847; EP 98,110; and Japanese Patent 5,792,435; which patents are incorporated herein by reference.

### Targeted Mutation of the NPC1L1 Gene

Based on the present disclosure of polynucleotide molecules, genetic constructs can be prepared for use in disabling or otherwise mutating a mammalian NPC1L1 gene. For example, the mouse NPC1L1 gene can be mutated using an appropriately designed genetic construct in combination with genetic techniques currently known or to be developed in the future. In another instance, the mouse NPC1L1 gene can be mutated using a genetic construct that functions to: (i) delete all or a portion of the coding sequence or regulatory sequence of the NPC1L1 gene; (ii) replace all or a portion of the coding sequence or regulatory sequence of the NPC1L1 gene with a different nucleotide sequence; (iii) insert into the coding sequence or regulatory sequence of the NPC1L1 gene one or more nucleotides, or an oligonucleotide molecule, or polynucleotide molecule, which can comprise a nucleotide sequence from the same species or from a heterologous source; or (iv) carry out some combination of (i), (ii) and (iii).

Cells, tissues and animals that are mutated for the NPC1L1 gene are useful for a number of purposes, such as further studying the biological function of NPC1L1, and conducting screens to identify therapeutic compounds that selectively modulate NPC1L1 expression and/or activity. In a preferred embodiment, the mutation serves to partially or completely disable the NPC1L1 gene, or partially or completely disable the protein encoded by the NPC1L1 gene. In this context, a NPC1L1 gene or protein is considered to be partially or completely disabled if either no protein product is made (for example, where the gene is deleted), or a protein product is made that can no longer carry out its normal biological function or can no longer be transported to its normal cellular location, or a protein product is made that carries out its normal biological function but at a significantly reduced level.

In a non-limiting embodiment, a genetic construct of the present invention is used to mutate a wild-type NPC1L1 gene by replacement of at least a portion of the coding or regulatory sequence of the wild-type gene with a different nucleotide sequence such as, e.g., a mutated coding sequence or mutated regulatory region, or portion thereof. A mutated NPC1L1 gene sequence for use in such a genetic construct can be produced by any of a variety of known methods, including by use of error-prone PCR, or by cassette mutagenesis. For example, oligonucleotide-directed mutagenesis can be employed to alter the coding or regulatory sequence of a wild-type NPC1L1 gene in a defined way, e.g., to introduce a frame-shift or a termination codon at a specific point within the sequence. A mutated nucleotide sequence for use in the genetic construct of the present invention can be prepared by insertion into the coding or regulatory (e.g., promoter) sequence of one or more nucleotides, oligonucleotide molecules or polynucleotide molecules, or by replacement of a portion of the coding sequence or regulatory sequence with one or more different nucleotides, oligonucleotide molecules or polynucleotide molecules. Such oligonucleotide molecules or polynucleotide molecules can be obtained from any naturally occurring source or can be synthetic. The inserted sequence can serve simply to disrupt the reading frame of the NPC1L1 gene, or can further encode a heterologous gene product such as a selectable marker.

In one embodiment, NPC1L1 can be mutated in the transmembrane-spanning region, putative sterol sensing domain, amino-terminal ‘NPC1 domain’ domain, and/or ER/Goli targeting signal.

Mutations to produce modified cells, tissues and animals that are useful in practicing the present invention can occur anywhere in the NPC1L1 gene, including the open reading frame, the promoter or other regulatory region, or any other portion of the sequence that naturally comprises the gene or ORF. Such cells include mutants in which a modified form of the NPC1L1 protein normally encoded by the NPC1L1 gene is produced, or in which no protein normally encoded by the NPC1L1 gene is produced. Such cells can be null, conditional or leaky mutants.

Alternatively, a genetic construct can comprise nucleotide sequences that naturally flank the NPC1L1 gene or ORF in situ, with only a portion or no nucleotide sequences from the actual coding region of the gene itself. Such a genetic construct can be useful to delete the entire NPC1L1 gene or ORF.

Methods for carrying out homologous gene replacement are known in the art. For targeted gene mutation through homologous recombination, the genetic construct is preferably a plasmid, either circular or linearized, comprising a mutated nucleotide sequence as described above. In a non-limiting embodiment, at least about 200 nucleotides of the mutated sequence are used to specifically direct the genetic construct of the present invention to the particular targeted NPC1L1 gene for homologous recombination, although shorter lengths of nucleotides may also be effective. In addition, the plasmid preferably comprises an additional nucleotide sequence encoding a reporter gene product or other selectable marker constructed so that it will insert into the genome in operative association with the regulatory element sequences of the native NPC1L1 gene to be disrupted. Reporter genes that can be used in practicing the invention are known in the art, and include those encoding CAT, green fluorescent protein, and β-galactosidase, among others. Nucleotide sequences encoding selectable markers are also known in the art, and include those that encode gene products conferring resistance to antibiotics or anti-metabolites, or that supply an auxotrophic requirement.

In view of the present disclosure, methods that can be used for creating the genetic constructs of the present invention will be apparent, and can include in vitro recombinant techniques, synthetic techniques, and in vivo genetic recombination, as described, among other places, in Ausubel et al., 1989, above; Sambrook et al., 1989, above; Innis et al., 1995, above; and Erlich, 1992, above.

Mammalian cells can be transformed with a genetic construct of the present invention in accordance with known techniques, such as, e.g., by electroporation. Selection of transformants can be carried out using standard techniques, such as by selecting for cells expressing a selectable marker associated with the construct. Identification of transformants in which a successful recombination event has occurred and the particular target gene has been disabled can be carried out by genetic analysis, such as by Southern blot analysis, or by Northern analysis to detect a lack of mRNA transcripts encoding the particular protein, or by the appearance of cells lacking the particular protein, as determined, e.g., by immunological analysis, or some combination thereof.

The present invention thus provides modified mammalian cells in which the native NPC1L1 gene has been mutated. The present invention further provides modified animals in which the NPC1L1 gene has been mutated.

### Genetically Modified Animals

Genetically modified animals can be produced for studying the biological function of the NPC1L1 of the present invention in vivo and for screening and/or testing candidate compounds, e.g., inhibitors, such as antisense nucleic acids, shRNAs, siRNAs, or ribozymes, small molecules, or antibodies, for their ability to affect, e.g., inhibit, the expression and/or activity of NPC1L1 as potential therapeutics for treating disorders of lipid metabolism, such as hyperlipidemia, e.g., hypercholesterolemia, obesity, type II diabetes, cardiovascular disease, and stroke. Other candidate compounds, e.g., NPC1L1 agonists, may be identified and/or tested for their ability to enhance or increase the expression and/or activity of NPC1L1 as potential therapeutics for treating disorders such as anorexia, cachexia, and wasting, using the genetically modified animals described herein.

To investigate the function of NPC1L1 in vivo in animals, NPC1L1-encoding polynucleotides or NPC1L1-inhibiting antisense nucleic acids, shRNAs, siRNAs, or ribozymes can be introduced into test animals, such as mice or rats, using, e.g., viral vectors or naked nucleic acids. Alternatively, transgenic animals can be produced. Specifically, “knock-in” animals with the endogenous NPC1L1 gene substituted with a heterologous gene or an ortholog from another species or a mutated NPC1L1 gene, or “knockout” animals with NPC1L1 gene partially or completely inactivated, or transgenic animals expressing or overexpressing a wild-type or mutated NPC1L1 gene (e.g., upon targeted or random integration into the genome) can be generated.

NPC1L1-encoding nucleic acids can be introduced into animals using viral delivery systems. Exemplary viruses for production of delivery vectors include without limitation adenovirus, herpesvirus, retroviruses, vaccinia virus, and adeno-associated virus (AAV). See, e.g., Becker et al., Meth. Cell Biol. 1994; 43: 161-89; Douglas and Curiel, Science & Medicine 1997; 4: 44-53; Yeh and Perricaudet, FASEB J. 1997; 11: 615-623; Kuo et al., Blood 1993; 82: 845; Markowitz et al., J. Virol. 1988; 62: 1120; Mann et al., Cell 1983; 33: 153; U.S. Pat. Nos. 5,399,346; 4,650,764; 4,980,289; 5,124,263; and International Publication No. WO 95/07358.

In an alternative method, a NPC1L1-encoding nucleic acid can be introduced by liposome-mediated transfection, a technique that provides certain practical advantages, including the molecular targeting of liposomes to specific cells. Directing transfection to particular cell types (also possible with viral vectors) is particularly advantageous in a tissue with cellular heterogeneity, such as the brain, pancreas, liver, and kidney. Lipids may be chemically coupled to other molecules for the purpose of targeting. Targeted peptides (e.g., hormones or neurotransmitters), proteins such as antibodies, or non-peptide molecules can be coupled to liposomes chemically.

In another embodiment, target cells can be removed from an animal, and a nucleic acid can be introduced as a naked construct. The transformed cells can be then re-implanted into the body of the animal. Naked nucleic acid constructs can be introduced into the desired host cells by methods known in the art, e.g., transfection, electroporation, microinjection, transduction, cell fusion, DEAE dextran, calcium phosphate precipitation, use of a gene gun or use of a DNA vector transporter. See, e.g., Wu et al., J. Biol. Chem. 1992; 267: 963-7; Wu et al., J. Biol. Chem. 1988; 263: 14621-4.

In yet another embodiment, NPC1L1-encoding nucleic acids can be introduced into animals by injecting naked plasmid DNA containing a NPC1L1-encoding nucleic acid sequence into the tail vein of animals, in particular mammals (Zhang et al., Hum. Gen. Ther. 1999, 10:1735-7). This injection technique can also be used to introduce siRNA targeted to NPC1L1 into animals, in particular mammals (Lewis et al., Nature Genetics 2002, 32: 105-106).

As specified above, transgenic animals can also be generated. Methods of making transgenic animals are well-known in the art (for transgenic mice see Gene Targeting: A Practical Approach, 2^{nd} Ed., Joyner ed., IRL Press at Oxford University Press, New York, 2000; Manipulating the Mouse Embryo: A Laboratory Manual, Nagy et al. eds., Cold Spring Harbor Press, New York, 2003; Teratocarcinomas and Embryonic Stem Cells: A Practical Approach, Robertson ed., IRL Press at Oxford University Press, 1987; Transgenic Animal Technology: A Laboratory Handbook, Pinkert ed., Academic Press, New York, 1994; Hogan, Manipulating the Mouse Embryo, Cold Spring Harbor Laboratory Press, Cold Spring Harbor, N.Y., 1986; Brinster et al., Proc. Nat. Acad. Sci. USA 1985; 82: 4438-4442; Capecchi, Science 1989; 244: 1288-1292; Joyner et al., Nature 1989; 338: 153-156; U.S. Pat. Nos. 4,736,866; 4,870,009; 4,873,191; for particle bombardment see U.S. Pat. No. 4,945,050; for transgenic rats see, e.g., Hammer et al., Cell 1990; 63: 1099-1112; for non-rodent transgenic mammals and other animals see, e.g., Pursel et al., Science 1989; 244: 1281-1288 and Simms et al., Bio/Technology 1988; 6: 179-183; and for culturing of embryonic stem (ES) cells and the subsequent production of transgenic animals by the introduction of DNA into ES cells using methods such as electroporation, calcium phosphate/DNA precipitation and direct injection see, e.g., Teratocarcinomas and Embryonic Stem Cells, A Practical Approach, Robertson ed., IRL Press, 1987). Clones of the nonhuman transgenic animals can be produced according to available methods (see e.g., Wilmut et al., Nature 1997; 385: 810-813 and International Publications No. WO 97/07668 and WO 97/07669).

In one embodiment, the transgenic animal is a “knockout” animal having a heterozygous or homozygous alteration in the sequence of an endogenous NPC1L1 gene that results in a decrease of NPC1L1 function, preferably such that NPC1L1 expression is undetectable or insignificant. Knockout animals are typically generated by homologous recombination with a vector comprising a transgene having at least a portion of the gene to be knocked out. Typically a deletion, addition or substitution has been introduced into the transgene to functionally disrupt it.

Knockout animals can be prepared by any method known in the art (see, e.g., Snouwaert et al., Science 1992; 257: 1083; Lowell et al., Nature 1993; 366: 740-42; Capecchi, Science 1989; 244: 1288-1292; Palmiter et al., Ann. Rev. Genet. 1986; 20: 465-499; Bradley, Current Opinion in Bio/Technology 1991; 2: 823-829; and International Publications No. WO 90/11354, WO 91/01140, WO 92/0968, and WO 93/04169). Preparation of a knockout animal typically requires first introducing a nucleic acid construct (a “knockout construct”), that will be used to decrease or eliminate expression of a particular gene, into an undifferentiated cell type termed an embryonic stem (ES) cell. The knockout construct is typically comprised of: (i) DNA from a portion (e.g., an exon sequence, intron sequence, promoter sequence, or some combination thereof) of a gene to be knocked out; and (ii) a selectable marker sequence used to identify the presence of the knockout construct in the ES cell. The knockout construct is typically introduced (e.g., electroporated or microinjected) into ES cells so that it can homologously recombine with the genomic DNA of the cell in a double crossover event. This recombined ES cell can be identified (e.g., by Southern hybridization or PCR reactions that show the genomic alteration) and is then injected into a mammalian embryo at the blastocyst stage. In a preferred embodiment where the knockout animal is a mammal, a mammalian embryo with integrated ES cells is then implanted into a foster mother for the duration of gestation (see, e.g., Zhou et al., Genes and Dev. 1995; 9: 2623-34).

In a specific embodiment, the knockout vector is designed such that, upon homologous recombination, the endogenous NPC1L1-related gene is functionally disrupted (i.e., no longer encodes a functional protein). Alternatively, the vector can be designed such that, upon homologous recombination, the endogenous NPC1L1-related gene is mutated or otherwise altered but still encodes functional protein (e.g., the upstream regulatory region can be altered to thereby alter the expression of the NPC1L1-related polypeptide). In the homologous recombination vector, the altered portion of NPC1L1-related gene is preferably flanked at its 5′ and 3′ ends by additional nucleic acid of the NPC1L1-related gene to allow for homologous recombination to occur between the exogenous NPC1L1-related gene carried by the vector and an endogenous NPC1L1-related gene in an embryonic stem cell. The additional flanking NPC1L1-related nucleic acid is of sufficient length for successful homologous recombination with the endogenous gene. Typically, several kilobases of flanking DNA (at both the 5′ and 3′ ends) are included in the vector (see, e.g., Thomas and Capecchi, Cell 1987; 51: 503). The vector is introduced into an ES cell line (e.g., by electroporation), and cells in which the introduced NPC1L1-related gene has homologously recombined with the endogenous NPC1L1-related gene are selected (see, e.g., Li et al., Cell 1992; 69: 915). The selected cells are then injected into a blastocyst of an animal (e.g., a mouse) to form aggregation chimeras (see, e.g., Bradley, in Teratocarcinomas and Embryonic Stem Cells: A Practical Approach, Robertson ed., IRL, Oxford, 1987, pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene.

The phenotype of knockout animals can be predictive of the in vivo function of the gene and of the effects or lack of effect of its antagonists or agonists. Knockout animals can also be used to study the effects of the NPC1L1 protein in models of disease, including, hyperlipidemia and other lipid-mediated disorders. In a specific embodiment, knockout animals, such as mice harboring the NPC1L1 gene knockout, may be used to produce antibodies against the heterologous NPC1L1 protein (e.g., human NPC1L1) (Claesson et al., Scan. J. Immunol. 1994; 0: 257-264; Declerck et al., J. Biol. Chem. 1995; 270: 8397-400).

Genetically modified animals expressing or harboring NPC1L1-specific antisense polynucleotides, shRNA, siRNA, or ribozymes can be used analogously to knockout animals described above.

In another embodiment of the invention, the transgenic animal is an animal having an alteration in its genome that results in altered expression (e.g., increased or decreased expression) of the NPC1L1 gene, e.g., by introduction of additional copies of NPC1L1 gene in various parts of the genome, or by operatively inserting a regulatory sequence that provides for altered expression of an endogenous copy of the NPC1L1 gene. Such regulatory sequences include inducible, tissue-specific, and constitutive promoters and enhancer elements. Suitable promoters include metallothionein, albumin (Pinkert et al., Genes Dev. 1987; 1: 268-76), and K-14 keratinocyte (Vassar et al., Proc. Natl. Acad. Sci. USA 1989; 86: 1563-1567) gene promoters. Overexpression or underexpression of the wild-type NPC1L1 polypeptide, polypeptide fragment or a mutated version thereof may alter normal cellular processes, resulting in a phenotype that identifies a tissue in which NPC1L1 expression is functionally relevant and may indicate a therapeutic target for the NPC1L1, its agonists or antagonists. For example, a transgenic test animal can be engineered to overexpress or underexpress a full-length NPC1L1 sequence, which may result in a phenotype that shows similarity with human diseases.

Transgenic animals can also be produced that allow for regulated (e.g., tissue-specific) expression of the transgene. One example of such a system that may be produced is the Cre-Lox recombinase system of bacteriophage P1 (Lakso et al., Proc. Natl. Acad. Sci. USA 1992; 89: 6232-6236; U.S. Pat. Nos. 4,959,317 and 5,801,030). If the Cre-Lox recombinase system is used to regulate expression of a transgene, animals containing transgenes encoding both the Cre recombinase and a selected protein are required. Such animals can be provided through the construction of “double” transgenic animals, e.g., by mating two transgenic or gene-targeted animals, one containing a transgene encoding a selected protein or containing a targeted allele (e.g., a loxP flanked exon), and the other containing a transgene encoding a recombinase (e.g., a tissue-specific expression of Cre recombinase). Another example of a recombinase system is the FLP recombinase system of Saccharomyces cerevisiae (O'Gorman et al., Science 1991; 251: 1351-1355; U.S. Pat. No. 5,654,182). In another embodiment, both Cre-Lox and Flp-Frt are used in the same system to regulate expression of the transgene, and for sequential deletion of vector sequences in the same cell (Sun et al., Nat. Genet. 2000; 25: 83-6). Regulated transgenic animals can be also prepared using the tet-repressor system (see, e.g., U.S. Pat. No. 5,654,168).

The in vivo function of NPC1L1 can be also investigated through making “knock-in” animals. In such animals the endogenous NPC1L1 gene can be replaced, e.g., by a heterologous gene, by a NPC1L1 ortholog or by a mutated NPC1L1 gene. See, for example, Wang et al., Development 1997; 124: 2507-2513; Zhuang et al., Mol. Cell. Biol. 1998; 18: 3340-3349; Geng et al., Cell 1999; 97: 767-777; Baudoin et al., Genes Dev. 1998; 12: 1202-1216. Thus, a non-human transgenic animal can be created in which: (i) a human ortholog of the non-human animal NPC1L1 gene has been stably inserted into the genome of the animal; and/or (ii) the endogenous non-human animal NPC1L1 gene has been replaced with its human counterpart (see, e.g., Coffman, Semin. Nephrol. 1997; 17: 404; Esther et al., Lab. Invest. 1996; 74: 953; Murakami et al., Blood Press. Suppl. 1996; 2: 36). In one aspect of this embodiment, a human NPC1L1 gene inserted into the transgenic animal is the wild-type human NPC1L1 gene. In another aspect, the NPC1L1 gene inserted into the transgenic animal is a mutated form or a variant of the human NPC1L1 gene.

Included within the scope of the present invention are transgenic animals, preferably mammals (e.g., mice) in which, in addition to the NPC1L1 gene, one or more additional genes (preferably, associated with hyperlipidemia or related disorders) have been knocked out, or knocked in, or overexpressed. Such animals can be generated by repeating the procedures set forth herein for generating each construct, or by breeding two animals of the same species (each with a different single gene manipulated) to each other, and screening for those progeny animals having the desired genotype.

### Inhibition of NPC1L1

As specified above, the NPC1L1-encoding nucleic acid molecules of the can be used to inhibit the expression of NPC1L1 genes (e.g., by inhibiting transcription, splicing, transport, or translation or by promoting degradation of corresponding mRNAs). Specifically, the nucleic acid molecules of the invention can be used to “knock down” or “knock out” the expression of the NPC1L1 genes in a cell or tissue (e.g., in an animal model or in cultured cells) by using their sequences to design antisense oligonucleotides, RNA interference (RNAi) molecules, ribozymes, nucleic acid molecules to be used in triplex helix formation, etc. Preferred methods to inhibit gene expression are described below.

In one embodiment the transcription of NPC1L1 mRNA is inhibited by targeting NPC1L1 promoter transcription factors using an agonist or antagonist to these factors. In this embodiment the specific agonist or antagonist is identified by its ability to downregulate the expression of a reporter gene (such as luciferase or green fluorescence protein) driven by the promoter for NPC1L1, e.g., the mouse, rat or human promoter.

RNA Interference (RNAi). RNA interference (RNAi) is a process of sequence-specific post-transcriptional gene silencing by which double stranded RNA (dsRNA) homologous to a target locus can specifically inactivate gene function in plants, fungi, invertebrates, and vertebrates, including mammals (Hammond et al., Nature Genet. 2001; 2: 110-119; Sharp, Genes Dev. 1999; 13: 139-141). This dsRNA-induced gene silencing is mediated by short double-stranded small interfering RNAs (siRNAs) generated from longer dsRNAs by ribonuclease III cleavage (Bernstein et al., Nature 2001; 409: 363-366 and Elbashir et al., Genes Dev. 2001; 15: 188-200). RNAi-mediated gene silencing is thought to occur via sequence-specific mRNA degradation, where sequence specificity is determined by the interaction of an siRNA with its complementary sequence within a target mRNA (see, e.g., Tuschl, Chem. Biochem. 2001; 2: 239-245).

For mammalian systems, RNAi commonly involves the use of dsRNAs that are greater than 500 bp; however, it can also be activated by introduction of either siRNAs (Elbashir, et al., Nature 2001; 411: 494-498) or short hairpin RNAs (shRNAs) bearing a fold back stem-loop structure (Paddison et al., Genes Dev. 2002; 16: 948-958; Sui et al., Proc. Natl. Acad. Sci. USA 2002; 99: 5515-5520; Brummelkamp et al., Science 2002; 296: 550-553; Paul et al., Nature Biotechnol. 2002; 20: 505-508).

The siRNAs to be used in the methods of the present invention are preferably short double stranded nucleic acid duplexes comprising annealed complementary single stranded nucleic acid molecules. In preferred embodiments, the siRNAs are short dsRNAs comprising annealed complementary single strand RNAs. However, the invention also encompasses embodiments in which the siRNAs comprise an annealed RNA:DNA duplex, wherein the sense strand of the duplex is a DNA molecule and the antisense strand of the duplex is a RNA molecule. In one embodiment, an siRNA of the invention is set forth as SEQ ID NO: 23 or SEQ ID NO: 24.

Preferably, each single stranded nucleic acid molecule of the siRNA duplex is of from about 19 nucleotides to about 27 nucleotides in length. In preferred embodiments, duplexed siRNAs have a 2 or 3 nucleotide 3′ overhang on each strand of the duplex. In preferred embodiments, siRNAs have 5′-phosphate and 3′-hydroxyl groups.

The RNAi molecules to be used in the methods of the present invention comprise nucleic acid sequences that are complementary to the nucleic acid sequence of a portion of the target locus. In certain embodiments, the portion of the target locus to which the RNAi probe is complementary is at least about 15 nucleotides in length. In preferred embodiments, the portion of the target locus to which the RNAi probe is complementary is at least about 19 nucleotides in length. The target locus to which an RNAi probe is complementary may represent a transcribed portion of the NPC1L1 gene or an untranscribed portion of the NPC1L1 gene (e.g., intergenic regions, repeat elements, etc.).

The RNAi molecules may include one or more modifications, either to the phosphate-sugar backbone or to the nucleoside. For example, the phosphodiester linkages of natural RNA may be modified to include at least one heteroatom other than oxygen, such as nitrogen or sulfur. In this case, for example, the phosphodiester linkage may be replaced by a phosphothioester linkage. Similarly, bases may be modified to block the activity of adenosine deaminase. Where the RNAi molecule is produced synthetically, or by in vitro transcription, a modified ribonucleoside may be introduced during synthesis or transcription.

According to the present invention, siRNAs may be introduced to a target cell as an annealed duplex siRNA, or as single stranded sense and anti-sense nucleic acid sequences that, once within the target cell, anneal to form the siRNA duplex. Alternatively, the sense and anti-sense strands of the siRNA may be encoded on an expression construct that is introduced to the target cell. Upon expression within the target cell, the transcribed sense and antisense strands may anneal to reconstitute the siRNA.

The shRNAs to be used in the methods of the present invention comprise a single stranded “loop” region connecting complementary inverted repeat sequences that anneal to form a double stranded “stem” region. Structural considerations for shRNA design are discussed, for example, in McManus et al., RNA 2002; 8: 842-850. In certain embodiments the shRNA may be a portion of a larger RNA molecule, e.g., as part of a larger RNA that also contains U6 RNA sequences (Paul et al., supra).

In preferred embodiments, the loop of the shRNA is from about 1 to about 9 nucleotides in length. In preferred embodiments the double stranded stem of the shRNA is from about 19 to about 33 base pairs in length. In preferred embodiments, the 3′ end of the shRNA stem has a 3′ overhang. In particularly preferred embodiments, the 3′ overhang of the shRNA stem is from 1 to about 4 nucleotides in length. In preferred embodiments, shRNAs have 5′-phosphate and 3′-hydroxyl groups.

Although the RNAi molecules useful according to the invention preferably contain nucleotide sequences that are fully complementary to a portion of the target locus, 100% sequence complementarity between the RNAi probe and the target locus is not required to practice the invention.

RNA molecules useful for RNAi may be chemically synthesized, for example using appropriately protected ribonucleoside phosphoramidites and a conventional DNA/RNA synthesizer. RNAs produced by such methodologies tend to be highly pure and to anneal efficiently to form siRNA duplexes or shRNA hairpin stem-loop structures. Following chemical synthesis, single stranded RNA molecules are deprotected, annealed to form siRNAs or shRNAs, and purified (e.g., by gel electrophoresis or HPLC).

Alternatively, standard procedures may used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been described (Donzé and Picard, Nucleic Acids Res. 2002; 30: e46; and Yu et al., Proc. Natl. Acad. Sci. USA 2002; 99: 6047-6052). Similarly, an efficient in vitro protocol for preparation of shRNAs using T7 RNA polymerase has been described (Yu et al., supra). The sense and antisense transcripts may be synthesized in two independent reactions and annealed later, or may be synthesized simultaneously in a single reaction.

RNAi molecules may be formed within a cell by transcription of RNA from an expression construct introduced into the cell. For example, both a protocol and an expression construct for in vivo expression of siRNAs are described in Yu et al., supra. Similarly, protocols and expression constructs for in vivo expression of shRNAs have been described (Brummelkamp et al., supra; Sui et al., supra; Yu et al., supra; McManus et al., supra; Paul et al., supra).

The expression constructs for in vivo production of RNAi molecules comprise RNAi encoding sequences operably linked to elements necessary for the proper transcription of the RNAi encoding sequence(s), including promoter elements and transcription termination signals. Preferred promoters for use in such expression constructs include the polymerase-III HI-RNA promoter (see, e.g., Brummelkamp et al., supra) and the U6 polymerase-III promoter (see, e.g., Sui et al., supra; Paul, et al. supra; and Yu et al., supra). The RNAi expression constructs can further comprise vector sequences that facilitate the cloning of the expression constructs. Standard vectors that maybe used in practicing the current invention are known in the art (e.g., pSilencer 2.0-U6 vector, Ambion Inc., Austin, Tex.).

Antisense Nucleic Acids. In a specific embodiment, to achieve inhibition of expression of a NPC1 L1 gene, the nucleic acid molecules of the invention can be used to design antisense oligonucleotides. An antisense oligonucleotide is typically 18 to 25 bases in length (but can be as short as 13 bases in length) and is designed to bind to a selected NPC1L1 mRNA. This binding prevents expression of that specific NPC1L1 protein. The antisense oligonucleotides of the invention comprise at least 6 nucleotides and preferably comprise from 6 to about 50 nucleotides. In specific aspects, the antisense oligonucleotides comprise at least 10 nucleotides, at least 15 nucleotides, at least 25, at least 30, at least 100 nucleotides, or at least 200 nucleotides.

The antisense nucleic acid oligonucleotides of the invention comprise sequences complementary to at least a portion of the corresponding NPC1L1 mRNA. However, 100% sequence complementarity is not required so long as formation of a stable duplex (for single stranded antisense oligonucleotides) or triplex (for double stranded antisense oligonucleotides) can be achieved. The ability to hybridize will depend on both the degree of complementarity and the length of the antisense oligonucleotides. Generally, the longer the antisense oligonucleotide, the more base mismatches with the corresponding mRNA can be tolerated. One skilled in the art can ascertain a tolerable degree of mismatch by use of standard procedures to determine the melting point of the hybridized complex.

The antisense oligonucleotides can be DNA or RNA or chimeric mixtures, or derivatives or modified versions thereof, and can be single-stranded or double-stranded. The antisense oligonucleotides can be modified at the base moiety, sugar moiety, or phosphate backbone, or a combination thereof. For example, a NPC1L1-specific antisense oligonucleotide can comprise at least one modified base moiety selected from a group including but not limited to 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the NPC1L1-specific antisense oligonucleotide comprises at least one modified sugar moiety, e.g., a sugar moiety selected from arabinose, 2-fluoroarabinose, xylulose, and hexose.

In yet another embodiment, the NPC1L1-specific antisense oligonucleotide comprises at least one modified phosphate backbone selected from a phosphorothioate, a phosphorodithioate, a phosphoramidothioate, a phosphoramidate, a phosphordiamidate, a methylphosphonate, an alkyl phosphotriester, and a formacetal or analog thereof.

The antisense oligonucleotide can include other appending groups such as peptides, or agents facilitating transport across the cell membrane (see, e.g., Letsinger et al., Proc. Natl. Acad. Sci. USA 1989; 86: 6553-6556; Lemaitre et al., Proc. Natl. Acad. Sci. USA 1987; 84: 648-652; PCT Publication No. WO 88/09810) or blood-brain barrier (see, e.g., PCT Publication No. WO 89/10134), hybridization-triggered cleavage agents (see, e.g., Krol et al., BioTechniques 1988; 6: 958-976), intercalating agents (see, e.g., Zon, Pharm. Res. 1988; 5: 539-549), etc.

In another embodiment, the antisense oligonucleotide can include α-anomeric oligonucleotides. An α-anomeric oligonucleotide forms specific double-stranded hybrids with complementary RNA in which, contrary to the usual β-units, the strands run parallel to each other (Gautier et al., Nucl. Acids Res. 1987; 15: 6625-6641).

In yet another embodiment, the antisense oligonucleotide can be a morpholino antisense oligonucleotide (i.e., an oligonucleotide in which the bases are linked to 6-membered morpholine rings, which are connected to other morpholine-linked bases via non-ionic phosphorodiamidate intersubunit linkages). Morpholino oligonucleotides are resistant to nucleases and act by sterically blocking transcription of the target mRNA.

Similar to the above-described RNAi molecules, the antisense oligonucleotides of the invention can be synthesized by standard methods known in the art, e.g., by use of an automated synthesizer. Antisense nucleic acid oligonucleotides of the invention can also be produced intracellularly by transcription from an exogenous sequence. For example, a vector can be introduced in vivo such that it is taken up by a cell within which the vector or a portion thereof is transcribed to produce an antisense RNA. Such a vector can remain episomal or become chromosomally integrated, so long as it can be transcribed to produce the desired antisense RNA. Such vectors can be constructed by recombinant DNA technology methods standard in the art. Vectors can be plasmid, viral, or others known in the art, used for replication and expression in mammalian cells. In another embodiment, “naked” antisense nucleic acids can be delivered to adherent cells via “scrape delivery”, whereby the antisense oligonucleotide is added to a culture of adherent cells in a culture vessel, the cells are scraped from the walls of the culture vessel, and the scraped cells are transferred to another plate where they are allowed to re-adhere. Scraping the cells from the culture vessel walls serves to pull adhesion plaques from the cell membrane, generating small holes that allow the antisense oligonucleotides to enter the cytosol.

The present invention thus provides a method for inhibiting the expression of a NPC1L1 gene in a eukaryotic, preferably mammalian, and more preferably rat, mouse or human cell, comprising providing the cell with an effective amount of a NPC1L1-inhibiting antisenseoligonucleotide.

Ribozyme Inhibition. In another embodiment, the expression of NPC1L1 genes of the present invention can be inhibited by ribozymes designed based on the nucleotide sequence thereof. Ribozyme molecules catalytically cleave mRNA transcripts and can be used to prevent expression of the gene product. Ribozymes are enzymatic RNA molecules capable of catalyzing the sequence-specific cleavage of RNA (for a review, see Rossi, Current Biology 1994; 4: 469-471). The mechanism of ribozyme action involves sequence-specific hybridization of the ribozyme molecule to complementary target RNA, followed by an endonucleolytic cleavage event. The composition of ribozyme molecules must include: (i) one or more sequences complementary to the target gene mRNA; and (ii) a catalytic sequence responsible for mRNA cleavage (see, e.g., U.S. Pat. No. 5,093,246).

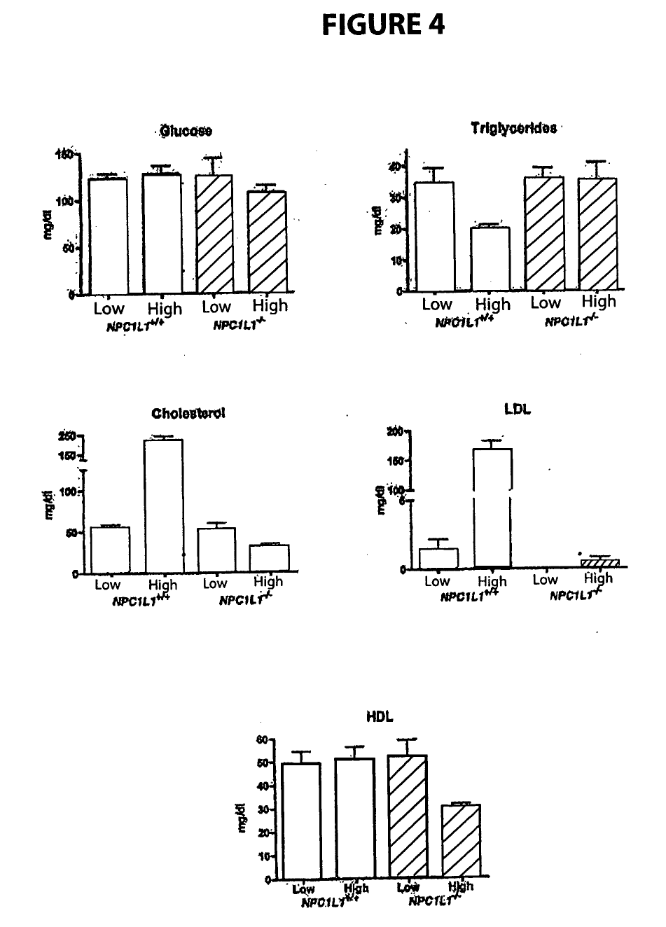

According to the present invention, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction of hammerhead ribozymes is known in the art, and described more fully in Myers, Molecular Biology and Biotechnology: A Comprehensive Desk Reference, VCH Publishers, New York, 1995 (see especially FIG. 4, page 833) and in Haseloff and Gerlach, Nature 1988; 334: 585-591.

Preferably, the ribozymes of the present invention are engineered so that the cleavage recognition site is located near the 5′ end of the corresponding mRNA, i.e., to increase efficiency and minimize the intracellular accumulation of non-functional mRNA transcripts.

As in the case of RNAi and antisense oligonucleotides, ribozymes of the invention can be composed of modified oligonucleotides (e.g., for improved stability, targeting, etc.). These can be delivered to mammalian cells, and preferably mouse, rat, or human cells, which express the target NPC1L1 protein in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the protein and inhibit translation. Because ribozymes, unlike antisense molecules, are catalytic, a lower intracellular concentration may be required to achieve an adequate level of efficacy.

Ribozymes can be prepared by any method known in the art for the synthesis of DNA and RNA molecules, as discussed above. Ribozyme technology is described further in Intracellular Ribozyme Applications: Principals and Protocols, Rossi and Couture eds., Horizon Scientific Press, 1999.

Triple Helix Formation. Nucleic acid molecules useful to inhibit NPC1L1 gene expression via triple helix formation are preferably composed of deoxynucleotides. The base composition of these oligonucleotides is typically designed to promote triple helix formation via Hoogsteen base pairing rules, which generally require sizeable stretches of either purines or pyrimidines to be present on one strand of a duplex. Nucleotide sequences may be pyrimidine-based, resulting in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., those containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, sequences can be targeted for triple helix formation by creating a so-called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Similarly to NPC1L1-specific RNAi, antisense oligonucleotides, and ribozymes, triple helix molecules of the invention can be prepared by any method known in the art. These include techniques for chemically synthesizing oligodeoxyribonucleotides and oligoribonucleotides such as, e.g., solid phase phosphoramidite chemical synthesis. Alternatively, RNA molecules can be generated by in vitro or in vivo transcription of DNA sequences “encoding” the particular RNA molecule. Such DNA sequences can be incorporated into a wide variety of vectors that incorporate suitable RNA polymerase promoters such as the T7 or SP6 polymerase promoters.

### Other NPC1L1 Antagonists

NPC1L1 inhibitors also include small molecules inhibitors. For example, several NPC1L1 inhibitors have been identified and are set forth in Example 10. These inhibitors include, for example, 4-phenyl-4-piperidinecarbonitrile hydrochloride, 1-butyl-N-(2,6-dimethylphenyl)-2 piperidinecarboxamide, 1-(1-naphthylmethyl)piperazine, 3{1-[(2-methylphenyl)amino]ethylidene}-2,4(3H, 5H)-thiophenedione, 3 {1-[(2-hydroxyphenyl)amino]ethylidene}-2,4(3H, 5H)-thiophenedione, 2-acetyl-3-[(2-methylphenyl)amino]-2-cyclopenten-1-one, 3-[(4-methoxyphenyl)amino]-2-methyl-2-cyclopenten-1-one, 3-[(2-methoxyphenyl)amino]-2-methyl-2-cyclopenten-1-one, and N-(4-acetylphenyl)-2-thiophenecarboxamide, or derivatives thereof. Additional NPC1L1 antagonists, e.g., small molecule antagonists, may be identified using, for example, the assays described herein.

### Diagnostic Methods

A variety of methods can be employed for the diagnostic evaluation of lipid disorders, such as hyperlipidemia and other diseases and disorders associated with or mediated by NPC1L1, such as obesity, type II diabetes, cardiovascular disease, and stroke, and for the identification and evaluation of subjects experiencing or at risk for developing hyperlipidemia, e.g., cholesterolemia and NPC1L1-associated conditions such as obesity, type II diabetes, cardiovascular disease, and stroke. These methods may also be employed for the diagnostic evaluation of diseases and disorders associated with decreased NPC1L1 such as anorexia, cachexia, and wasting.

These methods may utilize reagents such as the polynucleotide molecules and oligonucleotides of the present invention. The methods may alternatively utilize a NPC1L1 protein or a fragment thereof, or an antibody or antibody fragment that binds specifically to a NPC1L1 protein. Such reagents can be used for: (i) the detection of either an over- or an under-expression of the NPC1L1 gene relative to its expression in an unaffected state (e.g., in a subject or individual not having a disease or disorder associated with or mediated by NPC1L1); or (ii) the detection of either an increase or a decrease in the level of the NPC1L1 protein relative to its level in an unaffected state; or (iii) the detection of an aberrant NPC1L1 gene product activity relative to the unaffected state; or (iv) the mislocalization of vesicular proteins such as caveolin or annexin.

In a preferred embodiment, a diagnostic method of the present invention utilizes quantitative hybridization (e.g., quantitative in situ hybridization, Northern blot analysis or microarray hybridization) or quantitative PCR (e.g., TaqMan®) using a NPC1L1-specific nucleic acid of the invention as a hybridization probe and PCR primers, respectively.

The present invention also provides a method for detecting cells which may have altered lipid or glucose metabolism in a test cell subjected to a treatment or stimulus or suspected of having been subjected to a treatment or stimulus, said method comprising:

(a) determining the expression level in the test cell of a nucleic acid molecule encoding a NPC1L1 protein; and
(b) comparing the expression level of the NPC1L1-encoding nucleic acid molecule in the test cell to the expression level of the same nucleic acid molecule in a control cell not subjected to a treatment or stimulus;
wherein a detectable change in the expression level of the NPC1L1-encoding nucleic acid molecule in the test cell compared to the expression level of the NPC1L1-encoding nucleic acid molecule in the control cell indicates that the test cell may have altered lipid or glucose metabolism.

According to the present invention, the detectable change in the expression level is any statistically significant change and preferably at least a 1.5-fold change as measured by any available technique such as hybridization or quantitative PCR (see the Definitions Section, above).

The test and control cells are preferably the same type of cells from the same species and tissue, and can be any cells useful for conducting this type of assay where a meaningful result can be obtained. Any cell type in which a NPC1L1-encoding nucleic acid molecule is ordinarily expressed, or in which a NPC1L1-encoding nucleic acid is expressed in connection with a treatment or stimulus affecting lipid or glucose metabolism may be used. For example, the test cell can be any cell derived from a tissue of an organism experiencing hyperlipidemia or another disease or disorder associated with or mediated by NPC1L1. Alternatively, the test cell can be any cell grown in vitro under specific conditions. When the test cell is derived from a tissue of an organism experiencing hyperlipidemia or another disease or disorder associated with or mediated by NPC1L1, it may or may not be known to be located in the region associated with disorder.

In one embodiment, the test and control cells are cells from the gastrointestinal system. Preferably, the test and control cells are enterocyte cells from the epithelium of the small intestine. The test and control cells can be derived from any appropriate organism, but are preferably human or mouse cells. In a specific embodiment, the test and control cells are from an animal model of lipid pathogenesis (e.g., a mouse model of hyperlipidemia) or any related disorder (e.g., obesity, cardiovascular disease, or diabetes) and may or may not be isolated from that animal model. In another embodiment, the first cell is from a subject, such as a human or companion animal, for which the test is being conducted to determine the state of lipid or glucose metabolism that subject, and the second cell is an appropriate control cell. The first cell may or may not be isolated from the subject being tested. Both the test cell and the control cell must have the ability to express NPC1 L1.

The control cell can be any cell which is known to have not been subjected to any treatment or stimulus associated with lipid or glucose metabolism. Preferably, the control cell is otherwise similar and treated identically to the test cell. For example, when the test cell is derived from a tissue of an animal experiencing hyperlipidemia or another disease or disorder associated with or mediated by NPC1L1, the control cell can be derived from an identical tissue or body part of a different animal from, preferably, the same species (or, alternatively, a closely related species) which animal is not experiencing hyperlipidemia or another disease or disorder associated with or mediated by NPC1L1. Alternatively, the control cell can be derived from an identical tissue or body part of the same animal from which the test cells are derived. However if this is the case, it should be established that the identical tissue or body part has not been subjected to any treatment or stimulus associated with lipid or glucose metabolism within the timeframe of the experiment. When the test cell is a cell grown in vitro under specific conditions, the control cell can be a similar cell grown in vitro in identical conditions but in the absence of the treatment or stimulus.

In one embodiment, the test cell has been exposed to a treatment or stimulus that simulates or mimics a lipid-related condition prior to determining the expression level of the nucleic acid molecule encoding the NPC1L1 protein, and the control cell is useful as an appropriate comparator cell to allow a determination of whether or not the test cell is exhibiting a lipid response. For example, where the test cell has been exposed to a treatment or stimulus that is, or that simulates or mimics, hyperlipidemia or another disease or disorder associated with or mediated by NPC1L1, the control cell has not been exposed to such a treatment or stimulus. In another embodiment, the test cell has been exposed to a compound that is being tested to determine whether it simulates or mimics hyperlipidemia or another disease or disorder associated with or mediated by NPC1L1.

In one embodiment, the nucleic acid molecule the expression of which is being determined according to this method encodes a mammalian NPC1L1 polypeptide. In a specific embodiment, the nucleic acid molecule encodes a mouse NPC1L1 polypeptide comprising the amino acid sequence of SEQ ID NO: 3.

In one embodiment, the expression level of the nucleic acid molecule in each of the test and control cells is determined by quantifying the amount of NPC1L1-encoding mRNA present in the two cells. In another embodiment, the expression level of the nucleic acid molecule in each of the test and control cells is determined by quantifying the amount of NPC1L1 protein present in each of the two cells. Where the test cell has a detectable change in the expression level of the NPC1L1-encoding nucleic acid molecule compared to the expression level of the NPC1L1-encoding nucleic acid molecule in the control cell, a lipid response in the test cell has been detected.

To assay levels of a NPC1L1-encoding nucleic acid in a sample, a variety of standard nucleic acid isolation and quantification methods can be employed. As specified above, in a preferred embodiment, a diagnostic method of the present invention utilizes quantitative hybridization (e.g., quantitative in situ hybridization, Northern blot analysis or microarray hybridization) or quantitative PCR (e.g., TaqMan®) using NPC1L1-specific nucleic acids of the invention as hybridization probes and PCR primers, respectively.

In PCR-based assays, gene expression can be measured after extraction of cellular mRNA and preparation of cDNA by reverse transcription (RT). A sequence within the cDNA can then be used as a template for a nucleic acid amplification reaction. Nucleic acid molecules of the present invention can be used to design NPC1L1-specific RT and PCR oligonucleotide primers (such as, e.g., SEQ ID NOS: 4-7). Preferably, the oligonucleotide primers are at least about 9 to about 30 nucleotides in length. The amplification can be performed using, e.g., radioactively labeled or fluorescently-labeled nucleotides, for detection. Alternatively, enough amplified product may be made such that the product can be visualized simply by standard ethidium bromide or other staining methods.

A preferred PCR-based detection method of the present invention is quantitative real time PCR (e.g., TaqMan® technology, Applied Biosystems, Foster City, Calif.). This method is based on the observation that there is a quantitative relationship between the amount of the starting target molecule and the amount of PCR product produced at any given cycle number. Real time PCR detects the accumulation of amplified product during the reaction by detecting a fluorescent signal produced proportionally during the amplification of a PCR product.

For more details on quantitative real time PCR, see Gibson et al., Genome Res. 1996; 6: 995-1001; Heid et al., Genome Res. 1996; 6: 986-994; Livak et al., PCR Methods Appl. 1995; 4: 357-362; Holland et al., Proc. Natl. Acad. Sci. USA 1991; 88: 7276-7280.

SYBR Green Dye PCR (Molecular Probes, Inc., Eugene, Oreg.), competitive PCR as well as other quantitative PCR techniques can also be used to quantify NPC1L1 gene expression according to the present invention.

NPC1L1 gene expression detection assays of the invention can also be performed in situ (e.g., directly upon sections of fixed or frozen tissue collected from a subject, thereby eliminating the need for nucleic acid purification). Nucleic acid molecules of the invention or portions thereof can be used as labeled probes or primers for such in situ procedures (see, e.g., Nuovo, PCR in situ Hybridization: Protocols And Application, Raven Press, New York, 1992). Alternatively, if a sufficient quantity of the appropriate cells can be obtained, standard quantitative Northern analysis can be performed to determine the level of gene expression using the nucleic acid molecules of the invention or portions thereof as labeled probes.

For in vitro cell cultures or in vivo animal models, the diagnostic reagents of the invention can be used in screening assays as surrogates lipid condition to identify compounds that affect expression of the NPC1L1 gene. For example, probes for the mouse NPC1L1 gene can be used for diagnosing individuals suspected of having a condition associated with abnormal lipid or glucose metabolism, and also for monitoring the effectiveness therapy used to treat such condition.

Various techniques can be used to measure the levels of NPC1L1 protein in a sample, including the use of anti-NPC1L1 antibodies or antibody fragments described above. For example, anti-NPC1L1 antibodies or antibody fragments can be used to screen test compounds to identify those compounds that can modulate NPC1L1 protein production. For example, anti-NPC1L1 antibodies or antibody fragments can be used to detect the presence of the NPC1L1 protein by, e.g., immunofluorescence techniques employing a fluorescently labeled antibody coupled with light microscopic, flow cytometric or fluorimetric detection methods. Such techniques are particularly preferred for detecting the presence of the NPC1L1 protein on the surface of cells. In addition, protein isolation methods such as those described by Harlow and Lane (Antibodies: A Laboratory Manual, Cold Spring Harbor Laboratory Press, Cold Spring Harbor, N.Y., 1988) can also be employed to measure the levels of NPC1L1 protein in a sample.

Antibodies or antigen-binding fragments thereof may also be employed histologically, e.g., in immunofluorescence or immunoelectron microscopy techniques, for in situ detection of the NPC1L1 protein. In situ detection may be accomplished by, e.g., removing a tissue sample from a patient and applying to the tissue sample a labeled antibody or antibody fragment of the present invention. This procedure can be used to detect both the presence of the NPC1L1 protein and its distribution in the tissue. Additionally, antibodies or antigen-binding fragments may be used to detect NPC1L1 protein in the serum of cells, tissues, or animals that produce NPC1L1 protein.

### Screening Methods

The present invention further provides a method for identifying a lead compound useful for modulating the expression of a NPC1L1-encoding nucleic acid, said method comprising:

(a) contacting a first cell with a test compound for a time period sufficient to allow the cell to respond to said contact with the test compound;
(b) determining the expression level of a NPC1L1-encoding nucleic acid molecule in the cell prepared in step (a); and
(c) comparing the expression level of the NPC1L1-encoding nucleic acid molecule determined in step (b) to the expression level of the NPC1L1-encoding nucleic acid molecule in a second (control) cell that has not been contacted with the test compound;
wherein a detectable change in the expression level of the NPC1L1-encoding nucleic acid molecule in the first cell in response to contact with the test compound compared to the expression level of the NPC1L1-encoding nucleic acid molecule in the second (control) cell that has not been contacted with the test compound, indicates that the test compound modulates the expression of the NPC1L1-encoding nucleic acid and is a candidate compound for the treatment of a disorder associated with abnormal lipid or glucose metabolism.

In one embodiment, the candidate compound decreases the expression of the NPC1L1-encoding nucleic acid molecule. In another embodiment, the candidate compound increases the expression of the NPC1L1-encoding nucleic acid molecule. In another embodiment, the first and second cells are incubated under conditions that induce the expression of a NPC1L1-encoding nucleic acid molecule, but the test compound is tested for its ability to inhibit or reduce the induction of such expression in the first cell. In another embodiment, the first and second cells are incubated under conditions that induce the expression of a NPC1L1-encoding nucleic acid molecule, but the test compound is tested for its ability to potentiate the induction of such expression in the first cell.

The test compound can be, without limitation, a small organic or inorganic molecule, a polypeptide (including an antibody, antibody fragment, or other immunospecific molecule), an oligonucleotide molecule, a polynucleotide molecule, or a chimera or derivative thereof. Test compounds that specifically bind to a NPC1L1-encoding nucleic acid molecule or to a NPC1L1 protein of the present invention can be identified, for example, by high-throughput screening (HTS) assays, including cell-based and cell-free assays, directed against individual protein targets. Several methods of automated assays that have been developed in recent years enable the screening of tens of thousands of compounds in a short period of time (see, e.g., U.S. Pat. Nos. 5,585,277, 5,679,582, and 6,020,141). Such HTS methods are particularly preferred.

The first and second cells are preferably the same types of cells, and can be any cells useful for conducting this type of assay where a meaningful result can be obtained. Such cells can be prokaryotic, but are preferably eukaryotic. Such eukaryotic cells are preferably mammalian cells, and more preferably mouse or human cells. Both the first and second cell must have the ability to express NPC1L1. In one non-limiting embodiment, the first and second cells are cells that have been genetically modified to express or over-express a NPC1L1 nucleic acid molecule. In another non-limiting embodiment, the first and second cells are cells that express a NPC1L1 nucleic acid molecule, either naturally (e.g., cells lining the small intestine) or in response to an appropriate stimulus. In one embodiment, the first and second cells have been exposed to a condition or stimulus that is, or that simulates or mimics, a lipid condition prior to, or at the same time as, exposing the cells to the test compound to determine the effect of the test compound on the expression level of the nucleic acid molecule encoding the NPC1L1 polypeptide.

In one embodiment, the first and second cells are from an animal model of a disease or disorder associated with or mediated by NPC1L1 (e.g., mouse model of hypercholestolemia, obesity, diabetes, stroke or cardiovascular disease), and may or may not be isolated from that animal model. In another embodiment, the first cell is from a subject, such as a human or companion animal, and the second cell is an appropriate control cell. The first cell may or may not be isolated from the subject being tested.

In one embodiment, the nucleic acid molecule the expression of which is being determined according to this method encodes a mammalian NPC1L1 polypeptide. In a specific embodiment, the nucleic acid molecule encodes a mouse NPC1L1 polypeptide. In another embodiment, the mouse NPC1L1 polypeptide comprises the amino acid sequence of SEQ ID NO:3.

The expression level of the nucleic acid molecule in each of the first and second cells can be determined by quantifying and comparing the amount of NPC1L1-encoding mRNA present in each of the first and second cells. Alternatively, the expression level of the nucleic acid molecule in each of the first and second cells can be determined by quantifying and comparing the amount of NPC1L1 protein present in the first and second cells. Where the first cell has a detectable change in the expression level of the nucleic acid encoding a NPC1L1 protein compared to the expression level of the nucleic acid encoding the NPC1L1 protein in the second cell, the test compound is identified as a candidate compound useful for modulating the expression of a NPC1L1-encoding nucleic acid.

The present invention also provides a method for identifying a candidate compound that modulates an NPC1L1 polypeptide. In one embodiment, the present invention provides a method for identifying a ligand or other binding partner to the NPC1L1 protein of the present invention, which comprises bringing a labeled test compound in contact with the NPC1L1 protein or a fragment thereof and measuring the amount of the labeled test compound bound to the NPC1L1 protein or to the fragment thereof.

In another embodiment, the present invention provides a method for identifying a ligand or other binding partner to the NPC1L1 protein of the present invention, which comprises bringing a labeled test compound in contact with cells or cell membrane fraction containing the NPC1L1 protein, and measuring the amount of the labeled test compound bound to the cells or the membrane fraction.

In yet a third embodiment, the present invention provides a method for identifying a ligand or other binding partner to the NPC1L1 polypeptide of the present invention, which comprises culturing a transfected cell containing the DNA encoding the NPC1L1 protein under conditions that permit or induce expression of the NPC1L1 protein, bringing a labeled test compound in contact with the NPC1L1 protein expressed on a membrane of said cell, and measuring the amount of the labeled test compound bound to the NPC1L1 protein.

For example, the ligand or binding partner of the NPC1L1 protein of the present invention can be determined by the following procedures. First, a standard NPC1L1 preparation can be prepared by suspending cells or membranes containing the NPC1L1 protein in a buffer appropriate for use in the determination method. Any buffer can be used so long as it does not inhibit the ligand-NPC1L1 binding. Such buffers include, e.g., a phosphate buffer or a Tris-HCl buffer having pH of 4 to 10 (preferably pH of 6 to 8). For the purpose of minimizing non-specific binding, a surfactant such as CHAPS, Tween-80™ (manufactured by Kao-Atlas Inc.), digitonin or deoxycholate, and various proteins such as bovine serum albumin or gelatin, may optionally be added to the buffer. For the purpose of suppressing degradation of the NPC1L1 or ligand by proteases, a protease inhibitor such as PMSF, leupeptin, E-64 (manufactured by Peptide Institute, Inc.) and pepstatin can be added. A given amount (e.g., 5,000 to 500,000 cpm) of the test compound labeled with [³H], [¹²⁵I], [¹⁴C], [³⁵S] or the like can be added to about 0.01 ml to 10 ml of the solution containing NPC1L1. To determine the amount of non-specific binding (NSB), a reaction tube containing an unlabeled test compound in a large excess is also prepared. The reaction is carried out at about 0 to 50° C., preferably about 4 to 37° C. for about 20 minutes to about 24 hours, preferably about 30 minutes to about 3 hours. After completion of the reaction, the cells or membranes containing any bound ligand are separated, e.g., the reaction mixture is filtered through glass fiber filter paper and washed with an appropriate volume of the same buffer. The residual radioactivity on the glass fiber filter paper can be measured by means of a liquid scintillation counter or λ-counter. A test compound exceeding 0 cpm obtained by subtracting NSB from the total binding (B) (B minus NSB) may be selected as a ligand or binding partner of the NPC1L1 protein of the present invention.

Additionally, any of a variety of known methods for detecting protein-protein interactions may also be used to detect and/or identify proteins that bind to a NPC1L1 gene product. For example, co-immunoprecipitation, chemical cross-linking and yeast two-hybrid systems as well as other techniques known in the art may be employed. As an example in a yeast two-hybrid assay, a host cell harbors a construct that expresses a NPC1L1 protein or fragment thereof fused to a DNA binding domain and another construct that expresses a potential binding-partner fused to an activation domain. The host cell also includes a reporter gene that is expressed in response to binding of the NPC1L1 protein-partner complex (formed as a result of binding of binding-partner to the NPC1L1 protein) to an expression control sequence operatively associated with the reporter gene. Reporter genes for use in the yeast two-hybrid assay of the invention encode detectable proteins, including, but by no means limited to, chloramphenicol transferase (CAT), β galactosidase (β gal), luciferase, green fluorescent protein (GFP), alkaline phosphatase, and other genes that can be detected, e.g., immunologically (by antibody assay). See the Mammalian MATCHMAKER Two-Hybrid Assay Kit User Manual from Clontech (Palo Alto, Calif.) for further details on mammalian two-hybrid methods.

All of the screening methods described herein can be modified for use in high-throughput screening, e.g., using microarrays.

### Microarrays

Protein arrays. Protein arrays are solid-phase, ligand binding assay systems using immobilized proteins on surfaces that are selected from glass, membranes, microtiter wells, mass spectrometer plates, and beads or other particles. The ligand binding assays using these arrays are highly parallel and often miniaturized. Their advantages are that they are rapid, can be automated, are capable of high sensitivity, are economical in their use of reagents, and provide an abundance of data from a single experiment.

Automated multi-well formats are the best-developed HTS systems. Automated 96-well plate-based screening systems are the most widely used. The current trend in plate based screening systems is to reduce the volume of the reaction wells further, thereby increasing the density of the wells per plate (96 wells to 384 wells, and 1,536 wells per plate). The reduction in reaction volumes results in increased throughput, dramatically decreased bioreagent costs, and a decrease in the number of plates that need to be managed by automation. For a description of protein arrays that can be used for HTS, see, e.g., U.S. Pat. Nos. 6,475,809; 6,406,921; and 6,197,599; and International Publications No. WO 00/04389 and WO 00/07024.

For construction of arrays, sources of proteins include cell-based expression systems for recombinant proteins, purification from natural sources, production in vitro by cell-free translation systems, and synthetic methods for peptides. For capture arrays and protein function analysis, it is important that proteins are correctly folded and functional. This is not always the case, e.g., where recombinant proteins are extracted from bacteria under denaturing conditions, whereas other methods (isolation of natural proteins, cell free synthesis) generally retain functionality. However, arrays of denatured proteins can still be useful in screening antibodies for cross-reactivity, identifying auto-antibodies, and selecting ligand binding proteins.

The immobilization method used should be reproducible, applicable to proteins of different properties (size, hydrophilic, hydrophobic), amenable to high throughput and automation, and compatible with retention of fully functional protein activity. Both covalent and non-covalent methods of protein immobilization can be used. Substrates for covalent attachment include, e.g., glass slides coated with amino- or aldehyde-containing silane reagents (Telechem). In the Versalinx™ system (Prolinx), reversible covalent coupling is achieved by interaction between the protein derivatized with phenyldiboronic acid, and salicylhydroxamic acid immobilized on the support surface. Covalent coupling methods providing a stable linkage can be applied to a range of proteins. Non-covalent binding of unmodified protein occurs within porous structures such as HydroGel™ (PerkinElmer), based on a 3-dimensional polyacrylamide gel.

Cell-Based Arrays. Cell-based arrays combine the technique of cell culture in conjunction with the use of fluidic devices for measurement of cell response to test compounds in a sample of interest, screening of samples for identifying molecules that induce a desired effect in cultured cells, and selection and identification of cell populations with novel and desired characteristics. High-throughput screens (HTS) can be performed on fixed cells using fluorescent-labeled antibodies, biological ligands and/or nucleic acid hybridization probes, or on live cells using multicolor fluorescent indicators and biosensors. The choice of fixed or live cell screens depends on the specific cell-based assay required.

There are numerous single- and multi-cell-based array techniques known in the art. Recently developed techniques such as micro-patterned arrays (described, e.g., in International PCT Publications WO 97/45730 and WO 98/38490) and microfluidic arrays provide valuable tools for comparative cell-based analysis. Transfected cell microarrays are a complementary technique in which array features comprise clusters of cells overexpressing defined cDNAs. Complementary DNAs cloned in expression vectors are printed on microscope slides, which become living arrays after the addition of a lipid transfection reagent and adherent mammalian cells (Bailey et al., Drug Discov. Today 2002; 7(18 Suppl): S113-8). Cell-based arrays are described in detail in, e.g., Beske, Drug Discov. Today 2002; 7(18 Suppl): S131-5; Sundberg et al., Curr. Opin. Biotechnol. 2000; 11: 47-53; Johnston et al., Drug Discov. Today 2002; 7: 353-63; U.S. Pat. Nos. 6,406,840 and 6,103,479, and U.S. published patent application No. 2002/0197656. For cell-based assays specifically used to screen for modulators of ligand-gated ion channels, see Mattheakis et al., Curr. Opin. Drug Discov. Devel. 2001; 1: 124-34; and Baxter et al., J. Biomol. Screen. 2002; 7: 79-85.

For detection of molecules using screening assays, a molecule (e.g., an antibody or polynucleotide probe) can be detectably labeled with an atom (e.g., radionuclide), detectable molecule (e.g., fluorescein), or complex that, due to its physical or chemical property, serves to indicate the presence of the molecule. A molecule can also be detectably labeled when it is covalently bound to a “reporter” molecule (e.g., a biomolecule such as an enzyme) that acts on a substrate to produce a detectable product. Detectable labels suitable for use in the present invention include any composition detectable by spectroscopic, photochemical, biochemical, immunochemical, electrical, optical or chemical means. Labels useful in the present invention include, but are not limited to, biotin for staining with labeled avidin or streptavidin conjugate, magnetic beads (e.g., Dynabeads™), fluorescent dyes (e.g., fluorescein, fluorescein-isothiocyanate (FITC), Texas red, rhodamine, green fluorescent protein, enhanced green fluorescent protein, lissamine, phycoerythrin, Cy2, Cy3, Cy3.5, Cy5, Cy5.5, Cy7, Fluor X [Amersham], SyBR Green I & II [Molecular Probes], and the like), radiolabels (e.g., 3H, 125I, 35S, 14C, or 32P), enzymes (e.g., hydrolases, particularly phosphatases such as alkaline phosphatase, esterases and glycosidases, or oxidoreductases, particularly peroxidases such as horse radish peroxidase, and the like), substrates, cofactors, inhibitors, chemiluminescent groups, chromogenic agents, and colorimetric labels such as colloidal gold or colored glass or plastic (e.g., polystyrene, polypropylene, latex, etc.) beads. Examples of patents describing the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. For example, radiolabels and chemiluminescent labels can be detected using photographic film or scintillation counters; fluorescent markers can be detected using a photo-detector to detect emitted light (e.g., as in fluorescence-activated cell sorting); and enzymatic labels can be detected by providing the enzyme with a substrate and detecting, e.g., a colored reaction product produced by the action of the enzyme on the substrate.

### Activity Assays

The present invention further provides a method for studying additional biological activities of the NPC1L1 protein. The biological activity of the NPC1L1 protein can be studied using intact cells that express the NPC1L1 protein (either naturally, e.g., as a result of a stimulus or treatment, or heterologously), membrane fractions comprising the NPC1L1 protein, the isolated NPC1L1 protein, soluble NPC1L1 fragments, or NPC1L1 fusion proteins. For example, a biological activity of the NPC1L1 protein can be studied by measuring in a cell that heterologously expresses the NPC1L1 protein the activities that promote or suppress the production of an “index substance”, change in cell membrane potential, phosphorylation of intracellular proteins, activation of c-fos, pH reduction, etc.

NPC1L1-mediated activities can be determined by any known method. For example, cells containing the NPC1L1 protein can first be cultured on a multi-well plate, etc. Prior to the activity determination, the medium can be replaced with fresh medium or with an appropriate non-cytotoxic buffer, followed by incubation for a given period of time in the presence of a test compound, etc. Subsequently, the cells can be extracted or the supernatant can be recovered and the resulting product can be quantified by appropriate procedures. Where it is difficult to detect the production of the “index substance” for the cell-stimulating activity due to a degrading enzyme contained in the cells, an inhibitor against such a degrading enzyme may be added prior to the assay. For detecting activities such as the cAMP production suppression activity, the baseline production in the cells is increased by forskolin or the like and the suppressing effect on the increased baseline.

### Methods of Treatment

The present invention provides methods for treating, e.g., ameliorating, preventing, inhibiting, reducing the symptoms of, or delaying a condition that can be treated by modulating expression of a NPC1L1-encoding nucleic acid molecule or a NPC1L1 protein, comprising administering to a subject in need of such treatment a therapeutically effective amount of a compound that modulates expression of a NPC1L1-encoding nucleic acid molecule or a NPC1L1 protein.

Conditions that can be treated or prevented using the methods disclosed herein include those in which there are abnormalities in regulating lipid metabolism or responses, including cellular influx or efflux, endocytosis, or intracellular trafficking, transport, or localization of lipids, e.g., cholesterol, fatty acids, triglycerides, and sphingolipids. Such conditions include those that are associated with hyperlipidemia, including diet-induced hypercholesterolemia, obesity, cardiovascular disease, and stroke. In addition, conditions associated with aberrant glucose metabolism and transport, e.g., diabetes (e.g., type II diabetes) can also be treated using the methods disclosed herein. Furthermore, conditions associated with decreased NPC1L1 expression or activity, such as anorexia, cachexia, and wasting, may also be treated or prevented using the methods disclosed herein.

The term “therapeutically effective amount” is used here to refer to: (i) an amount or dose of a compound sufficient to detectably change the level of expression of a NPC1L1-encoding nucleic acid in a subject; or (ii) an amount or dose of a compound sufficient to detectably change the level of activity of a NPC1L1 protein in a subject; or (iii) an amount or dose of a compound sufficient to cause a detectable improvement in a clinically significant symptom or condition (e.g., amelioration of hypercholesterolemia) in a subject.

In a preferred embodiment, the therapeutically effective amount of a compound reduces or inhibits the expression or activity of an NPC1L1 nucleic acid or polypeptide.

### Formulations and Administration

A candidate compound useful in conducting a therapeutic method of the present invention is advantageously formulated in a pharmaceutical composition with a pharmaceutically acceptable carrier. The candidate compound may be designated as an active ingredient or therapeutic agent for the treatment of dietary hypercholesterolemia or other disorder involving lipid or glucose metabolism or transport.

The concentration of the active ingredient depends on the desired dosage and administration regimen, as discussed below. Suitable dose ranges of the active ingredient are from about 0.01 mg/kg to about 1500 mg/kg of body weight per day.

Therapeutically effective compounds can be provided to the patient in standard formulations, and may include any pharmaceutically acceptable additives, such as excipients, lubricants, diluents, flavorants, colorants, buffers, and disintegrants. The formulation may be produced in useful dosage units for administration by oral, parenteral, transmucosal, intranasal, rectal, vaginal, or transdermal routes. Parental routes include intravenous, intra-arteriole, intramuscular, intradermal, subcutaneous, intraperitoneal, intraventricular, intrathecal, and intracranial administration.

The pharmaceutical composition may also include other biologically active substances in combination with the candidate compound. Such substances include but are not limited to lovastin and ezetimibe.

The pharmaceutical composition can be added to a retained physiological fluid such as blood or synovial fluid. For CNS administration, a variety of techniques are available for promoting transfer of the therapeutic agent across the blood brain barrier, including disruption by surgery or injection, co-administration of a drug that transiently opens adhesion contacts between CNS vasculature endothelial cells, and co-administration of a substance that facilitates translocation through such cells.

In another embodiment, the active ingredient can be delivered in a vesicle, particularly a liposome.

In another embodiment, the therapeutic agent can be delivered in a controlled release manner. For example, a therapeutic agent can be administered using intravenous infusion with a continuous pump, in a polymer matrix such as poly-lactic/glutamic acid (PLGA), in a pellet containing a mixture of cholesterol and the active ingredient (SilasticR™; Dow Corning, Midland, Mich.; see U.S. Pat. No. 5,554,601), by subcutaneous implantation, or by transdermal patch

### EXAMPLES

The present invention is further described by way of the following particular examples. However, the use of such examples is illustrative only and is not intended to limit the scope or meaning of this invention or of any exemplified term. Nor is the invention limited to any particular preferred embodiment(s) described herein. Indeed, many modifications and variations of the invention will be apparent to those skilled in the art upon reading this specification, and such “equivalents” can be made without departing from the invention in spirit or scope. The invention is therefore limited only by the terms of the appended claims, along with the full scope of equivalents to which the claims are entitled.

### Example 1

### Intracellular Localization of the NPC1L1 Protein

Previous studies have revealed localization of NPC1 to the late endosome compartment of cells. The presence of NPC1 in this critical sorting region is consistent with the molecular etiology of Niemann-Pick C1 disease, which includes disruptions of cholesterol trafficking, storage, and secretion. Whether the NPC1L1 of the present invention localizes to the same region, however, is unclear. Although NPC1 and NPC1L1 have a number of common structural and functional domains, they also have different targeting sequences, suggesting distinct patterns of localization in the cell. In addition, another group has suggested that NPC1L1 molecule is present on the plasma membrane of enterocytes lining the small-intestine, a location consistent with their proposal that NPC1L1 is a transporter of dietary cholesterol and target of the anti-cholesterol drug ezitimibe. However, a recent study by Smart et al. (PNAS (2004) 101:345-3455, which presents evidence in both zebrafish and mouse systems that the target of ezitimibe is an annexin—caveolin heterocomplex, which is implicated as key mediator in the intestinal transport and trafficking of cholesterol. The present invention addresses this issue with a set of reagents and approaches to determine NPC1L1 localization.

### Methods

Production and purification of NPC1L1 antigen. A specific fragment of human NPC1L1 was amplified by PCR using the primers:

(SEQ ID NO: 4)

5′-GCGGGATCCGAACCGGTCCAGCTACAGGTA-3′
and

(SEQ ID NO: 5)

5′-GCGGAATTCCTCGAGGATGGGCAGGTCTTCAG-3′

spanning nucleotides 1302-1961 of SEQ ID NO: 2 and amino acids 416-635 of SEQ ID NO: 3. The amplified fragment was inserted into the pET-TRX expression vector, and the resulting recombinant plasmid was introduced into the host cell line, E. coli B121 (DE3) plysS. Purified NPC1L1 polypeptide was obtained by induced expression of the transformed cells followed by nickel affinity chromatography on a BioCAD system (Perseptive Biosystems, Framingham, Mass.).

Production and purification of anti-NPC1L1 antibodies. The NPC1L1 polypeptide was injected into two rabbits and polyclonal antisera was subsequently collected. Antiserum was sequentially purified in two affinity chromatography steps: (i) removal of Trx antibodies on a Trx-Affigel 10 column (BioRad, Hercules, Calif.); and (ii) purification of IgG antibodies on a Protein A-Sepharose column (Amersham Biosciences, Piscataway, N.J.).

Construction of NPC1L1 fusion vectors and RFP-reporter constructs. Monomeric (m) YFP and CFP were generated using eYFP and eCFP plasmids (Clontech) as templates. The L221K and Q69M mutations for mYFP and the L221K mutation in mCFP were created using the megaprimer PCR mutagenesis method and verified by sequencing. To generate mYFP and mCFP fusions with NPC1 L1, the stop codon of the human NPC1L1 sequence (GenBank accession number AY515256 was removed by PCR amplification and the resulting cDNA was verified by sequencing and fused to the mYFP and mCFP cDNAs. To introduce a Flag tag into NPC1L1, an adapter encoding the Flag tag amino acid sequence DYKDDDDK (SEQ ID NO: 29) was ligated in frame into the NPC1L1 at the unique BsmI restriction site. To generate a construct of RFP driven by the human ABCA1 promoter the genomic sequence of the promoter was amplified (nucleotides −189 to +32) and inserted into the pDsRed-Express vector (Clontech).

Tissue culture, transfection, and immunofluorescence studies. All cells, including COS7, NT2 and Caco-2 cells, obtained from ATCC (Manassas, Va.), were grown in DMEM supplemented with 2 mM glutamine, 10% FCS and Gentamicin at 37° C. and 5%, CO₂ in a humidified incubator. Cells were transfected using 4 ul Lipofectamine and 6 μl Plus reagent (Invitrogen, Carlsbad. CA), according to the manufacturer's recommendations. At 24 hr post-transfection the cells were either viewed live or they were fixed with ice-cold methanol at 4° C. for 6 min. Cells were processed for immunofluorescence using standard procedures and 1 μg/ml of rabbit polyclonal antibody or 2 ug/ml of M2 anti-Flag antibody (Sigma, St. Louis, Mo.), followed by a 1:1000 dilution of the appropriate secondary antibody, either goat anti-rabbit IgG-Alexa 488 (Molecular Probes, Eugene, Oreg.) or sheep anti-mouse IgG-FITC (Jackson Immunoresearch Laboratories, West Grove, Pa.). Cells were mounted in Fluoromount-G (Southern Biotechnology Associates, Birmingham, Ala.) and photographed using a Nikon Eclipse microscope equipped with a CCD camera.

Plasma membrane labeling assay. COS7 cells transfected with either Flag-tagged NPC1L1 or CD32 were labeled for 1 hr at 37° C. with 100 μCi ^{S35}-Met/^{S35}-Cys in cell medium deficient in these amino acids. Following a 2 hr chase period in DMEM complete medium, cells were removed from dishes using PBS containing 1 mM EDTA, washed in PBS and split equally into two eppendorfs. 2 μg of anti-Flag or anti-CD32 antibodies were added to half the samples and incubated on a rotating mixer at 4° C. for 30 min. Cells were washed twice with cold PBS and all samples were lysed in 500 μl lysis buffer (NPC1L1: 100 mM sodium phosphate pH 7.5, 150 mM NaCl, 2 mM EDTA, 1% igepal, 0.01% SDS; CD32: 50 mM Tris pH 7.4, 120 mM NaCl, 25 mM KCl, 0.2% Triton X100) containing proteinase inhibitor cocktail for 1 hr 30 min at 4° C. Lysates were cleared by centrifugation at 20,000 g for 10 min at 4° C. Samples previously incubated with antibody were transferred to tubes containing 20 μl protein G-agarose beads (Roche Applied Science, Indianapolis, Ind.) and incubated overnight at 4° C. Remaining samples were incubated at 4° C. for 1 hr with 3 μg anti-Flag/anti-CD32 antibodies, after which they were transferred to tubes containing protein G-agarose and incubated overnight at 4° C. Samples were washed four times in CD32 lysis buffer and once in NET1 buffer (50 mM Tris pH 7.4, 0.5M NaCl, 1 mM EDTA, 0.1% igepal, 0.25% gelatin, 0.02% sodium azide) and electrophoresed on a 4-20% bis-tris NUPAGE gel (Invitrogen, Carlsbad, Calif.) using the MOPS buffer system, until adequate separation was achieved. Gels were fixed in a solution of 10% acetic acid, 20% methanol for 10 min and soaked in Amplify solution for 15 min, before drying and exposing to film.

### Results

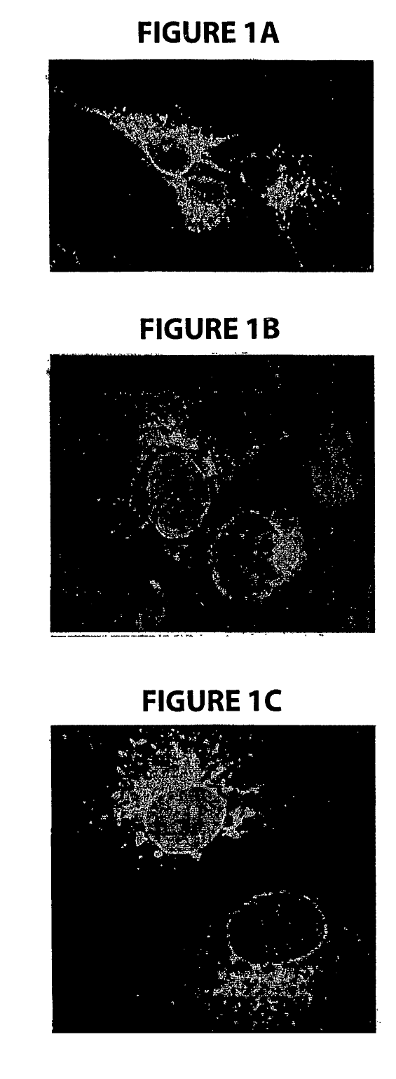

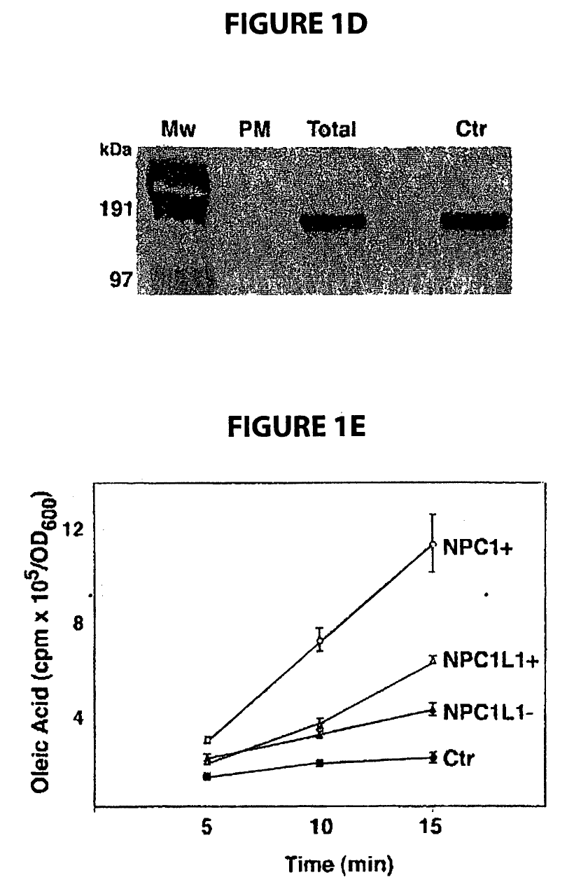

In one set of experiments, the purified anti-NPC1L1 polyclonal antibodies were used to determine the in situ localization of endogenous NPC1L1 in the human NT2 cell line. As visualized by indirect immunofluorescence, endogenous NPC1L1 showed a perinuclear, ER to Golgi distribution (FIG. 1a). Colocalization studies with various subcellular organelle markers (data not shown) confirmed the presence of NPC1L1 in the ER and Golgi. Notably, endogenous NPC1L1 was not present in the late endosomal/lysosomal compartment—in sharp contrast to the previously established residence of NPC1 in late endosomes (Higgins et al., (1999) Mol. Genet. Metab. 68: 1-13).

In another experiment, COS7 cells were visualized by fluorescent microscopy, following transient transfection of the expression vector comprising NPC1L1 fused to the Flag epitope. Consistent with the NT2 studies, the NPC1L1-flag fusion protein also localized predominantly to the ER and Golgi (FIG. 1b).

In addition, live Caco-2 cells were visualized by fluorescent microscopy, following transient transfection of the expression vector comprising NPC1L1 fused to mYFP. Again, the results reveal predominant localization of the NPC1L1 fusion to the ER and Golgi (FIG. 1c).

In addition, colocalization experiments (shown in Davies et al., J Biol. Chem. 2005) revealed that NPC1L1 localizes in an intracellular vesicular compartment with the marker protein Rab5.

In a final experiment, the membrane labeling assay was used as a sensitive detection method to confirm the intracellular localization of NPC1L1. In accord with the other findings, very little NPC1L1 can be labeled on the plasma membrane.

### Example 2

### NPC1L1 mRNA Expression in Human and Mouse Tissues

### Methods

Real time PCR quantitation. Human and mouse multiple tissue cDNA panels that had been normalized to four different control genes by the manufacturer (BD Biosciences Clontech, Palo Alto, Calif.) were amplified to detect only the full-length form of NPC1L1. Real-time PCR amplification was achieved using the Lightcycler 2 (Roche Applied Sciences). Data analysis was carried out using the accompanying software (v. 4.0). The primers used for amplifying mouse NPC1L1 were: 5′-GCTTCTTCCGCAAGATATACACTCCC-3′ (SEQ ID NO: 6) and 5′-GAGGATGCAGCAATAGC CACATAAGAC-3′ (SEQ ID NO: 7). The primers used for human NPC1L1 were 5′-TATCTTCCCTGGTTCCTGAACGAC-3′ (SEQ ID NO: 8) and 5′-CCGCAGAGCTTCTGTGTAATCC-3′ (SEQ ID NO: 9). For both the amplification cycles used were 95° C. for 10 sec, 58° C. for 20 sec and 72° C. for 20 sec. Relative quantitation was carried out using external standards and a linear fit method and each sample was amplified in three separate experiments. All statistical calculations were obtained using Microsoft Excel.

### Results

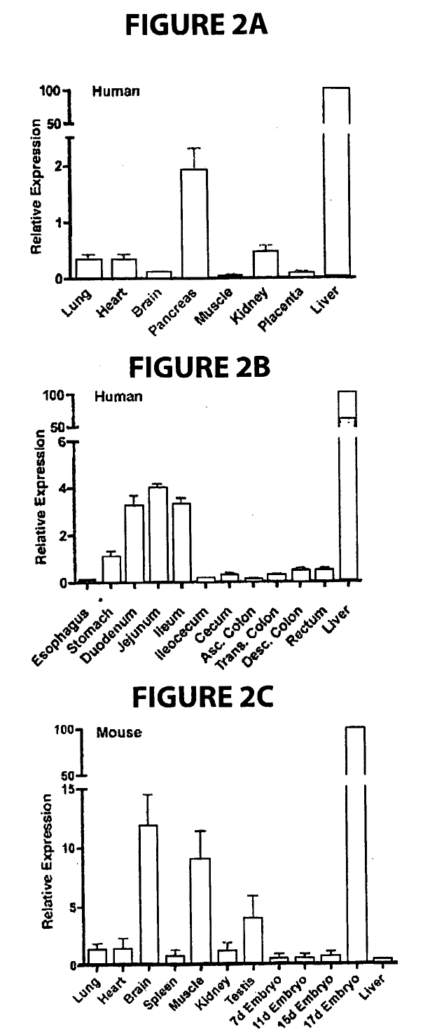

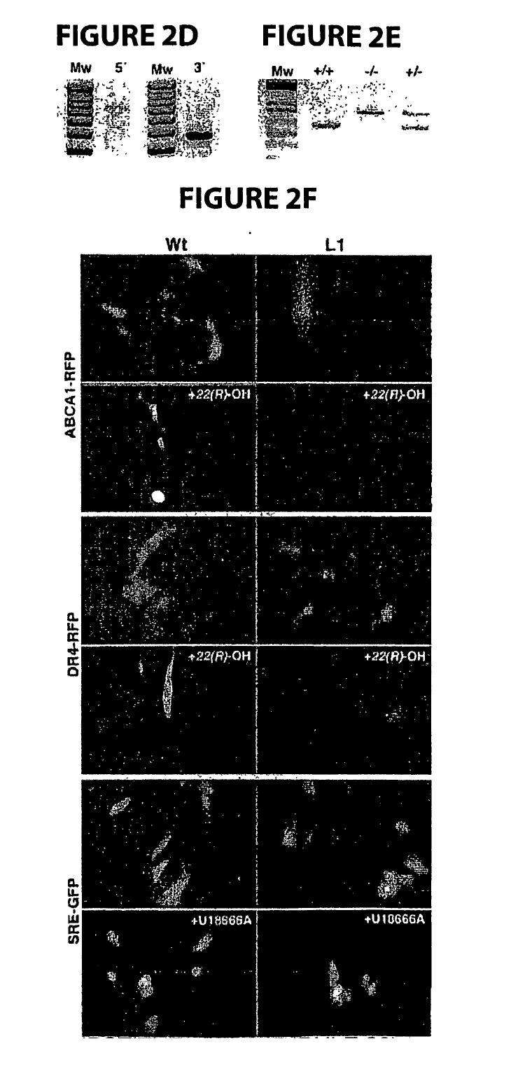

To further the functional studies of NPC1L1 the distribution of NPC1L1 mRNA expression was examined in both human and mouse tissues. In human tissues NPC1L1 is predominantly expressed in liver with detectable levels in lung, heart, brain, pancreas and kidney, ranging in expression from about 0.5 to 3% of liver expression (FIG. 2). Since it has been reported that mouse NPC1L1 is predominantly expressed in the small intestine (Higgins et al., 2001), analyses using a human panel of digestive tract tissues were also carried out. Human NPC1L1 is expressed in the small intestine at 1-4% of the levels expressed in liver (FIG. 2a-c) suggesting that there are significant differences between the expression of human and mouse NPC1L1. Interestingly, analyses of mouse tissues suggests a predominant role for NPC1L1 in embryogenesis since its highest expression is found in 17-day embryos; low but detectable expression was found in lung, heart, spleen and kidney and elevated expression in brain, muscle and testis (FIG. 2a-c).

### Example 3

### Lipid Uptake Function of NPC1L1 Function

### Introduction

NPC1L1 and NPC1 share a number of key structural features, including thirteen membrane spanning regions and a putative sterol sensitive motif. Accordingly, an important question is whether NPC1L1 shares some of the same functional properties as NPC1L1, specifically in the transport and movement of lipids. The present invention addresses the issue with respect to assays in bacterial cells.

### Methods

E. coli fatty acid transport assays. The predicted signal peptide of human NPC1L1, amino acids 1-33, was removed and the remaining full-length sequence, encoding amino-acids 33-1359, was cloned in-frame with the amino-terminal E. coli Omp A signal peptide sequence in the vector pIN III OmpA, as previously described for NPC1 (Davies et al., 2000). NPC1L1 was then expressed in the 2.1.1 strain of E. coli, as previously described (Davies et al., 2000) Briefly, E. coli cultures grown to log phase were induced to express NPC1L1 using 1 mM IPTG and grown for 1-2 hours. They were then diluted to an OD600 of 0.1 and incubated at 37° C. for 5-15 min in saline containing 0.1M TRIS, Ph7.5, 1 nM ³H sodium oleate and 105 nM cold sodium oleate. Cell pellets were resuspended in water and ³H sodium oleate was quantitated by scintillation counting.

### Results

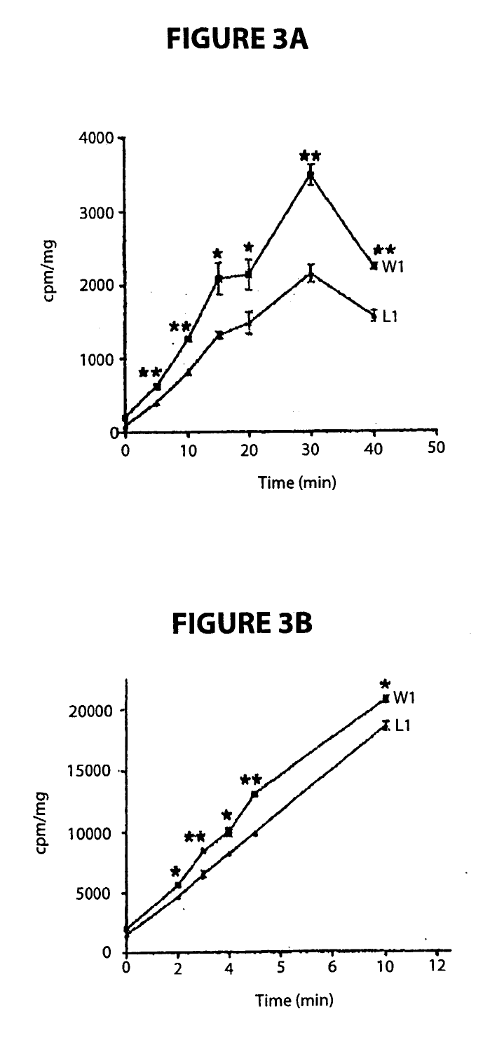

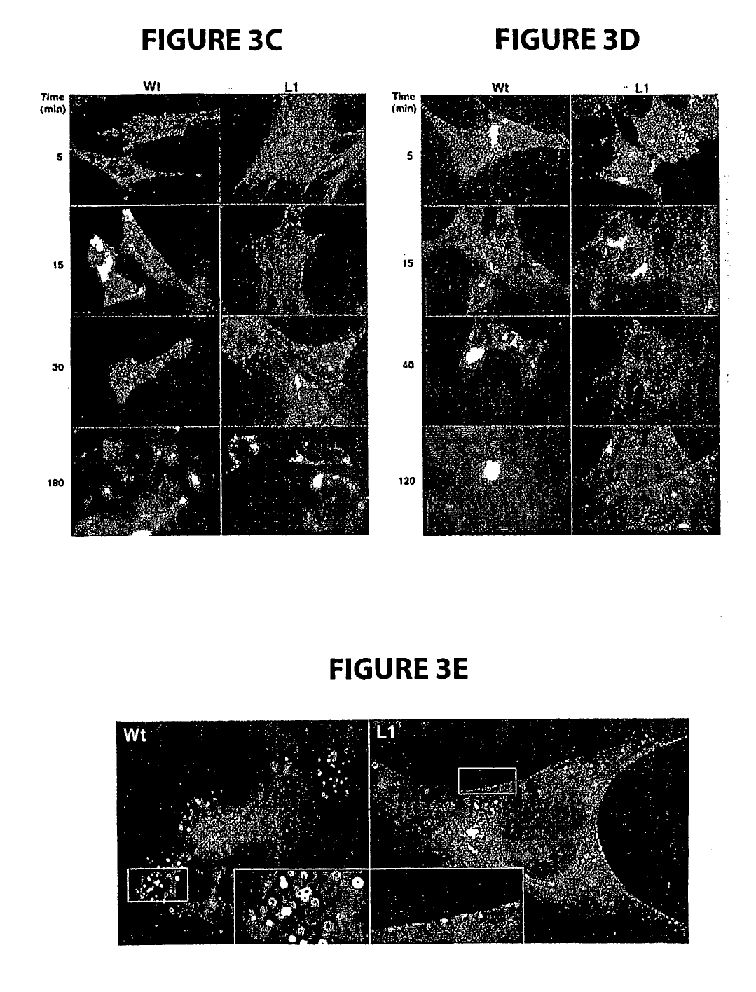

NPC1L1 was expressed in an engineered E. coli strain, designed for lipid transport studies (Davies et al., 2000). E. coli cells exhibited an increase in fatty acid accumulation compared to cells harboring a vector control (FIG. 3), albeit at a lower level than cells expressing NPC1 “indicating that NPC1L1 might have a function similar to that of NPC1 in a different intracellular location. These and other data (Davies et al., J Biol Chem 2005) indicate that NPC1L1 is a Rab5 colocalized intracellular protein that appears to share lipid permease activity with NPC1.”

### Example 4

### Generation of NPC1L1 Knockout Mice

### Introduction

Unlike NPC1, no human disease arising from mutations in NPC1L1 is currently known. To address this issue, the present invention discloses the isolation of the mouse NPC1L1 gene and its targeted disruption in the appropriate mouse strain. In this regard, the C57BL6 strain was chosen, given its established utility in the study of cholesterol-related diseases, including atherosclerosis.

### Methods

Isolation of mouse NPC1L1 gene. The genomic databases for BACs containing the mouse genomic sequence were searched and one clone that contained the mouse NPC1L1 promoter and entire coding region was identified. This clone, BAC RP23 64P22, accession number AC079435, from a C57BL6/J female mouse library, was obtained from BacPac Resources, Children's Hospital Oakland Research Institute (Oakland, Calif.). DNA was isolated using a BAC DNA isolation kit, as recommended (InCyte Genomics, St Louis, Mo.).

The mouse genomic nucleic acid sequence is provided in SEQ ID NO: 1. (The human genomic sequence is also provided in SEQ ID NO: 20. The NPC1L1 human cDNA is also presented in SEQ ID NO: 21 (GenBank Accession No. NM_{—}013389), and corresponding amino acid in SEQ ID NO: 22 (GenBank Accession No.: NP_{—}037521).

Targeted disruption of the endogenous NPC1L1 locus. A pGem7zf+(Promega)-based construct was engineered to contain nucleotides 84689 to 96003 of the mouse NPC1L1 gene (accession number AC079435), spanning the promoter region to intron 6. The gene was disrupted at the unique Afe I restriction enzyme site in exon 2 of the mouse NPC1L1 sequence (at 91263) by insertion of phosphoglycerate kinase neomycin phosphotransferase hybrid gene (PGK-neo), in an antisense direction. This disrupts the coding sequence after cDNA nucleotide 601 so that no more than 200 amino acids of NPC1L1 can be expressed. Thus the expression of all alternatively spliced forms of the gene is abrogated. Homologous recombination and selection for neomycin resistant knockout clones using C57BL6 ES cells (Taconic, Germantown, N.Y.) was carried out by Cell and Molecular Technologies (Phillipsburg, N.J.).

About 150 neo-resistant ES clones were obtained, 4 of which were correctly targeted by homologous recombination of the neomycin cassette into the NPC1L1 gene, clones 13, 19, 44 and 144. These were identified by PCR screening using two sets of primers, each containing one primer outside the NPC1L1 targeting cassette and one within the neomycin gene hybrid. At the 5′ end, these were 5′-CCTCCCTATTCCCCAAGATGTATGC-3′ (SEQ ID NO: 10) in the NPC1L1 gene at 83538 and 5′-GGAGAGGCTATTCGGCTATGAC-3′ (SEQ ID NO: 11) in the neomycin cassette. At the 3′ end these were: 5′-CTGGGCTCCCTCTTAGAATAACCTA-3′ SEQ ID NO: 12) at 96815 and 5′-GGAGAGGCTATTCGGCTATGAC-5′ (SEQ ID NO: 13) in the neomycin cassette. Long-range amplifications were achieved using the Failsafe PCR system (Epicentre, Madison, Wis.) with buffer F and 30 cycles of: 94° C. for 30 sec; annealing at 54° C. or 58° C. for the 5′ or 3′ end regions respectively; and 30 sec and 72° C. for 8 min. Correct products yield a 9 kb or a 5.5 kb product for the 5′ and 3′ regions respectively.

Chimeric mice were created by injecting knockout clone 13 C57BL6 ES cells into blastocysts that were then implanted into pseudopregnant BALB/c mice. Chimeric males were identified by coat color and one male that gave almost 100% germ-line transmission of ES cell-derived material was crossed with wild-type C57BL6 females. Mice that were heterozygous for the knockout allele were identified by long-range PCR.

Multiplex genotype analysis. For routine genotype analysis DNA was extracted from the mouse tail tissue using standard purification procedures and this was screened by multiplex PCR using the following primers: one primer in the neomycin sequence, 5′-CTCTGAGCCCAGAAAGCGAAG-3′ (SEQ ID NO: 14); and two primers within the NPC1L1 exon 2 sequence, NPC1L1a, 5′-GACCAGAGCCTCTTCATCAATGT-3′ (SEQ ID NO: 15) and NPC1L1b, 5′-GAGAATCTGCGCTTACGAGGGA-3′ (SEQ ID NO: 16) that flanked the neomycin insertion. The neomycin and NPC1L1b primer pair amplifies the knockout allele to produce a PCR product of 815 bp while the NPC1L1a and NPC1L1b primers amplify the 601 bp wildtype allele. PCR amplification used 30 cycles of denaturation at 94° C. for 40 sec, annealing at 58° C. for 30 sec and extension at 72° C. for 1 min.

### Results

Chimeric C57BL6 ES cell/BALBc mice were successfully generated and crossed with C57BL6 females. Homozygous NPC1L1−/− mice were identified by long-range PCR-amplification to verify that the neomycin/NPC1L1 gene knockout cassette was correctly inserted by homologous recombination (FIG. 3d). Mice were routinely screened by PCR to determine their genotype.

The resulting NPC1L1−/− mice were found to breed normally and showed no obvious phenotype when compared with their wild-type NPC1L1+/+counterparts. This was surprising considering that mice lacking NPC1 are generally sterile. These results do not exclude the possibility of subtle defects, such as those giving rise to minor abnormalities in the nervous system.

### Example 5

### Analysis of Lipid Uptake and Trafficking in Wild-Type and NPC1L1 Knockout Mouse Cells

### Introduction

NPC1L1 and NPC1 share a number of key structural features, including thirteen membrane spanning regions and a putative sterol sensitive motif. Accordingly, an important question is whether NPC1L1 shares some of the same functional properties as NPC1L1, specifically in the transport and movement of lipids. The present invention addresses the issue with a genetic-based approach in normal and NPC1L1-deficient mouse cells.

### Methods

Generation of SV40-immortalized cell lines. Wild-type and NPC1L1 knockout mice that were 3-6 days old were euthanized in a sterile environment and liver tissue was removed and minced into 3-4 mm pieces. These were washed in PBS, transferred to 1 ml of ice-cold 0.25% trypsin/100 mg tissue and incubated at 4° C. for 16 hours. The trypsin was removed and the tissue incubated at 37° C. for 10-30 min. DMEM medium containing 10% FBS and 2 mM L-glutamine was added, the cells were dispersed by pipetting and then kept in culture until they began to proliferate. Cells were transfected with the pTTKneo plasmid as previously described (Smart et al., 2004). Clones of SV40-transformed cells were picked and expression of the SV40 antigen was confirmed by immunofluorescence analysis using an anti-SV40 T antigen monoclonal antibody (BD biosciences pharmingen, San Diego, Calif.).

Fatty Acid Uptake Assays. Fatty acid uptake was carried out essentially as described (Pohl et al., 2002), using wild-type and NPC1L1 knockout mouse cells grown to confluency. Briefly, cells grown in 6 well dishes were washed in PBS and then incubated at 37° C. with 1 ml of prewarmed DMEM medium containing 173 μM BSA:173 μM sodium oleate with 0.43 μM ³H sodium oleate (23 Ci/mmol, Perkin Elmer, Wellesley, Mass.). The assay was stopped by the addition of 2 ml ice-cold DMEM containing 200 μM phloretin and 0.5% BSA and the cells incubated on ice for 2 min. The cells were then washed six times with ice cold DMEM and lysed in 1 ml of 1M NaOH. Protein concentrations were determined using the fluorescamine assay (Bishop et al., 1978). Scintillation counting was used to measure the ³H sodium-oleate in 100 μl of lysate. All samples were assayed in triplicate. A similar procedure was used to measure cholesterol uptake. ³H-cholesterol was solubilized using cyclodextrin essentially as described (Sheets et al., 1999). Briefly, a mixture containing 110 μl of ¹⁴C-cholesterol (52.9 mCi/mmol, Perkin Elmer), 1 mg cholesterol and methyl-β-cyclodextrin solution (mβCD/Chol 8:1 mol/mol) was sonicated in a bath sonicator for 15 min prior to an overnight incubation at 37° C. Confluent cells were incubated with 1 ml of DMEM containing 10 μl of solubilized cholesterol at 37° C. for 0-40 min.

NBD-Cholesterol and NBD-LacCer Uptake. The fluorescent sphingolipid NBDLacCer was obtained complexed to BSA (Molecular Probes) and incubated with subconfluent cultures in serum-free media for 5-10 min. The fluorescent probe was removed and fresh media containing serum was added. Cells were imaged live using a fluorescent microscope equipped with a CCD camera. NBD-cholesterol was complexed with cyclodextrin as described above for ³H-cholesterol. The cholesterol/cyclodextrin complex was added to cells as described above for NBD-LacCer. Cells were processed and imaged as above.

Construction of mYFP-caveolin and fluorescent reporter vectors. To generate an mYFP-Caveolin fusion vector, caveolin-1 (GenBank accession number NM_{—}001753) was amplified from a cDNA pool generated using human fibroblast mRNA, using the primers 5′-GCGAATTCTATGTCTGGGGGCAAATACGTAGA-3′ (SEQ ID NO: 17) and 5′-GCGGATCCTTATAT TTCTTTCTGCAAGTTGATGCGGA-3′ (SEQ ID NO: 18) Caveolin-1 was cloned at the 3′ end of mYFP cDNA (described above) to generate the mYFP-Caveolin-1 fusion. The SRE-GFP vector was as previously described. To generate the DR4-GFP vectors the SRE element was removed from SREGFP and replaced by 3 copies of a DR4 element 5 encoded by a double stranded oligonucleotide, 5′-TTGGGGTCATTGTCGGGCATTGGGGTCATTGTCGGGCATTGGGGTCATTGTC GGGCA-3′ (SEQ ID NO: 19) To generate a construct of RFP driven by the human ABCA1 promoter the genomic sequence of the promoter was amplified (nucleotides −189 to +32) (Walter et al., 2002) and inserted into the pDsRed-Express vector (Clontech).

### Results

To further characterize the role of NPC1L1 in lipid transport, mouse fibroblasts were isolated from NPC1L1+/+(Wt) and NPC1L11−/− (L1) mice and were immortalized by expression of the SV40 large T antigen 6. To characterize the response of these cells to changing lipid levels vectors were constructed in which the expression of GFP or RFP is controlled either by the ATP binding cassette transporter A1 (ABCA1) promoter, a dual DR4 element or a dual sterol-regulatory (SRE) element. Expression of these constructs in the Wt and L1 cells indicated that the L1 cells are unable to express RFP driven by the ABCA1 promoter or DR4 element (FIG. 2f). Both cell lines however, could express the SRE-driven GFP construct (FIG. 2f) and responded identically to the LDL-derived sterol transport inhibitor U18666A. These results provided evidence that the L1 cells have a normal SRE response but they are unable to sense or regulate their lipid efflux response.

To evaluate the extent of this transport defect it was next determined whether the absorption and endocytosis of lipids at the plasma membrane was also altered. To assess cholesterol influx rates, radio labeled cholesterol was incubated with cells for 0-40 min. Both cell lines exhibited saturatable uptake but transport into the L1 cells was reduced by 30% (FIG. 3a). Similarly, incubation with oleic acid revealed that L1 cells had a 5-10% decrease in uptake (FIG. 3b). Next cells were labeled as above with a fluorescent cholesterol analog and chased for various lengths of time. Initially, cholesterol decorates the plasma membrane of both Wt and L1 cells in a punctate manner (FIG. 3c). However, by 180 min, in Wt cells, NBD-cholesterol was localized at a single intracellular site, presumably Golgi, whereas in the L1 cells cholesterol accumulated in multiple intracellular pools (FIG. 3c).

In addition, incubation with the fluorescent sphingolipid NBD-lactosylceramide indicated that in addition to differences in the transport of cholesterol and fatty acids, L1 cells are also defective in their transport of sphingolipids. After 15 min of chase, NBD-lactosylceramide localized to the Golgi apparatus of Wt cells and this localization was complete by 40 min (FIG. 3d). However, in L1 cells NBDlactosylceramide was trapped in intracellular vesicular structures and did not reach the Golgi complex even after 120 min of chase (FIG. 3d). Intriguingly, this phenotype has recently been described in NPC1-defective cells (Puri et al., 1999), lending further support to the notion that NPC1 and NPC1L1 may perform similar functions.

The differences in lipid endocytosis between Wt and L1 cells suggested that the lack of NPC1L1 activity causes a generalized lipid transport block that may involve deregulation of caveolae formation and/or internalization. The caveolin family of small transmembrane proteins includes caveolin-1/VIP21, caveolin-2, and a muscle-specific isoform caveolin-3. Caveolin-1 spans the plasma membrane twice forming a hairpin structure on the surface and forms homo- and hetero-oligomers with caveolin-2. Caveolins are the principle constituents of caveolae (small non-clathrin coated invaginations in plasma membrane). They preferentially associate with inactive signaling molecules such as Src and Ras family proteins and have been proposed to act as a scaffold for the assembly of signaling complexes. Caveolin-1 colocalizes and associates with the integrin receptors in vivo. It regulates binding of the Src family kinases to the integrin receptors to promote adhesion and anchorage-dependent growth. Other proposed functions for caveolins include regulation of cell proliferation and tumor suppression.

Expression of a mYFPcaveolin construct showed that in Wt cells caveolin localizes in a perinuclear Golgi area and in peri-plasma membrane ring structures (Pohl et al., 2004; Westerman et al., 1999) (FIG. 3e). In striking contrast, the caveolin L1 cells appears to be trapped at the plasma membrane (FIG. 3e), suggesting that lack of NPC1L1 activity causes its aberrant trafficking or mislocalization. The inability of L1 cells to endocytose caveolae may partially explain their multiple lipid transport defects.

To determine whether NPC1L1 is active in caveolae colocalization studies were carried out between mYFP-caveolin and NPC1L1-mCFP. No significant colocalization between the two proteins was detected (data not shown) suggesting that the effects seen in L1 cells are not a direct effect of the lack of NPC1L1 activity in caveolae.

### Example 6

### Studies of Lipid Physiology in Wild-Type and NPC1L1-Knockout Mice

### Methods

Animal Care. All mice were housed in the Mount Sinai animal care facility with controlled humidity and temperature levels and with 12 hour alternating light and dark cycles. Experiments were carried out according to protocols approved by the Institutional Animal Care and use Committee (IACUC). For colony maintenance the mice were given a regular chow diet (Lab Diet rodent diet 20, PMI Nutritional International Richmond, Ind.) and water ad libitum. For studying the effects of an atherogenic diet the Paigen high cholesterol, high fat dietl was administered (Research Diets, cat. no. D12336) and contained 12.5 gm % cholesterol, 5 gm % sodium cholic acid and a fat content of 35 kcal %. The matched low fat diet (cat. no. D12337) contained 0.3 gm % cholesterol, no cholic acid and a fat content of 10 kcal %.

Plasma lipid Assays. For plasma lipid assays, mice were given the high and low cholesterol diets for 14 weeks and then fasted for 16 hours. They were euthanized using a lethal dose of the anesthetic Avertin and total body blood was withdrawn from the inferior vena cava. Four male and four female mice were used for each diet.

Histology. Livers from mice fed a high cholesterol diet were excised and fixed in 4% paraformaldehyde in PBS. They were embedded in paraffin, deparaffenized, rehydrated and 5 □m sections were stained using 0.1% hematoxylin and 0.25% alcoholic eosin. These were mounted in Permount and examined using a Nikon light microscope.

### Results

The NPC1L1+/+ and NPC1L1−/− mice were placed on a high cholesterol diet for 14 weeks. When serum lipid levels from these mice were evaluated, no significant differences were observed between NPC1L1+/+ and NPC1L1−/− mice on normal low cholesterol diet. As expected, Wt mice on the high fat diet exhibited an increase in total cholesterol and LDL-cholesterol and a decrease in their triglycerides whereas HDL-cholesterol was similar to those of animals kept on the low fat diet. However, the NPC1L1−/− mice given a high fat diet showed no elevation in total and LDL-cholesterol and in fact showed a significant decrease in total cholesterol. These animals had a decrease in HDL levels and had similar triglyceride levels to mice kept on the low fat diet. In addition, NPC1L1−/−mice on the high fat diet had a significant decrease in plasma glucose compared to NPC1L1+/+mice, which has a small but significant increase in plasma glucose (assayed following overnight fasting).

Histochemical analysis of liver tissues from these animals showed that NPC1L1+/+ mice on the high fat diet had larger, fat-laden livers, while livers from the knockout mice were normal but smaller than the Wt high-fat livers, indicating that these animals resisted the diet-induced fatty liver. Liver sections from NPC1L1+/+ and NPC1L1−/− mice confirmed the lipid-laden status of the NPC1L1+/+ livers and the resistance of NPC1L1−/− animals to this diet induced lipid accumulation. Also, gall bladders from Wt and NPC1L1−/− mice on the high fat diet were dramatically different with NPC1L1+/+ gall bladder tissues, showing obvious signs of lipid-induced cholestasis that were absent in the NPC1L1−/− mouse. Together, these data show that inactivation of the NPC1L1 protein has a protective effect against diet-induced hypercholesterolemia in these animals and suggest that NPC1L1 has a critical role in regulating lipid or glucose metabolism.

### Example 7

### Screening Assays for the Identification of NPC1L1 Modulators

A number of assays have been developed for the monitoring of NPC1L1 function. These assays include, for example, prokaryotic in vivo assays; prokaryotic in vitro assays; eukaryotic in vivo assays; and reconstitution.

All of these assays are amenable to high-throughput screening and offer four diverse ways for screening small molecule libraries. Below is a description of the various approaches.

Prokaryotic Assay

NPC1L1 has been successfully expressed in a prokaryotic host (E. coli). In these bacteria the protein is imbedded into the inner membrane. The engineering of the expression construct involved the replacement of the NPC1L1 ER-targeting signal sequence with that of the E. coli protein OmpA1. An IPTG-inducible promoter drives the expression of NPC1L1.

The expression host is a derivative of E. coli K12. This host was engineered to lack the prokaryotic permease AcrB (a permease that has homology with NPC1L1). The host was then engineered to also lack a second component of this system a protein called TolC, by homologous recombination deletion. This host has a tremendous advantage for our studies since the AcrB/TolC system in E. coli is very efficient and can work to mask or confuse the results of transporter expression studies.

In vivo: Using the above host the transport of specific substrates is able to measured by looking at growth rates and/or resistance to various compounds added to the growth media since NPC1L1 transports these substances into the bacteria where they exert a toxic effect. These assays can be done on semisolid or liquid media.

In vitro: Using the above cells we can produce membrane vesicles of the inner membrane that contain the NPC1L1 protein. These vesicles can be produced with the NPC1L1 protein facing the inside (10; inside out) or the outside (RO; right site out) of the vesicle. This is extremely useful since one can measure material going into the vesicles or coming out of the vesicles depending on need.

Thus, one can use the above system as a high throughput screening for either activators (agonists) or inhibitors of NPC1L1.

Eukaryotic In Vivo:

Mammalian: Cell-lines have been generated that express NPC1L1 or and cell-lines have been generated that lack NPC1L1 activity. Cells lacking NPC1L1 exhibit a number of differences with cells that express NPC1L1. These differences are measurable and can be monitored in live cells by fluorescence detection and/or microscopy. Thus, the effects or activity of various small molecules on the activity of NPC1L1 can be evaluated in a high-throughput screening system.

Baculovirus: A very high-level expression system has been produced based on baculovirus that expresses NPC1L1 tagged at the C-terminus with a dual histidine-HA tag in insect cells. This provides an efficient and quick way to purify large quantities of recombinant NPC1L1 for reconstitution studies/screening (see below). In addition, these cells can be used to confirm results or candidate molecule identified by one of the methods described above.

Reconstitution:

Purified NPC1L1 from insect cells: Purified material from the above (baculo) can be used to form vesicles in vitro using various lipid compositions including the one that NPC1L1 resides in (Golgi membranes). Fluorescent or radioactive probes can be incorporated into the membrane of these vesicles or captured into their interior hydrophilic core. Probes will be identified on their ability to change location within these vesicles dependent on the activity of NPC1L1. And therefore, their movement can be monitored in the presence of compounds that change (increase or decrease) the activity of NPC1L1.

A mammalian cell assay for screening potential NPC1L1 is described herein (see ricin assay as described in Example 10, below) and a prokaryotic system for screening potential NPC1L1 inhibitors is described in Example 8.

### Example 8

### Assay for Inhibitor Screening for NPC1 and NPC1L1 and Identification of 4-Phenylpiperidines as Potent Inhibitors of NPC1

In order to devise an assay for inhibitor screening a system where some potential activity of NPC1 or NPC1L1 can be detected and monitored is needed. Also, further complications are added by the fact that expression of these proteins in mammalian cells is usually not tolerated and sometimes lethal.

### Methods

The present inventors have devised a prokaryotic expression system for both NPC1 and NPC1L1 based on the expression of these proteins with prokaryotic secretion signals for targeting the E. coli inner membrane. The engineering of the expression construct involved the replacement of the NPC1 and NPC1L1 ER-targeting signal sequences with that of the E. coli protein OmpA1. An IPTG-inducible promoter drives the expression of NPC1 and NPC1L1. This system for expression of NPC1 has been described by the inventors (see Davies, Chen and Ioannou, Science 290: 2295-98, 2000).

In addition, hosts have been engineered to allow for the efficient detection of any potential activities as described in Example 9, below.

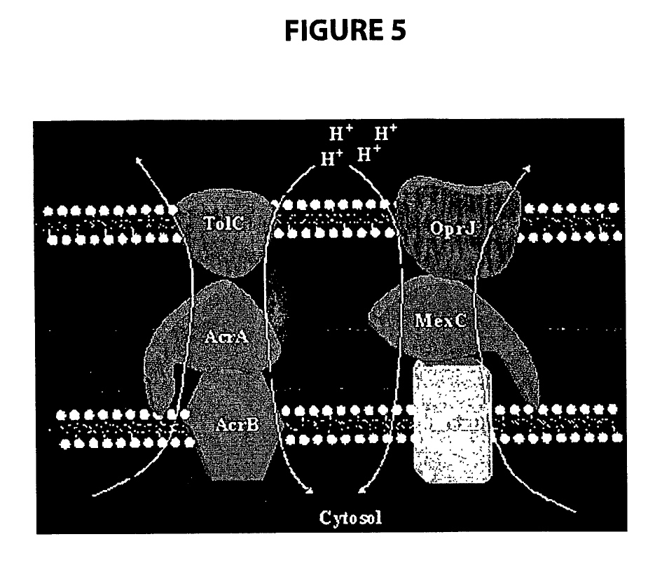

The expression host is a derivative of E. coli K12. This host was engineered to lack the prokaryotic permease AcrB (a permease that NPC1 and NPC1L1 have homology with), and was a gift from Dr. Tomofusa Tsuchiya (Antimicrob. Agents and Chemoth. 42: 1778, 1998). The host was engineered to lack a second component of this system a protein called TolC, by homologous recombination deletion (FIG. 5). This host has a tremendous advantage for these studies since the AcrB/TolC system in E. coli is a very efficient drug efflux system and can work to mask or confuse the results of transporter expression studies.

The final improvement made was introducing into these strains mutations that make their outer membrane leaky. The E. coli outer membrane is a strong barrier of lipophilic molecules and thus prevents any assays to be carried out that involve lipophilic substrates. Since the predicted substrates of NPC1 and NPC1L1 are lipophilic it is critical to engineer a strain that has a leaky outer membrane. In this manner lipophilic molecules can cross the outer membrane so that they can interact with the expressed NPC1 and NPC1L1 proteins residing on the inner membrane of the bacteria.

Utilizing this bacterial host it was discovered that these mutants are unable to grow in the presence of 5 mM concentration of a short chain fatty acid (decanoate; a 10 carbon length fatty acid). However, bacteria expressing NPC1 are able to overcome this block and grow in the presence of decanoic acid. In one type of assay bacteria are plated onto a dish to form a lawn. Small filter disks (about 8 mm diameter) are soaked in decanoate and placed onto the bacterial lawn. Dishes are incubated overnight at 37° C. and inspected the next morning. The substance (decanoate or other test material) diffuses from the filter in a radial manner into the bacterial lawn and will inhibit bacterial growth. The diameter of the inhibition ring (around the filter) will be directly related to the sensitivity of the bacteria to the test substance; the more resistant the bacteria are to the test substance the closer to the filter they will grow forming a smaller diameter ring.

This assay works equally well in liquid cultures; decanoate is added to liquid cultures and bacteria are grown at 37° C. with shaking for 4-6 hours. At the end of the incubation period an optical density measurement at 600 nm (OD₆₀₀) determines the ability of the culture to grow. Using the above cultures it was determined that control bacteria grew at an OD=0.9 whereas NPC1-expressing bacteria grew to saturation of OD>3.0.

### Results

The above assays were used to search for inhibitors of NPC1 and NPC1L1. On the plate assay various inhibitors, as set forth below, were added to the cultures before plating and searched for molecules that did not interfere with the growth of control bacteria in the presence of decanoate. In the NPC1-expressing cells an increase in the diameter of the growth inhibition ring was observed, suggesting that the NPC1 protein is inhibited and leads to these bacteria regaining their sensitivity to decanoate. A number of molecules were screened and a number of candidate inhibitors identified (set forth below). The two most promising candidates were validated in mammalian cell cultures.

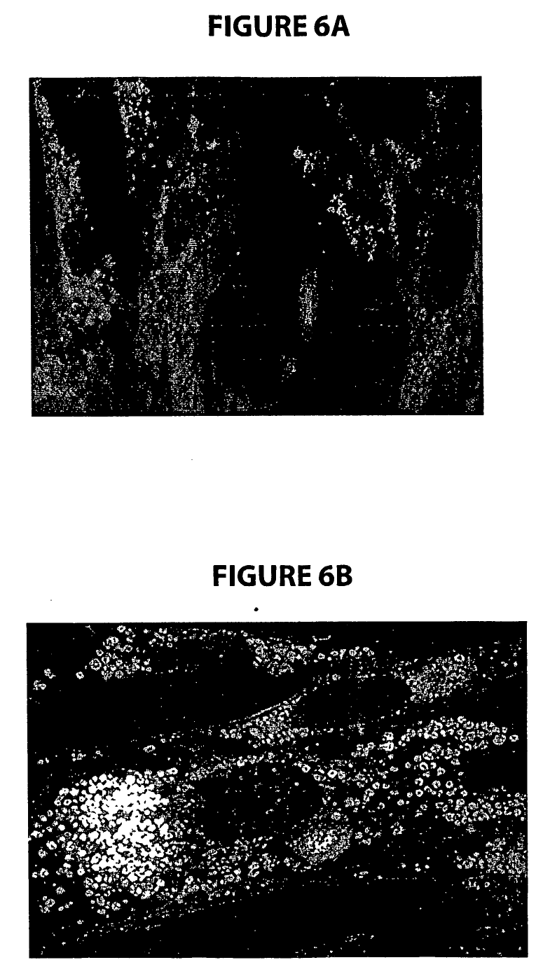

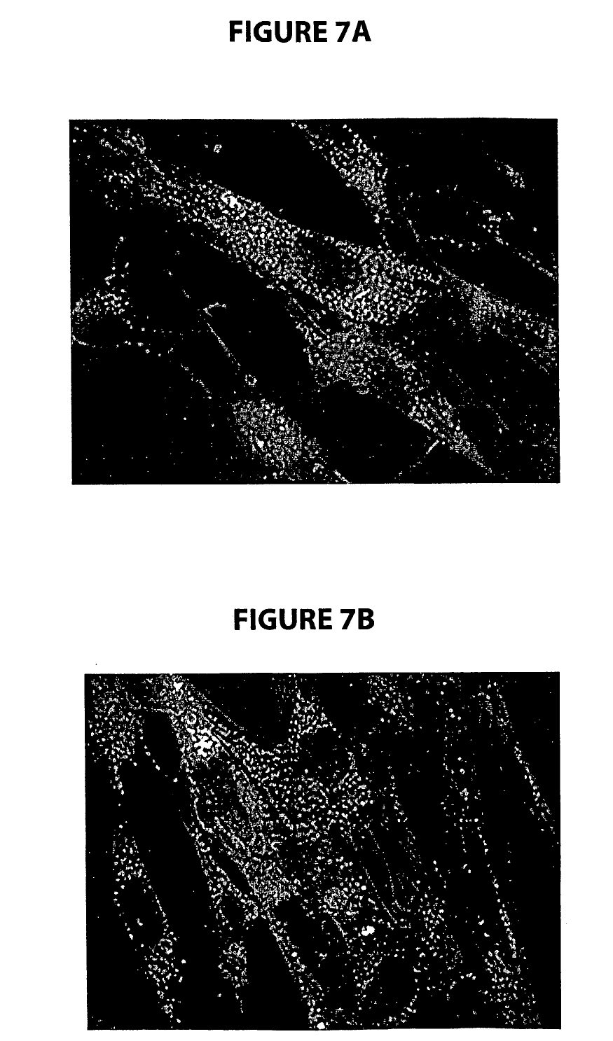

Cells were treated with these inhibitors and cholesterol storage was monitored. Cells treated with molecule #5 (4-butyryl-4-phenylpiperidine hydrochloride-see) overnight. In the presence of these inhibitors, mammalian cells should exhibit a disease phenotype (the human lipidosis Niemann-Pick C is due to a deficiency of NPC1). Cells from NPC1 patients store cholesterol in their lysosomes, which can be easily visualized by staining cells with a fluorescent probe that recognizes cholesterol. Results are shown in FIG. 6 and FIG. 7. No significant staining for lysosomal cholesterol can be seen in normal human fibroblasts (FIG. 6A). However, the same fibroblasts incubated overnight with inhibitor #5 have distinguishable lysosomes filled with cholesterol (FIG. 6B).

Molecule #2 (4-methylpiperidine) was a weaker NPC1 inhibitor, although fibroblasts treated with this inhibitor still exhibit cholesterol-filled lysosomes (FIG. 7A). Molecule #1 (4-phenyl-4-phenylpiperidine hydrochloride) did not demonstrate any NPC1 inhibition, as shown by an absence of cholesterol build-up in the lysosomes (FIG. 7B). The molecules identified as potential NPC1 inhibitors may also be effective as NPC1L1 inhibitors. For example, Molecule #1 (4-phenyl-4-phenylpiperidine hydrochloride), has been identified as an inhibitor of NPC1L1, even though it did not demonstrate any NPC1 inhibition.

### Candidate Inhibitors Identified Using the Above-Described Assay:

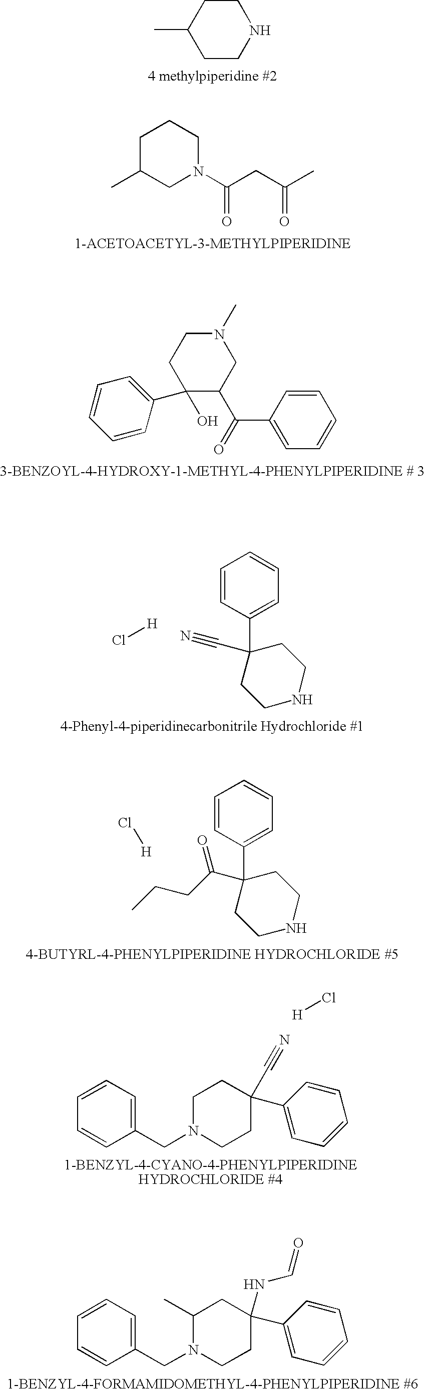

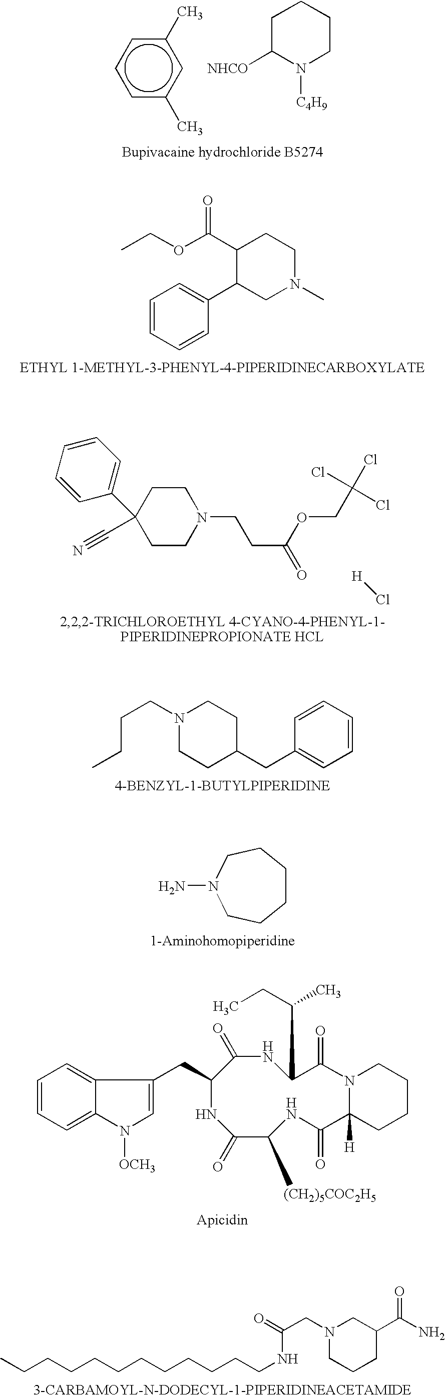

### Example 9

### Engineered E. coli Hosts for High-Level Expression of Mammalian Transporters

Expression of NPC1 in bacteria as previously described by the inventors (Davies et al., 2000 Science 290, 2295-2298) was limited by the fact that E. coli bacteria have a number of efflux pumps that belong in the Resistance-Nodulation-Division (RND) family. These pumps transport molecules away from the E. Coli cytosol in direct opposition to the direction of transport by NPC1 and NPC1 L1. This in turn complicates analysis of experimental data generated in this system. Thus, an AcrB mutant strain has been obtained which lacks one of the major RND permeases part of the AcrA, AcrB and TolC complex.

First, using this strain the TolC gene has been mutated by homologous recombination using the approach recently described (Link et al., 1997 J. Bacteriology 179, 6228-6237). The TolC gene forms the channel on the E. coli outer membrane and it is shared by most of the RND permeases in E. coli. Thus, inactivating this gene effectively inactivates most if not all, E. coli RND permeases.

Second, following construction of the double AcrB, TolC mutant strain, these bacteria were mutagenized and selected for strains with a “leaky” outer membrane similar to the previously described selection procedure (Davies et al., 2000 Science 290, 2295-2298). This mutagenesis produced an AcrB/TolC/permeable strain.

Third, this triple mutant, (AcrB/TolC/Perm), was used to select for expression of large transmembrane proteins. This selection is accomplished by allowing NPC1-expressing and NPC1L1-expressing bacteria to spontaneously mutate on agar plates (as described by Miroux and Walker, 1996 J Mol Biol 260, 289-298; Shaw and Miroux, (2003). A general approach to heterologous membrane protein expression in escherichia coli. In Membrane Protein Protocols, B. S. Selinsky, ed. (Totowa, N.J., Humana Press), pp. 23-35). Colonies that can grow and continuously express NPC1 and NPC1L1 were isolated and cured of the NPC1 or NPC1L1 expression plasmids. This selection produced two strains:

a. AcrB/Tolc/Perm/N1; and

b. AcrB/Tolc/Perm/L1.

### Example 10

### NPC1L1 Assay Based on Ricin Endocytosis

Following the observation that human liver has the highest expression of NPC1L1, the human liver derived cell line Huh7 was characterized. These cells express significant amounts of NPC1L1 as seen by mRNA and protein levels and were chosen for subsequent studies.

First, stable clones were generated that expressed higher levels of NPC1L1 by introducing the human NPC1 L1 cDNA into these cells. About 30 clones were characterized and clone number 3 had about a five-fold increase in NPC1L1 protein expression.

Next, a number of siRNAs were designed that targeted the NPC1L1 mRNA at various positions. These siRNAs were tested and it was found that two siRNAs targeted NPC1L1 very efficiently. The sequence of these siRNAs are set forth as follows:

| | |
|---|---|
| 1165: TGGTCTTTACAGAACTCACTA | (SEQ ID NO: 23) |
| 1484: TCCGGACAATACCAGTCTCTA. | (SEQ ID NO: 24) |

The numbers 1165 and 1484 refer to the nucleotide position of the human NPC1L1 cDNA (set forth as SEQ ID NO:21), which is the first nucleotide of each siRNA.

Below are the actual construct sequences that were included in the siRNA expression vector (commercially available from GenScript™). The sequences were cloned into a BamHI-HindIII sites.

NPC1L1 Si RNA 1165

(SEQ ID NO: 25)

```
GGATCCCGTAGTGAGTTCTGTAAAGACCATTGATATCCGTGGTCTTT
BamHI Antisense Loop Sense
ACAGAACTCACTATTTTTTCCAAAAGCTT.
                              Terminator
NPC1L1 Si RNA 1484
(SEQ ID NO: 26)
GGATCCCGTAGAGACTGGTATTGTCCGGATTGATATCCGTCCGG
BamHI Antisense Loop Sense
ACAATACCAGTCTCTATTTTTTCCAAAAGCTT.
                                      Terminator
```

Both of these siRNAs were introduced into a vector and stable cell-lines were generated. More than 50 of these cell lines were characterized and four were chosen to be characterized further. Si6 was found to be the best cell-line. Si6 has greater than a 90% decrease in the NPC1L1 mRNA making this clone effectively null for NPC1L1 protein expression.

To further characterize these clones, a number of experiments were carried out using lipid uptake and various toxins to probe their transport. Fluorescent lipids ceramide, cholesterol and LacCer were incubated with cells for 60 minutes at 4° C. and then chased at 37° C. for 30 minutes. All lipids exhibited altered uptake and localization when compared between the NPC1L1 positive clone number 3 and the NPC1L1 negative si6 clone. In particular, there was pronounced Golgi localization of all lipids in the NPC1L1 negative si6 cells.

The endocytosis of a number of toxins such as Ricin, Diphtheria toxin and Verotoxin were then tested. In the case of ricin, the si6 cells appear to target this toxin to the Golgi much more rapidly than either the wild type cells or the clone number 3 cells. To confirm that these results are not due to something unique to clone si6, the ricin uptake experiment was repeated with other, independent siRNA clones. All of these clones, with the exception of clone siS6, which was probably not a good siRNA clone, gave the same result with respect to ricin endocytosis.

A time course experiment was then carried out to determine the optimal time for detecting these differences in endocytosis. It was determined that as early as 15 minutes following addition of the toxin, the different in endocytosis is apparent. Si6 cells show a dramatic Golgi staining with the toxin whereas the wild type and number 3 clone cells exhibit only a punctate type of staining.

Finally, to capitalize on these differences the viability of these cells to Ricin, Diphtheria toxin and Verotoxin intoxication was tested. As predicted from the above results the si6 cells are much more sensitive to the toxins since they appear to target these toxins to their Golgi more efficiently. Si6 cells exhibit higher sensitivity to Ricin following incubation with Ricin overnight.

Alternatively, higher amounts of toxin (5 ug/ml) were incubated with the cells for different amounts of time. With this approach, similar to the above, a two-fold difference in Ricin sensitivity was seen.

In conclusion, the number 3 clone and Ricin intoxication can be used in an assay to measure an increase in the number 3 clone's sensitivity to Ricin based on NPC1L1 inhibition.

The above described mammalian cell assay has been used to screen a library of 3,000 compounds. Molecules that are inhibitors of NPC1L1 activity have been identified (see inhibitors below). A prokaryotic system for screening potential NPC1 L1 inhibitors is also described herein (see Example 8).

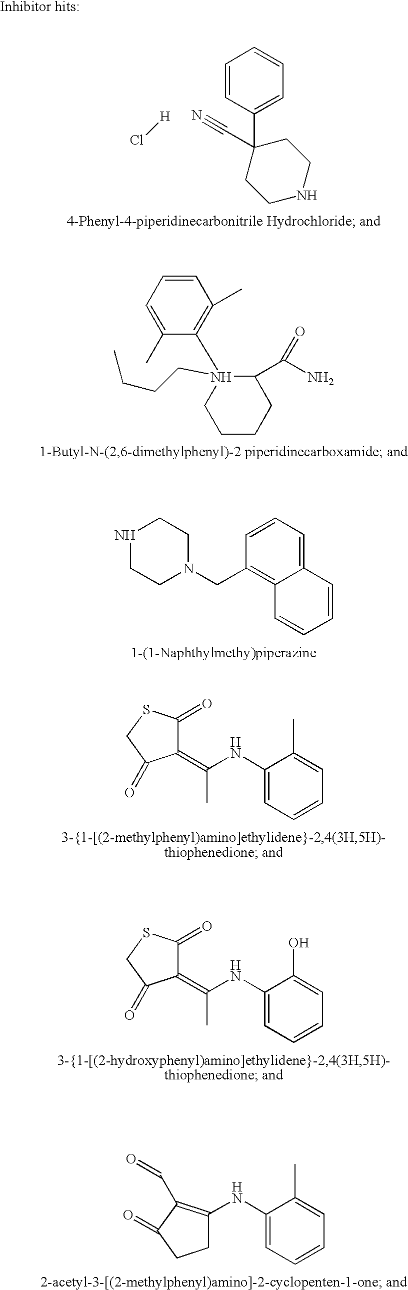

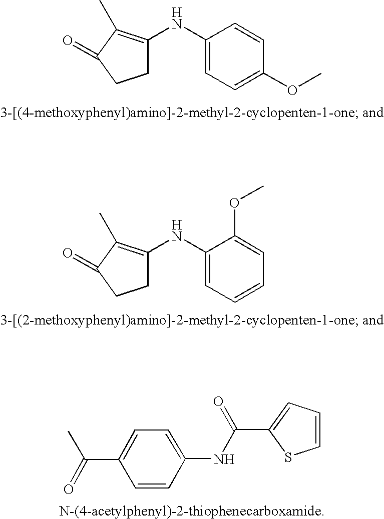

### Example 11

### Assay of NPC1L1 Function by Measuring Expression of the NPC1L1 Promoter

The inventors observed that the NPC1L1 knockout mice described herein have high levels of truncated NPC1L1 mRNA. This suggests that lack of NPC1L1 activity induces expression of NPC1L1. This observation can therefore be used to develop an assay for screening for NPC1L1 inhibitors.

Reporter vectors were constructed that place expression of the luciferase gene under the control of the human NPC1L1 promoter or the mouse NPC1L1 promoter. To validate this, the human construct was transfected into three human liver cell lines.

The promoter sequences of human and mouse NPC1L1 are set forth as SEQ ID NO: 27 (human) and SEQ ID NO: 28 (mouse). These sequences are in the constructs driving the expression of luciferase in vector pGL3 (Promega Corp™). These sequences also include the start codon and a short piece of protein coding region from the 5′ end of the genes and are cloned in-frame with firefly luciferase, thus creating luciferase with a short piece of NPC1L1 fused to it's 5′ end. The start codon region is included because a potential transcription factor, YY1, is known to be involved in the regulation of several key lipid homeostasis genes; in the human NPC1L1 promoter the transcription factor site covers the ATG in an antisense orientation and may possibly inhibit transcription of the gene from this start site.

As predicted, expression of luciferase in Wt Huh7 (wild type; human liver) cells was detectable since these cells express NPC1L1 and therefore are expected to also express luciferase driven by the NPC1L1 promoter. When the construct was introduced into the Huh7 cells where expression of NPC1L1 is inhibited by an siRNA (Si6 as described above), expression is up regulated. In contrast, expression in cells that overexpress NPC1L1 (L1 3+) is down regulated compared to wild type cells (Wt) and even more so compared to the cells that do not express NPC1L1 (Si6 cells).

These results indicate that NPC1L1 is unique in that it regulates its own expression. That is, when cells sense that there is lack of NPC1L1 activity the cells up-regulate the NPC1L1 promoter and when levels of NPC1L1 protein rise the cells down-regulate NPC1L1 expression. Thus, the L1 3+ cell-line can also be used for screening NPC1L1 inhibitors. Inhibitors of NPC1L1 induce expression of the luciferase gene driven by the NPC1L1 promoter to the levels detected in the Si6 cells, e.g., about 4-5 fold higher.

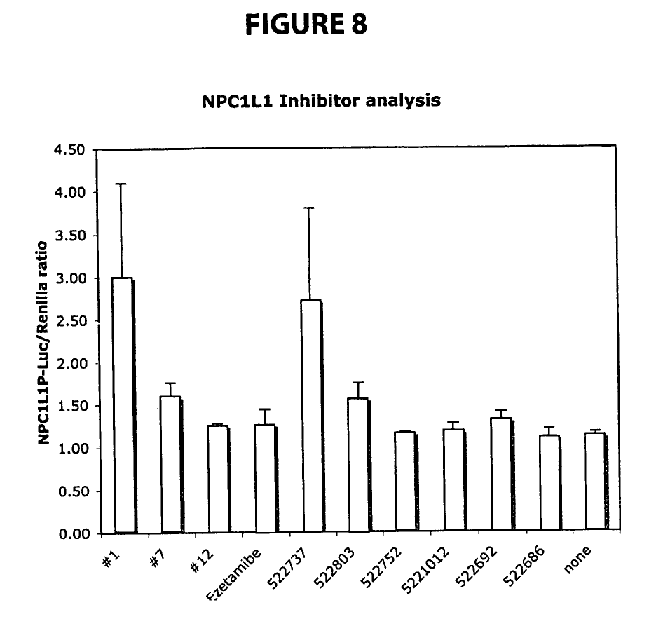

The inhibitors identified using the ricin intoxication assay (Example 10) were tested in utilizing the above assay whereby upregulation of the NPC1L1 promoter was used to detect the inhibition of the NPC1L1 protein. As shown in FIG. 8, 4-Phenyl-4-piperidinecarbonitrile Hydrochloride (#1), (1-Butyl-N(2,6-diemethylphenyl)2 piperidine carboxamide) #7,2-acetyl-3-[(2-methylphenyl)amino]-2-cyclopenten-1-one, 3 {1-[(2-hydroxyphenyl)amino]ethylidene}-2,4(3H, 5H)-thiophenedione and gave a positive signal compared to control (none). Note that Ezetamibe did not inhibit NPC1L1 in this assay.

### Example 12

### Comparison of NPC1L1 (−/−) Knockout and C57BL6 Wild-Type Mice Fed a High Fat Diet

Wild-type C57BL6 mice are known to be susceptible to diet induced obesity, followed by the development of type II (non-insulin dependent) diabetes. Administration of a diabetogenic high fat diet can induce these symptoms in wild-type C57BL6 mice.

Obesity is strongly associated with diabetes and as the mice become progressively more obese there is an increase in lipid deposition in adipose tissue, along with ectopic deposition of lipid in key peripheral tissues such as skeletal muscle, the liver and pancreas. Elevated amounts of plasma lipids, such as fatty acids are also observed. The peripheral tissues eventually fail to respond to insulin, leading to insulin resistance, glucose intolerance and elevated plasma glucose. The pancreatic β-cells attempt to compensate for the insulin resistance and glucose intolerance by producing more insulin, leading to hyperinsulinemia. Overt diabetes occurs when the pancreatic β-cells fail to secrete adequate amounts of insulin to lower plasma glucose levels and pancreatic cell damage occurs.

Under normal conditions, insulin regulates glucose by stimulating glucose uptake and metabolism in adipose and skeletal muscle tissues. It also inhibits gluconeogenesis in the liver. In the pre-diabetic and in patients with overt diabetes, this regulation is impaired so that plasma glucose can no longer be effectively maintained at the required levels.

The studies below compare the effect of the NPC1L1 gene knockout (−/−) with wild-type C57BL6 mice that become obese and develop type II diabetes, during administration of a high fat diet. Mice that were 7-8 weeks of age were placed on a high fat diet for these studies.

The NPC1L1 (−/−) knockout mice were protected against the diet-induced obesity and diabetic symptoms observed in wild-type (wt) C57BL6 mice. Therefore, inhibitors of NPC1L1 may be useful for the treatment and/or prevention of obesity and diabetes.

### 1. Body Weight of Two Sets of Mice Fed a High Fat Diet

The following experiments show that whilst the wild-type (wt) C57BL6 mice become obese when fed a high fat diet, the NPC1L1(−/−) knockout mice resist the development of obesity. Data is from two independently analyzed sets of mice identified as mouse set 1 and mouse set 2.

In the first experiment, NPC1L1 gene knockout (−/−) and wild-type (wt) mice were fed a high fat diet for 0-245 days and weighed on a weekly basis for most of the time-course. There were 5 knockout mice and 6-7 wild-type mice used in this experiment.

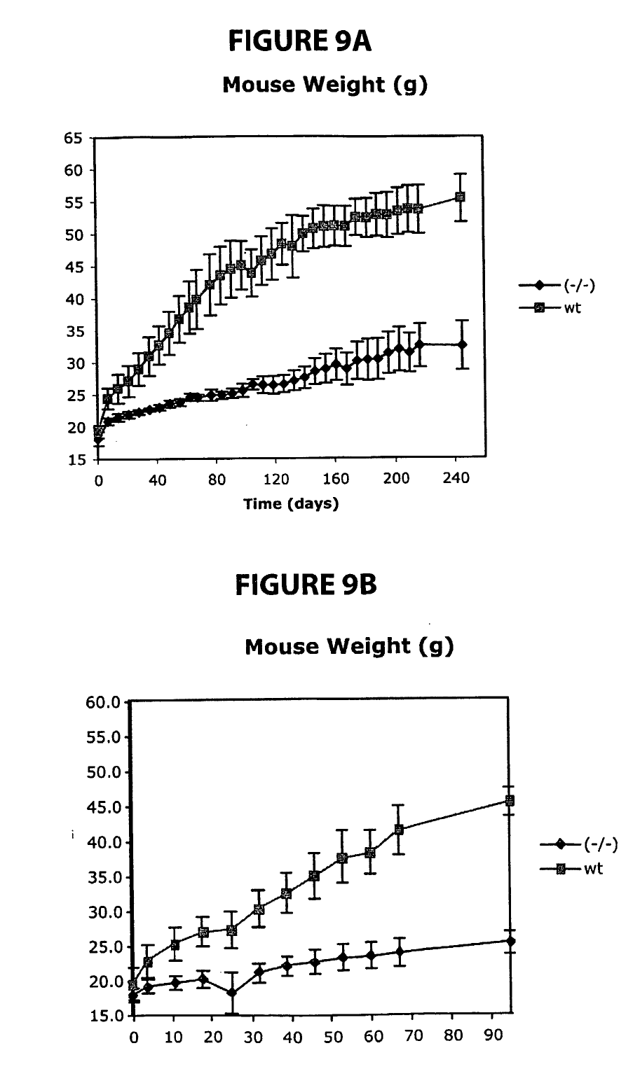

As shown in FIG. 9A, the wild-type mice became obese whilst the knockout mice resisted the weight gain. By 245 days the knockout mice had an average weight of 32.5 g whilst the wild-type mice were 55.4 g.

In a second experiment, NPC1L1 gene knockout (−/−) and wild-type (wt) mice were fed a high fat diet for 0-95 days and weighed on a weekly basis for most of the time-course. There were 7 knockout mice and 7 wild-type mice used in this experiment.

As shown in FIG. 9B, the wild-type mice became obese whilst the knockout mice resisted the weight gain. By 245 days the knockout mice had an average weight of 25.3 g whilst the wild-type mice were 45.4 g.

### 2. Glucose Tolerance Tests on Mice

The data below shows that on a regular chow diet, at 7 weeks of age, the wild-type (wt) C57BL6 and NPC1L1(−/−) knockout mice have a normal and similar ability to clear blood glucose.

When fed the high fat diet, the NPC1L1(−/−) knockout mice, although showing slightly impaired glucose tolerance, are able to effectively regulate their blood glucose, in contrast to the wild-type mice, which show classic glucose intolerance at both 102 and 262 days of high fat diet administration.

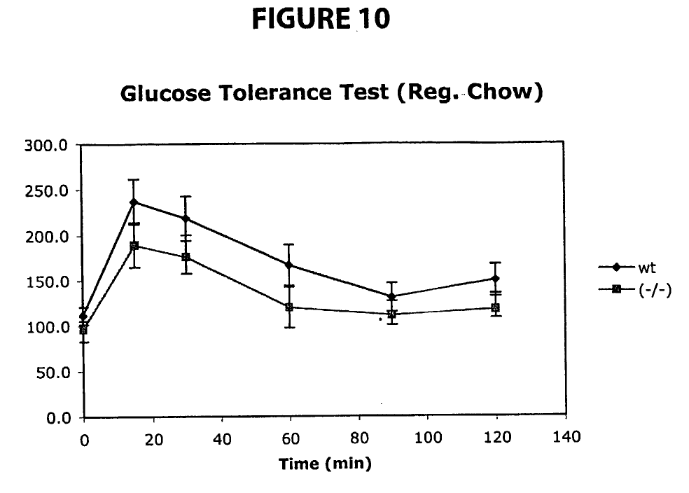

After weaning, 7 wild-type and 5 knockout mice (age-matched) were fed a regular chow diet. At 7 weeks of age the mice were fasted overnight and then injected intraperitoneally with glucose. Blood glucose was measured from 0-120 min. There is no significant difference in the glucose tolerance of these wild-type and NPC1L1 (−/−) knockout mice as both show efficient clearance of excess blood glucose (see FIG. 10).

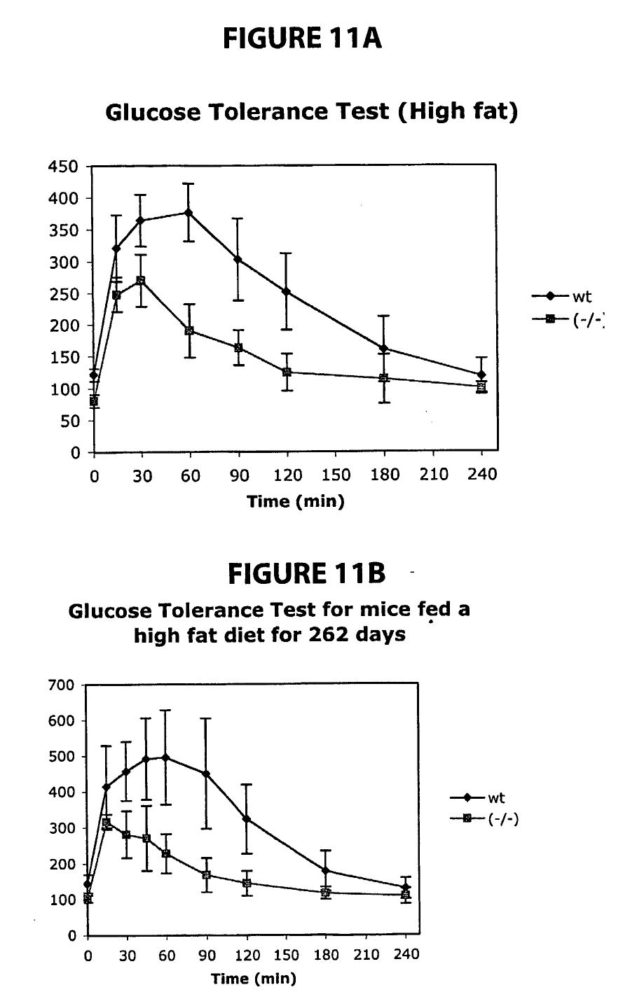

In a second experiment, mice were placed on a high fat diet at 7-8 weeks of age and, after 102 days of feeding the high fat diet, glucose tolerance was tested in 6 wild-type and 5 gene knockout mice. The mice were fasted overnight and then injected intraperitoneally with glucose. Blood glucose was measured at 0-240 min after injection. The wild-type mice are significantly intolerant to intraperitoneal glucose injection, with slow clearance. In contrast, the gene knockout mice effectively clear the injected glucose. The glucose intolerance observed in the wild-type mice is a sign of the onset of type II diabetes and is likely to be associated with the weight gain seen in these mice. The gene knockout mice seem to be protected against this symptom of diabetes (see FIG. 11A).

In a third experiment, mice were placed on a high fat diet at 7-8 weeks of age and, after 262 days of feeding the high fat diet, glucose tolerance was tested in 6 wild-type and 5 gene knockout mice. The mice were fasted overnight and then injected intraperitoneally with glucose. Blood glucose was measured at 0-240 min after injection. At 262 days of feeding on a high fat diet the wild-type mice were significantly more intolerant to intraperitoneal glucose injection, with severely slowed clearance, compared with the NPC1L1 (−/−) gene knockout mice, which effectively reduce the elevated glucose. The glucose intolerance observed in the wild-type mice is indicative of type II diabetes. The NPC1L1 (−/−) gene knockout mice, although not completely normal in their glucose clearance time, are not nearly as severely affected as the wild-type mice (see FIG. 11B).

### 3. Insulin Tolerance Test in Mice

Normally, insulin stimulates glucose uptake and metabolism in adipose and skeletal muscle tissues as well as inhibiting gluconeogenesis in the liver, thus lowering blood glucose levels. The data below shows that when insulin is administered to the wild-type C57BL6 mice fed a high fat diet there is little effect on the blood glucose levels in these mice, indicating that they have become intolerant to the effects of insulin in lowering blood glucose. The NPC1L1(−/−) knockout mice respond to the insulin administration with a decrease in blood glucose, as expected in insulin responsive animals.

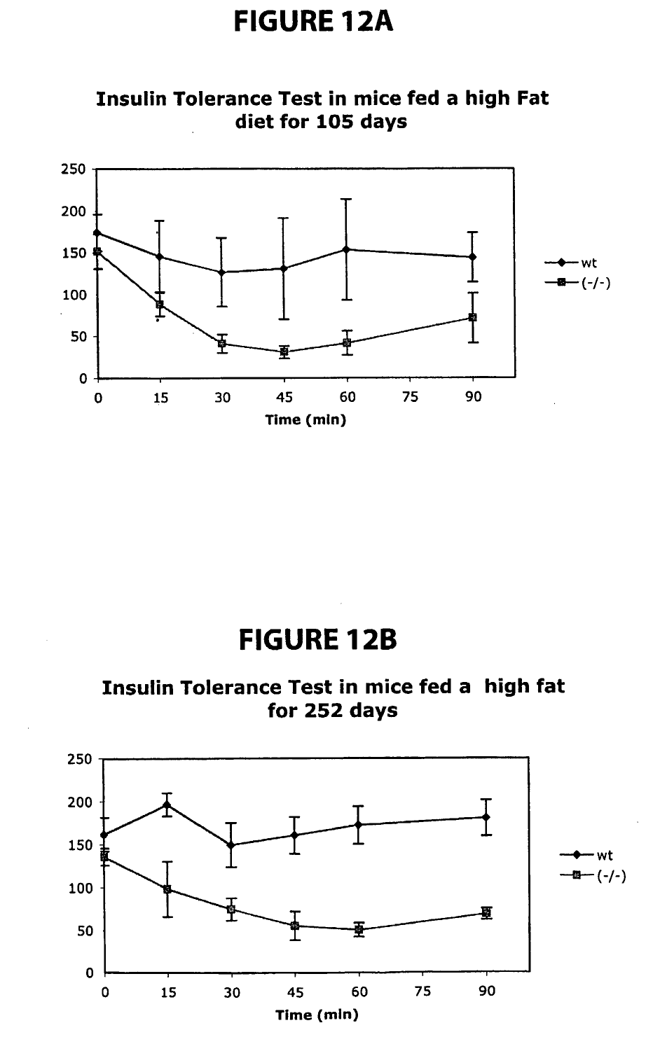

In a first experiment, mice were fed a high fat diet for 105 days (7 wild-type and 7 knockout mice). After a 3 hour fast, mice were injected intraperitoneally with insulin and their blood glucose was measured. The decrease in blood glucose caused by insulin administration was clear in the NPC1L1 (−/−) gene knockout mice, with a rapid decrease in glucose levels. In the wild-type mice there was a muted, almost non-existent response to insulin injection as the glucose levels remained high (see FIG. 12A). This insulin resistance observed in the wild-type C57BL6 mice is characteristic of mice in a pre-diabetic or overtly diabetic state.

In a second experiment, mice were fed a high fat diet for 252 days (6 wild-type and 5 knockout mice). After a 3 hour fast, mice were injected intraperitoneally with insulin and their blood glucose was measured. As at 105 days, the decrease in blood glucose caused by insulin administration was clear in the NPC1L1 (−/−) gene knockout mice, with a decrease in glucose levels. In the wild-type mice there was a muted, almost non-existent response to insulin injection as the glucose levels remained high (see FIG. 12B). This insulin resistance observed in the wild-type C57BL6 mice is characteristic of mice in a pre-diabetic or overtly diabetic state.

4. Insulin Measurements in Nice Injected with Glucose

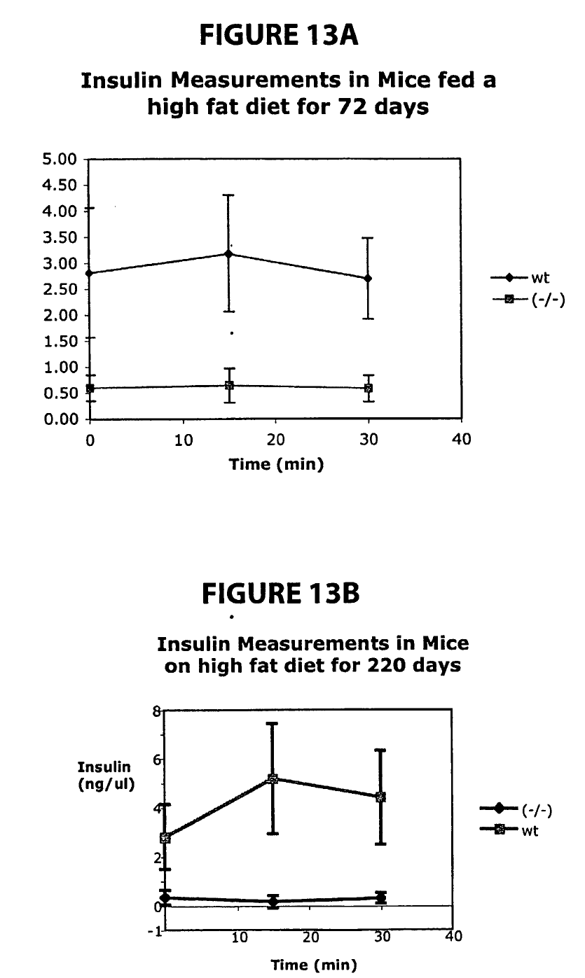

In a first experiment, glucose was injected intraperitoneally into 7 wild-type and NPC1L1 (−/−) gene knockout mice that had been fed a high fat diet for 72 days and then fasted overnight. Plasma insulin was measured at 0-30 min. In the knockout mice the pre-injection plasma insulin was low and the increase in insulin caused by glucose injection was presumably short-lived as it was not detected at 15 minutes, the first measurement post-glucose injection, results that would be expected in non-diabetic mice (see FIG. 13A). The wild-type mice have hyperinsulinemia and the elevated insulin levels are maintained throughout the course of the experiment and this is characteristic of a pre-diabetic and diabetic disease state.

In a second experiment, glucose was injected intraperitoneally into 6 wild-type and 5 NPC1L1 (−/−) gene knockout mice that had been fed a high fat diet for 220 days and then fasted overnight. Plasma insulin was measured at 0-30 min. As at 72 days, in the knockout mice the pre-injection plasma insulin was low and the increase in insulin caused by glucose injection was presumably short-lived as it was not detected at 15 minutes, the first measurement post-glucose injection, results that would be expected in non-diabetic mice (see FIG. 13B). The wild-type mice have hyperinsulinemia and the elevated insulin levels are maintained throughout the course of the experiment and this is characteristic of a pre-diabetic and diabetic disease state.

### 5. Plasma Lipoprotein Profiles in the Mice at 120 and 268 Days of High Fat Diet

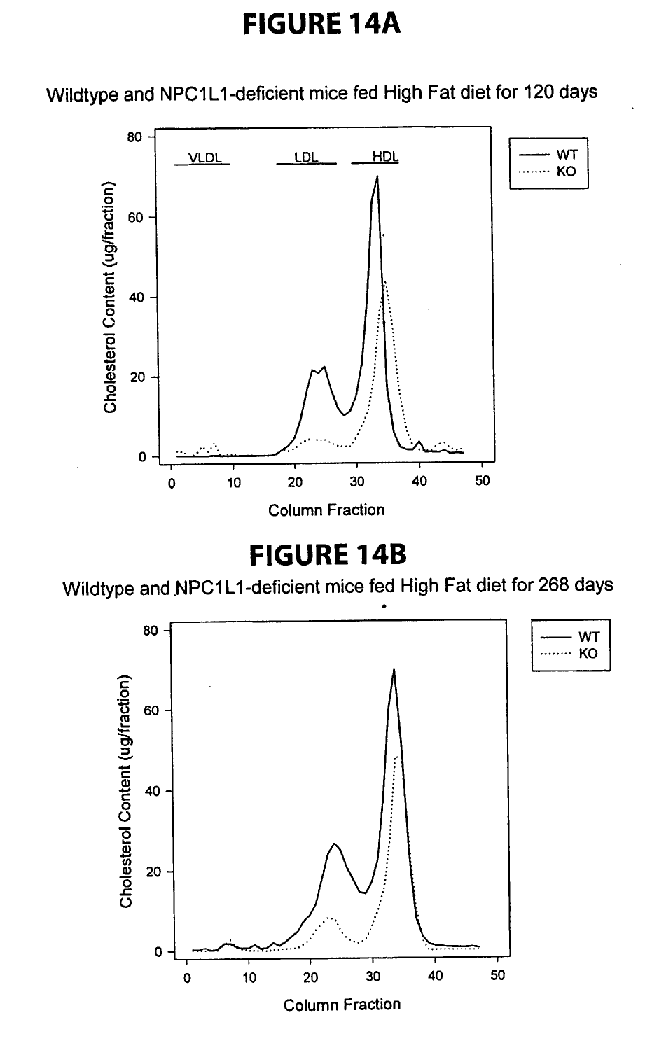

Plasma lipid profiles were analyzed in wild type and NPC1L1(−/−) mice. The knockout mice significantly lower plasma LDL and HDL and total cholesterol than the wild-type mice. The plasma triglyceride levels were similar in both groups (see FIGS. 14A and 14B).

### Example 13

### Comparison of Food Intake of NPC1L1 (−/−) Knockout and C57BL6 Wild-Type Mice

Food intake of mice lacking NPC1L1 (NPC1L1 knockout mice) has been investigated by the inventors. It has been found that there is no difference between wild-type and knockout mice with respect to the amount of food consumed. This indicates that lack of NPC1L1 (or inhibition of NPC1L1) does not suppress appetite.

Since NPC1L1 appears to regulate the flow of lipids (and possibly other nutrients) from the plasma membrane (uptake) to the various cellular organelles such as Golgi and ER it was hypothesized that lack (or decreased) NPC1L1 activity could have a number of effects on cellular homeostasis: 1) limit the amount of nutrients (lipids, proteins, sugars) that become available for cellular processes, 2) alter signaling cascades that tell the cell to behave as if nutrients are plentiful, and 3) stimulate a limited nutrient response.

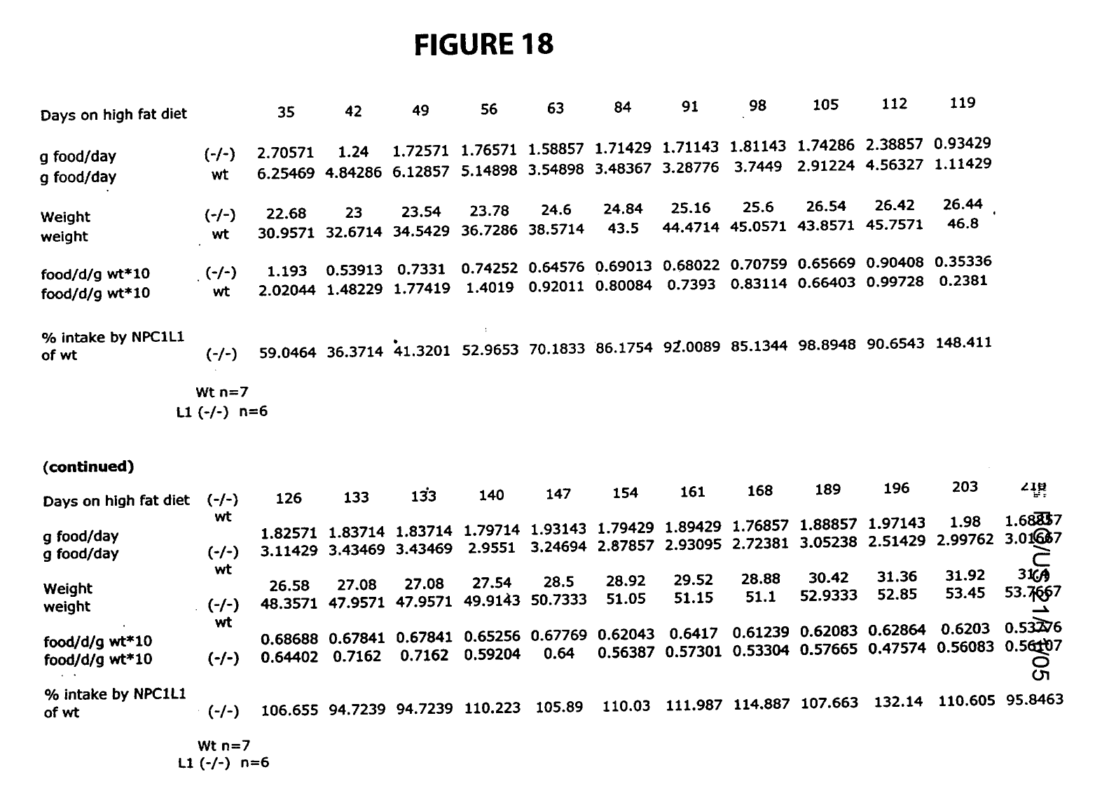

However, when mice are challenged with a high fat diet (60 kcal % fat; Diet D12492, available from Research Diets, Inc.™, New Brunswick, N.J.) the results are interesting. In the beginning stages of the high fat diet, the NPC1L1 knockout mice are eating less (about 60% of the wild-type mice). As they are challenged longer >90 days their intake becomes similar to wild-type mice. Importantly, even after 90 days, the knockout mice still do not gain as much weight as the wild-type animals (see FIG. 18).

### Example 14

### White Adipose Tissue Has Significant Expression Levels of NPC1L1

Previous real-time PCR data have shown that NPC1L1 is elevated in the small intestine of both mice and humans and in addition, is high in the human liver. The data described herein shows that adipose tissue expresses a significant amount of NPC1L1. Since the absence of NPC1L1 is protective against obesity and type II diabetes and adipose tissue plays a role in the development of both of these diseases, finding significant expression in these tissues is of considerable interest.

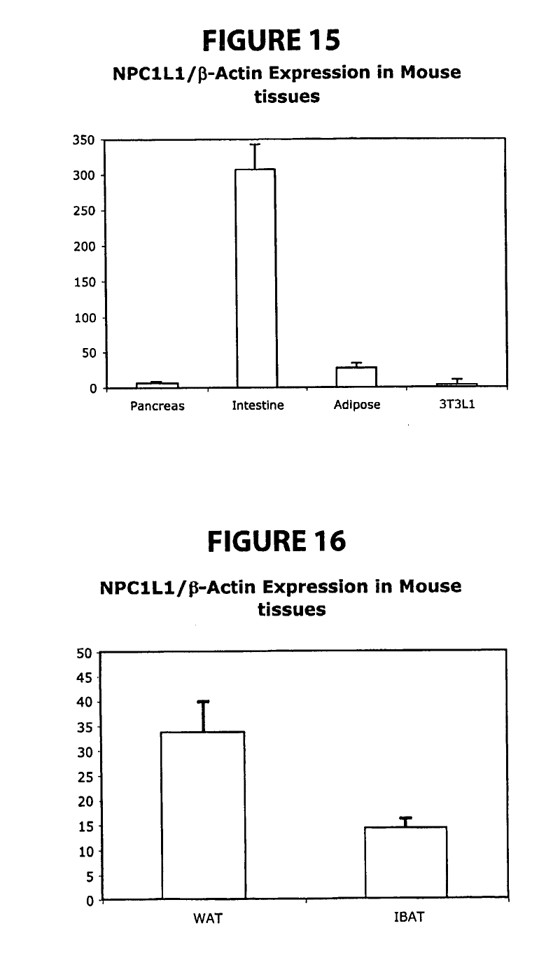

NPC1L1 transcript was measured by semi-quantitative real-time PCR, normalized to β-actin expression. As shown in FIG. 15, in mouse white adipose (gonadal) tissue, NPC1L1 is expressed at 9% of the amount detected in the small intestine, which has the most abundant expression of NPC1L1. This is a significant amount compared with other tissues (for example, pancreas has only 2% of small the amount found in the small intestine). The pre-adipocyte mouse cell line 3T3L1 does not express NPC1L1.

NPC1L1 transcript was measured by semi-quantitative real-time PCR in mouse white (gonadal) adipose (WAT) and interscapular brown adipose tissue (IBAT), normalized to β-actin expression. As shown in FIG. 16, expression of NPC1L1 is higher in white adipose tissue and the amount in brown adipose is 42% of that found in the white tissue.

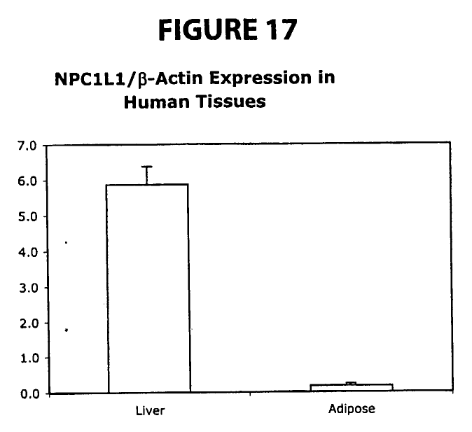

NPC1L1 transcript was also measured by semi-quantitative real-time PCR in human liver and white adipose tissue, normalized to β-actin expression. As shown in FIG. 17, the expression in human white adipose tissue was 3% of that detected in human liver. Previously, it was found that human jejunum (the highest expressing human intestine tissue) had 4% of the NPC1L1 transcript found in human liver and so a value of 3% for adipose is a significant amount of NPC1L1. Many other tissues have less than 1% of the NPC1L1 detected in liver.

### Example 15

### Creation of NPC1L1 Transgenic Mice that Overexpress NPC1L1

### Rationale:

The NPC1L1 knockout mouse was instrumental in deciphering the lipid transport function of this protein and its critical role in intestinal cholesterol and other lipid transport. A powerful tool in drug discovery and drug testing (to determine is a drug acts directly on NPC1L1) is a mouse that overexpresses NPC1L1. There are a number of considerations in developing such as model. First, these mice must be able to tolerate higher expression of NPC1L1 so that its expression does not cause lethality. Second, given that the mouse NPC1L1 gene is not expressed in all mouse tissues, a system must be designed that expresses the protein at high levels but only in the appropriate tissues.

The first consideration can only be determined once the transgenic mice are generated and evaluated to see if they can pass the NPC1L1 genes to their progeny. To address the second consideration the mouse complete gene (genomic sequence as described below) was used. In this manner, the promoter and all regulatory elements are maintained and provided the tissue specificity required.

### Results:

The entire mouse gene sequence of NPC1L1 was cleaved from a Bac vector, clone RP23-64P22 (from female mouse library), obtained from BacPac Resources™, Oakland Calif., which contains the unordered genomic fragments given in GenBank Accession number AC079435. The complete, ordered, gene sequence is given in GenBank sequence, accession number AL607152. According to this ordered sequence (GenBank Accession number AL607152) the gene spans nucleotides 37338 (5′ end) to 18610 (3′ end) in an antisense orientation.

A region spanning the complete gene was excised using the restriction endonuclease enzyme MfeI, which cleaves the region from nucleotides 6656-46736, of GenBank Accession number AL607152, containing the entire NPC1L1 gene and almost 10 kb of sequence upstream of the start codon and therefore including the entire NPC1L1 promoter region for regulated gene expression.

The MfeI fragment was cloned into the 6.8 kb vector pSMARTVC (Lucigen Corporation™) at its EcoRI site.

The NPC1L1/pSMARTVC vector was cleaved using AscI and PmeI and a linearized NPC1L1 fragment, with short, flanking vector arms was isolated by sucrose gradient separation to allow removal of most of the pSMART vector.

The isolated NPC1L1 gene fragment was then injected into fertilized mouse eggs and these placed into pseudopregnant C57BL6 mice (Taconic™). Transgenic mice were created by incorporation of the transgene into these mice. The mice were screened by PCR amplification of both their 5′ and 3′ ends, using one primer that contained the NPC1L1 gene sequence and a second primer that contained the short flanking pSMART vector arm sequence.

The primers used to amplify the 5′ end of transgenic NPC1L1 have the following sequence: pSMART 5′ CTATACGAAGTTATGTCAAGCGG (SEQ ID NO: 30) and mNPC1L1 BAC 46043(+) CTTGCACCTGACTTCCTCATATAAG (SEQ ID NO: 31).

The primers used to amplify the 3′ end of transgenic NPC1L1 have the following sequence: pSMART 3′ AAAGAAGGAAAGCGGCCGCCAGG (SEQ ID NO: 32); and mNPC1L1 BAC 7568 (−) AGGAACCGTACTGAGCGCATACCAA (SEQ ID NO: 33). Therefore, presence of the 5′ and 3′ ends of the NPC1L1 transgene in the progeny mice was confirmed, indicating that at least one additional copy of the mouse NPC1L1 gene had been inserted.

Two transgenic mouse lines have been created and one has successfully transmitted the transgene to its offspring (3 out of 7). Both of the parental original transgenic mice have an increased body weight, compared to the average weight of C57BL6 mice (Both transgenic mice were overweight). Male mouse #2 (which has successfully produced offspring) was 34 grams at 5.5 months of age. Female mouse #6 was 37 grams at 4 months of age (no offspring)). The average weight of a normal mouse at 4-6 months of age is about 25 grams.

Also, when genotyping these mice, the DNA was prepared by proteinase K digestion to produce crude, unpurified DNA for PCR-analysis. Unusually, there appeared to be lipid floating on the top of the extract and the OD abnormal, most likely due to excess tissue lipids.

### CONCLUSION

The NPC1L1 gene was identified, based on its structural homology to NPC1. Cell-based studies of the NPC1L1 indicate that NPC1L1 has a predominant intracellular localization, with concentration in the Golgi and ER compartments. mRNA expression profiling of NPC1L1 reveals significant differences in RNA transcript levels between mouse and man, with highest expression levels found in human liver. Isolation of the mouse NPC1L1 gene allowed implementation of a knockout model of NPC1L. Mice lacking a functional NPC1L1 have multiple lipid transport defects. Surprisingly, lack of NPC1L1 exerts a protective effect against diet-induced hyercholesterolemia. When compared with wild-type controls, NPC1L1-deficient mice also show a different response in levels of glucose, LDL-cholesterol, and HDL-cholesterol following a shift from a low-fat to high-fat diet. Further characterization of cell lines generated from wild-type and knockout mice reveals that, in contrast to wild-type cells, NPC1L1-deficient cells show aberrations in both plasma membrane uptake and subsequent transport of a variety of lipids, including cholesterol, fatty acids, and sphingolipids. Furthermore, cells lacking NPC1L1 reveal aberrant caveolin transport and localization, suggesting that the observed lipid defects may result from an inability of NPC1L1 to properly target and regulate caveloin expression. Furthermore, comparison of NPC1L1 knock-out mice to wild type mice fed on a high fat diet indicates that the absence of NPC1L1 is protective against obesity and type II diabetes. In addition, it has been found that NPC1L1 is highly expressed in white adipose tissue, which is involved in the development of obesity as well as diabetes. Thus, inhibitors of NPC1L1 would be capable of treating obesity and diabetes in a subject, in addition to hyperlipidemia and other lipid-related disorders such as cardiovascular disease. Several inhibitors of NPC1L1 have been identified, as set forth above. In addition, a transgenic mouse that overexpresses NPC1L1 has been created. This transgenic animal is useful for the identification and validation of agents that modulate NPC1L1.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, publications, product descriptions, Accession Nos., and protocols are cited throughout this application, the disclosures of which are incorporated herein by reference in their entireties for all purposes.

What is claimed:

1. An isolated nucleic acid encoding a Niemann-Pick C1-like protein (NPC1L1) wherein the nucleic acid comprises a nucleotide sequence that hybridizes under normal conditions to the complement of the nucleotide sequence set forth in SEQ ID NO: 2.

2. An isolated nucleic acid encoding a NPC1L1 polypeptide, wherein the nucleic acid comprises the nucleotide sequence set forth in SEQ ID NO: 2.

3. An isolated NPC1L1 nucleic acid comprising a nucleotide sequence having at least 95% identity with the nucleotide sequence set forth in SEQ ID NO: 2.

4. An isolated nucleic acid comprising a nucleotide sequence encoding an NPC1L1 polypeptide having an amino acid sequence set forth in SEQ ID NO: 3.

5. An isolated nucleic acid comprising a nucleotide sequence encoding an NPC1L1 polypeptide having an amino acid sequence having at least 95% identity with the amino acid sequence set forth in SEQ ID NO: 3, wherein the encoded polypeptide has a lipid permease function.

6. An isolated NPC1L1 polypeptide comprising an amino acid sequence encoded by the nucleic acid sequence of claim 1.

7. An isolated NPC1L1 polypeptide comprising the amino acid sequence set forth in SEQ ID NO: 3.

8. An isolated NPC1L1 polypeptide comprising an amino acid sequence having at least 95% identity with the amino acid sequence set forth in SEQ ID NO: 3, wherein the NPC1L1 polypeptide has a lipid permease function.

9.-14. (canceled)

15. The isolated nucleic acid of claim 2 comprising a mutation in at least one nucleotide that results in defective expression or activity of the NPC1L1 protein product.

16. The isolated nucleic acid of claim 15, wherein defective expression of NPC1L1 results in a disorder in glucose metabolism.

17. The isolated nucleic acid of claim 15, wherein defective expression of NPC1L1 results in a disorder in lipid metabolism.

18. The isolated nucleic acid of claim 17, wherein the lipid is selected from the group consisting of cholesterol, triglycerides, and sphingolipids.

19. The isolated nucleic acid of claim 18, wherein the lipid is cholesterol.

20.-32. (canceled)

33. A method for identifying a test compound that binds to an NPC1L1 polypeptide, which method comprises:
(i) contacting a host cell that expresses an NPC1L1 polypeptide with a test compound; and
(ii) identifying a test compound that binds to said host cell but not to a control cell that does not express NPC1L1 polypeptide.

34. A method for identifying a test compound that modulates the activity of an NPC1L1 polypeptide, which method comprises:
(i) providing a host cell that expresses a functional NPC1L1 polypeptide,
(ii) contacting said host cell with a test compound under conditions that would otherwise activate the activity of said functional NPC1L1 polypeptide; and
(iii) determining whether said host cell contacted with said test compound exhibits a modulation in activity of said functional NPC1L1 polypeptide.

35. A method for identifying an agent useful in the prevention or treatment of an NPC1L1-mediated disease or disorder, which method comprises determining the effect of the substance on a biological activity of an NPC1L1 polypeptide by:
(a) contacting a test cell which expresses a functional NPC1L1 polypeptide with the test agent in the presence of extracellular cholesterol under conditions where uptake of the cholesterol would be effected; and
(b) observing the effect of the addition of the agent on the test cell, in comparison with the effect of a control cell expressing a functional NPC1L1 polypeptide not contacted with the test agent, wherein inhibition of cholesterol uptake in the test cell compared to the control cell is indicative that the test agent is useful for the treatment of an NPC1L1-mediated disease or disorder.

36.-46. (canceled)
